(19) United States
(12) Patent Application Publication
(10) Pub. No.: US 2022/0275029 A1
(43) Pub. Date: Sep. 1, 2022

(54) COMPOSITIONS AND PARTICLES FOR PAYLOAD DELIVERY

(71) Applicant: Applied Molecular Transport Inc., South San Francisco, CA (US)

(72) Inventors: Randall J. MRSNY, Los Altos Hills, CA (US); Derek MACLEAN, Los Altos, CA (US); Michael SEKAR, Palo Alto, CA (US); Kevin YIN, Palo Alto, CA (US); Tahir Mahmood, Burlingame, CA (US); Thomas Carl Hunter, Mountain View, CA (US); Keyi Liu, Mountain View, CA (US)

(21) Appl. No.: 17/673,603

(22) Filed: Feb. 16, 2022

Related U.S. Application Data
(63) Continuation of application No. PCT/US2020/046547, filed on Aug. 14, 2020.
Application No. PCT/US2020/046547 is a continuation-in-part of application No. PCT/US2019/050708, filed on Sep. 11, 2019.
Application No. PCT/US2019/050708 is a continuation-in-part of application No. PCT/US2019/060356, filed on Nov. 7, 2019.
(60) Provisional application No. 62/887,933, filed on Aug. 16, 2019.
(60) Provisional application No. 62/887,963, filed on Aug. 16, 2019.
(60) Provisional application No. 62/888,133, filed on Aug. 16, 2019.
(60) Provisional application No. 62/888,144, filed on Aug. 16, 2019.
(60) Provisional application No. 62/888,238, filed on Aug. 16, 2019.
(60) Provisional application No. 62/888,282, filed on Aug. 16, 2019.
(60) Provisional application No. 62/888,237, filed on Aug. 16, 2019.
(60) Provisional application No. 62/888,400, filed on Aug. 16, 2019.
(60) Provisional application No. 62/898,729, filed on Sep. 11, 2019.
(60) Provisional application No. 62/898,934, filed on Sep. 11, 2019.
(60) Provisional application No. 62/898,899, filed on Sep. 11, 2019.
(60) Provisional application No. 62/898,709, filed on Sep. 11, 2019.
(60) Provisional application No. 62/899,064, filed on Sep. 11, 2019.
(60) Provisional application No. 62/935,615, filed on Nov. 14, 2019.
(60) Provisional application No. 62/939,495, filed on Nov. 22, 2019.
(60) Provisional application No. 62/970,627, filed on Feb. 5, 2020.
(60) Provisional application No. 62/971,126, filed on Feb. 6, 2020.
(60) Provisional application No. 62/986,557, filed on Mar. 6, 2020.
(60) Provisional application No. 62/986,579, filed on Mar. 6, 2020.
(60) Provisional application No. 63/013,309, filed on Apr. 21, 2020.
(60) Provisional application No. 63/020,996, filed on May 6, 2020.
(60) Provisional application No. 63/021,029, filed on May 6, 2020.
(60) Provisional application No. 63/033,151, filed on Jun. 1, 2020.
(60) Provisional application No. 63/033,180, filed on Jun. 1, 2020.
(60) Provisional application No. 63/033,077, filed on Jun. 1, 2020.
(60) Provisional application No. 63/055,886, filed on Jul. 23, 2020.

(51) Int. Cl. C07K 14/195; A61K 9/00; A61K 47/64; A61K 47/65; A61P 1/00; C07K 16/24; C07K 17/06

(52) U.S. Cl. CPC C07K 14/195; A61K 9/0053; A61K 47/6415; A61K 47/65; A61P 1/00; C07K 16/241; C07K 17/06

(57) ABSTRACT

The present disclosure provides complexes and compositions comprising particles, microparticles or nanoparticles, for delivery of payloads into a cell or across a polarized epithelial cell. The compositions can comprise a payload in a pill or tablet for delivery of the payload into or across a polarized epithelial cell.

### CROSS-REFERENCE

This application is a continuation application of International Patent Application No. PCT/US2020/046547, filed Aug. 14, 2020, which claims the benefit of U.S. Provisional Application No. 62/887,933, filed Aug. 16, 2019, U.S. Provisional Application No. 62/887,963, filed Aug. 16, 2019, U.S. Provisional Application No. 62/888,133, filed Aug. 16, 2019, U.S. Provisional Application No. 62/888,144, filed Aug. 16, 2019, U.S. Provisional Application No. 62/888,238, filed Aug. 16, 2019, U.S. Provisional Application No. 62/888,282, filed Aug. 16, 2019, U.S. Provisional Application No. 62/888,237, filed Aug. 16, 2019, U.S. Provisional Application No. 62/888,400, filed Aug. 16, 2019, U.S. Provisional Application No. 62/898,729, filed Sep. 11, 2019, U.S. Provisional Application No. 62/898,934, filed Sep. 11, 2019, U.S. Provisional Application No. 62/898,899, filed Sep. 11, 2019, U.S. Provisional Application No. 62/898,709, filed Sep. 11, 2019, U.S. Provisional Application No. 62/899,064, filed Sep. 11, 2019, U.S. Provisional Application No. 62/935,615, filed Nov. 14, 2019, U.S. Provisional Application No. 62/939,495, filed Nov. 22, 2019, U.S. Provisional Application No. 62/970,627, filed Feb. 5, 2020, U.S. Provisional Application No. 62/971,126, filed Feb. 6, 2020, U.S. Provisional Application No. 62/986,557, filed Mar. 6, 2020, U.S. Provisional Application No. 62/986,579, filed Mar. 6, 2020, U.S. Provisional Application No. 63/013,309, filed Apr. 21, 2020, U.S. Provisional Application No. 63/020,996, filed May 6, 2020, U.S. Provisional Application No. 63/021,029, filed May 6, 2020, U.S. Provisional Application No. 63/033,151, filed Jun. 1, 2020, U.S. Provisional Application No. 63/033,180 filed Jun. 1, 2020, U.S. Provisional Application No. 63/033,077, filed Jun. 1, 2020, U.S. Provisional Application No. 63/055,886, filed Jul. 23, 2020; International Patent Application No. PCT/US2020/046547 is a continuation-in-part application of PCT/US2019/050708, filed Sep. 11, 2019; International Patent Application No. PCT/US2020/046547 application is also a continuation-in-part application of PCT/US2019/060356, filed Nov. 7, 2019, each of which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Feb. 13, 2022, is named 40566-725_301_SL.txt and is 222,267 bytes in size

### BACKGROUND

The inability of large macromolecules and/or protein biopharmaceuticals to be readily absorbed across the epithelium, respiratory epithelium or intestinal epithelium, can be a limiting factor in developing commercially viable formulations of these agents. For example, due to this inability to be absorbed, labile materials can remain in the intestinal lumen until resident enzymes degrade them, and stable materials can remain in the intestinal lumen until they are passed from the body. Furthermore, there can be challenges with pulmonary delivery of therapeutic agents.

While there have been promising results from clinical studies evaluating various biologically active agents for the treatment of diseases, e.g., cancer, inflammatory diseases, immune diseases, growth deficiency disorders, etc., these agents can fail to reach their optimum potential. There can be marginal or inadequate overall efficacy due to inherent limitations such as short biological half-life which can prevent the delivery of optimal therapeutically effective dosages, and/or detrimental side effects and toxicities observed at the therapeutically effective doses. Moreover, these agents can require multiple dosing regimens, which can necessitate continuous administration intravenously or by frequent subcutaneous injections, which can be burdensome on the patients and caregivers.

Thus, there is a need for new methods and compositions that can be used to administer therapeutic agents to a human subject. There is also a need for improved methods and compositions for delivery of glucose regulating agents to a subject.

### SUMMARY

Described herein, in certain embodiments, are composition comprising a carrier capable of entering a polarized epithelial cell or transcytosing across a polarized epithelial cell; and a heterologous payload, wherein a molar ratio of the heterologous payload to the carrier is greater than 1:1. In some embodiments, the composition comprises a transition metal cation. In some embodiments, the transition metal cation is selected from the group consisting of Fe2+, Mn2+, Zn2+, Co2+, Ni2+, and Cu2+. In some embodiments, the transition metal cation is Zn2+.

In some embodiments, the composition comprises a polycation. In some embodiments, the polycation is protamine.

In some embodiments, the carrier comprises a portion of a Cholix polypeptide. In some embodiments, the carrier consists of a portion of a Pseudomonas exotoxin A. In some embodiments, the carrier consists of a portion of a Cholix polypeptide. In some embodiments, the Cholix polypeptide consists of an amino acid sequence with a C-terminus at any one of amino acid positions 206 to 425 of SEQ ID NO: 7 In some embodiments, the Cholix polypeptide consists of an amino acid sequence with a C-terminus at any one of amino acid positions 150 to 205 of SEQ ID NO: 7. In some embodiments, the Cholix polypeptide consists of an amino acid sequence with a C-terminus at any one of amino acids 150 to 195 of SEQ ID NO: 7. In some embodiments, the Cholix polypeptide consists of an amino acid sequence with an N-terminus at any one of amino acid positions 1 to 41 of SEQ ID NO: 7. In some embodiments, the Cholix polypeptide consists of an amino acid sequence with an N-terminus at any one of amino acid positions 35 to 40 of SEQ ID NO: 7. In some embodiments, the Cholix polypeptide consists of an amino acid sequence from amino acid position 40 of the sequence set forth in SEQ ID NO: 7 to any one of amino acid positions 150 to 205 of the sequence set forth in SEQ ID NO: 7. In some embodiments, the Cholix polypeptide has a C-terminus at any one of amino acid positions 150 to 187 of the sequence set forth in SEQ ID NO: 7. In some embodiments, the Cholix polypeptide consists of the amino acid sequence set forth in SEQ ID NO: 8. In some embodiments, the Cholix polypeptide consists of the amino acid sequence set forth in SEQ ID NO: 9 or SEQ ID: NO: 10. In some embodiments, amino acids positions are numbered based on alignment of the Cholix polypeptide to the sequence set forth in SEQ ID NO: 7, wherein amino acid positions are numbered from N-terminus to C-terminus and starting with position 1 at the N-terminus.

In some embodiments, the heterologous payload is selected from the group consisting of a macromolecule, small molecule, peptide, polypeptide, nucleic acid, mRNA, miRNA, shRNA, siRNA, antisense molecule, antibody, DNA, plasmid, vaccine, polymer nanoparticle, and a catalytically-active material. In some embodiments, the heterologous payload is a therapeutic payload.

In some embodiments, the heterologous payload is selected from the group consisting of a dye and radiopharmaceutical, hormone, cytokine, anti-TNF agent, glucose lowering agent, tumor associated antigen, peptide, and polypeptide. In some embodiments, the heterologous payload is a polypeptide that is a modulator of inflammation in a gastrointestinal tract.

In some embodiments, the heterologous payload is a glucagon-like peptide-2 (GLP-2) analog. In some embodiments, the GLP-2 analog is Teduglutide.

In some embodiments, the heterologous payload is a cytokine. In some embodiments, the cytokine is selected from the group consisting of IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, and IL-30. In some embodiments, the cytokine is IL-10. In some embodiments, the cytokine is IL-22. In some embodiments, the cytokine lacks a native secretion signal. In some embodiments, the therapeutic payload is a hormone. In some embodiments, the hormone is a human growth hormone (hGH).

In some embodiments, the heterologous payload is a glucose-lowering agent. In some embodiments, the heterologous payload is an incretin, glucagon proprotein, glucagon peptide, glucagon-like peptide 1, glucagon-like peptide 2, glicentin, glicentin-related polypeptide, gastric inhibitory polypeptide preprotein, gastric inhibitory polypeptide, dipeptidyl peptidase 4, glucose transporter member 4, preproglucagon, insulin receptor substrate 1, insulin, insulin analog, apolipoprotein A-II, solute carrier family 2, facilitated glucose transporter member 1, glycogen synthase 1, glycogen synthase 2, tyrosine-protein phosphatase non-receptor type 1, RAC-alpha serine threonine-protein kinase, peroxisome proliferator-activated receptor gamma, hexokinase 3, phosphatidylinositol-3,4,5-triphosphate 3-phosphatase and dual-specificity protein, pyruvate dehydrogenase kinase 1, calcium-binding and coiled-coil domain-containing protein 1, Max-like protein X, fructose-bisphosphate aldolase A, glucagon-like peptide 1 receptor, glucagon-like peptide 2 receptor, gastric inhibitory polypeptide receptor, insulin-like growth factor 1 receptor, insulin-like growth factor 2 receptor, insulin receptor, GLP-1 agonist-exenatide, GLP-1 agonist-Liraglutide, exenatide, exendin-4, exendin-3, GIPR agonist (Des-Ala2-GIP1-30), GIPR agonist-Truncated GIP1-30, GLP-1R agonist (aa 1-37 of GIP), GLP-1R agonist (aa 7-36 of GIP), lixisenatide (tradenames Adlyxin®, and Lyxumia®, Sanofi), liraglutide (tradename Victoza®, Novo Nordisk A/S), semaglutide (tradename Ozempic®, Novo Nordisk A/S), albiglutide (tradename Tanzeum®, GlaxoSmithKline; GLP-1 dimer fused to albumin), dulaglutide (tradename Trulicity®, Eli Lilly), glucose-dependent insulinotropic polypeptide, multi-specific peptide agonist, Tirzepatide (Eli Lilly), SAR425899 (Sanofi), dual amylin calcitonin receptor agonist DACRA-089, glargine/Lantus®, glulisin/Apidra®, glarine/Toujeo®, Insuman®, detemir/Levemir®, lispro/Humalog®/Liprolog®, Degludec/DegludecPlus, insulin aspart, insulin and analogues (e.g. LY-2605541, LY2963016, NN1436), PEGylated insulin Lispro, Humulin®, Linjeta, SuliXen®, NN1045, Insulin plus Symlin™, PE0139, fast-acting and short-acting insulins (e.g. Linjeta, PH20, NN1218, HinsBet), (APC-002) hydrogel, oral, inhalable, transdermal and sublingual insulins (e.g. Exubera®, Nasulin®, Afrezza®, Tregopil®, TPM 02, Capsulin, Oral-lyn®, Cobalamin®, oral insulin, ORMD-0801, NN1953, NN1954, NN1956, VlAtab, and Oshadi oral insulin), or an exendin-4 analog, wherein the exendin-4 analog is desPro36-exendin-4(1-39)-Lys6NH2; H-des(Pro36, 37)-exendin-4-Lys4-NH2; H-des(Pro36, 37)-exendin-4-Lys5-NH2; desPro36[Asp28]exendin-4 (1-39); desPro36[IsoAsp28]exendin-4 (1-39); desPro36[Met(O)14, Asp28]exendin-4 (1-39); desPro36[Met(O)14, IsoAsp28]exendin-4 (1-39); desPro36[Trp(O2) 26, Asp28]exendin-4 (1-39); or desPro36[Trp(O2) 25, IsoAsp28]exendin-4 (1-39); desPro36[Met(O)14 Trp(O2)25, Asp28]exendin-4 (1-39); or desPro36[Met(O)14 Trp(O2)25, IsoAsp28]exendin-4 (1-39).

In some embodiments, the heterologous payload is insulin or an insulin analog. In some embodiments, the heterologous payload is exenatide. In some embodiments, the heterologous payload comprises a fluorescent label. In some embodiments, the fluorescent label is a fluorescein.

In some embodiments, the heterologous payload is an exenatide-fluorescein complex. In some embodiments, the composition is resistant to cleavage by a pancreatic enzyme. In some embodiments, at least 50% of the carrier is intact at 2 hours in a pancreatin assay, wherein the pancreatin assay comprises incubating the composition comprising 100 μg of the carrier with 10 μg of pancreatin in 100 μL phosphate buffered saline (PBS) at 37° C. In some embodiments, a molar ratio of the transition metal cation to the carrier is from about 100:1 to about 300,000:1. In some embodiments, a molar ratio of the transition metal cation to the carrier is from about 1000:1 to about 30,000:1. In some embodiments, a molar ratio of the transition metal cation to the carrier is from about 1000:1 to about 10,000:1.

In some embodiments, a molar ratio of the protamine to the carrier is from about 10:1 to about 0.01:1. In some embodiments, the molar ratio of the heterologous payload to the carrier is from about 2:1 to about 6000:1. In some embodiments, a molar ratio of the protamine to the carrier is about 1:1 In some embodiments, the molar ratio of the heterologous payload to the carrier is from about 2:1 to about 10:1. In some embodiments, the molar ratio of the heterologous payload to the carrier is about 7:1. In some embodiments, the molar ratio of the heterologous payload to the carrier is about 7.16:1.

In some embodiments, the heterologous payload is a polypeptide comprising the sequence of SEQ ID NO: 11 or SEQ ID NO: 14. In some embodiments, the heterologous payload is a polypeptide comprising the sequence of SEQ ID NO: 18 or SEQ ID NO: 19. In some embodiments, the heterologous payload is a polypeptide comprising the sequence of SEQ ID NO: 20. In some embodiments, the heterologous payload is a polypeptide comprising the sequence of SEQ ID NO: 21. In some embodiments, the heterologous payload is a polypeptide comprising the sequence of SEQ ID NO: 22.

In some embodiments, the composition is encapsulated. In some embodiments, the encapsulated composition is configured to release the heterologous payload under a first condition but not under a second condition. In some embodiments, the encapsulated composition is configured to release the heterologous payload at a high pH but not at a low pH. In some embodiments, the encapsulated composition comprises an enteric coating. In some embodiments, the composition is a particle. In some embodiments, the composition comprises a polycation. In some embodiments, the carrier is capable of transporting the heterologous payload in the polarized epithelial cell or transcytosing the heterologous payload across a polarized epithelial cell. In some embodiments, the carrier is coupled to the polycation. In some embodiments, the polycation is protamine, poly-lysine, poly-ornithine, poly-ethylene-imine (PEI), prolamin, protamine, polyvinyl pyrrolidone (PVP), polyarginine, polyvinylamine, or a combination thereof. In some embodiments, the polycation is a protamine salt. In some embodiments, the protamine salt is protamine sulfate, protamine acetate, protamine bromide, protamine chloride, protamine caproate, protamine trifluoroacetate, protamine HCO3, protamine propionate, protamine lactate, protamine formate, protamine nitrate, protamine citrate, protamine monohydrogenphosphate, protamine dihydrogenphosphate, protamine tartrate, or protamine perchlorate. In some embodiments, the protamine salt is protamine sulfate.

In some embodiments, described herein are carriers is capable of entering a polarized epithelial cell or transcytosing across a polarized epithelial cell, wherein at least 60% of the carrier is intact at 0.5 hours in a pancreatin assay, wherein the pancreatin assay comprises incubating the composition comprising 100 μg of the carrier with 10 μg of pancreatin in 100 pi phosphate buffered saline (PBS) at 37° C.

In some embodiments, at least 90% of the carrier is intact at 2 hours in the pancreatin assay. In some embodiments, the composition further comprises a cation. In some embodiments, the cation is a metal cation or a polycation. In some embodiments, the cation is a metal cation. In some embodiments, the metal cation is a transition metal cation. In some embodiments, the carrier consists of a portion of a Pseudomonas exotoxin A. In some embodiments, the carrier consists of a portion of a Cholix polypeptide. In some embodiments, the Cholix polypeptide consists of an amino acid sequence with a C-terminus at any one of amino acid positions 206 to 425 of SEQ ID NO: 7. In some embodiments, the Cholix polypeptide consists of an amino acid sequence with a C-terminus at any one of amino acid positions 150 to 205 of SEQ ID NO: 7. In some embodiments, the Cholix polypeptide consists of an amino acid sequence with a C-terminus at any one of amino acids 150 to 195 of SEQ ID NO: 7. In some embodiments, the Cholix polypeptide consists of an amino acid sequence with an N-terminus at any one of amino acid positions 1 to 41 of SEQ ID NO: 7. In some embodiments, the Cholix polypeptide consists of an amino acid sequence with an N-terminus at any one of amino acid positions 35 to 40 of SEQ ID NO: 7.

In some embodiments, the Cholix polypeptide consists of an amino acid sequence from amino acid position 40 of the sequence set forth in SEQ ID NO: 7 to any one of amino acid positions 150 to 205 of the sequence set forth in SEQ ID NO: 7. In some embodiments, the Cholix polypeptide has an C-terminus at any one of amino acid positions 150 to 187 of the sequence set forth in SEQ ID NO: 7. In some embodiments, the Cholix polypeptide consists of the amino acid sequence set forth in SEQ ID NO: 8. In some embodiments, the Cholix polypeptide consists of the amino acid sequence set forth in SEQ ID NO: 9 or SEQ ID: NO: 10. In some embodiments, amino acids positions are numbered based on alignment of the Cholix polypeptide to the sequence set forth in SEQ ID NO: 7, wherein amino acid positions are numbered from N-terminus to C-terminus and starting with position 1 at the N-terminus.

In some embodiments, described herein are compositions comprising a Cholix variant ending at position 195-347 and a heterologous payload, wherein the heterologous payload is a glucose regulating agent. In some embodiments, the ending position of the Cholix variant is determined relative to SEQ ID NO: 7. In some embodiments, the carrier is capable of transcytosing the heterologous payload across a polarized epithelial cell. In some embodiments, the glucose regulating agent is a glucose-lowering agent. In some embodiments, the glucose-lowering agent is an incretin, glucagon proprotein, glucagon peptide, glucagon-like peptide 1, glucagon-like peptide 2, glicentin, glicentin-related polypeptide, gastric inhibitory polypeptide preprotein, gastric inhibitory polypeptide, dipeptidyl peptidase 4, glucose transporter member 4, preproglucagon, insulin receptor substrate 1, insulin, insulin analog, apolipoprotein A-II, solute carrier family 2, facilitated glucose transporter member 1, glycogen synthase 1, glycogen synthase 2, tyrosine-protein phosphatase non-receptor type 1, RAC-alpha serine threonine-protein kinase, peroxisome proliferator-activated receptor gamma, hexokinase 3, phosphatidylinositol-3,4,5-triphosphate 3-phosphatase and dual-specificity protein, pyruvate dehydrogenase kinase 1, calcium-binding and coiled-coil domain-containing protein 1, Max-like protein X, fructose-bisphosphate aldolase A, glucagon-like peptide 1 receptor, glucagon-like peptide 2 receptor, gastric inhibitory polypeptide receptor, insulin-like growth factor 1 receptor, insulin-like growth factor 2 receptor, insulin receptor, GLP-1 agonist-exenatide, GLP-1 agonist-Liraglutide, exenatide, exendin-4, exendin-3, GIPR agonist (Des-Ala2-GIP1-30), GIPR agonist-Truncated GIP1-30, GLP-1R agonist (aa 1-37 of GIP), GLP-1R agonist (aa 7-36 of GIP), lixisenatide (tradenames Adlyxin®, and Lyxumia®, Sanofi), liraglutide (tradename Victoza®, Novo Nordisk A/S), semaglutide (tradename Ozempic®, Novo Nordisk A/S), albiglutide (tradename Tanzeum®, GlaxoSmithKline; GLP-1 dimer fused to albumin), dulaglutide (tradename Trulicity®, Eli Lilly), glucose-dependent insulinotropic polypeptide, multi-specific peptide agonist, Tirzepatide (Eli Lilly), SAR425899 (Sanofi), dual amylin calcitonin receptor agonist DACRA-089, glargine/Lantus®, glulisin/Apidra®, glarine/Toujeo®, Insuman®, detemir/Levemir®, lispro/Humalog®/Liprolog®, Degludec/DegludecPlus, insulin aspart, insulin and analogues (e.g. LY-2605541, LY2963016, NN1436), PEGylated insulin Lispro, Humulin®, Linjeta, SuliXen®, NN1045, Insulin plus Symlin™, PE0139, fast-acting and short-acting insulins (e.g. Linjeta, PH20, NN1218, HinsBet), (APC-002) hydrogel, oral, inhalable, transdermal and sublingual insulins (e.g. Exubera®, Nasulin®, Afrezza®, Tregopil®, TPM 02, Capsulin, Oral-lyn®, Cobalamin®, oral insulin, ORMD-0801, NN1953, NN1954, NN1956, VlAtab, and Oshadi oral insulin), or an exendin-4 analog, wherein the exendin-4 analog is desPro36-exendin-4(1-39)-Lys6NH2; H-des(Pro36, 37)-exendin-4-Lys4-NH2; H-des(Pro36, 37)-exendin-4-Lys5-NH2; desPro36[Asp28]exendin-4 (1-39); desPro36[IsoAsp28]exendin-4 (1-39); desPro36[Met(O)14, Asp28]exendin-4 (1-39); desPro36[Met(O)14, IsoAsp28]exendin-4 (1-39); desPro36[Trp(O2) 26, Asp28]exendin-4 (1-39); or desPro36[Trp(O2) 25, IsoAsp28]exendin-4 (1-39); desPro36[Met(O)14 Trp(O2)25, Asp28]exendin-4 (1-39); or desPro36[Met(O)14 Trp(O2)25, IsoAsp28]exendin-4 (1-39).

In some embodiments, the heterologous payload comprises an incretin. In some embodiments, the heterologous payload comprises exenatide or insulin. In some embodiments, the composition comprises a particle. In some embodiments, the heterologous payload is exendin-3, efpeglenatide, semaglutide, GLP-1R agonist (aa 1-37 of GIP), GLP-1R agonist (aa 7-36 of GIP), tirzepatide, oxyntomodulin, GIPR agonist-truncated GIP1-30, dual amylin calcitonin receptor agonist, preproinsulin, insulin aspart, insulin glargine, or insulin lispro.

In some embodiments, described herein are compositions comprising a carrier derived from a bacterial toxin capable of entering a polarized epithelial cell or transcytosing across a polarized epithelial cell; and a transition metal cation or a polycation, wherein the polycation is molecule or chemical complex having more than 2 positive charges. In some embodiments, the composition comprises a transition metal cation. In some embodiments, the transition metal cation is selected from the group consisting of Fe2+, Mn2+, Zn2+, Co2+, Ni2+, and Cu2+. In some embodiments, the transition metal cation is Zn2+. In some embodiments, the composition comprises the polycation.

In some embodiments, the polycation is protamine, poly-lysine, poly-ornithine, poly-ethylene-imine (PEI), prolamin, protamine, polyvinyl pyrrolidone (PVP), polyarginine, polyvinylamine, or a combination thereof. In some embodiments, the polycation is a protamine salt. In some embodiments, the protamine salt is protamine sulfate, protamine acetate, protamine bromide, protamine chloride, protamine caproate, protamine trifluoroacetate, protamine HCO3, protamine propionate, protamine lactate, protamine formate, protamine nitrate, protamine citrate, protamine monohydrogenphosphate, protamine dihydrogenphosphate, protamine tartrate, or protamine perchlorate. In some embodiments, the protamine salt is protamine sulfate. In some embodiments, the composition further comprises a heterologous payload. In some embodiments, the heterologous payload comprises exenatide, insulin, or human growth hormone.

In some embodiments, the carrier consists of Pseudomonas exotoxin A or a portion of a Pseudomonas exotoxin A. In some embodiments, the carrier consists of a polypeptide of SEQ ID NO: 69 or a portion of a SEQ ID NO: 69. In some embodiments, the carrier consists of a portion of a Cholix polypeptide. In some embodiments, the Cholix polypeptide consists of an amino acid sequence with a C-terminus at any one of amino acid positions 206 to 425 of SEQ ID NO: 1. In some embodiments, the Cholix polypeptide consists of an amino acid sequence with a C-terminus at any one of amino acid positions 150 to 205 of SEQ ID NO: 1. In some embodiments, the Cholix polypeptide consists of an amino acid sequence with a C-terminus at any one of amino acids 150 to 195 of SEQ ID NO: 1.

In some embodiments, the Cholix polypeptide consists of an amino acid sequence with an N-terminus at any one of amino acid positions 1 to 41 of SEQ ID NO: 1. In some embodiments, the Cholix polypeptide consists of an amino acid sequence with an N-terminus at any one of amino acid positions 35 to 40 of SEQ ID NO: 1. In some embodiments, the Cholix polypeptide consists of an amino acid sequence from amino acid position 40 of the sequence set forth in SEQ ID NO: 7 to any one of amino acid positions 150 to 205 of the sequence set forth in SEQ ID NO: 7. In some embodiments, the Cholix polypeptide has a C-terminus at any one of amino acid positions 150 to 187 of the sequence set forth in SEQ ID NO: 7. In some embodiments, the Cholix polypeptide consists of the amino acid sequence set forth in SEQ ID NO: 8. In some embodiments, the Cholix polypeptide consists of the amino acid sequence set forth in SEQ ID NO: 9 or SEQ ID: NO: 10. In some embodiments, amino acids positions are numbered based on alignment of the Cholix polypeptide to the sequence set forth in SEQ ID NO: 7, wherein amino acid positions are numbered from N-terminus to C-terminus and starting with position 1 at the N-terminus. In some embodiments, the composition comprises a particle.

In some embodiments, described herein are compositions comprising insulin, wherein at least 20% of the insulin is intact at 1 hour in a pancreatin assay comprising incubating the composition comprising insulin with pancreatin in PBS at 37° C. In some embodiments, described herein are compositions comprising insulin, wherein at least 20% of the insulin is intact at 1 hour in a simulated intestinal fluid assay comprising incubating insulin containing particles in simulated intestinal fluid USP (Rica Pharmaceuticals R7109000-500A, 4× dilution, pH 6.8) at 142 ug/ml (Insulin content at 37° C. for 14 hours.

In some embodiments, the composition further comprises a carrier derived from a bacterial toxin, wherein the carrier is capable of transporting into the polarized epithelial cell or transcytosing across a polarized epithelial cell. In some embodiments, the carrier consists of a portion of a Cholix polypeptide. In some embodiments, the composition comprises a transition metal cation. In some embodiments, the transition metal cation is selected from the group consisting of Fe2+, Mn2+, Zn2+, Co2+, Ni2+, and Cu2+. In some embodiments, the transition metal cation is Zn2+. In some embodiments, the composition comprises a polycation. In some embodiments, the polycation is protamine. In some embodiments, the composition comprises a particle. In some embodiments, the particle comprises a microparticle. In some embodiments, the microparticle is formed by spray drying. In some embodiments, the particle has a diameter of about 50 nm to about 20 μm.

In some embodiments, described herein are pharmaceutical compositions comprising the compositions described herein. In some embodiments, described herein are pharmaceutical compositions comprising the compositions described herein and a preservative. In some embodiments, described herein are pharmaceutical compositions comprising the compositions described herein and a pharmaceutically acceptable excipient. In some embodiments, described herein are methods comprising administering to a subject a pharmaceutical composition described herein. In some embodiments, the subject has an inflammatory disease, an autoimmune disease, a cancer, or a metabolic disorder. In some embodiments, the subject has a metabolic disorder.

In some embodiments, the metabolic disorder is diabetes, diabetes as a consequence of obesity, hyperglycemia, dyslipidemia, hypertriglyceridemia, syndrome X, insulin resistance, impaired glucose tolerance (IGT), diabetic dyslipidemia, hyperlipidemia, fatty liver disease, nonalcoholic steatohepatitis (NASH), hepatitis, obesity, vascular disease, heart disease, stroke, impaired glucose tolerance, raised fasting glucose, insulin resistance, urinary albumin secretion, central obesity, hypertension, elevated triglycerides, elevated LDL cholesterol and reduced HDL cholesterol, hyperglycemia, hyperinsulinemia, dyslipidemia, ketosis, hypertriglyceridemia, syndrome X, insulin resistance, impaired fasting glucose, impaired glucose tolerance (IGT), diabetic dyslipidemia, gluconeogenesis, excess glycogenolysis, diabetic ketoacidosis, hypertriglyceridemia, hypertension, diabetic nephropathy, renal insufficiency, renal failure, hyperphagia, muscle wasting, diabetic neuropathy, diabetic retinopathy, diabetic coma, arteriosclerosis, coronary heart disease, peripheral artery disease, or hyperlipidemia.

In some embodiments, described herein are methods comprising combining a bacterial derived carrier with a heterologous payload and a cation to produce a particle. In some embodiments, the heterologous payload is selected from the group consisting of a dye, radiopharmaceutical, hormone, cytokine, anti-TNF agent, glucose lowering agent, tumor associated antigen, peptide, and polypeptide. In some embodiments, the method further comprises spray drying the bacterial derived carrier, the heterologous payload and the cation. In some embodiments, the method comprises (a) preparing a mixture comprising the isolated carrier and payload; (b) preparing a mixture comprising protamine sulfate and NaPO4; and (c) combining the mixture of (a) to the mixture of (b) and allowing the combined mixture to set overnight at room temperature. In some embodiments, the method further comprises step (d) disrupting the particles from step (c) into smaller particles by increasing the ionic strength of the combined mixture from step (c). In some embodiments, the preparation of step (a) does not comprise ZnCl2. In some embodiments, the preparation of step (a) comprises ZnCl2.

An aspect of the present disclosure is a composition comprising a carrier capable of entering a polarized epithelial cell or transcytosing across a polarized epithelial cell; and a heterologous payload, wherein a molar ratio of the heterologous payload to the carrier is greater than 1:1.

The composition can be a particle. The particle can be a microparticle. The microparticle can be formed by spray drying. The particle or microparticle can have a diameter of about 50 nm to about 20 μm.

In some cases, the composition comprises the transition metal cation. The transition metal cation can be selected from the group consisting of Fe²⁺, Mn²⁺, Zn²⁺, Co²⁺, Ni²⁺, and Cu²⁺. The transition metal cation can be Zn²⁺.

In some cases, the composition comprises a polycation. The polycation can be protamine.

The composition can comprise a carrier derived from a bacterial toxin. In some cases, the carrier is capable of entering a polarized epithelial cell or transcytosing across a polarized epithelial cell. In some cases, the carrier may be coupled to the polycation.

The carrier can comprise a portion of a Pseudomonas exotoxin A. In some cases, the carrier consists of a portion of a Pseudomonas exotoxin A. The carrier can comprise a portion of a Cholix polypeptide. In some cases, the carrier consists of a portion of a Cholix polypeptide. The Cholix polypeptide can consist of an amino acid sequence with a C-terminal truncation at any one of amino acid positions 206 to 425 of SEQ ID NO: 7. The Cholix polypeptide can consist of an amino acid sequence with a C-terminus truncation at any one of amino acid positions 150 to 205 of SEQ ID NO: 7. The Cholix polypeptide can consist of an amino acid sequence with a C-terminus truncation at any one of amino acid positions 150 to 195 of SEQ ID NO: 7. The Cholix polypeptide can consist of an amino acid sequence with an N-terminal truncation at any one of amino acid positions 1 to 41 of SEQ ID NO: 7. The Cholix polypeptide can consist of an amino acid sequence with an N-terminal truncation at any one of amino acid positions 35 to 40 of SEQ ID NO: 7. The Cholix polypeptide can consist of an amino acid sequence from amino acid position 40 of the sequence set forth in SEQ ID NO: 7 to any one of amino acid positions 150 to 205 of the sequence set forth in SEQ ID NO: 7. The Cholix polypeptide can terminate at amino acid position 150 or at amino acid position 187 of the sequence set forth in SEQ ID NO: 7. The Cholix polypeptide can consist of the amino acid sequence set forth in SEQ ID NO: 8. The Cholix polypeptide can consist of the amino acid sequence set forth in SEQ ID NO: 9 or SEQ ID: NO: 10. The amino acids can be numbered based on alignment of the Cholix polypeptide to the sequence set forth in SEQ ID NO: 7, wherein amino acid positions can be numbered from N-terminus to C-terminus and starting with position 1 at the N-terminus.

In some cases, the composition comprises a heterologous payload. The heterologous payload can be selected from the group consisting of a macromolecule, small molecule, peptide, polypeptide, nucleic acid, messenger RNA (mRNA), micro RNA (miRNA), small hairpin RNA(shRNA), small interfering RNA (siRNA), CRISPR RNA (e.g., guide RNA, e.g., single guide RNA (sgRNA)), antisense molecule, protein, antibody, DNA, plasmid, vaccine, polymer nanoparticle, and a catalytically-active material.

The heterologous payload can be a therapeutic payload. The heterologous payload can be selected from the group consisting of a dye and radiopharmaceutical, hormone, cytokine, anti-TNF agent, glucose lowering agent, tumor associated antigen, peptide, and polypeptide. The heterologous payload can be a polypeptide that is a modulator of inflammation in a gastrointestinal tract. The therapeutic payload can be a glucagon-like peptide-2 (GLP-2) analog. The GLP-2 analog can be Teduglutide.

The heterologous payload can be a cytokine. The cytokine can be selected from the group consisting of IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, and IL-30. The cytokine can be IL-10. The cytokine can be IL-22. The cytokine can lack its native secretion signal.

The heterologous payload can be a hormone. The hormone can be a human growth hormone (hGH).

The heterologous payload can be a glucose-lowering agent. The heterologous can be an incretin, glucagon proprotein, glucagon peptide, glucagon-like peptide 1, glucagon-like peptide 2, glicentin, glicentin-related polypeptide, gastric inhibitory polypeptide preprotein, gastric inhibitory polypeptide, dipeptidyl peptidase 4, glucose transporter member 4, preproglucagon, insulin receptor substrate 1, insulin, insulin analog, apolipoprotein A-II, solute carrier family 2, facilitated glucose transporter member 1, glycogen synthase 1, glycogen synthase 2, tyrosine-protein phosphatase non-receptor type 1, RAC-alpha serine threonine-protein kinase, peroxisome proliferator-activated receptor gamma, hexokinase 3, phosphatidylinositol-3,4,5-triphosphate 3-phosphatase and dual-specificity protein, pyruvate dehydrogenase kinase 1, calcium-binding and coiled-coil domain-containing protein 1, Max-like protein X, fructose-bisphosphate aldolase A, glucagon-like peptide 1 receptor, glucagon-like peptide 2 receptor, gastric inhibitory polypeptide receptor, insulin-like growth factor 1 receptor, insulin-like growth factor 2 receptor, insulin receptor, GLP-1 agonist-exenatide, GLP-1 agonist-Liraglutide, exenatide, exendin-4, exendin-3, GIPR agonist (Des-Ala2-GIP1-30), GIPR agonist-truncated GIP1-30, GLP-1R agonist (aa 1-37 of GIP), GLP-1R agonist (aa 7-36 of GIP), lixisenatide (tradenames Adlyxin®, and Lyxumia®, Sanofi), liraglutide (tradename Victoza®, Novo Nordisk A/S), semaglutide (tradename Ozempic®, Novo Nordisk A/S), albiglutide (tradename Tanzeum®, GlaxoSmithKline; GLP-1 dimer fused to albumin), dulaglutide (tradename Trulicity®, Eli Lilly), glucose-dependent insulinotropic polypeptide, multi-specific peptide agonist, Tirzepatide (Eli Lilly), SAR425899 (Sanofi), Dual Amylin Calcitonin Receptor Agonist DACRA-089, Glargine/Lantus®, glulisin/Apidra®, glarine/Toujeo®, Insuman®, detemir/Levemir®, lispro/Humalog®/Liprolog®, degludec/DegludecPlus, insulin aspart, insulin and analogues (e.g. LY-2605541, LY2963016, NN1436), PEGylated insulin lispro, Humulin®, Linjeta, SuliXen®, NN1045, Insulin plus Symlin™, PE0139, fast-acting and short-acting insulins (e.g. Linjeta, PH20, NN1218, HinsBet), (APC-002) hydrogel, oral, inhalable, transdermal and sublingual insulins (e.g. Exubera®, Nasulin®, Afrezza®, Tregopil®, TPM 02, Capsulin, Oral-lyn®, Cobalamin®, oral insulin, ORMD-0801, NN1953, NN1954, NN1956, VlAtab, and Oshadi oral insulin), or exendin-4 analog, wherein the exendin-4 analog can be desPro36-exendin-4(1-39)-Lys6NH2; H-des(Pro36, 37)-exendin-4-Lys4-NH2; H-des(Pro36, 37)-exendin-4-Lys5-NH2; desPro36[Asp28]exendin-4 (1-39); desPro36[IsoAsp28]exendin-4 (1-39); desPro36[Met(O)14, Asp28]exendin-4 (1-39); desPro36[Met(O)14, IsoAsp28]exendin-4 (1-39); desPro36[Trp(O2) 26, Asp28]exendin-4 (1-39); desPro36[Trp(O2) 25, IsoAsp28]exendin-4 (1-39); desPro36[Met(O)14 Trp(O2)25, Asp28]exendin-4 (1-39); or desPro36[Met(O)14 Trp(O2)25, IsoAsp28]exendin-4 (1-39). The heterologous can be insulin or an insulin analog. The heterologous can be exenatide.

The heterologous payload can comprise a fluorescent label. The fluorescent label can be a fluorescein. The heterologous payload can be an exenatide-fluorescein complex.

In some cases, the composition is resistant to cleavage by a pancreatic enzyme. At least 50% of the carrier can remain intact after exposure to the pancreatic enzyme for 2 hrs. At least 50% of the carrier can remain intact after 2 hours in a pancreatin assay, wherein the pancreatin assay comprises incubating the composition comprising 100 μg of the carrier with 10 μg of pancreatin in 100 μL phosphate buffered saline (PBS) at 37° C.

A molar ratio of the transition metal cation to the carrier may be from about 100:1 to about 300,000:1. A molar ratio of the transition metal cation to the carrier can be from about 1000:1 to about 30,000:1. A molar ratio of the transition metal cation to the carrier can be from about 1000:1 to about 10,000:1.

A molar ratio of the protamine to the carrier can be from about 10:1 to about 0.01:1. The molar ratio of the heterologous payload to the carrier can be from about 2:1 to about 6000:1. A molar ratio of the protamine to the carrier can be about 1:1

The molar ratio of the heterologous payload to the carrier can be from about 2:1 to about 10:1. The molar ratio of the heterologous payload to the carrier can be about 7:1. The molar ratio of the heterologous payload to the carrier can be about 7.16:1.

The heterologous payload can be a polypeptide comprising the sequence of SEQ ID NO: 11 or SEQ ID NO: 14. The heterologous payload can be a polypeptide comprising the sequence of SEQ ID NO: 18 or SEQ ID NO: 19. The heterologous payload can be a polypeptide comprising the sequence of SEQ ID NO: 20. The heterologous payload can be a polypeptide comprising the sequence of SEQ ID NO: 21. The heterologous payload can be a polypeptide comprising the sequence of SEQ ID NO: 22.

The composition can be encapsulated. The encapsulated composition can be configured to release the heterologous payload under a first condition but not under a second condition. The encapsulated composition can be configured to release the heterologous payload at a high pH but not at a low pH. The encapsulated composition can comprise an enteric coating.

Another aspect of the present disclosure is a pharmaceutical composition comprising any herein-disclosed composition.

In some cases, the polycation is protamine, poly-lysine, poly-ornithine, poly-ethylene-imine (PEI), prolamin, protamine, polyvinyl pyrrolidone (PVP), polyarginine, polyvinylamine, or a combination thereof. In some cases, the polycation is a protamine salt. In some cases, the protamine salt is protamine sulfate, protamine acetate, protamine bromide, protamine chloride, protamine caproate, protamine trifluoroacetate, protamine HCO3, protamine propionate, protamine lactate, protamine formate, protamine nitrate, protamine citrate, protamine monohydrogenphosphate, protamine dihydrogenphosphate, protamine tartrate, or protamine perchlorate. In some cases, the protamine salt is protamine sulfate.

An aspect of the present disclosure is a composition comprising a carrier, wherein the carrier is capable of entering a polarized epithelial cell or transcytosing across a polarized epithelial cell, wherein at least 60% of the carrier is intact at 0.5 hours in a pancreatin assay, wherein the pancreatin assay comprises incubating the composition comprising 100 μg of the carrier with 10 μg of pancreatin in 100 μL phosphate buffered saline (PBS) at 37° C. In some cases, at least 90% of the carrier is intact at 2 hours in the pancreatin assay.

In some cases, the composition further comprises a cation. In some cases, the cation is a metal cation or a polycation. In some cases, the cation is a metal cation. In some cases, the metal cation is a transition metal cation. In some cases, the carrier consists of a portion of a Pseudomonas exotoxin A. In some cases, the carrier consists of a portion of a Cholix polypeptide. In some cases, the Cholix polypeptide consists of an amino acid sequence with a C-terminus at any one of amino acid positions 206 to 425 of SEQ ID NO: 7. In some cases, the Cholix polypeptide consists of an amino acid sequence with a C-terminus at any one of amino acid positions 150 to 205 of SEQ ID NO: 7. In some cases, the Cholix polypeptide consists of an amino acid sequence with a C-terminus at any one of amino acids 150 to 195 of SEQ ID NO: 7. In some cases, the Cholix polypeptide consists of an amino acid sequence with an N-terminus at any one of amino acid positions 1 to 41 of SEQ ID NO: 7. In some cases, the Cholix polypeptide consists of an amino acid sequence with an N-terminus at any one of amino acid positions 35 to 40 of SEQ ID NO: 7. In some cases, the Cholix polypeptide consists of an amino acid sequence from amino acid position 40 of the sequence set forth in SEQ ID NO: 7 to any one of amino acid positions 150 to 205 of the sequence set forth in SEQ ID NO: 7. In some cases, the Cholix polypeptide has an C-terminus at any one of amino acid positions 150 to 187 of the sequence set forth in SEQ ID NO: 7. In some cases, the Cholix polypeptide consists of the amino acid sequence set forth in SEQ ID NO: 8. In some cases, the Cholix polypeptide consists of the amino acid sequence set forth in SEQ ID NO: 9 or SEQ ID: NO: 10. In some cases, amino acids positions are numbered based on alignment of the Cholix polypeptide to the sequence set forth in SEQ ID NO: 7, wherein amino acid positions are numbered from N-terminus to C-terminus and starting with position 1 at the N-terminus.

Another aspect of the present disclosure is a composition comprising a Cholix variant ending at position 195-347 and a heterologous payload, wherein the heterologous payload is a glucose regulating agent. In some cases, the ending position of the Cholix variant is determined relative to SEQ ID NO: 7. In some cases, the carrier is capable of transcytosing the heterologous payload across a polarized epithelial cell. In some cases, the glucose regulating agent is a glucose-lowering agent. In some cases, the glucose-lowering agent is an incretin, glucagon proprotein, glucagon peptide, glucagon-like peptide 1, glucagon-like peptide 2, glicentin, glicentin-related polypeptide, gastric inhibitory polypeptide preprotein, gastric inhibitory polypeptide, dipeptidyl peptidase 4, glucose transporter member 4, preproglucagon, insulin receptor substrate 1, insulin, insulin analog, apolipoprotein A-II, solute carrier family 2, facilitated glucose transporter member 1, glycogen synthase 1, glycogen synthase 2, tyrosine-protein phosphatase non-receptor type 1, RAC-alpha serine threonine-protein kinase, peroxisome proliferator-activated receptor gamma, hexokinase 3, phosphatidylinositol-3,4,5-triphosphate 3-phosphatase and dual-specificity protein, pyruvate dehydrogenase kinase 1, calcium-binding and coiled-coil domain-containing protein 1, Max-like protein X, fructose-bisphosphate aldolase A, glucagon-like peptide 1 receptor, glucagon-like peptide 2 receptor, gastric inhibitory polypeptide receptor, insulin-like growth factor 1 receptor, insulin-like growth factor 2 receptor, insulin receptor, GLP-1 agonist-exenatide, GLP-1 agonist-Liraglutide, exenatide, exendin-4, exendin-3, GIPR agonist (Des-Ala2-GIP1-30), GIPR agonist-Truncated GIP1-30, GLP-1R agonist (aa 1-37 of GIP), GLP-1R agonist (aa 7-36 of GIP), lixisenatide (tradenames Adlyxin®, and Lyxumia®, Sanofi), liraglutide (tradename Victoza®, Novo Nordisk A/S), semaglutide (tradename Ozempic®, Novo Nordisk A/S), albiglutide (tradename Tanzeum®, GlaxoSmithKline; GLP-1 dimer fused to albumin), dulaglutide (tradename Trulicity®, Eli Lilly), glucose-dependent insulinotropic polypeptide, multi-specific peptide agonist, Tirzepatide (Eli Lilly), SAR425899 (Sanofi), dual amylin calcitonin receptor agonist DACRA-089, glargine/Lantus®, glulisin/Apidra®, glarine/Toujeo®, Insuman®, detemir/Levemir®, lispro/Humalog®/Liprolog®, Degludec/DegludecPlus, insulin aspart, insulin and analogues (e.g. LY-2605541, LY2963016, NN1436), PEGylated insulin Lispro, Humulin®, Linjeta, SuliXen®, NN1045, Insulin plus Symlin™, PE0139, fast-acting and short-acting insulins (e.g. Linjeta, PH20, NN1218, HinsBet), (APC-002) hydrogel, oral, inhalable, transdermal and sublingual insulins (e.g. Exubera®, Nasulin®, Afrezza®, Tregopil®, TPM 02, Capsulin, Oral-lyn®, Cobalamin®, oral insulin, ORMD-0801, NN1953, NN1954, NN1956, VlAtab, and Oshadi oral insulin), or an exendin-4 analog, wherein the exendin-4 analog is desPro36-exendin-4(1-39)-Lys6NH2; H-des(Pro36, 37)-exendin-4-Lys4-NH2; H-des(Pro36, 37)-exendin-4-Lys5-NH2; desPro36[Asp28]exendin-4 (1-39); desPro36[IsoAsp28]exendin-4 (1-39); desPro36[Met(O)14, Asp28]exendin-4 (1-39); desPro36[Met(O)14, IsoAsp28]exendin-4 (1-39); desPro36[Trp(O2) 26, Asp28]exendin-4 (1-39); or desPro36[Trp(O2) 25, IsoAsp28]exendin-4 (1-39); desPro36[Met(O)14 Trp(O2)25, Asp28]exendin-4 (1-39); or desPro36[Met(O)14 Trp(O2)25, IsoAsp28]exendin-4 (1-39). In some cases, the heterologous payload comprises an incretin. In some cases, the heterologous payload comprises exenatide or insulin. In some cases, the composition comprises a particle. In some cases, the heterologous payload is exendin-3, efpeglenatide, semaglutide, GLP-1R agonist (aa 1-37 of GIP), GLP-1R agonist (aa 7-36 of GIP), tirzepatide, oxyntomodulin, GIPR agonist-truncated GIP1-30, dual amylin calcitonin receptor agonist, preproinsulin, insulin aspart, insulin glargine, or insulin lispro.

An aspect of the present disclosure is a composition comprising a carrier derived from a bacterial toxin capable of entering a polarized epithelial cell or transcytosing across a polarized epithelial cell; and a transition metal cation or a polycation, wherein the polycation is molecule or chemical complex having more than 2 positive charges. In some cases, the composition comprises a transition metal cation. In some cases, the transition metal cation is selected from the group consisting of Fe2+, Mn2+, Zn2+, Co2+, Ni2+, and Cu2+. In some cases, the transition metal cation is Zn2+. In some cases, the composition comprises the polycation. In some cases, the polycation is a protamine salt. In some cases, the composition further comprises a heterologous payload. In some cases, the heterologous payload comprises exenatide, insulin, or human growth hormone. In some cases, the carrier consists of Pseudomonas exotoxin A or a portion of a Pseudomonas exotoxin A. In some cases, the carrier consists of a polypeptide of SEQ ID NO: 69 or a portion of a SEQ ID NO: 69. In some cases, the carrier consists of a portion of a Cholix polypeptide. In some cases, the Cholix polypeptide consists of an amino acid sequence with a C-terminus at any one of amino acid positions 206 to 425 of SEQ ID NO: 1. In some cases, the Cholix polypeptide consists of an amino acid sequence with a C-terminus at any one of amino acid positions 150 to 205 of SEQ ID NO: 1. In some cases, the Cholix polypeptide consists of an amino acid sequence with a C-terminus at any one of amino acids 150 to 195 of SEQ ID NO: 1. In some cases, the Cholix polypeptide consists of an amino acid sequence with an N-terminus at any one of amino acid positions 1 to 41 of SEQ ID NO: 1. In some cases, the Cholix polypeptide consists of an amino acid sequence with an N-terminus at any one of amino acid positions 35 to 40 of SEQ ID NO: 1. In some cases, the Cholix polypeptide consists of an amino acid sequence from amino acid position 40 of the sequence set forth in SEQ ID NO: 7 to any one of amino acid positions 150 to 205 of the sequence set forth in SEQ ID NO: 7. In some cases, the Cholix polypeptide has a C-terminus at any one of amino acid positions 150 to 187 of the sequence set forth in SEQ ID NO: 7. In some cases, the Cholix polypeptide consists of the amino acid sequence set forth in SEQ ID NO: 8. In some cases, the Cholix polypeptide consists of the amino acid sequence set forth in SEQ ID NO: 9 or SEQ ID: NO: 10. In some cases, amino acids positions are numbered based on alignment of the Cholix polypeptide to the sequence set forth in SEQ ID NO: 7, wherein amino acid positions are numbered from N-terminus to C-terminus and starting with position 1 at the N-terminus. In some cases, the composition comprises a particle.

An aspect of the present disclosure is a composition comprising insulin, wherein at least 20% of the insulin is intact at 1 hour in a pancreatin assay comprising incubating the composition comprising insulin with pancreatin in at 37° C. In some cases, 10 μg of insulin is incubated for 1 hour with 10 μg pancreatin in at 37° C. In some cases, the composition further comprises a carrier derived from a bacterial toxin, wherein the carrier is capable of transporting into the polarized epithelial cell or transcytosing across a polarized epithelial cell. In some cases, the carrier consists of a portion of a Cholix polypeptide. In some cases, the composition comprises a transition metal cation. In some cases, the transition metal cation is selected from the group consisting of Fe2+, Mn2+, Zn2+, Co2+, Ni2+, and Cu2+. In some cases, the transition metal cation is Zn2+. In some cases, the composition comprises a polycation. In some cases, the polycation is protamine. In some cases, the composition comprises a particle. In some cases, the particle comprises a microparticle. In some cases, the microparticle is formed by spray drying. In some cases, the particle has a diameter of about 50 nm to about 20 μm.

Another aspect of the present disclosure is a pharmaceutical composition comprising any herein-disclosed composition and a preservative.

Another aspect of the present disclosure is a pharmaceutical composition comprising any herein-disclosed composition and a pharmaceutically acceptable excipient.

In an aspect, the present disclosure provides a method of treating a disease in a subject comprising administering to the subject any herein-disclosed pharmaceutical composition.

The disease can be an inflammatory disease, an autoimmune disease, a cancer, or a metabolic disorder.

The disease can be a metabolic disorder. The metabolic disorder can be diabetes (T1D or T2D), diabetes as a consequence of obesity, hyperglycemia, dyslipidemia, hypertriglyceridemia, syndrome X, insulin resistance, impaired fasting glucose, impaired glucose tolerance (IGT), diabetic dyslipidemia, hyperlipidemia, fatty liver disease, nonalcoholic steatohepatitis, hepatitis, obesity, vascular disease, heart disease, stroke, impaired glucose tolerance, raised fasting glucose, insulin resistance, urinary albumin secretion, central obesity, hypertension, elevated triglycerides, elevated LDL cholesterol and reduced HDL cholesterol, hyperglycemia, hyperinsulinemia, dyslipidemia, ketosis, hypertriglyceridemia, syndrome X, insulin resistance, impaired fasting glucose, impaired glucose tolerance (IGT), diabetic dyslipidemia, gluconeogenesis, excess glycogenolysis, diabetic ketoacidosis, hypertriglyceridemia, hypertension, diabetic nephropathy, renal insufficiency, renal failure, hyperphagia, muscle wasting, diabetic neuropathy, diabetic retinopathy, diabetic coma, arteriosclerosis, coronary heart disease, peripheral artery disease, or hyperlipidemia.

The composition can be a particle, wherein the composition comprises a heterologous payload, and wherein the composition comprises the polycation, wherein the carrier is capable of transporting the heterologous payload in the polarized epithelial cell or transcytosing the heterologous payload across a polarized epithelial cell.

The carrier can be coupled to the polycation. The polycation can be protamine, poly-lysine, poly-ornithine, poly-ethylene-imine (PEI), prolamin, protamine, polyvinyl pyrrolidone (PVP), polyarginine, polyvinylamine, or a combination thereof. The polycation can be a protamine salt. The protamine salt can be protamine sulfate, protamine acetate, protamine bromide, protamine chloride, protamine caproate, protamine trifluoroacetate, protamine bicarbonate (HCO3), protamine propionate, protamine lactate, protamine formate, protamine nitrate, protamine citrate, protamine monohydrogenphosphate, protamine dihydrogenphosphate, protamine tartrate, or protamine perchlorate. The protamine salt can be protamine sulfate.

An aspect of the present disclosure is a pharmaceutical composition comprising any herein-disclosed particle and in which the heterologous payload is a glucose-lowering agent.

In some cases, the glucose-lowering agent is selected from the group consisting of insulin and an insulin analog. In some cases, the encapsulated particle releases the payload at a high pH but not at a low pH.

Another aspect of the present disclosure is a pharmaceutical composition comprising any herein-disclosed particle and a preservative. In some cases, the pharmaceutical composition further comprises a tonicity modifier.

In an aspect, the present disclosure provides a composition comprising a carrier derived from a bacterial toxin, wherein the carrier is capable of transporting into a polarized epithelial cell or transcytosing across a polarized epithelial cell, in which at least 30% of the carrier is intact at 0.5 hours in a pancreatin assay comprising incubating the composition comprising 100 μg of the carrier with 10 μg of pancreatin in 100 μL PBS at 37° C.

At least 40% of the carrier can be intact at 0.5 hours in a pancreatin assay comprising incubating the composition comprising 100 μg of the carrier with 10 μg of pancreatin in 100 μL PBS at 37° C. At least 50% of the carrier can be intact at 0.5 hours in a pancreatin assay comprising incubating the composition comprising 100 μg of the carrier with 10 μg of pancreatin in 100 μL PBS at 37° C. At least 60% of the carrier can be intact at 0.5 hours in a pancreatin assay comprising incubating the composition comprising 100 μg of the carrier with 10 μg of pancreatin in 100 μL PBS at 37° C. At least 90% of the carrier can be intact at 2 hours in a pancreatin assay comprising incubating the composition comprising 100 μg of the carrier with 10 μg of pancreatin in 100 μL PBS at 37° C.

The composition can further comprise a cation. The cation can be a metal cation or a polycation. The cation can a metal cation. The metal cation can be a transition metal cation.

An aspect of the present disclosure is a composition comprising (a) a carrier derived from a bacterial toxin and (b) a transition metal cation or a polycation, wherein the carrier is capable of entering a polarized epithelial cell and/or transcytosing across a polarized epithelial cell.

An aspect of the present disclosure includes a method comprising combining a bacterial derived carrier with a heterologous payload and a cation to produce a particle. In some cases, the heterologous payload is selected from the group consisting of a dye, radiopharmaceutical, hormone, cytokine, anti-TNF agent, glucose lowering agent, tumor associated antigen, peptide, and polypeptide. In some cases, the method further comprises spray drying the bacterial derived carrier, the heterologous payload and the cation. In some cases, the method comprises (a) preparing a mixture comprising the isolated carrier and payload; (b) preparing a mixture comprising protamine sulfate and NaPO4; and (c) combining the mixture of (a) to the mixture of (b) and allowing the combined mixture to set overnight at room temperature. In some cases, the method further comprises step (d) disrupting the particles from step (c) into smaller particles by increasing the ionic strength of the combined mixture from step (c). In some cases, the preparation of step (a) does not comprise ZnCl2. In some cases, the preparation of step (a) comprises ZnCl2.

In one aspect, provided herein is a delivery construct comprising a Cholix variant that does not comprise amino acids 1-348 of SEQ ID NO: 75, and is not SEQ ID NO: 76, complexed with a heterologous payload, wherein the heterologous payload is a glucose regulating agent, and wherein the carrier is capable of a) transcytosing the heterologous payload across a polarized epithelial cell or b) transporting the heterologous payload into the polarized epithelial cell. The payload can be an incretin or an incretin mimetic. The payload can comprise a GLP-1 receptor agonist. The payload can comprise any one of SEQ ID NOs: 26-34 or 17. The payload can consist of SEQ ID NO: 15. The payload can consist of SEQ ID NO: 29. The payload can consist of SEQ ID NO: 16. The payload can consist of SEQ ID NO: 17. The payload can consist of SEQ ID NO: 33. The payload can consist of SEQ ID NO: 34. The payload can be a GIP receptor agonist. The payload can comprise any one of SEQ ID NO: 33 or 36-38.

The payload can be an insulin. The insulin can comprise SEQ ID NO: 43 and SEQ ID NO: 44; SEQ ID NO: 45 and SEQ ID NO: 46; SEQ ID NO: 47 and SEQ ID NO: 48; or SEQ ID NO: 49 and SEQ ID NO: 50. The insulin can comprise SEQ ID NO: 47 and SEQ ID NO: 48. The insulin can comprise SEQ ID NO: 49 and SEQ ID NO: 50. The payload can be any one of SEQ ID NOs: 51-63.

The Cholix variant can be a sequence set forth in SEQ ID NO: 81, or a fragment or sequence variant thereof. The carrier can be noncovalently bound to the heterologous payload. The carrier can be covalently bound to the heterologous payload.

The delivery construct can be a single polypeptide. The delivery construct can further comprise a cleavable linker, wherein cleavage of the linker releases the payload from the carrier. The carrier can be located at a C-terminus of the polypeptide and the payload can be at an N-terminus of the polypeptide. The carrier can comprise SEQ ID NO: 6; SEQ ID NO: 2; SEQ ID NO: 65; or SEQ ID NO: 73. The carrier can have at least 90% amino acid identity to a C-terminally-truncated variant of SEQ ID NO: 1. The carrier can comprise a C-terminally-truncated variant of SEQ ID NO: 81. The carrier can consist of the first 195, 206, 244, 250, 266, 386, or 415 amino acid residues of SEQ ID NO: 1, SEQ ID NO: 12, or SEQ ID NO: 81. The heterologous payload can be configured to bind a receptor.

Also provided herein is a method of treating a metabolic disorder of a subject comprising administering to the subject an effective amount of any one of the above delivery constructs. The metabolic disorder can be diabetes and/or obesity.

Any aspect or case disclosed herein may be combined with any other aspect of case disclosed herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A is a digital image depicting self-assembling fusion molecule-protamine microparticles, prepared as described in Example 1, suspended in low ionic strength buffer (0.05 M). FIG. 1B is a digital image depicting self-assembling fusion molecule-protamine microparticles, prepared as described in Example 1, suspended in high ionic strength buffer (>1 M). FIG. 1C is a digital image depicting the microparticles of FIG. 1B returned to a low ionic strength buffer (0.05 M). Particles were imaged using the GE Cytel system in high power (10×) bright field mode.

FIG. 2A is a digital image depicting self-assembling insulin-protamine microparticles, prepared as described in Example 2, in bright field mode. FIG. 2B is a digital image depicting self-assembling insulin-protamine microparticles, prepared as described in Example 2, which exhibit FITC fluorescence in blue field mode. FIG. 2C is a digital image depicting the merged image of the bright field and blue field images shown in FIGS. 2A and 2B. The merged image shows FITC fluorescence of insulin microparticles. Particles were imaged using the GE Cytel system in high power (10×) bright or blue field mode. FITC-fluorescence was imaged by exciting the sample at 381 nm and recording the fluorescent emission at 435 nm. The blue arrow indicates an approximately 150 μM particle.

FIG. 3A is a digital image depicting self-assembling fusion molecule-protamine-zinc microparticles, prepared as described in Example 3, in red field mode.

FIG. 3B is a digital image of the same fusion molecule-protamine-zinc microparticles in bright field mode. Particles were imaged using the GE Cytel system in high power (10×) bright or red field mode. Red-fluorescence was imaged by exciting the sample at 481 nm and recording the fluorescent emission at 535 nm.

FIG. 4A is a digital image depicting self-assembling fusion molecule-protamine (without ZnCl2) microparticles, prepared as described in Example 3, in bright field mode. FIG. 4B is a digital image of the same particles in red field mode. Particles were imaged using the GE Cytel system in high power (10×) bright or red field mode. Red-fluorescence was imaged by exciting the sample at 481 nm and recording the fluorescent emission at 535 nm.

FIG. 5A shows a dissolution profile for formulation 37-156 at pH 2 and pH 7, and upon elevation of pH from pH 2-pH 7.

FIG. 5B shows a dissolution profile for formulation 37-167 at pH 2 and pH 7, and upon elevation of pH from pH 2-pH 7.

FIG. 5C shows a dissolution profile for a formulated nanoparticle at pH 2 and pH 7, and upon elevation of pH from pH 2-pH 7.

FIG. 6 shows insulin stability upon exposure different nanoparticle formulations to pancreatin.

FIG. 7A shows sections of rat small intestine 15 minutes after intraluminal injection of 37-49 hGH/SEQ ID NO: 3/Eudragit FS particles. hGH is seen in green and SEQ ID NO: 3 in red. FIG. 7B shows sections of rat small intestine 30 minutes after intraluminal injection of 37-49 hGH/SEQ ID NO: 3/Eudragit FS particles. hGH is seen in green and SEQ ID NO: 3 in red.

FIG. 7C shows sections of rat small intestine 45 minutes after intraluminal injection of 37-49 hGH/SEQ ID NO: 3/Eudragit FS particles. hGH is seen in green and SEQ ID NO: 3 in red.

FIG. 7D shows sections of rat small intestine 60 minutes after intraluminal injection of 37-49 hGH/SEQ ID NO: 3/Eudragit FS particles. hGH is seen in green and SEQ ID NO: 3 in red.

FIG. 8 shows serum levels of hGH in a rat administered hGH containing particles by oral gavage.

FIG. 9 show levels of hGH transported through Caco-2 cells when delivered in different formulations.

FIG. 10A shows in vitro release of exenatide, assayed by reverse phase liquid chromatography (RPLC), from 5 particles of Table 3 at pH 5, pH 7, pH 7.5 and pH 10 at the indicated time points.

FIG. 10B shows in vitro release of exenatide, assayed by RPLC, from 5 further particles of Table 3 at pH 5, pH 7, pH 7.5 and pH 10 at the indicated time points.

FIG. 10C shows in vitro release of exenatide, assayed by size exclusion chromatography (SEC), from 5 particles of Table 3 at pH 5, pH 7, pH 7.5 and pH 10 at the indicated time points.

FIG. 10D shows in vitro release of exenatide, assayed by size exclusion chromatography (SEC), from 5 further particles of Table 3 at pH 5, pH 7, pH 7.5 and pH 10 at the indicated time points.

FIGS. 11A-C show pancreatin stability of SEQ ID NO: 3 and/or exenatide in different formulations described herein. FIG. 11A shows pancreatin stability of SEQ ID NO: 3 and/or exenatide in zinc-containing formulations, FIG. 11B shows pancreatin stability of SEQ ID NO: 3 and/or exenatide in protamine-containing formulations, and FIG. 11C shows pancreatin stability of exenatide in a formulation of zinc and exenatide.

FIG. 12A shows a confocal microscopy image of a formulation of SEQ ID NO: 3, FITC-exenatide and zinc, with the FITC-exenatide visualized.

FIG. 12B shows a confocal microscopy image of a formulation of SEQ ID NO: 3, FITC-exenatide and zinc, with SEQ ID NO: 3 visualized by Alexa 647 labeled anti SEQ ID NO: 3 antibodies.

FIG. 12C shows a merged image of FIGS. 12A and 12B.

FIG. 13 shows the distribution of particle sizes of the formulation in FIG. 12A-C.

FIG. 14 shows transport of formulations described herein through SMI-100 cells.

FIG. 15 shows a schematic example of a method of producing an acid resistant particle comprising a Cholix carrier, zinc, and exenatide.

FIG. 16 shows methods in which the passage of microparticles through different mucus and epithelial layers can be detected using in vitro or in vivo methods.

FIG. 17A shows pancreatin stability of SEQ ID NO: 3 and/or exenatide in different formulations described herein.

FIG. 17B shows pancreatin stability of SEQ ID NO: 3 and/or FITC-exenatide in different formulations described herein.

FIG. 18 shows a reverse phase chromatogram (RPLC) showing the presence of SEQ ID NO: 3 at the retention time of 6.8 min and exenatide at 7.5 min.

FIG. 19 shows in vitro release of exenatide, assayed by RPLC from formulations E0, E14, E18, E0-FITC, E14-FITC, and E18-FITC at pH 1, pH 5, and pH 7 at the indicated time points at 37° C.

FIG. 20 shows serum concentrations of exenatide in rats which were intraluminally administered formulations E0, E14 and E18.

FIG. 21 shows serum concentrations of exenatide in rats which were intravenously administered exenatide.

FIG. 22 shows the purity of SEQ ID NO: 70-Exenatide (SEQ ID NO: 70 crosslinked to Exenatide) run on a Coomassie Blue-stained SDS-PAGE gel.

FIG. 23 shows in vivo transcytosis of Exenatide crosslinked to carriers SEQ ID NO: 80 or SEQ ID NO: 70 across the jejunum of Sprague Dawley Rats. The amount (in pM) of Exenatide transported across intestinal tissues was measured at 10 minutes and 40 minutes post treatment. The data shows that both SEQ ID NO: 80-Exenatide and SEQ ID NO: 70-Exenatide are capable of transport at a higher rate than Exenatide alone at 10 minutes and at 40 minutes.

FIG. 24 shows a time-concentration profile of blood sugar after a glucose challenge. The effects of SEQ ID NO: 70-Exenatide administered by oral gavage were compared with a negative-control treatment (Oral buffer) and Exenatide administered by intraperitoneal injection as a positive control. These results demonstrate that SEQ ID NO: 70-Exenatide reduces the rise in blood glucose levels after a glucose challenge.

FIG. 25 shows that SEQ ID NO: 11 can bind to a GLP-1 receptor.

### DETAILED DESCRIPTION

### I. Overview

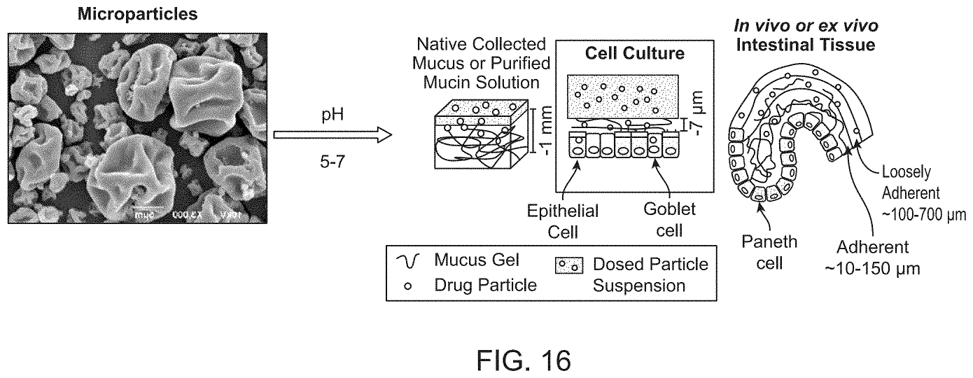

Provided herein are methods and compositions that can comprise (a) a carrier derived from a bacterial toxin and (b) a transition metal cation or a polycation, wherein the carrier is capable of entering into a polarized epithelial cell or transcytosing across a polarized epithelial cell. The carrier derived from a bacterial toxin can be, e.g., a Cholix polypeptide (e.g., from Vibrio cholerae) or Pseudomonas exotoxin (PE) A, e.g., from Pseudomonas aeruginosa. The compositions can further comprise a payload, e.g., a heterologous payload (e.g., the payload is not the carrier, e.g., Cholix or PE), e.g., a therapeutic payload. The compositions can be in the form of particle, e.g., a microparticle or a nanoparticle and can be generated, e.g., by spraying drying and/or lyophilization. Methods provided herein include administering the compositions to a subject. In some cases, the compositions can be formulated to pass through the acidic environment of the stomach intact (e.g., the particles can be acid-resistant microparticles). FIG. 16 provides a schematic illustrating penetration of particles through mucus samples, a thin mucus layer over cultured cells, and a healthy mucus layer over epithelial cells in vivo. The particles can become soluble at pH 5-7 and, e.g., can support binding or transport in a reconstituted mucus layer assessed in a passage study using cultured endothelial tissue layers or through intestinal tissue in either in vitro or in vivo models. In some cases, the compositions provided herein can be formulated for delivery to a subject by other routes, e.g., respiratory delivery.

Also provided herein are methods and compositions comprising a carrier capable of entering a polarized epithelial cell or transcytosing across a polarized epithelial cell and a heterologous payload, wherein a molar ratio of the heterologous payload to the carrier is greater than 1:1.

Provided herein are methods and compositions comprising a carrier, wherein the carrier is capable of entering a polarized epithelial cell or transcytosing across a polarized epithelial cell, wherein at least 60% of the carrier is intact at 0.5 hours in a pancreatin assay, wherein the pancreatin assay comprises incubating the composition comprising 100 μg of the carrier with 10 μg of pancreatin in 100 μL phosphate buffered saline (PBS) at 37° C. Methods of making these compositions and methods of administering these compositions to a subject are provided herein.

Also provided herein are methods and compositions comprising a Cholix variant ending at position 195-347 and a heterologous payload, wherein the heterologous payload is a glucose regulating agent. Also provided herein are compositions comprising a carrier coupled to a glucose regulating agent.

Also provided herein are pharmaceutical compositions comprising the compositions provided herein, methods of making the compositions provided herein, and methods of administering the compositions provided herein to a subject, e.g., a subject with a defect in glucose regulation.

### II. Particles

Compositions provided herein can comprise one or more particles. The one or more particles can comprise one or more microparticles or nanoparticles. The one or more particles can comprise one or more carriers, one or more payloads, (e.g. heterologous payloads), and/or one or more cations. The one or more cation can be one or more polycations, e.g., a protamine. The one or more cations can be one or more metal cations. The one or more metal cations can be one or more transition metal cation. The one or more metal cations can be one or more divalent metal cations. The one or more transition metal cations can be Fe2+, Mn2+, Zn2+, Co2+, Ni2+ and Cu2+. The one or more transition metal cations can be a zinc cation. The one or more divalent metal cation can be calcium (Ca2+), chromium (Cr2+), cobalt (Co2+), iron (Fe2+), magnesium (Mg2+), manganese (Mn2+), nickel (Ni2+), copper (Cu2+), or zinc (Zn2+). The one or more cations can be protamine, poly-lysine, poly-ornithine, poly-ethylene-imine (PEI), prolamin, polyvinyl pyrrolidone (PVP), polyarginine, polyvinylamine, or a combination thereof. The one or more cations can be a protamine salt.

The one or more particles, e.g., one or more microparticles or nanoparticles can be one or more self-assembling particles, e.g., a stable self-assembling particle, e.g., microparticle or nanoparticle.

The compositions provided herein can comprise one or more carriers and one or more payloads and/or one or more cations, e.g., one or more polycations. The one or more carriers and one or more payloads can be coupled directly or indirectly, covalently or non-covalently. The one or more carriers and one or more payloads can be in the form of a fusion molecule. In some cases, the one or more carriers and one or more payloads are not in a fusion molecule. The carrier-payload fusion molecule can transport the one or more payload molecules (e.g., one or more therapeutic payloads) into epithelial cells (e.g., polarized gut epithelial cells). The carrier can be capable of transporting the payload into or across epithelial cells using endogenous trafficking pathways. Utilization of endogenous trafficking pathways, as opposed to use of passive diffusion, can allow the carrier to shuttle the payload rapidly and efficiently into or across epithelial cells without impairing the barrier function of these cells or the biological activity of the payload. The one or more payloads can be a polypeptide comprising, consisting of, or consisting essentially of the sequence set forth in any of SEQ ID NOs: 11 or 14-64 (see Table 12).

The one or more carriers and one or more cations can be coupled directly or indirectly, covalently or non-covalently. The one or more carriers and one or more cations can be in the form of a fusion molecule. In some cases, the fusion molecule can assemble into a microparticle or nanoparticle with the one or more payloads. The carrier-cation fusion molecule can transport the one or more payload molecules (e.g., one or more therapeutic payloads) into epithelial cells (e.g., polarized gut epithelial cells). In some cases, the one or more carriers and one or more cations are not coupled directly or covalently attached. In some cases, the one or more carriers and one or more cations are not part of a fusion molecule.

In some cases, the one or more cations is protamine or a protamine salt. Protamine can refer to a group of strongly basic proteins present in sperm cells in salt-like combination with nucleic acids. Protamines can be obtained from salmon (salmine), rainbow trout (iridine), herring (clupeine), sturgeon (sturine), or Spanish mackerel or tuna (thynnine). The peptide composition of a specific protamine can vary depending of which family, genera or species of fish it is obtained from. Protamine can contain four major components, e.g., single-chain peptides containing about 30-32 residues of which about 21-22 are arginines. The N-terminal residue can be proline for each of the four main components. Therefore, chemical modification of protamine by a particular salt can be expected to be homogenous. The protamine salt can be from salmon. The protamine salt can be from herring. The protamine salt can be from rainbow trout. The protamine salt can be from tuna.

The one or more particles, e.g., one or more microparticles or nanoparticles can comprise a protamine salt selected from the group consisting of protamine sulfate, protamine acetate, protamine bromide, protamine chloride, protamine caproate, protamine trifluoroacetate, protamine bicarbonate (HCO3), protamine propionate, protamine lactate, protamine formate, protamine nitrate, protamine citrate, protamine monohydrogenphosphate, protamine dihydrogenphosphate, protamine tartrate, protamine perchlorate, and mixtures of any two protamine salts. The protamine salt may be combined with the other components in a liquid medium prior to particle formation, for example by precipitation or spray drying. The protamine salt in the liquid medium prior to particle formation can be at a concentration from about 0.05 mg/mL to about 0.10 mg/mL, from about 0.10 mg/mL to about 0.15 mg/mL, from about 0.15 mg/mL to about 0.20 mg/mL, from about 0.20 mg/mL to about 0.25 mg/mL, from about 0.25 mg/mL to about 0.30 mg/mL, from about 0.30 mg/mL to about 0.35 mg/mL, from about 0.35 mg/mL to about 0.40 mg/mL, from about 0.40 mg/mL to about 0.45 mg/mL, or from about 0.45 mg/mL to about 0.5 mg/mL. The protamine salt can be a mixture of two different salts, wherein one salt is sulfate and the other salt is acetate, propionate, lactate, formate, or nitrate salts of protamine. The molar ratio between the two different salts can from about 0.1 to about 1, from about 0.2 to about 1, from about 0.3 to about 1, from about 0.4 to about 1, from about 0.5 to about 1, from about 0.6 to about 1, from about 0.7 to about 1, from about 0.8 to about 1, and from about 0.9 to about 1.

The particle can further comprise a divalent metal ion, e.g., zinc, cobalt, magnesium, or calcium, or combinations of these ions. The metal ion can be zinc.

In some examples, the particles can be formed by combining a carrier and a cation at a ratio of from about 1:0.001 to about 1:2000 by weight (carrier:cation). In some cases, the carrier and the cation can be combined in a ratio of from about 1:0.01 to about 1:500 by weight. In some cases, the carrier and the cation can be combined in a ratio of from about 1:0.08 to about 1:173 by weight. In some cases, the carrier and the cation can be combined in a ratio of from about 1:0.1 to about 1:200 by weight. In some cases, the carrier and the cation can be combined in a ratio of from about 1:0.1 to about 1:5 by weight. In some cases, the carrier and the cation can be combined in a ratio of from about 1:0.08 to about 1:1.6 by weight. In some cases, the carrier and the cation can be combined in a ratio of from about 1:8 to about 1:180 by weight. In other examples, the particles can be formed by combining a carrier and a cation at a ratio of from about 0.01:1 to about 0.2:1 by weight. In some cases, the carrier and the cation can be combined in a ratio of about 1:1 by weight. In some cases, the carrier and the cation can be combined in a ratio of at least about 1:0.001, 1:0.01, 1:0.08, 1:0.1, 1:0.16, 1:0.2, 1:0.3, 1:0.32, 1:0.4, 1:0.8, 1:1, 1:1.6, 1:2, 1:3, 1:4, 1:5, 1:6, 1:7, 1:8, 1:8.6, 1:9, 1:10, 1:11, 1:12, 1:13, 1:14, 1:15, 1:16, 1:17, 1:18, 1:19, 1:20, 1:25, 1:30, 1:35, 1:40, 1:45, 1:50, 1:60, 1:70, 1:80, 1:86, 1:90, 1:100, 1:150, 1:172, 1:200, or 1:2000, by weight. In some cases, the carrier and the cation can be combined in a ratio of less than about 1:0.001, 1:0.01, 1:0.08, 1:0.1, 1:0.16, 1:0.2, 1:0.3, 1:0.32, 1:0.4, 1:0.8, 1:1, 1:1.6, 1:2, 1:3, 1:4, 1:5, 1:6, 1:7, 1:8, 1:8.6, 1:9, 1:10, 1:11, 1:12, 1:13, 1:14, 1:15, 1:16, 1:17, 1:18, 1:19, 1:20, 1:25, 1:30, 1:35, 1:40, 1:45, 1:50, 1:60, 1:70, 1:80, 1:86, 1:90, 1:100, 1:150, 1:172, or 1:200, 1:2000, by weight.

In some examples, the particles can be formed by combining a payload fusion molecule and a cation at a ratio of from about 1:0.001 to about 1:2000 by weight (payload fusion molecule:cation). In some cases, the payload fusion molecule and the cation can be combined in a ratio of from about 1:0.01 to about 1:500 by weight. In some cases, the payload fusion molecule and the cation can be combined in a ratio of from about 1:0.08 to about 1:173 by weight. In some cases, the payload fusion molecule and the cation can be combined in a ratio of from about 1:0.1 to about 1:200 by weight. In some cases, the payload fusion molecule and the cation can be combined in a ratio of from about 1:0.1 to about 1:5 by weight. In some cases, the payload fusion molecule and the cation can be combined in a ratio of from about 1:0.08 to about 1:1.6 by weight. In some cases, the payload fusion molecule and the cation can be combined in a ratio of from about 1:8 to about 1:180 by weight. In other examples, the particles can be formed by combining a payload fusion molecule and a cation at a ratio of from about 0.01:1 to about 0.2:1 by weight. In some cases, the payload fusion molecule and the cation can be combined in a ratio of about 1:1 by weight. In some cases, the payload fusion molecule and the cation can be combined in a ratio of at least about 1:0.001, 1:0.01, 1:0.08, 1:0.1, 1:0.16, 1:0.2, 1:0.3, 1:0.32, 1:0.4, 1:0.8, 1:1, 1:1.6, 1:2, 1:3, 1:4, 1:5, 1:6, 1:7, 1:8, 1:8.6, 1:9, 1:10, 1:11, 1:12, 1:13, 1:14, 1:15, 1:16, 1:17, 1:18, 1:19, 1:20, 1:25, 1:30, 1:35, 1:40, 1:45, 1:50, 1:60, 1:70, 1:80, 1:86, 1:90, 1:100, 1:150, 1:172, or 1:200, 1:2000, by weight. In some cases, the payload fusion molecule and the cation can be combined in a ratio of less than about 1:0.001, 1:0.01, 1:0.08, 1:0.1, 1:0.16, 1:0.2, 1:0.3, 1:0.32, 1:0.4, 1:0.8, 1:1, 1:1.6, 1:2, 1:3, 1:4, 1:5, 1:6, 1:7, 1:8, 1:8.6, 1:9, 1:10, 1:11, 1:12, 1:13, 1:14, 1:15, 1:16, 1:17, 1:18, 1:19, 1:20, 1:25, 1:30, 1:35, 1:40, 1:45, 1:50, 1:60, 1:70, 1:80, 1:86, 1:90, 1:100, 1:150, 1:172, or 1:200, 1:2000, by weight.

In some examples, the particles can be formed by combining a carrier and a payload at a ratio of from about 0.01:1 to about 1:2, from about 0.0116:1 to about 1:1, from about 0.2:1 to about 1:1 by weight. In some cases, the carrier to the payload can be combined at a ratio of at least 0.01:1, 0.0116:1, 0.02:1, 0.0232:1, 0.03:1, 0.04:1, 0.05:1, 0.06:1, 0.07:1, 0.08:1, 0.09:1, 0.1:1, 0.2:1, 0.232:1, 0.3:1, 0.4:1, 0.5:1, 0.6:1, 0.7:1, 0.8:1, 0.9:1, 1:1, 1.1:1, 1.2:1, 1.3:1, 1.4:1, 1.5:1, 1.6:1, 1.7:1, 1.8:1, 1.9:1 or 2:1 by weight. In some cases, the carrier to the payload can be combined at a ratio of less than 0.01:1, 0.0116:1, 0.02:1, 0.0232:1, 0.03:1, 0.04:1, 0.05:1, 0.06:1, 0.07:1, 0.08:1, 0.09:1, 0.1:1, 0.2:1, 0.232:1, 0.3:1, 0.4:1, 0.5:1, 0.6:1, 0.7:1, 0.8:1, 0.9:1, 1:1, 1.1:1, 1.2:1, 1.3:1, 1.4:1, 1.5:1, 1.6:1, 1.7:1, 1.8:1, 1.9:1 or 2:1 by weight.

In some examples, the particle, or the composition can comprise a carrier and a cation (e.g., Zn2+ or protamine) at a ratio of carrier to cation that is from about 5:1 to about 1:5 by mole, from about 10:1 to 1:10 by mole, from about 2:1 to about 1:2 by mole, from about 1:10 to about 1:1000 by mole, from about 1:30 to about 1:700 by mole, from about 1:100 to about 1:300,000 by mole, or from about 1:1000 to about 1:30,000 by mole. The ratio of carrier to cation can be about 10:1, 5:1, 2:1, 1:1, 1:2, 1:3, 1:4, 1:5, 1:6, 1:7, 1:8, 1:9, 1:10, 1:25, 1:50, 1:100, 1:500, 1:650, 1:1000, 1:2000, 1:5000, 1:10,000, 1:20,000, 1:30,000, 1:40,000, 1:50,000, 1:75000, 1:100,000, 1:200,000, or 1:300,000 by mole. In some cases, the ratio of carrier to cation is at least 1:300,000, 1:200,000, 1:100000, 1:50000, 1:30000, 1:25000, 1:20000, 1:15000, 1:10000, 1:5000, 1:3000, 1:2800, 1:2000, 1:1500, 1:1000, 1:500, 1:100, 1:50, 1:25, 1:10, 1:5, 1:2, 1:1.12, 1:1 or 10:1 by mole. In some cases, the ratio of carrier to cation is less than 1:300,000, 1:200,000, 1:100000, 1:50000, 1:30000, 1:25000, 1:20000, 1:15000, 1:10000, 1:5000, 1:3000, 1:2800, 1:2000, 1:1500, 1:1000, 1:500, 1:2, 1:1.12, or 1:1 by mole. In some cases, the carrier may be present in the particle as a monomer, dimer, trimer, or other complex. The ratio of carrier to cation may be calculated based on the moles of the monomers of the carrier. If the cation is in a salt, the amount or moles of cation itself, rather than the amount or moles of a salt containing the cation, can be used to determine a ratio.

The particle, or the composition, can comprise a carrier and a payload at a ratio of carrier to payload that is from about 1:1 to about 1:10 by mole, from about 10:1 to about 1:1000 by mole, from about 1:1 to about 1:1000 by mole, from about 1:10 to about 1:1000 by mole, from about 1:10 to about 1:6000 per mole, from about 1:1 to about 1:100 by mole, from about 1:5 to about 1:20 by mole, about 1:7 by mole, or about 1:716 by mole. In some cases, the particle, or the composition, can comprise a carrier and a payload at a ratio of carrier to payload that is less than about 1:1, about 1:2, about 1:5, about 1:7, about 1:10, about 1:20, about 1:30, about 1:60, about 1:70, about 1:80, about 1:90, about 1:100, about 1:125, about 1:150, about 1:200, about 1:300, about 1:400, about 1:500, about 1:600, about 1:750, about 1:1000, about 1:2500, about 1:5000, about 1:6000, or about 1:50 by mole. In some cases, the ratio of carrier to payload is at least about 1:6000, 1:5000, 1:4000, 1:3000, 1:2000, 1:1000, 1:900, 1:800, 1:700, 1:620, 1:617.06, 1:600, 1:500, 1:400, 1:310, 1:308.53, 1:200, 1:150, 1:125, 1:100, 1:90, 1:80, 1:70, 1:60, 1:50, 1:40, 1:30, 1:20, 1:10, 1:5, or 1:1 by mole. In some cases, the ratio of carrier to payload is less than about 1:6000, 1:5000, 1:4000, 1:3000, 1:2000, 1:1000, 1:900, 1:800, 1:700, 1:620, 1:617.06, 1:600, 1:500, 1:400, 1:310, 1:308.53, 1:200, 1:150, 1:125, 1:100, 1:90, 1:80, 1:70, 1:60, 1:50, 1:40, 1:30, 1:20, 1:10, 1:5, or 1:1 by mole. The ratio of carrier to payload can be about 1:6000, 1:5000, 1:4000, 1:3000, 1:2000, 1:1000, 1:900, 1:800, 1:700, 1:620, 1:617.06, 1:600, 1:500, 1:400, 1:310, 1:308.53, 1:200, 1:150, 1:125, 1:100, 1:90, 1:80, 1:70, 1:60, 1:50, 1:40, 1:30, 1:20, 1:10, 1:9, 1:8, 1:7, 1:6, 1:5, 1:4, 1:3, 1:2, 1:1, 2:1, 5:1, or 10:1 by mole. In some cases, the carrier or the payload, or both, may be present as monomers, dimers, trimers, or other complexes. The ratio by mole may be calculated by comparing the monomers of carrier with the monomers of payload.

In some instances, a carrier is indirectly and non-covalently coupled to a payload. In such instances, particles (e.g., liposomes, microparticles, nanoparticles, metallic nanoparticles, polymer-based nanoparticles, etc.) can be loaded (e.g., on the inside and/or on the surface of the particle) with payload molecules (e.g., IL-10, IL-22, GLP-1, etc.), and carriers, e.g., Cholix derived or PE derived carrier molecule(s) can be coupled to such nanoparticles (e.g., onto its surface). In some cases, particles can be formed which comprise payload molecules and carrier molecules, e.g., a cholix derived or PE derived molecule.

In some instances, a ratio (e.g., molar ratio) of payload to carrier in a composition provided herein (e.g., a particle) can be at least about 15000:1, 10000:1, 5000:1, 2500:1, 1000:1, 500:1, 250:1, 100:1, 50:1, 25:1, 10:1, 5:1, 2.5:1, 1:1. This ratio can allow transport of such payload-containing particles (e.g., nanoparticles) into or across polarized epithelial cells (e.g., polarized gut epithelial cells) using the carrier, e.g., PE or Cholix derived carrier, attached to the surface. In some cases, the particle (e.g., nanoparticle) can release the payload following transcytosis or intracellular delivery. In cases where the particle (e.g., nanoparticle) is transported across epithelial cells, the released payload can bind to receptors within submucosal tissue (e.g., lamina propria) and/or can enter the systemic circulation and thus provide a certain function (e.g., a therapeutic or diagnostic function) systemically. In other cases, where a particle (e.g., nanoparticle) releases the payload inside an epithelial cell, the payload (e.g., a nucleic acid) may provide certain intracellular functions, e.g., production of transgenes within these cells, modulation of gene expression, etc.

The particle, or the composition, e.g., pharmaceutical composition, can further comprise a preservative, e.g., phenol, m-cresol, methyl p-hydroxybenzoate, propyl p-hydroxybenzoate, 2-phenoxyethanol, butyl p-hydroxybenzoate, 2-phenylethanol, benzyl alcohol, chlorobutanol, or thiomerosal, or mixtures thereof.

The composition, e.g., pharmaceutical composition, can further comprise a tonicity modifier, e.g., a sugar or sugar alcohol, an amino acid (e.g. L-glycine, L-histidine, arginine, lysine, isoleucine, aspartic acid, tryptophan, threonine), an alditol (e.g. glycerol (glycerine), 1,2-propanediol (propyleneglycol), 1,3-propanediol, 1,3-butanediol) polyethyleneglycol (e.g. PEG400), glycerol, mannitol, propylene glycol, dimethyl sulfone, methyl sulfonyl methane, trehalose, sucrose, sorbitol, saccarose, lactose, or mixtures thereof.

The composition, e.g., pharmaceutical composition, can further comprise a buffer, e.g., sodium acetate, sodium carbonate, citrate, glycylglycine, histidine, glycine, lysine, arginine, sodium dihydrogen phosphate, disodium hydrogen phosphate, sodium phosphate, and tris(hydroxymethyl)-aminomethane, or mixtures thereof.

The formulations of the present disclosure can be prepared, e.g., as described in Remington's Pharmaceutical Sciences, 1985 or in Remington: The Science and Practice of Pharmacy, 19th edition, 1995, where techniques of the pharmaceutical industry involve dissolving and mixing the ingredients as appropriate to give the desired end product.

In another aspect, provided herein is method of preparing particles, e.g., microparticles or nanoparticles, e.g., self-assembling microparticles or nanoparticles containing a carrier, a payload, and a cation. The carrier and payload can form a fusion molecule. The method can comprise: (a) preparing a mixture comprising the isolated carrier and payload (e.g., as a fusion molecule) and, optionally, ZnCl₂; (b) preparing a mixture comprising protamine sulfate and NaPO₄; and (c) combining the mixture of (a) to the mixture of (b) and allowing the combined mixture to set overnight at room temperature. The method can further comprise step (d) disrupting the particles from step (c) into smaller particles by increasing the ionic strength of the combined mixture from step (c). In some cases, the preparation of step (a) does not comprise ZnCl₂. In some cases, the preparation of step (a) does comprise ZnCl₂.

In some cases, the carrier and payload do not form a fusion molecule. The method can comprise: (a) preparing a mixture comprising the isolated carrier and isolated payload and, optionally, ZnCl₂; (b) preparing a mixture comprising protamine sulfate and NaPO₄; and (c) combining the mixture of (a) to the mixture of (b) and allowing the combined mixture to set overnight at room temperature. The method can further comprise step (d) disrupting the particles from step (c) into smaller particles by increasing the ionic strength of the combined mixture from step (c). In some cases, the preparation of step (a) does not comprise ZnCl2. In some cases, the preparation of step (a) does comprise ZnCl₂. In some cases the combined mixture of (a) and (b) can be spray dried to form microparticles or nanoparticles. In some cases, microparticles or nanoparticles can be formed using a commercially available spray dryer.

In some cases, the carrier and cation can form a fusion molecule. The method can comprise: (a) preparing a mixture comprising the isolated carrier and cation fusion molecule, and the isolated payload and, optionally, ZnCl₂; (b) preparing a mixture comprising the isolated payload and NaPO4; and (c) combining the mixture of (a) to the mixture of (b) and allowing the combined mixture to set overnight at room temperature. The method can further comprise step (d) disrupting the particles from step (c) into smaller particles by increasing the ionic strength of the combined mixture from step (c). In some cases, the preparation of step (a) does not comprise ZnCl₂. In some cases, the preparation of step (a) does comprise ZnCl₂. In some cases the combined mixture of (a) and (b) can be spray dried to form microparticles or nanoparticles. In some cases, microparticles or nanoparticles can be formed using a commercially available spray dryer.

The Nano Spray Dryer B-90 (Buchi, Switzerland) can produce protein particles from sub micron to 2 micron, with a narrow size distribution and controlled release of the active pharmaceutical ingredient. Proteins such as, hGH, Insulin can be spray dried in a continuous, single step process. Counter ions and polymer excipients can be used for encapsulation and to achieve slow release of the active pharmaceutical ingredient. The system can be compatible with water soluble excipients and water based emulsion. The sample feed rate may be between 1 ml/min and 3 ml/min. The gas flow may be between 100 L/min and 160 L/min. The pressure may be atmospheric pressure.

The protein concentration may be between about 0.02 μmole and 1 μmole. The feed rate may be between about 100 L/min and 200 L/min. The inlet temperature may be between about 80° C. and 140° C. The head temperature may be between about 30° C. and 70° C. Many different buffer solutions can be used such as, for example, carbonate, acetate, lactate, succinate, phosphate and tris buffers. Examples of excipients which can be used include hydroxypropylmethylcellulose (HPMC), carboxymethylcellulose (CMC), alginate, Eudragits, chitosan, dextran, poly(lactic-co-glycolic acid) (PLGA), pluronic, arabic gum, and polysorbate 20. Non-limiting examples of counter ions, or molecules that include counter ions, include protamine, SEQ ID NO: 3 construct, cationic cell-penetrating peptides, polyglutamate and hyaluronic acid.

The particles, e.g., self-assembled particles or spray dried particles, e.g., protamine particles, zinc particles, or zinc and protamine particles, can have a size of no greater than about 200 nm, no greater than about 300 nm, no greater than about 400 nm, no greater than about 500 nm, no greater than about 600 nm, no greater than about 700 nm, no greater than about 800 nm, or no greater than about 900 nm. The self-assembled microparticles, e.g., protamine microparticles, zinc particles, zinc and protamine particles, can have a size of no greater than about 10 nm, 20 nm, 30 nm, 40 nm, 50 nm, 60 nm, 70 nm, 80 nm, 90 nm, 100 nm, 110 nm, 120 nm, 130 nm, 140 nm, 150 nm, 160 nm, 170 nm, 180 nm, 190 nm, 200 nm, 250 nm, 300 nm, 350 nm, 400 nm, 450 nm, 500 nm, 600 nm, 700 nm, 800 nm, 900 nm, 1 μm, no greater than about 5 μm, no greater than about 10 μm, no greater than about 15 μm, no greater than about 20 μm, no greater than about 25 μm, no greater than about 30 μm, no greater than about 35 μm, no greater than about 40 μm, no greater than about 45 μm, no greater than about 50 μm, no greater than about 55 μm, no greater than about 60 μm, no greater than about 65 μm, no greater than about 70 μm, no greater than about 75 μm, no greater than about 80 μm, no greater than about 85 μm, no greater than about 90 μm, no greater than about 95 μm, no greater than about 100 μm, no greater than about 105 μm, no greater than about 110 μm, no greater than about 115 μm, no greater than about 120 μm, no greater than about 125 μm, no greater than about 130 μm, no greater than about 135 μm, no greater than about 140 μm, no greater than about 145 μm, and no greater than about 150 μm. In some cases, the particles have a size of from about 1 μm and about 20 μm. In some cases, the particles have an average size of about 5 μm±2 μm. In some cases, the particles have an average size of about 150 μm±50 μm. In some cases, the particles have an average size of from about 30 nm to about 6000 nm, from about 60 nm to about 6000 nm, from about 30 nm to about 3000 nm, from about 60 nm to about 3000 nm, from about 100 nm to about 1000 nm, from about 200 nm to about 800 nm, or from about 300 nm to about 600 nm. In some cases, the particles have an average size of at least about 30 nm, 40 nm, 50 nm, 60 nm, 70 nm, 80 nm, 90 nm, 100 nm, 150 nm, 200 nm, 250 nm, 300 nm, 350 nm, 400 nm, 450 nm, 500 nm, 550 nm, 600 nm, 650 nm, 700 nm, 750 nm, 800 nm, 850 nm, 900 nm, 950 nm, 1000 nm, 1100 nm, 1200 nm, 1300 nm, 1400 nm, 1500 nm, 2000 nm, 3000 nm, 4000 nm, 5000 nm, or 6000 nm.

The particles, e.g., microparticles and nanoparticles can be encapsulated. Natural and synthetic polymer matrices can be used for drug encapsulation and controlled release. Polymer matrices used for particle encapsulation can be chosen such that the particles, and drug contained within the particles, are released at a desired pH, temperature or time. Polymer matrices used for particle encapsulation can be chosen such that the particles, and drug contained within the particles, are protected while in a low pH environment and released upon pH elevation, for example protected in the stomach and then released in the intestine. Polymer matrices used for particle encapsulation can also be chosen to protect the particles from conditions in a first tissue or biological fluid, e.g. stomach acid. Eudragits can be used for pH- and time-controlled drug release, and chitosans, HPMC and hyluronic acid can be used for diffusion-controlled release. Eudragits include a diverse range of polymethacrylate-based copolymers. Examples of Eudragits which can be suitable for use with the methods and compositions of the present disclosure include Eudragit RS 30 D: Poly(ethyl acrylate-co-methyl methacrylate-co-trimethylammonioethyl methacrylate chloride) 1:2:0.1; Eudragit RL 30 D: Poly(ethyl acrylate-co-methyl methacrylate-co-trimethylammonioethyl methacrylate chloride) 1:2:0.2; Eudragit FS 30D: Poly(methyl acrylate-co-methyl methacrylate-co-methacrylic acid) 7:3:1; and Eudragit L 30 D-55: Poly(methacrylic acid-co-ethyl acrylate) 1:1.

The particles can be encapsulated by any method known in the art. In some cases, particles, e.g., microparticles or nanoparticles can be formed in a first step and encapsulated in a second step. For example the particles can be formed through precipitation as described above and then encapsulated in a second step by spray drying. In another example the particles can be formed in a first spray drying step and then encapsulated in a second spray drying step. In some cases, the particles can be formed and encapsulated in a single step. For example, the encapsulating polymer matrices can be mixed with a solution comprising the one or more carriers, payloads and cations prior to a spray drying step. In some cases, a solution comprising one or more encapsulating polymer matrices, carriers, payloads and cations can be spray dried to form nanoparticles or microparticles.

### III. Carriers

A carrier can be a protein or another type of molecule capable of transporting the heterologous payload across or into an epithelium (e.g., a polarized gut epithelium of a subject).

A carrier can be derived from a polypeptide secreted by a bacterium. Such carrier can be derived from a polypeptide secreted from Vibrio cholerae or Pseudomonas aeruginosa. The polypeptide secreted by Vibrio cholerae can be a Cholix polypeptide. The polypeptide secreted by Pseudomonas aeruginosa can be a Pseudomonas exotoxin (PE). A carrier derived from a Cholix polypeptide or PE can be naturally occurring or non-naturally occurring. A carrier derived from a Cholix polypeptide or PE can be a truncated variant of a naturally occurring Cholix peptide or PE or a mutated variant that is not naturally occurring. Mutations can include substitution, deletion, addition.

A. Cholix

A carrier herein can have a reduced (e.g., at least 50% reduced) or ablated ADP ribosylation activity (e.g., ribosylation of elongation factor 2) relative to the carrier set forth in SEQ ID NO: 1.

A carrier can be a polypeptide derived from Cholix or a variant thereof that is further truncated at any one of positions 206 to 415 for transcytosis or position 151-187 for endocytosis as compared to a reference sequence, such as SEQ ID NO: 1 or 12 or 7. Also contemplated herein are transcytosing carriers such having at least about 80%, 85%, 90%, 95%, 98% or 99% sequence identity to any of the carrier sequences shown in TABLE 14, or endocytosing carriers such having at least about 80%, 85%, 90%, 95%, 98% or 99% sequence identity, to any of the carrier sequences shown in TABLE 15. Any of the carriers herein can have a V1L substitution. In some cases, the carrier can be a Cholix polypeptide with a C-terminal truncation at any one of amino acids 206 to 425, 150 to 205, or 150-195 of SEQ ID NO: 1 or 7. In some cases, the carrier can be a Cholix polypeptide with a N-terminal truncation at any one of amino acids 1 to 40 or 35-40 of SEQ ID NO: 1 or 7. In some cases, the carrier consists of the amino acid residues from the N-terminal position 40 to any one of the C-terminal positions 150-205 of the sequence set forth in SEQ ID NO: 7. In some cases, the carrier can be a Cholix polypeptide with a N-terminal truncation at amino acids 266 of SEQ ID NO: 1 or 12 or 7. Such carrier can comprise, consist essentially of, or consist of the amino acid sequence set forth in any one of SEQ ID NOs: 65-67. In other instances, the carrier can have a C-terminus at positions 150 or 187 of the sequence set forth in SEQ ID NO: 7. In some cases, the carrier consists of the amino acid sequence set forth in SEQ ID NO: 8, 9, or 10. In some cases, the Cholix carrier consists of the amino acid sequence set forth in SEQ ID NO: 12 or 13. Position numbering can be based on alignment of the Cholix polypeptide to the sequence set forth in SEQ ID NO: 7, wherein positions are numbered from an N-terminus to a C-terminus starting with position 1 at the N-terminus. In some cases, the carrier can have the sequence of SEQ ID NO: 3.

A carrier can be a truncated variant of a naturally occurring Cholix polypeptide or a mutated variant that is not naturally occurring. Mutations can include substitutions, deletions, or additions. A truncated Cholix-derived carrier can, for example, consist of, consist essentially of, or comprise amino acid residues, 1-415, 1-386, 1-266 or 1-206 of SEQ ID NO: 1 or 7. Thus, the carrier can consist of, consist essentially of, or comprise the amino acid sequence set forth in SEQ ID NO: 6 (an example of Cholix¹⁻⁴¹⁵), SEQ ID NO: 2 (an example of Cholix¹⁻³⁸⁶), SEQ ID NO: 65 (an example of Cholix¹⁻²⁶⁶), or SEQ ID NO: 73 (an example of Cholix¹⁻²⁰⁶). Amino acid sequences are presented in Table 12.

A carrier can include one or more amino acids at its N-terminus that facilitate expression in various microorganisms (e.g., bacteria). For example, a carrier can include an N-terminal methionine, which can be a translational start site. Such a carrier can consist of, consist essentially of, or comprise the amino acid sequence set forth in SEQ ID NO: 65 (an example of M+Cholix¹⁻³⁸⁶). Additionally, any of the carriers herein can have a V1L substitution as set forth in SEQ ID NO: 2 (an example of V1L Cholix).

A carrier can be a fragment of SEQ ID NO: 81 and can comprise, consist essentially of, or consist of no more than 386 amino acids of the amino acid sequence of SEQ ID NO: 81. A carrier can comprise, consist essentially of, or consist of the amino acid residues from any one of the positions 1-38 to any one of the amino acid residues at positions 195-347 of SEQ ID NO: 81. Alternatively, a carrier can comprise, consist essentially of, or consist of the amino acid residues from position 1 to any of the amino acid residues at any one of the positions 195 to 347 of SEQ ID NO: 81.

A carrier can comprise, consist essentially of, or consist of amino acid residues 1-195, 1-206, 1-244, 1-266, 1-386, or 1-415 of SEQ ID NO: 1, SEQ ID NO: 12 or SEQ ID NO: 81. Likewise, a carrier can comprise, consist essentially of, or consist of the amino acid residues 1-275, 1-266, 1-265, 2-265, 3-265, 4-265, 5-265, 1-250, 2-250, 3-250, 4-250, 5-250, 1-245, 2-245, 3-245, 4-245, 5-245, 1-205, 2-205, 3-205, 4-205, and 5-205 of SEQ ID NO: 1, SEQ ID NO: 12 or SEQ ID NO: 81.

Carriers also include variants of any of the above having at least 80%, 85%, 90%, 95%, 98%, or 99% sequence identity to any of the sequences herein.

B. Pseudomonas Exotoxin A

The carrier can comprise a portion of a Pseudomonas exotoxin. Pseudomonas exotoxin A or “PE” can be secreted by Pseudomonas aeruginosa as a 67 kDa protein composed of three prominent globular domains (Ia, II, and III) and one small subdomain (Ib) connecting domains II and III (see e.g., Allured et. al., Proc. Natl. Acad. Sci. 83:1320 1324, 1986). Mature PE can be 613-residue protein, whose sequence is set forth in SEQ ID NO: 69. A example of a nucleic acid encoding mature PE as used herein is set forth in SEQ ID NO: 68.

A PE exotoxin domain I (e.g., SEQ ID NO: 82) can comprise amino acids 1-252 of SEQ ID NO: 69 and can be a receptor binding domain that can be a ligand for a cell surface receptor and mediates binding of PE to a cell. A carrier can have the amino acid sequence set forth in SEQ ID NO: 82. A carrier can comprise an amino acid sequence with greater than 50%, greater than 60%, greater than 70%, greater than 80%, greater than 90%, greater than 95%, or greater than 99% sequence homology or sequence identity to the sequence set forth in SEQ ID NO: 82. Also contemplated herein are carriers such having at least about 80%, 85%, 90%, 95%, 98% or 99% sequence identity to any of the portions of the carrier sequence of SEQ ID NO: 69 shown in TABLE 13.

In some cases, a carrier comprises a polypeptide with conservative or non-conservative substitutions in the amino acid sequence of SEQ ID NO: 7. The carrier can maintain the ability to bind a cell. The carrier can be a truncated version of SEQ ID NO: 69, e.g., SEQ ID NO: 82. The carrier can comprise a receptor binding domain polypeptide wherein one or more amino residues of SEQ ID NO: 82 are deleted. The carrier can comprise a receptor binding domain polypeptide wherein one or more amino residues of SEQ ID NO: 82 are substituted with another amino acid.

The carrier PE domain I can comprise an amino acid sequence having at least 80% identity to the amino acid sequence of SEQ ID NO: 82 or at least 80% identity to a functional fragment thereof. A carrier can comprise a deletion or mutation in one or more of amino acid residues 1-252 of the amino acid sequence of SEQ ID NO: 69, e.g., the amino acid sequence of SEQ ID NO: 82. A carrier can comprise an amino acid sequence having at least 90% sequence identity to the amino acid sequence of 1-252 of SEQ ID NO: 69 (e.g., the amino acid sequence of SEQ ID NO: 82) or at least 90% sequence identity to a functional fragment thereof. A carrier can comprise an amino acid sequence having at least 95% sequence identity to the amino acid sequence of 1-252 of SEQ ID NO: 69, e.g., the amino acid sequence of SEQ ID NO: 82 or at least 95% sequence identity to a functional fragment thereof. A carrier can comprise an amino acid sequence having at least 99% sequence identity to the amino acid sequence of 1-252 of SEQ ID NO: 69, e.g., the amino acid sequence of SEQ ID NO: 82 or at least 99% sequence identity to a functional fragment thereof. A carrier can comprise an amino acid sequence having 100% sequence identity to the amino acid sequence of 1-252 of SEQ ID NO: 69, e.g., the amino acid sequence of SEQ ID NO: 82 or 100% sequence identity to a functional fragment thereof.

The carrier can be coupled, directly or indirectly, covalently or noncovalently, to a payload. In some cases, the carrier and the payload form a fusion protein. The carrier can be coupled, directly or indirectly, covalently or noncovalently, to a cation. In some cases, the carrier and the cation form a fusion protein or complex. In various embodiments, the carrier, payload, carrier-payload complexes, or fusion molecules of the pharmaceutical compositions are encoded by nucleic acids that comprise a promoter, a regulatory element, a DNA sequence encoding a carrier or fragment or truncated variant of such carrier, and a DNA sequence encoding a payload or a cation. The regulatory element can contain transcription binding sites for transcription factors that activates or represses expression of the encoding DNA sequences. In some cases, the regulatory element is the endogenous regulatory element of the DNA sequence encoding the carrier or fragment or truncated variant of such carrier. In some cases, the regulatory element is the endogenous regulatory element of the DNA sequence encoding the payload. In some cases, the regulatory element regulates both the DNA sequence encoding the carrier or fragment or truncated variant of such carrier, and the DNA sequence encoding the payload or cation. In some cases, the carrier and payload are encoded on different nucleic acid molecules. In some cases, the carrier, payload, and cation are encoded on different nucleic molecules. In some cases, the carrier and payload are encoded on the same nucleic acid molecule. In some cases, the carrier, payload, and cation are encoded on the same nucleic acid molecule.

### IV. Payload

In addition to the carrier polypeptide, the compositions provided herein can comprise one or more payloads, e.g., one or more heterologous payloads, or one or more biologically-active payloads, for delivery to a subject. The one or more heterologous payloads can be one or more payloads that do not have a carrier sequence, e.g., a Cholix sequence or a PE sequence. The one or more payloads, e.g., heterologous payloads, can be a macromolecule, small molecule, small organic molecule, peptide, polypeptide, nucleic acid, mRNA, miRNA, shRNA, siRNA, PNA, antisense molecule, antibody, DNA, plasmid, polysaccharide, lipid, antigen, vaccine, polymer nanoparticle, or catalytically-active material. The one or more payloads, e.g., heterologous payloads, can be a polypeptide comprising, consisting of, or consisting essentially of the sequence set forth in any of SEQ ID NOs: 11 or 14-64 (see Table 12).

The one or more payloads, e.g. one or more biologically active payloads, can be a macromolecule that can perform a desirable biological activity when introduced to the bloodstream of the subject. For example, the one or more payloads can have receptor binding activity, enzymatic activity, messenger activity (i.e., act as a hormone, cytokine, neurotransmitter, clotting factor, growth factor, or other signaling molecule), luminescent or other detectable activity, or regulatory activity, or any combination thereof. In various diagnostic embodiments, the one or more payloads can be conjugated to or can itself be a pharmaceutically acceptable gamma-emitting moiety, including but not limited to, indium and technetium, magnetic particles, radiopaque materials such as air or barium and fluorescent compounds (e.g., Alexa-488 or a red fluorescent protein). In some cases, the one or more payloads, e.g., one or more biologically active payloads, do not enter the bloodstream of the subject. In some cases, the one or more payloads act at the lamina propria.

In various embodiments, the one or more payloads is a protein that comprises more than one polypeptide subunit. For example, the protein can be a dimer, trimer, or higher order multimer. In various embodiments, two or more subunits of the protein can be connected with a covalent bond, such as, for example, a disulfide bond. In other embodiments, the subunits of the protein can be held together with non-covalent interactions. One of skill in the art can identify such proteins and determine whether the subunits are properly associated using, for example, an immunoassay.

In various embodiments, the one or more payloads, e.g., one or more therapeutic payloads, are for example a dye, a radiopharmaceutical, a hormone, a cytokine, an anti-TNF agent, a glucose lowering agent, or a tumor associated antigen. In some cases, the one or more therapeutic payloads is a polypeptide that is a modulator of inflammation in the GI tract. In various embodiments, the one or more payloads to be delivered is a glucose-lowering agent for delivery to a subject. Examples of glucose-lowering agents include of incretins, glucagon, glucagon proprotein, glucagon peptide, glucagon-like peptide 1 (GLP-1), glucagon-like peptide 2 (GLP-2), a GLP-2 agonist, teduglutide, glicentin, glicentin-related polypeptide, gastric inhibitory polypeptide preprotein, gastric inhibitory polypeptide, dipeptidyl peptidase 4, glucose transporter member 4, preproglucagon, insulin receptor substrate 1, insulin, apolipoprotein A-II, solute carrier family 2, facilitated glucose transporter member 1, glycogen synthase 1, glycogen synthase 2, tyrosin-protein phosphatase non-receptor type 1, RAC-alpha serine threonine-protein kinase, peroxisome proliferator-activated receptor gamma, hexokinase 3, phosphatidylinositol-3,4,5-triphosphate 3-phosphatase and dual-specificity protein, pyruvate dehydrogenase kinase 1, calcium-binding and coiled-coil domain-containing protein 1, Max-like protein X, fructose-bisphosphate aldolase A, glucagon-like peptide 1 receptor, glucagon-like peptide 2 receptor, gastric inhibitory polypeptide receptor, insulin-like growth factor 1 receptor, insulin-like growth factor 2 receptor, insulin receptor, GLP-1 agonist-exenatide, GLP-1 agonist-liraglutide, exendin-4, exendin-3, gastric inhibitory peptide (GIP), GIPR agonist (Des-Ala2-GIP1-30), GIPR agonist-truncated GIP1-30, GLP-1R agonist (aa 1-37 of GIP), GLP-1R agonist (aa 7-36 of GIP), lixisenatide (tradenames Adlyxin®, and Lyxumia®, Sanofi), liraglutide (tradename Victoza®, Novo Nordisk A/S), semaglutide (tradename Ozempic®, Novo Nordisk A/S), albiglutide (tradename Tanzeum®, GlaxoSmithKline; GLP-1 dimer fused to albumin), dulaglutide (tradename Trulicity®, Eli Lilly), glucose-dependent insulinotropic polypeptide, multi-specific peptide agonists, tirzepatide (Eli Lilly), SAR425899 (Sanofi), dual amylin calcitonin receptor agonist DACRA-089, glargine/Lantus®, glulisin/Apidra®, glarine/Toujeo®, Insuman®, detemir/Levemir®, lispro/Humalog®/Liprolog®, degludec/degludecPlus, insulin aspart, insulin and analogues (e.g. LY-2605541, LY2963016, NN1436), PEGylated insulin Lispro (SEQ ID NOs: 40-41), Humulin®, Linjeta, SuliXen®, NN1045, insulin plus Symlin™, PE0139, fast-acting and short-acting insulins (e.g. Linjeta, PH20, NN1218, HinsBet), (APC-002) hydrogel, oral, inhalable, transdermal and sublingual insulins (e.g. Exubera®, Nasulin®, Afrezza®, Tregopil®, TPM 02, Capsulin, Oral-lyn®, Cobalamin®, oral insulin, ORMD-0801, NN1953, NN1954, NN1956, VlAtab, and Oshadi oral insulin), and exendin-4 analog selected from the group consisting of: desPro36-exendin-4(1-39)-Lys6NH2; H-des(Pro36, 37)-exendin-4-Lys4-NH2; H-des(Pro36, 37)-exendin-4-Lys5-NH2; desPro36[Asp28]exendin-4 (1-39); desPro36[IsoAsp28]exendin-4 (1-39); desPro36[Met(O)14, Asp28]exendin-4 (1-39); desPro36[Met(O)14, IsoAsp28]exendin-4 (1-39); desPro36[Trp(O2) 26, Asp28]exendin-4 (1-39); desPro36[Trp(O2) 25, IsoAsp28]exendin-4 (1-39); desPro36[Met(O)14 Trp(O2)25, Asp28 ]exendin-4 (1-39); and desPro36[Met(O)14 Trp(O2)25, IsoAsp28]exendin-4 (1-39). GLP-1 agonists contemplated for use in particles or delivery constructs disclosed herein include, e.g., exenatide (tradename Byetta®, Amylin/Astrazeneca, SEQ ID NO: 14, SEQ ID NO: 11); lixisenatide (tradenames Adlyxin®, and Lyxumia®, Sanofi, SEQ ID NO: 15); liraglutide (tradename Victoza®, Novo Nordisk A/S, SEQ ID NO: 16); semaglutide (tradename Ozempic®, Novo Nordisk A/S); albiglutide (tradename Tanzeum®, GlaxoSmithKline; GLP-1 dimer fused to albumin); and dulaglutide (tradename Trulicity®, Eli Lilly, SEQ ID NO: 17). The payload can have the ability to bind an incretin receptor, e.g., an incretin receptor in the gastrointestinal tissue or in the hepatic portal system.

In some cases, the one or more payloads, e.g., one or more therapeutic payloads, have the amino acid sequence of SEQ ID NO: 11 or any one or more of SEQ ID NOs: 14 to 64.

### Incretins

A carrier can be coupled to one or more incretins. Incretins belong to a class of gastrointestinal hormones that can increase insulin release from beta cells of the islets of Langerhans after eating, even before blood glucose levels are elevated. Incretins can slow the absorption rate of nutrients into the blood stream by reducing gastric emptying and may reduce food intake. Incretins can inhibit glucagon release from the alpha cells of the Islets of Langerhans. An incretin can be glucagon-like peptide-1 (GLP-1) or Gastric inhibitory peptide (GIP). Both GLP-1 and GIP can be rapidly inactivated by the enzyme dipeptidyl peptidase 4 (DPP-4).

The incretins or incretin mimetic peptides that can be used in the present disclosure may be naturally occurring incretins or incretin mimetic peptides or modified naturally occurring incretins or incretin mimetic peptides. The peptides may be chemically synthesized using standard techniques for peptide synthesis such as solid-phase peptide synthesis, or may be prepared using recombinant DNA techniques known in the art. The peptides thus produced may or may not be identical to the naturally occurring peptides. Analogs, fragments and conjugates of the naturally occurring incretins or incretin mimetic peptides are encompassed as payloads in the present disclosure so long as they retain one or more of the biological activities of the naturally occurring incretins or incretin mimetic peptides.

GLP-1 can be a naturally occurring incretin hormone synthesized in intestinal L-cells by tissue-specific post-translational processing of preproglucagon. GLP-1 has been implicated in the control of appetite and satiety. GLP-1 can act through the GLP-1 receptor (GLP-1R), a 463 amino-acid member of the G protein-coupled receptor (GPCR) superfamily (see e.g., Drucker D J et al., Mol Endocrinol, 17(2):161-171, 2003). Bioactive GLP-1 can exist in two equipotent molecular forms: GLP-1(7-37) and GLP-1(7-36) amide. Bioactive GLP-1 can be rapidly cleaved by diaminopeptidyl peptidase-4 (DPP-4), which can result in generation of the largely inactive GLP-1(9-37) and GLP-1(9-36) amide molecular forms. In some cases, the majority of GLP-1 leaving the intestinal venous circulation has already been cleaved by DPP-4 expressed in capillaries surrounding gut L cells. The in vivo half-life of GLP-1 has been estimated as 1-2 minutes (see e.g., Drucker D J, Gastroenterology, 122(2):531-544, 2002).

GLP-1 can refer to Glucagon-like peptide 1 from any source, including isolated, purified and/or recombinant GLP-1 produced from any source or chemical synthesis, for example using solid phase synthesis. For example, the GLP-1 can have the sequence of SEQ ID NO: 26. Also included herein are conserved amino acid substitutions of native GLP-1. For example, conservative amino acid changes may be made, which although they alter the primary sequence of the protein or peptide, do not normally alter its function. Many molecular derivatives of GLP-1 have been disclosed, including some of which are reported to have agonist activity and/or to have a longer half-life than native GLP-1, see e.g., U.S. Pat. Nos. 6,358,924; 6,344,180; 6,284,725; 6,277,819; 6,271,241; 6,268,343; and 6,191,102, the contents of which are incorporated herein by reference.

GLP-1 related molecules, e.g., proteins have also been disclosed and reported to be capable of inducing pancreatic endocrine differentiation, islet proliferation and an increase in (3-cell mass (Parkes et al., Metabolism 50:583, 2001). Exendin-4 (an example of which is SEQ ID NO: 14 or SEQ ID NO: 11, also known as Exenatide or Byetta®) and exendin-3 (an example of which is SEQ ID NO: 28) are 39 amino acid peptides (differing at residues 2 and 3) which are approximately 53% homologous to GLP-1 and have insulinotropic activity. Many molecular derivatives of exendin-3 and exendin-4 have been disclosed, including some of which are reported to have agonist activity, see, e.g., U.S. Pat. Nos. 5,424,286; 6,268,343; 6,384,016; 6,458,924; 6,858,576; 6,989,366; 7,115,569; 7,153,825; 7,223,725; 7,235,627; 7,297,761; 7,419,952; 7,521,423; 7,696,161; 7,700,549; 8,097,698; 8,853,160; 8,889,619; 9,012,398; US 20120283179; US 20140206608; US 20140206609; 20140221281; US 20140213513; US 20150164997; and U.S. Pat. No. 9,181,305, the contents of which are incorporated herein by reference. The GLP-1 agonist can be an exendin-4 analog which potently activates the GLP-1 and GIP receptor and optionally the glucagon receptor, and comprise, among other substitutions, a modification of Tyr at position 1 and a modification of Ile at position 12. Examples of such analogs contemplated for use include those described in, e.g., US 20140206608; US 20140206609; 20140221281; and US 20140213513, of which the contents of each is incorporated herein by reference.

The GLP-1 agonist can be e.g., exenatide (tradename Byetta®, Amylin/Astrazeneca, SEQ ID NO: 14, SEQ ID NO: 11); lixisenatide (tradenames Adlyxin®, and Lyxumia®, Sanofi, SEQ ID NO: 15); liraglutide (tradename Victoza®, Novo Nordisk A/S, SEQ ID NO: 16); semaglutide (tradename Ozempic®, Novo Nordisk A/S, SEQ ID NO: 30); albiglutide (tradename Tanzeum®, GlaxoSmithKline; GLP-1 dimer fused to albumin); and dulaglutide (tradename Trulicity®, Eli Lilly, SEQ ID NO: 17).

In some cases, the payload can be glucose-dependent insulinotropic polypeptide. Glucose-dependent insulinotropic polypeptide (also referred to as gastric inhibitory polypeptide; GIP) can be a member of the incretin class of molecules. GIP can be derived from a 153-amino acid proprotein encoded by the GIP gene and circulates as a biologically active 42-amino acid peptide comprising the amino acid sequence set forth in SEQ ID NO: 36. GIP can be synthesized by intestinal K cells, which can be found in the mucosa of the duodenum and the jejunum of the gastrointestinal tract. GIP receptors can be seven-transmembrane proteins found on beta-cells in the pancreas. Various GIP antagonists can inhibit the GIP-dependent release of insulin in vivo and can also enhance glucose tolerance in an oral glucose tolerance test. As such, the GIP antagonists can be used in methods of treatment for T2D (see, e.g., US 20070167363, the contents of which is incorporated by reference herein). A GIP receptor (GIPR) agonist in the form of a truncated GIP analog (amino acid residues 1-30 of GIP) and having a substitution of a D-alanine (Ala) at position 2 of SEQ ID NO: 38, referred to as D-Ala2-GIP1-30 (D-GIP1-30) and comprising the amino acid sequence set forth in SEQ ID NO: 37 can exhibit anti-diabetic actions, without pro-obesity effects (see e.g., Widenmaier et al, PloS ONE, March 2010|Volume 5|Issue 3|e9590). The glucose-regulating agent, e.g., glucose-lowering agent can be a GIPR agonist comprising the amino acid sequence set forth in SEQ ID NO: 60. The payload can be glucagon.

### Multi-Specific Peptide Agonists

In some cases, a composition provided herein, e.g., a particle or a delivery construct, can be designed to effect dual activation of the GLP-1 and GIP receptors, e.g. by combining the actions of GLP-1 and GIP in one preparation. An embodiment of a delivery construct is a composition with a carrier, or a composition with a carrier and a payload. This can lead to a therapy with significantly better reduction of blood glucose levels, increased insulin secretion and reduced body weight in mice with type 2 diabetes and obesity compared to the marketed GLP-1 agonist liraglutide (see e.g. V A Gault et al., Clin Sci (Lond), 121, 107-117, 2011).

In some cases, a carrier provided herein is coupled to a dual agonist, e.g., Tirzepatide (Eli Lilly, SEQ ID NO: 33) or SAR425899 (Sanofi).

A composition, e.g., a delivery construct, provided herein can activate the receptors for GLP-1, GIP, and glucagon. A carrier provided herein can be coupled to the GGG Tri-Agonist under investigation by Eli Lilly.

A carrier provided herein can be coupled to other peptides under investigation for the treatment of diabetes and obesity, including the Dual Amylin Calcitonin Receptor Agonist DACRA-089 (SEQ ID NO: 41, also known as KBP-089, Sanofi).

### Insulin and Insulin Analogs

A payload provided herein can be insulin, or an insulin analog, or a derivative of insulin. As used herein, the terms “insulin analog” or “insulin derivative” can refer to a polypeptide which has a molecular structure which formally can be derived from the structure of a naturally occurring insulin, for example that of human insulin (an example of which is insulin A-chain SEQ ID NO: 43 and insulin B-chain SEQ ID NO: 44, which can be linked by disulfide bonds), by deleting and/or exchanging at least one amino acid residue occurring in the naturally occurring insulin and/or adding at least one amino acid residue. The added and/or exchanged amino acid residue can either be codable amino acid residues or other naturally occurring residues or purely synthetic amino acid residues.

Insulin and insulin analogs/derivatives have been extensively described in the art (see e.g., US20150216981; U.S. Pat. Nos. 9,265,723; 8,633,156; 8,410,048; 8,048,854; 7,713,930; 7,696,162; 7,659,363; 7,291,132; 7,193,035; and references cited therein, which are all incorporated by reference herein). The insulin can have the native sequence of human insulin, with an A chain (SEQ ID NO: 43) and a B chain (SEQ ID NO: 44) linked by disulfide bonds.

In certain embodiments, the insulin comprises an A chain having the amino acid sequence set forth in SEQ ID NO: 43 and a B chain having the amino acid sequence set forth in SEQ ID NO: 44. Additional embodiments of insulin with beneficial fast-acting or slow-acting (basal) properties include Insulin Aspart having an A chain of SEQ ID NO: 45 and a B chain of SEQ ID NO: 46; Insulin glargine having an A chain of SEQ ID NO: 47 and a B chain of SEQ ID NO: 48; and Insulin lispro having an A chain of SEQ ID NO: 49 and a B chain of SEQ ID NO: 50.

The one or more payloads, e.g., heterologous payloads, can be an agent for the treatment of hemophilia, e.g., hemophilia A or hemophilia B. An agent for the treatment of hemophilia can be clotting factor VIII (e.g., a clotting factor VIII concentrate), clotting factor IX (e.g., clotting factor IX concentrate), factor VIIa, Hemlibra® (ACE 910 or emicizumab), DDAVP® or Stimate® (Desmopressin Acetate), an antifibrinolytic, e.g., Amicar® (Epsilon Amino Caproic Acid) or Lysteda® (tranexamic acid), ELOCTATE® [Antihemophilic factor (recombinant), Fc fusion protein] or Cryoprecipitate.

In some cases, the payload, e.g., heterologous payload, e.g., glucose regulating agent, e.g., glucose lowering agent is insulin or an insulin analog. In some cases, the payload, e.g., heterologous payload, e.g., glucose regulating agent, e.g., glucose lowering agent is exenatide. Exenatide (SEQ ID NO: 11) can be a peptide having GLP-1-like biological activity that is stabilized by a C-terminal amine and an N-terminal H.

In some cases, the one or more payloads, e.g., one or more heterologous payloads, used herein can include antineoplastic compounds (such as chemotherapy or anti-tumor agents), such as nitrosoureas, e.g., carmustine, lomustine, semustine, strepzotocin; methylhydrazines, e.g., procarbazine, dacarbazine; steroid hormones, e.g., glucocorticoids, estrogens, progestins, androgens, tetrahydrodesoxycaricosterone; immunoactive compounds such as immunosuppressives, e.g., pyrimethamine, trimethopterin, penicillamine, cyclosporine, azathioprine; and immunostimulants, e.g., levamisole, diethyl dithiocarbamate, enkephalins, endorphins; antimicrobial compounds such as antibiotics, e.g., beta.-lactam, penicillin, cephalosporins, carbapenims and monobactams, beta.-lactamase inhibitors, aminoglycosides, macrolides, tetracyclins, spectinomycin; antimalarials, amebicides; antiprotazoals; antifungals, e.g., amphotericin-beta, antivirals, e.g., acyclovir, idoxuridine, ribavirin, trifluridine, vidarbine, gancyclovir; parasiticides; antihalmintics; radiopharmaceutics; gastrointestinal drugs; hematologic compounds; immunoglobulins; blood clotting proteins, e.g., anti-hemophilic factor, factor IX complex; anticoagulants, e.g., dicumarol, heparin Na; fibrolysin inhibitors, e.g., tranexamic acid; cardiovascular drugs; peripheral anti-adrenergic drugs; centrally acting antihypertensive drugs, e.g., methyldopa, methyldopa HCL; antihypertensive direct vasodilators, e.g., diazoxide, hydralazine HCL; drugs affecting renin-angiotensin system; peripheral vasodilators, e.g., phentolamine; anti-anginal drugs; cardiac glycosides; inodilators, e.g., amrinone, milrinone, enoximone, fenoximone, imazodan, sulmazole; antidysrhythmics; calcium entry blockers; drugs affecting blood lipids, e.g., ranitidine, bosentan, rezulin; respiratory drugs; sypathomimetic drugs, e.g., albuterol, bitolterol mesylate, dobutamine HCL, dopamine HCL, ephedrine So, epinephrine, fenfluramine HCL, isoproterenol HCL, methoxamine HCL, norepinephrine bitartrate, phenylephrine HCL, ritodrine HCL; cholinomimetic drugs, e.g., acetylcholine HCl; anticholinesterases, e.g., edrophonium Cl; cholinesterase reactivators; adrenergic blocking drugs, e.g., acebutolol HCl, atenolol, esmolol HCl, labetalol HCl, metoprolol, nadolol, phentolamine mesylate, propanolol HCl; antimuscarinic drugs, e.g., anisotropine methylbromide, atropine, clinidium Br, glycopyrrolate, ipratropium Br, scopolamine HBr; neuromuscular blocking drugs; depolarizing drugs, e.g., atracurium besylate, hexafluorenium Br, metocurine iodide, succinylcholine Cl, tubocurarine Cl, vecuronium Br; centrally acting muscle relaxants, e.g., baclofen; neurotransmitters and neurotransmitter agents, e.g., acetylcholine, adenosine, adenosine triphosphate; amino acid neurotransmitters, e.g., excitatory amino acids, GABA, glycine; biogenic amine neurotransmitters, e.g., dopamine, epinephrine, histamine, norepinephrine, octopamine, serotonin, tyramine; neuropeptides, nitric oxide, K.sup.+channel toxins; antiparkinson drugs, e.g., amaltidine HCl, benztropine mesylate, carbidopa; diuretic drugs, e.g., dichlorphenamide, methazolamide, bendroflumethiazide, polythiazide; antimigraine drugs, e.g, carboprost tromethamine mesylate, doxorubicin, mitomycin, cisplatin, daunorubicin, bleomycin, actinomycin D, neocarzinostatin, and methysergide maleate.

In some cases, the one or more payloads, e.g., one or more heterologous payloads, contemplated to be used with the methods of this disclosure include lymphokine inhibitory factor, macrophage colony stimulating factor, platelet derived growth factor, stem cell factor, tumor growth factor-β, tumor necrosis factor, lymphotoxin, Fas, granulocyte colony stimulating factor, granulocyte macrophage colony stimulating factor, interferon-α, interferon-β, interferon-γ, growth factors and protein hormones such as erythropoietin, angiogenin, hepatocyte growth factor, fibroblast growth factor, keratinocyte growth factor, nerve growth factor, tumor growth factor-α, thrombopoietin, thyroid stimulating factor, thyroid releasing hormone, neurotrophin, epidermal growth factor, VEGF, ciliary neurotrophic factor, LDL, somatomedin, insulin growth factor, insulin-like growth factor I and II, chemokines such as ENA-78, ELC, GRO-α, GRO-β, GRO-γ, HRG, LEF, IP-10, MCP-1, MCP-2, MCP-3, MCP-4, MIP-1-α, MIP-1-β, MG, MDC, NT-3, NT-4, SCF, LIF, leptin, RANTES, lymphotactin, eotaxin-1, eotaxin-2, TARC, TECK, WAP-1, WAP-2, GCP-1, GCP-2; α-chemokine receptors, e.g., CXCR1, CXCR2, CXCR3, CXCR4, CXCR5, CXCR6, CXCR7; and β-chemokine receptors, e.g., CCR1, CCR2, CCR3, CCR4, CCR5, CCR6, and CCR7. Further examples of payloads include inhibitors of regulatory T cells (Tregs) such as Tregs that express CD4, CD25 and Foxp3, and Tregs such as Tr1, Th3, CD8+CD28-, Qa-1 restricted T cells, and IL-17 Treg cells.

In some cases, one or more payloads, e.g., one or more heterologous payloads, can be an E. coli heat-labile enterotoxin (Etx).

In some cases, one or more payloads, e.g., one or more heterologous payloads, used with the methods and compositions herein can be a dye or a radiopharmaceutical. The one or more dyes and radiopharmaceuticals can be Alexa488, fluorescent compounds, indium, technetium, magnetic particles, radiopaque materials, and red fluorescent protein (RFP).

In some cases, one or more payloads, e.g., one or more heterologous payloads, used with the methods and compositions disclosed herein can be a hormone. Examples of hormones include, but are not limited to, human growth hormone, NUTROPIN® (Genentech), HUMATROPE® (Lilly), GENOTROPIN® (Pfizer), NORDITROPIN® (Novo), SAIZEN® (Merck Serono, OMNITROPE® (Sandoz), SEROSTIM® (EMD Serono), ZORBITIVE® (Merck Serono), TEV-TROPIN® (Teva), pituitary hormones, e.g., chorionic gonadotropin, cosyntropin, menotropins, somatotropin, iorticotropin, protirelin, thyrotropin, vasopressin, lypressin; adrenal hormones, e.g., beclomethasone dipropionate, betamethasone, dexamethasone, triamcinolone; pancreatic hormones, e.g., glucagon, insulin; parathyroid hormone, e.g., dihydrochysterol; thyroid hormones, e.g., calcitonin etidronate disodium, levothyroxine Na, liothyronine Na, liotrix, thyroglobulin, teriparatide acetate; antithyroid drugs; estrogenic hormones; progestins and antagonists; hormonal contraceptives; testicular hormones; gastrointestinal hormones, e.g., cholecystokinin, enteroglycan, galanin, gastric inhibitory polypeptide, epidermal growth factor-urogastrone, gastric inhibitory polypeptide, gastrin-releasing peptide, gastrins, pentagastrin, tetragastrin, motilin, peptide YY, secretin, vasoactive intestinal peptide, or sincalide, somatotropin, synthetic human g hormone, synthetic human growth hormone partial, synthetic human growth hormone partial, human growth hormone 2, somatoliberin, appetite-regulating hormone, leptin, growth hormone receptor, growth hormone-releasing hormone receptor, growth hormone secretagogue receptor, growth hormone-releasing hormone receptor form a, and growth hormone receptor.

In further examples, the one or more payloads, e.g., one or more heterologous payloads, can be a cytokine. The one or more cytokines can be chemokines, interleukins, e.g., IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, and IL-30.

In further examples, the one or more payloads, e.g., one or more heterologous payloads, can be an anti-TNF agent. Examples of anti-TNF agents which can be used include anti-TNF antibodies, infliximab (Remicade), adalimumab (Humira), etanercept (ENBREL®), Tumor necrosis factor-a (“TNF-α”); NP_000585.2, lymphotoxin-a (“LT-a”), lymphotoxin-b (“LT-b”), CD30 ligand, CD40 ligand, CD70 ligand, OX40 ligand, 41BB ligand, Apo1 ligand (or FasL or CD95L), Apo2 ligand (or TRAIL, AIM-1 or AGP-1), Apo3 ligand (or TWEAK), APRIL, LIGHT, OPG ligand (or RANK ligand), BlyS (or THANK), BCMA, TACI, TNFR1, TNFR2, lymphotoxin-bR, CD40, CD95 (or FAS or APO-1), OPG, RANK, CD30, CD27, OX40 (or CD134), 41BB, NGFR, BCMA, TAC1, EDA2R, TROY, DR6, DR5 (or TRAILR2), DR4, DR3, HVEM, LTβR, GITR, DcR3, Fn14 (or TWEAKR), BAFF, Small Modular Immuno-Pharmaceuticals (SMIP), tetracyclines (e.g., tetracycline, doxycycline, lymecycline, oxytetracycline, minocycline), chemically modified tetracyclines (e.g., dedimethylamino-tetracycline), hydroxamic acid compounds, carbocyclic acids and derivatives, lazaroids, pentoxifylline, napthopyrans, amrinone, pimobendan, vesnarinone, phosphodiesterase inhibitors, and small molecule inhibitors of kinases. Small molecule kinase inhibitors include, without limitation, small molecule inhibitors of p38MAPK, COT, MK2, P13K, IKKa,b,g, MEKK1,2,3, IRAK1,4 and Akt kinase.

In some cases, the one or more payloads, e.g., one or more heterologous payloads, can be a tumor associated antigen. Examples of tumor associated antigens include Her2/neu, Her3, Her4, EGF, EGFR, CD2, CD3, CD5, CD7, CD13, CD19, CD20, CD21, CD23, CD30, CD33, CD34, CD38, CD46, CD55, CD59, CD69, CD70, CD71, CD97, CD117, CD127, CD134, CD137, CD138, CD146, CD147, CD152, CD154, CD195, CD200, CD212, CD223, CD253, CD272, CD274, CD276, CD278, CD279, CD309 (VEGFR2), DR6, PD-L1, Kv1.3, 5.00E+10, MUC1, uPA, SLAMF7 (CD319), MAGE 3, MUC 16 (CA-125), KLK3, K-ras, Mesothelin, p53, Survivin, G250 (Renal Cell Carcinoma Antigen), and PSMA.

In some cases, the one or more payloads, e.g., one or more heterologous payloads, can be an enzyme such as hyaluronidase, streptokinase, tissue plasminogen activator, urokinase, PGE-adenosine deaminase; intravenous anesthetics such as droperidol, etomidate, fetanyl citrate/droperidol, hexobarbital, ketamine HCl, methohexital Na, thiamylal Na, thiopental Na; antiepileptics, e.g., carbamazepine, clonazepam, divalproex Na, ethosuximide, mephenyloin, paramethadione, phenyloin, primidone. In various embodiments, the biologically active cargo is an enzyme selected from hyaluronidase, streptokinase, tissue plasminogen activator, urokinase, or PGE-adenosine deaminase.

### V. Coupling of the Payload or Cation to a Carrier

In some cases, the compositions provided herein comprise a carrier coupled to a payload, e.g., a heterologous payload. The payload, e.g., heterologous payload, can be coupled to the carrier by any method known by one of skill in the art without limitation. The payload may associate with the carrier by non-covalent interactions such as ionic interactions or assembly into nano-particles. The payload may be chemically cross-linked to the carrier via covalent interactions. In some cases, the one or more payloads are fused to a carrier. In a fusion molecule the one or more payloads or one or more cations of the fusion molecule can be attached to the remainder of the fusion molecule by any method known by one of skill in the art without limitation. The payload or cation can be introduced into any portion of the fusion molecule that does not disrupt the cell-binding or transcytosis activity of the carrier. In various embodiments, the payload or cation is directly coupled to the N-terminus or C-terminus of the carrier. In various embodiments, the payload or cation can be connected with a side chain of an amino acid of the carrier. The payload can be indirectly coupled to the carrier via a spacer or linker. In various embodiments, the payload or cation is coupled to the carrier with a cleavable linker such that cleavage at the cleavable linker(s) separates the payload or cation from the remainder of the fusion molecule. In various embodiments, the payload or cation is a polypeptide that can also comprise a short leader peptide that remains attached to the polypeptide following cleavage of the cleavable linker. For example, the payload or cation can comprise a short leader peptide of greater than 1 amino acid, greater than 5 amino acids, greater than 10 amino acids, greater than 15 amino acids, greater than 20 amino acids, greater than 25 amino acids, greater than 30 amino acids, greater than 50 amino acids, or greater than 100 amino acids. In some cases, biological active payload can comprise a short leader peptide of less than 100 amino acids, less than 50 amino acids, less than 30 amino acids, less than 25 amino acids, less than 20 amino acids, less than 15 amino acids, less than 10 amino acids, or less than 5 amino acids. In some cases, payload or cation can comprise a short leader peptide of between 1-100 amino acids, between 5-10 amino acids, between 10 to 50 amino acids, or between 20 to 80 amino acids.

In embodiments where the payload or cation is expressed together with another sequence as a fusion protein, the payload or cation can be inserted into the fusion molecule by any method known to one of skill in the art without limitation. For example, nucleic acids coding for amino acids corresponding to the payload or cation can be directly inserted into the nucleic acid coding for the other moiety or fusion molecule, with or without deletion of native amino acid sequences.

In embodiments where the payload or cation is not expressed together as a fusion protein, the payload or cation can be connected by any suitable method known by one of skill in the art, without limitation. More specifically, the exemplary methods described above for connecting a receptor-binding domain to the remainder of the molecule are equally applicable for connecting the payload or cation to the remainder of the molecule.

### VI. Production of Nucleic Acids Encoding Carriers and/or Payloads

In various embodiments, the carriers, payloads, and/or non-naturally occurring delivery constructs, e.g., fusion molecule of the present disclosure are prepared using the methodology described in, e.g., U.S. Pat. Nos. 9,090,691 and 7,713,737, each incorporated by reference herein in their entirety.

In various embodiments, the carriers, payloads, and/or non-naturally occurring fusion molecules are synthesized using recombinant DNA methodology. Generally this can involve creating a DNA sequence that encodes the carrier, payload, and/or fusion molecule, placing the DNA in an expression cassette under the control of a particular promoter, expressing the molecule in a host, isolating the expressed molecule and, if required, folding of the molecule into an active conformational form.

DNA encoding the carrier, payload, and/or fusion molecules described herein can be prepared by any suitable method, including, for example, cloning and restriction of appropriate sequences or direct chemical synthesis by methods such as the phosphotriester method of Narang et al. (1979) Meth. Enzymol. 68: 90-99; the phosphodiester method of Brown et al. (1979) Meth. Enzymol. 68: 109-151; the diethylphosphoramidite method of Beaucage et al. (1981) Tetra. Lett., 22: 1859-1862); the solid support method of U.S. Pat. No. 4,458,066, and the like.

Chemical synthesis can produce a single stranded oligonucleotide. This can be converted into double stranded DNA by hybridization with a complementary sequence or by polymerization with a DNA polymerase using the single strand as a template. Chemical synthesis can be used to generated DNA sequences of about 100 bases. Longer sequences can be obtained by the ligation of shorter sequences.

Alternatively, subsequences can be cloned and the appropriate subsequences cleaved using appropriate restriction enzymes. The fragments can then be ligated to produce the desired DNA sequence.

In various embodiments, DNA encoding carrier, payload, and/or fusion molecules of the present disclosure can be cloned using DNA amplification methods such as polymerase chain reaction (PCR). Thus, for example, the gene or genes for the one or more payloads, e.g. the one or more biologically-active payloads, is PCR amplified, using a sense primer containing the restriction site for, e.g., NdeI and an antisense primer containing the restriction site for HindIII. This can produce one or more nucleic acids encoding the one or more payload sequences and having terminal restriction sites. A carrier having “complementary” restriction sites can similarly be cloned and then ligated to the one or more nucleic acids encoding the one or more payloads and/or to a linker attached to the one or more nucleic acids encoding the one or more payloads. Ligation of the nucleic acid sequences and insertion into a vector produces a vector encoding the one or more payloads joined to the carrier/s.

### VII. Cleavable Linkers

In various embodiments, the one or more payloads, e.g., heterologous payloads, to be delivered to the subject is coupled to the carrier using one or more cleavable linkers. The number of cleavable linkers present in the fusion molecule depends, at least in part, on the location of the one or more payloads in relation to the carrier and the nature of the biologically active payload. When the one or more payloads can be separated from the remainder of the fusion molecule with cleavage at a single linker, the fusion molecules can comprise a single cleavable linker. Further, where the one or more payloads is, e.g., a dimer or other multimer, each subunit of the one or more payloads can be separated from the remainder of the fusion molecule and/or the other subunits of the one or more payloads by cleavage at the cleavable linker.

In various embodiments, the cleavable linkers are cleaved by a cleaving enzyme that is present at or near the basolateral membrane of an epithelial cell. By selecting the cleavable linker to be cleaved by such enzymes, the one or more payloads can be liberated from the remainder of the fusion molecule following transcytosis across the mucous membrane and release from the epithelial cell into the cellular matrix on the basolateral side of the membrane. Further, cleaving enzymes can be used that are present inside the epithelial cell, such that the cleavable linker is cleaved prior to release of the fusion molecule from the basolateral membrane, so long as the cleaving enzyme does not cleave the fusion molecule before the fusion molecule enters the trafficking pathway in the polarized epithelial cell that results in release of the fusion molecule and one or more payloads from the basolateral membrane of the cell.

In various embodiments, the cleavable linker exhibits a greater propensity for cleavage than the remainder of the delivery construct. As one skilled in the art is aware, many peptide and polypeptide sequences can be cleaved by peptidases and proteases. In various embodiments, the cleavable linker is selected so that it will be preferentially cleaved relative to other amino acid sequences present in the delivery construct during administration of the delivery construct. In various embodiments, the receptor-binding domain is substantially (e.g., about 99%, about 95%, about 90%, about 85%, about 80, or about 75%) intact following delivery of the delivery construct to the bloodstream of the subject. In various embodiments, the transcytosis activity is substantially (e.g., about 99%, about 95%, about 90%, about 85%, about 80, or about 75%) intact following delivery of the delivery construct to the bloodstream of the subject. In various embodiments, the macromolecule is substantially (e.g., about 99%, about 95%, about 90%, about 85%, about 80, or about 75%) intact following delivery of the delivery construct to the bloodstream of the subject. In various embodiments, the cleavable linker is substantially (e.g., about 99%, about 95%, about 90%, about 85%, about 80, or about 75%) cleaved following delivery of the delivery construct to the bloodstream of the subject.

In other embodiments, a cleaving enzyme found in the plasma of the subject can be used to cleave the cleavable linker. Any cleaving enzyme known by one of skill in the art to be present in the plasma of the subject can be used to cleave the cleavable linker.

In various embodiments, the cleavable linker is cleaved by a cleaving enzyme found in the plasma of the subject. Any cleaving enzyme known by one of skill in the art to be present in the plasma of the subject can be used to cleave the cleavable linker. In some cases, plasma cleaving enzymes can be used to cleave the delivery constructs. In other embodiments, the cleavable linker comprises a nucleic acid, such as RNA or DNA. In still other embodiments, the cleavable linker comprises a carbohydrate, such as a disaccharide or a trisaccharide.

In various embodiments, the cleavable linker can be a cleavable linker that is cleaved following a change in the environment of the fusion molecule. For example, the cleavable linker can be a cleavable linker that is pH sensitive and is cleaved by a change in pH that is experienced when the fusion molecule is released from the basal-lateral membrane of a polarized epithelial cell. For instance, the intestinal lumen can be strongly alkaline, while plasma can be essentially neutral. Thus, a cleavable linker can be a moiety that is cleaved upon a shift from alkaline to neutral pH. The change in the environment of the fusion molecule that cleaves the cleavable linker can be any environmental change that that is experienced when the fusion molecule is released from the basal-lateral membrane of a polarized epithelial cell known by one of skill in the art, without limitation.

### VIII. Non-Cleavable Linkers

In various embodiments, the carrier and one or more payloads can be separated by a linker. When a linker is used, a linker can include one or more amino acids. Examples of linkers contemplated herein include sequences such as S, (GS)x (SEQ ID NO: 85), (GGS)x (SEQ ID NO: 86), (GGGS)x (SEQ ID NO: 87), (GGGGS)x (SEQ ID NO: 88), or (GGGGGS)x (SEQ ID NO: 89), wherein x=1, 2, 3, 4, 5, 6, 7, 8, 9, 10, 11, 12, 13, 14, or 15. In some cases, a linker does not include a terminal S residue, e.g., SEQ ID NO: 4 (GGGGSGGGGSGGGG). Generally, a linker can have no specific biological activity other than to join the proteins or to preserve some minimum distance or other spatial relationship between them. In various embodiments, however, the constituent amino acids of the linker can be selected to influence some property of the molecule such as the folding, net charge, or hydrophobicity.

In various embodiments, the linker is capable of forming covalent bonds to both the carrier and to the biologically active payload. Suitable linkers include straight or branched-chain carbon linkers, heterocyclic carbon linkers, or peptide linkers. In various embodiments, the linker(s) can be joined to the constituent amino acids of the carrier and/or the one or more payloads through their side groups (e.g., through a disulfide linkage to cysteine). In various embodiments, the linkers are joined to the alpha carbon amino and/or carboxyl groups of the terminal amino acids of the carrier and/or the biologically active payload.

A bifunctional linker having one functional group reactive with a group on the carrier and another group reactive on the one or more payloads can be used to form the desired conjugate. Alternatively, derivatization can involve chemical treatment of the targeting moiety. Procedures for generation of, for example, free sulfhydryl groups on polypeptides, such as antibodies or antibody fragments, are known (See U.S. Pat. No. 4,659,839).

Many procedures and linker molecules for attachment of various compounds including radionuclide metal chelates, carriers and drugs to proteins such as antibodies are known. See, for example, European Patent Application No. 188,256; U.S. Pat. Nos. 4,671,958, 4,659,839, 4,414,148, 4,699,784; 4,680,338; 4,569,789; and 4,589,071; and Borlinghaus et al. (1987) Cancer Res. 47: 4071-4075.

### IX. Chemical Conjugation or Complexation of the Payload or Cation to the Carrier

In various embodiments, the payload to be delivered to the subject is chemically conjugated to the carrier. Means of chemically conjugating molecules are well known to those of skill.

The procedure for conjugating two molecules varies according to the chemical structure of the agent. Polypeptides typically contain variety of functional groups; e.g., carboxylic acid (COOH) or free amine (—NH2) groups, that are available for reaction with a suitable functional group on the other peptide, or on a linker to join the molecules thereto.

In various embodiments of the present disclosure, isolated carriers are prepared by bacterial fermentation and purified by established methods. The purified carrier is then modified at its C-terminus to allow direct chemical coupling through a free sulfhydryl residue located near the C-terminus of the protein. The C-terminal modification can include a cysteine-constrained loop harboring the consensus cleavage sequence ENLFQS (SEQ ID NO: 90) for the highly selective protease from the tobacco etch virus (TEV), a second cysteine, and a hexa-histidine (His6) tag (SEQ ID NO: 91) (e.g. SEQ ID NO: 77 and SEQ ID NO: 78). The second Cys is included to form a disulphide bridge with the Cys ultimately used for coupling. Adding a His6 sequence (SEQ ID NO: 91) to the protein can simplify the purification and the TEV cleavage sequence provides a mechanism to selectively remove the terminal Cys residue following mild reduction. TEV cleavage and mild reduction with 0.1 mM dithiotheitol following expression and isolation of the non-toxic bacterial toxin constructs allows for the direct chemical coupling of a glucose-regulating agent, e.g., glucose-lowering agent via a maleimide-based reaction as a generic mechanism of cargo attachment. Following TEV protease cleavage, reduction, and cargo coupling through a maleimide reaction with the free sulfhydryl, removal of the freed C-terminal sequence was achieved by a second Ni2+ column chromatography step.

In certain embodiments, the delivery construct comprises particles which are decorated covalently with the carrier, and wherein the payload is integrated into the particles. In certain embodiments, the particles can be smaller than ˜150 nm in diameter, smaller than ˜100 nm, or smaller than ˜50 nm.

### Transcytosis Testing

The function of the carrier can be tested based on its ability to pass the carrier, or a payload associated with the carrier, .e.g, in a particle, through an epithelial membrane. Because transcytosis can first require binding to the cell, these assays can also be used to assess the function of the cell recognition domain.

The transcytosis activity can be tested by any method known by one of skill in the art, without limitation. Transcytosis activity can be tested by assessing the ability of a composition, particle, to enter a non-polarized cell to which it binds. The same property that allows a carrier to pass through a polarized epithelial cell may also allow molecules bearing the carrier to enter non-polarized cells. Thus, the composition's ability to enter the cell can be assessed, for example, by detecting the physical presence of the composition (e.g., carrier or payload) in the interior of the cell. For example, the composition can be labeled with, for example, a fluorescent marker, and the delivery exposed to the cell. Then, the cells can be washed, removing any composition (e.g., carrier or payload, e.g., delivery construct that has not entered the cell, and the amount of label remaining determined. Detecting the label in this traction indicates that the composition has entered the cell.

The composition's transcytosis ability can be tested by assessing the carrier or payload (e.g., delivery construct's) ability to pass through a polarized epithelial cell. For example, the composition can be labeled with, for example, a fluorescent marker and contacted to the apical membranes of a layer of epithelial cells. Fluorescence detected on the basal-lateral side of the membrane formed by the epithelial cells indicates that the carrier is functioning properly.

### Cleavable Linker Cleavage Testing

The function of the cleavable linker can generally be tested in a cleavage assay. Any suitable cleavage assay known by one of skill in the art, without limitation, can be used to test the cleavable linkers. Both cell-based and cell-free assays can be used to test the ability of an enzyme to cleave the cleavable linkers.

An exemplary cell-free assay for testing cleavage of cleavable linkers comprises preparing extracts of polarized epithelial cells and exposing a labeled fusion molecule bearing a cleavable linker to the fraction of the extract that corresponds to membrane-associated enzymes. In such assays, the label can be attached to either the glucose-regulating agent, e.g., glucose-lowering agent to be delivered or to the remainder of the fusion molecule. Among these enzymes are cleavage enzymes found near the basal-lateral membrane of a polarized epithelial cell, as described above. Cleavage can be detected, for example, by binding the fusion molecule with, for example, an antibody and washing off unbound molecules. If label is attached to the glucose-regulating agent, e.g., glucose-lowering agent to be delivered, then little or no label should be observed on the molecule bound to the antibodies. Alternatively, the binding agent used in the assay can be specific for the glucose-regulating agent, e.g., glucose-lowering agent, and the remainder of the construct can be labeled. In either case, cleavage can be assessed.

Cleavage can also be tested using cell-based assays that test cleavage by polarized epithelial cells assembled into membranes. For example, a labeled fusion molecule, or portion of a fusion molecule comprising the cleavable linker, can be contacted to either the apical or basolateral side of a monolayer of suitable epithelial cells, such as, for example, Coco-2 cells, under conditions that permit cleavage of the linker. Cleavage can be detected by detecting the presence or absence of the label using a reagent that specifically binds the fusion molecule, or portion thereof. For example, an antibody specific for the fusion molecule can be used to bind a fusion molecule comprising a label distal to the cleavable linker in relation to the portion of the fusion molecule bound by the antibody. Cleavage can then be assessed by detecting the presence of the label on molecules bound to the antibody. If cleavage has occurred, little or no label should be observed on the molecules bound to the antibody. By performing such experiments, enzymes that preferentially cleave at the basolateral membrane rather than the apical membrane can be identified, and, further, the ability of such enzymes to cleave the cleavable linker in a fusion molecule can be confirmed.

Further, cleavage can also be tested using a fluorescence reporter assay as described in U.S. Pat. No. 6,759,207. Briefly, in such assays, the fluorescence reporter can be contacted to the basolateral side of a monolayer of suitable epithelial cells under conditions that allow the cleaving enzyme to cleave the reporter. Cleavage of the reporter changes the structure of the fluorescence reporter, changing it from a non-fluorescent configuration to a fluorescent configuration. The amount of fluorescence observed can indicate the activity of the cleaving enzyme present at the basolateral membrane.

Further, cleavage can also be tested using an intra-molecularly quenched molecular probe, such as those described in U.S. Pat. No. 6,592,847. Such probes generally comprise a fluorescent moiety that emits photons when excited with light of appropriate wavelength and a quencher moiety that absorbs such photons when in close proximity to the fluorescent moiety. Cleavage of the probe separates the quenching moiety from the fluorescent moiety, such that fluorescence can be detected, thereby indicating that cleavage has occurred. Thus, such probes can be used to identify and assess cleavage by particular cleaving enzymes by contacting the basolateral side of a monolayer of suitable epithelial cells with the probe under conditions that allow the cleaving enzyme to cleave the probe. The amount of fluorescence observed indicates the activity of the cleaving enzyme being tested.

### X. Methods of Use

The methods and compositions, e.g., pharmaceutical compositions, of the present disclosure can be used to treat diseases or conditions, e.g., medical conditions. The methods and compositions can be amenable for oral and/or intra-nasal formulation and delivery. The disease or condition can be an immunologic disease, a metabolic disease, or a central nervous system (CNS) disease. “Metabolic diseases or disorders” can refer to a combination of medical disorders that, when occurring together, increase the risk of diabetes and atherosclerotic vascular disease, e.g. heart disease and stroke. Defining medical parameters for the metabolic syndrome include diabetes mellitus, impaired glucose tolerance, raised fasting glucose, insulin resistance, urinary albumin secretion, central obesity, hypertension, elevated triglycerides, elevated LDL cholesterol and reduced HDL cholesterol.

The methods and compositions, e.g., pharmaceutical compositions, provided herein can be used to treat a neural condition, a condition related to immunology, and a condition related to endocrinology, a condition related to immuno-neurology, a condition related to neuro-endocrinology, or a condition related to immuno-endocrinology. The methods and compositions, e.g., pharmaceutical compositions, provided herein can be used to treat a cardiovascular condition, a rare disease, liver disease, inflammatory bowel disorder, respiratory condition, neurological condition, or a gastrointestinal condition, A composition provided herein can be a vaccine. The diseases or conditions include, e.g., viral disease or infections, cancer, metabolic diseases, obesity, autoimmune diseases, inflammatory diseases, allergy, graft-vs-host disease, systemic microbial infection, anemia, cardiovascular disease, psychosis, genetic diseases, neurodegenerative diseases, disorders of hematopoietic cells, diseases of the endocrine system or reproductive systems, gastrointestinal diseases. Further examples of diseases include, but are not limited to, diabetes, diabetes as a consequence of obesity, hyperglycemia, dyslipidemia, hypertriglyceridemia, syndrome X, insulin resistance, impaired glucose tolerance (IGT), diabetic dyslipidemia, hyperlipidemia, fatty liver disease, nonalcoholic steatohepatitis (NASH), hepatitis, Obesity, vascular disease, heart disease, stroke, impaired glucose tolerance, raised fasting glucose, insulin resistance, urinary albumin secretion, central obesity, hypertension, elevated triglycerides, elevated LDL cholesterol and reduced HDL cholesterol, hyperglycemia, hyperinsulinemia, dyslipidemia, ketosis, hypertriglyceridemia, syndrome X, insulin resistance, impaired fasting glucose, impaired glucose tolerance (IGT), diabetic dyslipidemia, gluconeogenesis, excess glycogenolysis, diabetic ketoacidosis, hypertriglyceridemia, hypertension, diabetic nephropathy, renal insufficiency, renal failure, hyperphagia, muscle wasting, diabetic neuropathy, diabetic retinopathy, diabetic coma, arteriosclerosis, coronary heart disease, peripheral artery disease, fibrosis, and hyperlipidemia. The disease or condition can be ulcerative colitis, Crohn's disease, pouchitis, psoriatic arthritis, rheumatoid arthritis, or psoriasis. The disease or condition can be a gastroenterological condition, e.g., short bowel syndrome (SBS). The disease or condition can be a growth hormone deficiency. In some cases, a composition provided herein comprises GLP-2, and the composition is administered, e.g., orally, to a subject with a gastroenterological condition, e.g., short bowel syndrome (SBS). In some cases, a composition provided herein comprises GLP-1, and the composition is administered, e.g., orally, to a subject to treat a metabolic disease; the composition can be formulated for oral delivery for local gastrointestinal and/or systemic exposure. In some cases, a composition provided herein comprises GLP-1 or a GLP-1 analog, and the composition is provided, e.g., orally, to a subject to treat a metabolic disorder (e.g., diabetes, obesity), or non-alcoholic steatohepatitis (NASH), or a central nervous system (CNS) condition, The composition can be formulated for oral delivery. In some cases, a composition provided herein comprises human growth hormone, and the composition is provided to a subject with a growth hormone deficiency or a related disorder to treat the growth hormone deficiency or related disorder. The composition can be formulated for oral delivery, and the target location for the payload can be systemic exposure. In some cases, the payload can reach the liver. In some cases, a composition provided herein comprises an incretin. In some cases, a composition provided herein is administered to a subject to regulate glycemic function.

In many chronic diseases, oral and/or intra-nasal formulations of the disclosure can be particularly useful because they can allow long-term patient care and therapy via home administration without reliance on injectable treatment or drug protocols. Formulations of this disclosure may be administered by oral administration, pulmonary administration, intra-nasal administration, buccal administration, or sublingual administration. Thus, in another aspect, the present disclosure relates to the use of a pharmaceutical composition which is a self-assembling particle, e.g., microparticle containing a carrier, payload (e.g., heterologous payload), and/or non-naturally occurring fusion molecule as a drug substance in a pill or tablet for oral delivery of a one or more payloads in the treatment of diseases and conditions for which use of the one or more payloads contained in such formulations is indicated.

The pharmaceutical compositions which are particles, e.g., microparticles, comprising carrier, payload (e.g., heterologous payload), and/or fusion molecules of the present disclosure can offer several advantages over conventional techniques for local or systemic delivery of macromolecules to a subject. Foremost among such advantages is the ability to deliver the one or more payloads to a subject without using a needle to puncture the skin of the subject. Many subjects require repeated, regular doses of macromolecules. Such subjects' quality of life would be greatly improved if the delivery of a macromolecule can be accomplished without injection, by avoiding pain or potential complications associated therewith.

In addition, coupling of the one or more payloads to the remainder of the fusion molecule with a linker that is cleaved by an enzyme present at a basolateral membrane of an epithelial cell can allow the one or more payloads to be liberated from the fusion molecule and released from the remainder of the fusion molecule soon after transcytosis across the epithelial membrane. Such liberation can reduce the probability of induction of an immune response against the payload, e.g., biologically active payload. It can also allow the one or more payloads to interact with its target free from the remainder of the fusion molecule.

Moreover, once transported across the GI epithelium, the fusion molecules of the disclosure will exhibit extended half-life in serum, that is, the one or more payloads of the fusion molecules can exhibit an extended serum half-life compared to the one or more payloads in its non-fused state, and oral administration of the fusion molecule can deliver a higher effective concentration of the delivered one or more payloads to the liver of the subject than is observed in the subject's plasma.

The constructs of the present disclosure can reduce the sensitivity of the payload to proteolytic destruction, aid in chimera refolding, and improve chimera stability during storage. As such, the fusion molecules can be used in the preparation of a new class of pharmaceutical compositions for oral administration of biologically active therapeutic agents.

As used herein, the terms “co-administration”, “co-administered” and “in combination with”, referring to the microparticles or nanoparticles of the disclosure and one or more other therapeutic agents, can include: simultaneous administration of such combination of microparticles or nanoparticles of the disclosure and therapeutic agent(s) to a patient in need of treatment, when such components are formulated together into a single dosage form which releases the components at substantially the same time to the patient; substantially simultaneous administration of such combination of microparticles or nanoparticles of the disclosure and therapeutic agent(s) to a patient in need of treatment, when such components are formulated apart from each other into separate dosage forms which are taken at substantially the same time by the patient, whereupon the components are released at substantially the same time to the patient; sequential administration of such combination of microparticles or nanoparticles of the disclosure and therapeutic agent(s) to a patient in need of treatment, when such components are formulated apart from each other into separate dosage forms which are taken at consecutive times by the patient with a significant time interval between each administration, whereupon the components are released at substantially different times to the patient; and sequential administration of such combination of microparticles of the disclosure and therapeutic agent(s) to a patient in need of treatment, when such components are formulated together into a single dosage form which releases the components in a controlled manner whereupon they are released in a concurrent, consecutive, and/or overlapping manner at the same and/or different times to the patient, where each part can be administered by either the same or a different route.

In various embodiments, the combination therapy comprises administering the isolated microparticle or nanoparticle composition and the second agent composition simultaneously, either in the same pharmaceutical composition or in separate pharmaceutical compositions. In various embodiments, isolated microparticle or nanoparticle composition and the second agent composition are administered sequentially, i.e., the isolated microparticle or nanoparticle composition is administered either prior to or after the administration of the second agent composition.

In various embodiments, the administrations of the particle, e.g., microparticle or nanoparticle composition and the second agent composition are concurrent, i.e., the administration period of the isolated particle, e.g., microparticle or nanoparticle composition and the second agent composition overlap with each other.

In various embodiments, the administrations of the particle, e.g., microparticle or nanoparticle composition and the second agent composition are non-concurrent. For example, in various embodiments, the administration of the isolated particle, e.g., microparticle or nanoparticle composition is terminated before the second agent composition is administered. In various embodiments, the administration second agent composition is terminated before the isolated f particle, e.g., microparticle or nanoparticle composition is administered.

In various embodiments, the therapeutically effective amount of microparticle or nanoparticle described herein will be administered in combination with one or more other therapeutic agents. Such therapeutic agents can be accepted in the art as a standard treatment for a particular disease state as described herein, such as metabolic disorder, fatty liver disease, inflammatory disease, autoimmune disease, cancer or growth hormone (GH) deficient growth disorder. Exemplary therapeutic agents contemplated include, but are not limited to, cytokines, growth factors, steroids, NSAIDs, DMARDs, anti-inflammatories, chemotherapeutics, radiotherapeutics, or other active and ancillary agents.

In another aspect, the present disclosure relates to methods of treating a subject classified as obese (e.g., having a body mass index (BMI) of 30 kg/m² or more) comprising administering to the subject a therapeutically effective amount of a composition, e.g., a particle comprising a carrier and payload (e.g., a delivery construct) of the present disclosure.

In another aspect, the present disclosure relates to methods for treating a subject diagnosed with type 1 diabetes (T1D) comprising orally administering a composition, e.g., a particle comprising a carrier and payload (e.g., a delivery construct) of the present disclosure in an amount sufficient to treat said disease, without insulin supplementation.

In another aspect, the present disclosure relates to methods for treating a subject diagnosed with type 1 diabetes (T1D) comprising (a) orally administering a composition, e.g., a particle comprising a carrier and payload (e.g. a delivery construct) of the present disclosure in an amount sufficient to treat said disease, and (b) insulin supplementation. In certain embodiments, the insulin supplementation comprises administering a dose of insulin that may be between about 70%-90%, between about 50%-70%, between about 30%-50%, between about 15%-30%, between about 10-15%, between about 5-10%, and between zero and 5%, including 4%, 3.5%, 3%, 2.5%, 2%, 1.5%, 1%, 0.5%, 0.4%, 0.3%, 0.2%, or 0.1% of the normal daily dosage of insulin.

In another aspect, the present disclosure relates to methods for treating a subject diagnosed with type 2 diabetes (T2D) comprising orally administering a composition, e.g., a particle comprising a carrier and payload (e.g. a delivery construct) of the present disclosure in an amount sufficient to treat said disease.

In another aspect, the present disclosure provides a method of treating a subject having a fatty liver disease (e.g., nonalcoholic fatty liver disease (NAFLD); nonalcoholic steatohepatitis (NASH)), a gastrointestinal disease, or a neurodegenerative disease, said method comprising orally administering a composition, e.g., a particle comprising a carrier and payload (e.g. a delivery construct) of the present disclosure in an amount sufficient to treat said disease.

### XI. Polynucleotides Encoding Carriers, Payloads, and Fusion Molecules

In another aspect, the disclosure provides polynucleotides comprising a nucleotide sequence encoding a carrier, a payload (e.g., a heterologous payload), and non-naturally occurring fusion molecules. These polynucleotides are useful, for example, for making the a carrier, a payload (e.g., a heterologous payload), and fusion molecules. In yet another aspect, the disclosure provides an expression system that comprises a recombinant polynucleotide sequence encoding a carrier, e.g., Cholix carrier or PE, and a polylinker insertion site for a polynucleotide sequence encoding a payload, e.g., a glucose regulating agent, e.g., a glucose-lowering agent or a cation. In various embodiments, the expression system can comprise a polynucleotide sequence that encodes a cleavable linker so that cleavage at the cleavable linker separates a payload, e.g., a glucose-lowering agent encoded by a nucleic acid inserted into the polylinker insertion site from the remainder of the encoded fusion molecule. Thus, in embodiments where the polylinker insertion site is at an end of the encoded construct, the polynucleotide comprises one nucleotide sequence encoding a cleavable linker between the polylinker insertion site and the remainder of the polynucleotide. In embodiments where the polylinker insertion site is not at the end of the encoded construct, the polylinker insertion site can be flanked by nucleotide sequences that each encode a cleavable linker.

Various in vitro methods that can be used to prepare a polynucleotide encoding a carrier, e.g., Cholix carrier or PE, payload, or fusion molecules of the disclosure include, but are not limited to, reverse transcription, the polymerase chain reaction (PCR), the ligase chain reaction (LCR), the transcription-based amplification system (TAS), the self-sustained sequence replication system (3SR) and the QP replicase amplification system (QB

Guidance for using these cloning and in vitro amplification methodologies are described in, for example, U.S. Pat. No. 4,683,195; Mullis et al., 1987, Cold Spring Harbor Symp. Quant. Biol. 51:263; and Erlich, ed., 1989, PCR Technology, Stockton Press, NY. Polynucleotides encoding a fusion molecule or a portion thereof also can be isolated by screening genomic or cDNA libraries with probes selected from the sequences of the desired polynucleotide under stringent, moderately stringent, or highly stringent hybridization conditions.

Construction of nucleic acids encoding the carriers, payloads, or fusion molecules of the disclosure can be facilitated by introducing an insertion site for a nucleic acid encoding the glucose-lowering agent into the construct.

Further, the polynucleotides can also encode a secretory sequence at the amino terminus of the encoded carrier, payload, or fusion molecule. Such constructs are useful for producing the carriers, payloads, or fusion molecules in mammalian cells as they simplify isolation of the immunogen.

Furthermore, the polynucleotides of the disclosure also encompass derivative versions of polynucleotides encoding a carrier, payload, or fusion molecule. For example, derivatives can be made by site-specific mutagenesis, including substitution, insertion, or deletion of one, two, three, five, ten or more nucleotides, of polynucleotides encoding the fusion molecule. Alternatively, derivatives can be made by random mutagenesis.

Accordingly, in various embodiments, the disclosure provides a polynucleotide that encodes a carrier, payload, or fusion molecule. The carrier, payload, or fusion molecule can comprise a modified carrier and a payload, e.g., a glucose regulating agent, e.g., a glucose-lowering agent to be delivered to a subject; and, optionally, a cleavable linker. Cleavage at the cleavable linker can separate the payload, e.g., glucose regulating agent, e.g., glucose-lowering agent from the remainder of the fusion molecule. The cleavable linker can be cleaved by an enzyme that is present at a basal-lateral membrane of a polarized epithelial cell of the subject or in the plasma of the subject.

In various embodiments, the polynucleotide hybridizes under stringent hybridization conditions to any polynucleotide of this disclosure. In further embodiments, the polynucleotide hybridizes under stringent conditions to a nucleic acid that encodes any carrier, payload, or fusion molecule of the disclosure.

In still another aspect, the disclosure provides expression vectors for expressing the carriers, payloads, or fusion molecules. Generally, expression vectors can be recombinant polynucleotide molecules comprising expression control sequences operatively linked to a nucleotide sequence encoding a polypeptide. Expression vectors can readily be adapted for function in prokaryotes or eukaryotes by inclusion of appropriate promoters, replication sequences, selectable markers, etc. to result in stable transcription and translation or mRNA. Techniques for construction of expression vectors and expression of genes in cells comprising the expression vectors are well known in the art. See, e.g., Sambrook et al., 2001, Molecular Cloning—A Laboratory Manual, 3.sup.rd edition, Cold Spring Harbor Laboratory, Cold Spring Harbor, N.Y., and Ausubel et al., eds., Current Edition, Current Protocols in Molecular Biology, Greene Publishing Associates and Wiley Interscience, NY.

The expression vectors can contain expression and replication signals compatible with the cell in which the carriers, payloads, or fusion molecules are expressed. The expression vectors can be introduced into the cell for expression of the carriers, payloads, or fusion molecules by any method known to one of skill in the art without limitation. The expression vectors can also contain a purification moiety that simplifies isolation of the carrier, payload, or fusion molecule.

In yet another aspect, the disclosure provides a cell comprising an expression vector for expression of the carriers, payloads, or fusion molecules, or portions thereof. The cell can be selected for its ability to express high concentrations of the carrier, payload, or fusion molecule to facilitate purification of the protein. In various embodiments, the cell is a prokaryotic cell, for example, E. coli. As described, e.g., in the examples, the carriers, payloads, and fusion molecules can be properly folded and can comprise the appropriate disulfide linkages when expressed in E. coli.

In other embodiments, the cell is a eukaryotic cell. Useful eukaryotic cells include yeast and mammalian cells. Any mammalian cell known by one of skill in the art to be useful for expressing a recombinant polypeptide, without limitation, can be used to express the carriers, payloads, or fusion molecules. For example, Chinese hamster ovary (CHO) cells can be used to express the carriers, payloads, or fusion molecules.

The carrier, payloads, or fusion molecules of the disclosure can be produced by recombination, as described below. However, the carrier, payloads, or fusion molecules can also be produced by chemical synthesis using methods known to those of skill in the art.

Methods for expressing and purifying the carriers, payloads, and fusion molecules of the disclosure are described extensively herein, e.g., in the examples below. Generally, the methods can rely on introduction of an expression vector encoding the carrier, payload, and/or fusion molecule to a cell that can express the carrier, payload, and/or fusion molecule from the vector. The carrier, payload, and/or fusion molecule can then be purified for administration to a subject, e.g, in the treatment of diseases and conditions for which use of the one or more payloads contained in such formulations is indicated.

### XII. Use of the Microparticle or Nanoparticle Pharmaceutical Compositions for Pulmonary Delivery

The particle compositions, e.g., microparticle pharmaceutical compositions disclosed herein can be used as drug substance for pulmonary delivery of the payload, e.g., biologically active payload. Pulmonary delivery methods may include nebulization or dry powder inhalation.

The particle, e.g., microparticle or nanoparticle pharmaceutical compositions can be formulated for pulmonary delivery. The pharmaceutical compositions formulated for pulmonary administration can be readily nebulized or aerosolized. In some cases, pharmaceutical compositions formulated for pulmonary administration take advantage of the carrier's ability to mediate transcytosis across the pulmonary epithelium. Intranasal administration can be used for pulmonary delivery and can include snorting or sniffing a powder.

### XIII. Use of the Compositions for Oral Delivery

The compositions, e.g., particle, e.g., microparticle or nanoparticle compositions, e.g., microparticle pharmaceutical compositions disclosed herein can be used as drug substance in a pill or tablet for oral delivery of the payload, e.g., biologically active payload, to an individual, e.g., in the treatment of diseases and conditions for which use of the one or more payloads contained in such formulations is indicated.

The compositions, e.g., microparticle or nanoparticle pharmaceutical compositions can be formulated for oral delivery. The pharmaceutical compositions formulated for oral administration can be resistant to degradation in the digestive tract.

In some cases, pharmaceutical compositions formulated for oral administration take advantage of the carrier's ability to mediate transcytosis across the gastrointestinal (GI) epithelium. Oral administration of these pharmaceutical compositions can result in absorption of the carrier and payload (e.g. as a fusion molecule) through polarized epithelial cells of the digestive mucosa, e.g., the intestinal mucosa, followed by release of the payload, e.g., one or more payloads at the basolateral side of the mucous membrane. Pulmonary administration of these pharmaceutical compositions can result in absorption of the carrier and payload through polarized epithelial cells of the lungs and airways. The epithelial cell may be a nasal epithelial cell, oral epithelial cell, intestinal epithelial cell, rectal epithelial cell, vaginal epithelial cell, or pulmonary epithelial cell. Pharmaceutical compositions of the disclosure can include the addition of a transcytosis enhancer to facilitate transfer of the fusion protein across the GI epithelium or pulmonary epithelium. Such enhancers are known in the art, see, e.g., Xia et al., (2000) J. Pharmacol. Experiment. Therap., 295:594-600; and Xia et al. (2001) Pharmaceutical Res., 18(2):191-195, each incorporated by reference in its entirety herein.

Once transported across the epithelium, the compositions, e.g., microparticle or nanoparticle pharmaceutical compositions of the disclosure can exhibit extended half-life in serum, that is, the payload, e.g., biologically active payload, (e.g., of the fusion molecules) can exhibit an extended serum half-life compared to the payload, e.g., biologically active payload, in its non-fused state. The oral formulations of the pharmaceutical compositions of the present disclosure can be prepared so that they are suitable for transport to the GI epithelium and protection of the carrier, payload, or fusion molecule in the stomach. Such formulations can include carrier and dispersant components and can be in any suitable form, including aerosols (for oral or pulmonary delivery), syrups, elixirs, tablets, including chewable tablets, hard or soft capsules, troches, lozenges, aqueous or oily suspensions, emulsions, cachets or pellets granulates, and dispersible powders. In various embodiments, the pharmaceutical compositions are employed in solid dosage forms, e.g., tablets, capsules, or the like, suitable for simple oral administration of precise dosages.

In various embodiments, the oral formulation comprises a microparticle pharmaceutical composition and one or more compounds that can protect the carrier, payload, or fusion molecule, or unfused carrier and payload molecules, while it is in the stomach. For example, the protective compound should be able to prevent acid and/or enzymatic hydrolysis of the molecules. In various embodiments, the oral formulation comprises a microparticle pharmaceutical composition and one or more compounds that can facilitate transit of the construct/s from the stomach to the small intestine. In various embodiments, the one or more compounds that can protect the carrier, payload, or fusion molecule from degradation in the stomach can also facilitate transit of the construct from the stomach to the small intestine. For example, inclusion of sodium bicarbonate can be useful for facilitating the rapid movement of intra-gastric delivered materials from the stomach to the duodenum as described in Mrsny et al., Vaccine 17:1425-1433, 1999. Other methods for formulating formulations so that the carriers, payloads, or fusion molecules can pass through the stomach and contact polarized epithelial membranes in the small intestine include, but are not limited to, enteric-coating technologies as described in DeYoung, Int J Pancreatol, 5 Suppl: 31-6, 1989 and the methods provided in U.S. Pat. Nos. 6,613,332, 6,174,529, 6,086,918, 5,922,680, and 5,807,832, each incorporated by reference in its entirety herein.

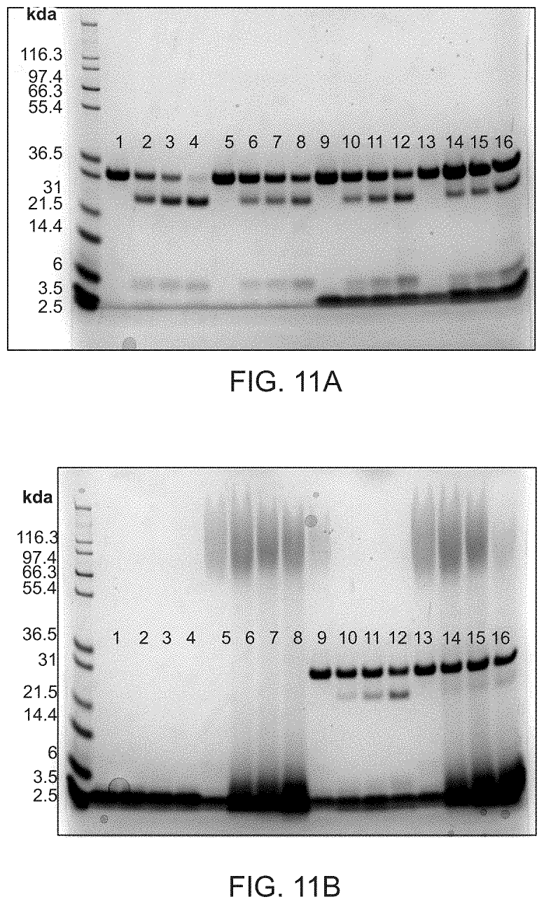

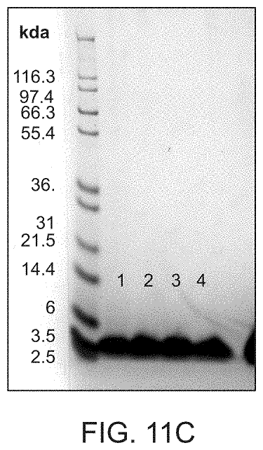

In some cases, the protective compound is a cation, which stabilizes an acid resistant microparticle or nanoparticle. In some cases, a particle comprising a carrier, a cation, and a heterologous payload can be resistant to pancreatic enzymes without need for an enteric coating or other stabilizing compound. For example, FIG. 11 shows the results of a pancreatin digestion assay performed on formulations containing a carrier protein (SEQ ID NO: 3), together with a cation (either zinc or protamine) and a heterologous payload (exenatide). FIG. 11A lanes 1-4 show pancreatin degradation of a composition of protein of SEQ ID NO: 3 after 0, 30, 60 and 120 minutes, after 120 minutes the protein is completely degraded. However, lanes 5-8 show a formulation of a protein of SEQ ID NO. 3 and a zinc salt, prepared by mixing the components at a 1:1 ratio (w/w carrier:zinc salt). Lane 8 of FIG. 11A shows that this formulation is only partially degraded after 120 minutes of incubation with pancreatin. Similar results are seen with formulations of SEQ ID NO: 3, zinc and exenatide missed at a 1:1:1 ratio or a 1:2:1 ratio. FIG. 11B shows similar results using protamine as the cation rather than zinc. FIG. 11C shows the result of pancreatin treatment on a formulation containing exenatide and zinc without SEQ ID NO: 3. In some cases, a composition described herein can be resistant to pancreatin cleavage. In some cases, a composition as described herein can be resistant to pancreatin cleavage such that at least 30%, 40%, 50%, 60%, 70%, 80% or 90% of the heterologous payload and/or carrier protein is intact after incubating the composition with pancreatin 30 minutes at 37° C. In some cases, a composition as described herein can be resistant to pancreatin cleavage such that at least 30%, 40%, 50%, 60%, 70%, 80% or 90% of the heterologous payload and/or carrier protein is intact after incubating the composition with pancreatin 60 minutes at 37° C. In some cases, a composition as described herein can be resistant to pancreatin cleavage such that at least 30%, 40%, 50%, 60%, 70%, 80% or 90% of the heterologous payload and/or carrier protein is intact after incubating the composition with pancreatin 120 minutes at 37° C. In some cases, at least 30%, 40%, 50%, 60%, 70%, 80% or 90% of the therapeutic protein is intact at 0.5 hours, 1 hour, or 2 hours in a pancreatin assay; wherein the pancreatin assay comprises incubating the composition comprising 100 μg of the therapeutic protein with 10 μg of pancreatin in 100 μL PBS at 37° C.

Pharmaceutical compositions intended for oral use can be prepared according to any method known to the art for the manufacture of pharmaceutical compositions and such formulations can contain one or more agents selected from the group consisting of sweetening agents in order to provide a pharmaceutically elegant and palatable preparation. For example, to prepare orally deliverable tablets, the microparticle pharmaceutical composition is mixed with at least one pharmaceutical excipient, and the solid formulation is compressed to form a tablet according to known methods, for delivery to the gastrointestinal tract. The tablet composition is typically formulated with additives, e.g. a saccharide or cellulose carrier, a binder such as starch paste or methyl cellulose, a filler, a disintegrator, or other additives typically usually used in the manufacture of medical preparations. To prepare orally deliverable capsules, DHEA is mixed with at least one pharmaceutical excipient, and the solid formulation is placed in a capsular container suitable for delivery to the gastrointestinal tract. Formulations comprising carriers, payloads, or fusion molecules can be prepared as described generally in Remington's Pharmaceutical Sciences, 18th Ed. 1990 (Mack Publishing Co. Easton Pa. 18042) at Chapter 89, which is herein incorporated by reference.

In various embodiments, the pharmaceutical compositions are formulated as orally-deliverable tablets containing microparticle pharmaceutical compositions in admixture with non-toxic pharmaceutically acceptable excipients, which are suitable for manufacture of tablets. These excipients can be inert diluents, such as calcium carbonate, sodium carbonate, lactose, calcium phosphate or sodium phosphate; granulating and disintegrating agents, for example, maize starch, gelatin or acacia, and lubricating agents, for example, magnesium stearate, stearic acid, or talc. The tablets can be uncoated or they can be coated with known techniques to delay disintegration and absorption in the gastrointestinal tract and thereby provide a sustained action over a longer period of time. For example, a time delay material such as glyceryl monostearate or glyceryl distearate alone or with a wax can be employed.

In various embodiments, the pharmaceutical compositions are formulated as hard gelatin capsules wherein the carrier, payload, and fusion molecule is mixed with an inert solid diluent, for example, calcium carbonate, calcium phosphate, or kaolin or as soft gelatin capsules wherein the carrier, payload, or fusion molecule is mixed with an aqueous or an oil medium, for example, arachis oil, peanut oil, liquid paraffin or olive oil.

In various embodiments, aqueous suspensions can contain a microparticle pharmaceutical composition in the admixture with excipients suitable for the manufacture of aqueous suspensions. Such excipients are suspending agents, for example, sodium carboxymethylcellulose, methylcellulose, hydroxypropylmethylcellulose, sodium alginate, polyvinylpyrrolidone, gum tragacanth and gum acacia; dispersing or wetting agents can be a naturally occurring phosphatide, for example, lecithin, or condensation products of an alkylene oxide with fatty acids, for example, polyoxyethylene stearate, or condensation products of ethylene oxide with long chain aliphatic alcohols, for example, heptadecylethyloxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyoxyethylene sorbitan monooleate. The aqueous suspensions can also contain one or more preservatives for example, ethyl or n-propyl p-hydroxybenzoate, one or more coloring agents, one or more flavoring agents and one or more sweetening agents such as sucrose or saccharin.

In various embodiments, oily suspensions can be formulated by suspending the microparticle pharmaceutical composition in a vegetable oil, for example, arachis oil, olive oil, sesame oil or coconut oil, or in a mineral oil such as liquid paraffin. The oil suspensions can contain a thickening agent, for example, beeswax, hard paraffin or cetyl alcohol. Sweetening agents, such as those set forth above, and flavoring agents can be added to provide a palatable oral preparation. These formulations can be preserved by the addition of an antioxidant such as ascorbic acid.

In various embodiments, the pharmaceutical compositions can be in the form of oil-in-water emulsions. The oil phase can be a vegetable oil, for example, olive oil or arachis oil, or a mineral oil for example, gum acacia or gum tragacanth, naturally-occurring phosphatides, for example soybean lecithin, and esters or partial esters derived from fatty acids and hexitol anhydrides, for example, sorbitan monooleate, and condensation products of the same partial esters with ethylene oxide, for example, polyoxyethylene sorbitan monooleate. The emulsions can also contain sweetening and flavoring agents.

Encapsulated or coated tablets can be used that release the one or more payloads in a layer-by-layer manner, thereby releasing one or more payloads over a pre-determined time frame while moving along the gastrointestinal tract. In addition, tablets comprising the one or more payloads can be placed within a larger tablet, thereby protecting the inner tablet from environmental and processing conditions, such as temperature, chemical agents (e.g., solvents), pH, and moisture. The outer tablet and coatings further serve to protect the one or more payloads in the gastric environment. In some cases, encapsulated particles can be placed within a larger tablet or capsule which is encapsulated such that the larger tablet or capsule dissolves in a first environment and the encapsulated particles dissolve in a second environment. In some cases, an encapsulated particle releases a payload under a first condition but not under a second condition. For example, the encapsulated particle can release a payload at a high pH but not at a low pH. In some cases, the encapsulated particle has an enteric coating.

Surface-active agents or surfactants promote absorption of polypeptides through mucosal membrane or lining. Useful surface-active agents or surfactants include fatty acids and salts thereof, bile salts, phospholipid, or an alkyl saccharide. Examples of fatty acids and salts thereof include sodium, potassium and lysine salts of caprylate (C8), caprate (C10), laurate (C12) and myristate (C14). Examples of bile salts include cholic acid, chenodeoxycholic acid, glycocholic acid, taurocholic acid, glycochenodeoxycholic acid, taurochenodeoxycholic acid, deoxycholic acid, glycodeoxycholic acid, taurodeoxycholic acid, lithocholic acid, and ursodeoxycholic acid. Examples of phospholipids include single-chain phospholipids, such as lysophosphatidylcholine, lysophosphatidylglycerol, lysophosphatidylethanolamine, lysophosphatidylinositol and lysophosphatidylserine; or double-chain phospholipids, such as diacylphosphatidylcholines, diacylphosphatidylglycerols, diacylphosphatidylethanolamines, diacylphosphatidylinositols and diacylphosphatidylserines. Examples of alkyl saccharides include alkyl glucosides or alkyl maltosides, such as decyl glucoside and dodecyl maltoside.

In another aspect, the present disclosure relates to methods of orally administering the pharmaceutical compositions of the disclosure. Oral administration of the microparticle pharmaceutical composition can result in absorption of the carrier, payload, or fusion molecule through polarized epithelial cells of the digestive mucosa, e.g., the intestinal mucosa, followed by (in some cases) cleavage of the fusion molecule and release of the one or more payloads at the basolateral side of the mucous membrane. The one or more payloads can then be transported directly to the liver via the hepatic portal vein. Thus, when the one or more payloads exerts a biological activity in the liver, such as, for example, activities mediated by one or more payloads binding to its cognate receptor, the one or more payloads is believed to exert an effect in excess of what would be expected based on the plasma concentrations observed in the subject, i.e., oral administration of the carrier, payload, or fusion molecule can deliver a higher effective concentration of the delivered one or more payloads to the liver of the subject than is observed in the subject's plasma.

In another aspect, the present disclosure relates to methods of orally administering the pharmaceutical compositions of the disclosure. Such methods can include, but are not limited to, steps of orally administering the formulations by the patient or a caregiver. Such administration steps can include administration on intervals such as once or twice per day depending on the carrier, payload, or fusion molecule, disease or patient condition or individual patient. Such methods also include the administration of various dosages of the individual carrier, payload, or fusion molecule. For instance, the initial dosage of a pharmaceutical composition can be at a higher level to induce a desired effect, such as reduction in blood glucose levels. Subsequent dosages can then be decreased once a desired effect is achieved. Such changes or modifications to administration protocols can be performed by the attending physician or health care worker.

These pharmaceutical compositions can be administered to the subject at a suitable dose. The dosage regimen would be determined by the attending physician based upon specific clinical factors. As is well known in the medical arts, dosages for any one patient depend upon many factors, including the patient's size, body surface area, age, sex, and general health, as well as the particular compound to be administered, time and route of administration, and other drugs being administered concurrently. The therapeutically effective amount for a given situation will readily be determined by routine experimentation and is within the skills and judgment of the ordinary clinician or physician. The skilled person knows that the effective amount of a pharmaceutical composition administered to an individual will, inter alia, depend on the nature of the biologically active payload. The length of treatment needed to observe changes and the interval following treatment for responses to occur vary depending on the desired effect. The particular amounts can be determined by conventional tests, which are well known to the person skilled in the art.

The amount of one or more payloads is an amount effective to accomplish the purpose of the particular active agent. The amount in the composition typically is a pharmacologically, biologically, therapeutically, or chemically effective amount. However, the amount can be less than a pharmacologically, biologically, therapeutically, or chemically effective amount when the composition is used in a dosage unit form, such as a capsule, a tablet or a liquid, because the dosage unit form can contain a multiplicity of carrier/biologically or chemically active agent formulations or can contain a divided pharmacologically, biologically, therapeutically, or chemically effective amount. The total effective amounts can then be administered in cumulative units containing, in total, pharmacologically, biologically, therapeutically or chemically active amounts of biologically active payload.

In some cases, intestinal administration of a composition of this disclosure comprising 10 μg of exenatide may result in a maximal plasma concentration of greater than about 15 ng/mL, or greater than about 30 ng/mL. In some cases, intestinal administration of a composition of this disclosure comprising 10 μg of exenatide may result in a maximal plasma concentration of about 17.3 ng/mL, or about 35.7 ng/mL. In some cases, intestinal administration of a composition of this disclosure comprising 10 μg of exenatide may result in a time to maximal plasma concentration of about 60 minutes, or about 45 minutes.

Unless otherwise defined herein, scientific and technical terms used in connection with the present disclosure shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry and hybridization described herein are those commonly used and well known in the art.

The term “about” as used herein can mean plus or minus 1%, 2%, 3%, 4%, 5%, or 10% of the number that the term refers to.

As described herein, the term “percent (%) sequence identity,” and terms related thereto, in the context of amino acid sequences, is the percentage of amino acid residues in a candidate sequence that are identical with the amino acid residues in a selected sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as Clustal Omega, BLAST, BLAST-2, ALIGN, ALIGN-2 or Megalign (DNASTAR) software. Those skilled in the art can determine appropriate parameters for measuring alignment, including any algorithms needed to achieve maximal alignment over the full-length of the sequences being compared.

### EXAMPLES

### Example 1

This example describes the preparation of stable, self-assembling microparticles containing a non-naturally occurring fusion molecule, for use as a drug substance in a pill or tablet for oral delivery of a biologically active payload. Specifically, a non-naturally occurring fusion molecule comprising a Cholix carrier molecule having the amino acid sequence of SEQ ID NO: 2 coupled to a human growth hormone (“hGH”) molecule having the amino acid sequence of SEQ ID NO: 5 was prepared using a protamine-zinc coacervate system as described below.

An exemplary fusion molecule expression vector comprising SEQ ID NO: 2 molecule and the hGH molecule was constructed as follows: first, the polypeptide genes are amplified by PCR, incorporating restriction enzymes pairs of NdeI and EcoRI, PstI and PstI, AgeI and EcoRI, or PstI and EcoRI sites at two ends of the PCR products. After restriction enzyme digestion, the PCR products are cloned into an appropriate plasmid for cellular expression, which was digested with the corresponding restriction enzyme pairs. The resulting constructs encode SEQ ID NO: 2 and hGH, and are also tagged with a 6-His motif (SEQ ID NO: 91) at the N-terminus of the polypeptide to facilitate purification. The final plasmids were verified by restriction enzyme digestions and DNA sequencing.

The fusion molecules were expressed as follows: E. coli BL21(DE3) pLysS competent cells (Novagen, Madison, Wis.) are transformed using a standard heat-shock method in the presence of the appropriate plasmid to generate fusion molecule expression cells, selected on ampicillin-containing media, and isolated and grown in Luria-Bertani broth (Difco; Becton Dickinson, Franklin Lakes, N.J.) with antibiotic, then induced for protein expression by the addition of 1 mM isopropyl-D-thiogalactopyranoside (IPTG) at OD 0.6. Two hours following IPTG induction, cells were harvested by centrifugation at 5,000 rpm for 10 min. Inclusion bodies were isolated following cell lysis and proteins were solubilized in the buffer containing 100 mM Tris-HCl (pH 8.0), 2 mM EDTA, 6 M guanidine HCl, and 65 mM dithiothreitol. Solubilized fusion molecule was refolded in the presence of 0.1 M Tris, pH=7.4, 500 mM L-arginine, 0.9 mM GSSG, 2 mM EDTA. The refolded proteins were purified by Q sepharose Ion Exchange and Superdex 200 Gel Filtration chromatography (Amersham Biosciences, Inc., Sweden). The purity of proteins was assessed by SDS-PAGE and analytic HPLC (Agilent, Inc. Palo Alto, Calif.). The resultant fusion molecule was prepared at a concentration of 1 mg/mL and stored in PBS at −80° C.

The generation of protamine-based microparticles was performed as follows: (a) a fusion molecule comprising SEQ ID NO:2 and SEQ ID NO: 5 was added to 0.1N HCl to final concentration of 1 mg/mL of the fusion molecule in a 1.5 mL microfuge tube, and then 0.02 mL ZnCl₂ (10 mg/mL in H20) was added to the fusion molecule solution; (b) 2.0 mL protamine sulfate (0.6 mg/mL) was added into 2.0 mL 0.1M NaPO4; and (c) the mixture of step (a) is combined with the mixture of step (b) and the combined mixture allowed to stand overnight at room temperature. A precipitate formed immediately upon mixing (a) and (b) as evidenced by a clear to cloudy transition.

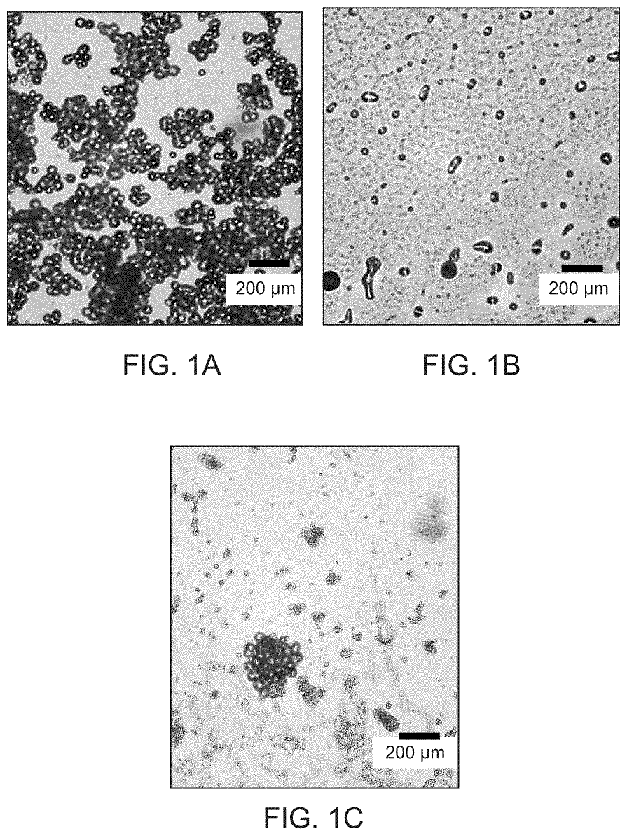

The following morning, particles were imaged using the GE Cytel system in high power (10×) bright field mode. Particles were approximately 50 μM in size and formed spontaneously at room temperature after mixing of reagents (see FIG. 1A). These particles can be disrupted into smaller units of approximately 5 μM upon increasing ionic strength of the solution by drop-wise addition of 5M NaCl (see FIG. 1B). Aggregates can re-associate by decreasing ionic strength by drop-wise addition of Milli-Q prepared water (see FIG. 1C).

These data demonstrate that a protamine-zinc coacervate system can be used to prepare stable, self-assembling microparticles containing a carrier-derived fusion molecule, and that the system will assemble into different species of particles depending upon the ionic strength of the buffer, and that association and dissociative processes are reversible for these particular molecular assemblies. The self-assembling microparticles prepared using this “tunable” system can be used as a drug substance for the preparation of a pill or tablet for oral delivery of a biologically-active payload.

### Example 2

Using the methodology of Example 1, stable insulin-containing microparticles and insulin-FITC-containing microparticles were prepared and evaluated as described below.

In one preparation, insulin and insulin-FITC was added in a 2:1 ratio to 1.0 mL 0.1N HCl to final concentration of 5 mg/mL in a 1.5 mL microfuge tube, and then 0.02 mL ZnCl₂ (10 mg/mL in H20) was added to the insulin solution. In a second preparation, insulin-FITC was added in a 2:1 ratio to 1.0 mL 0.1N HCl to final concentration of 5 mg/mL in a 1.5 mL microfuge tube, and then 0.02 mL ZnCl₂ (10 mg/mL in H20) was added to the insulin solution. In a separate step, 2.0 mL protamine sulfate (1 mg/mL) was added into 2.0 mL 0.1M NaPO4. The protamine solution was then added to the solution containing insulin and to the solution containing insulin-FITC, and each combined mixture was allowed to stand overnight at room temperature. A precipitate formed immediately upon mixing as evidenced by a clear to cloudy transition.

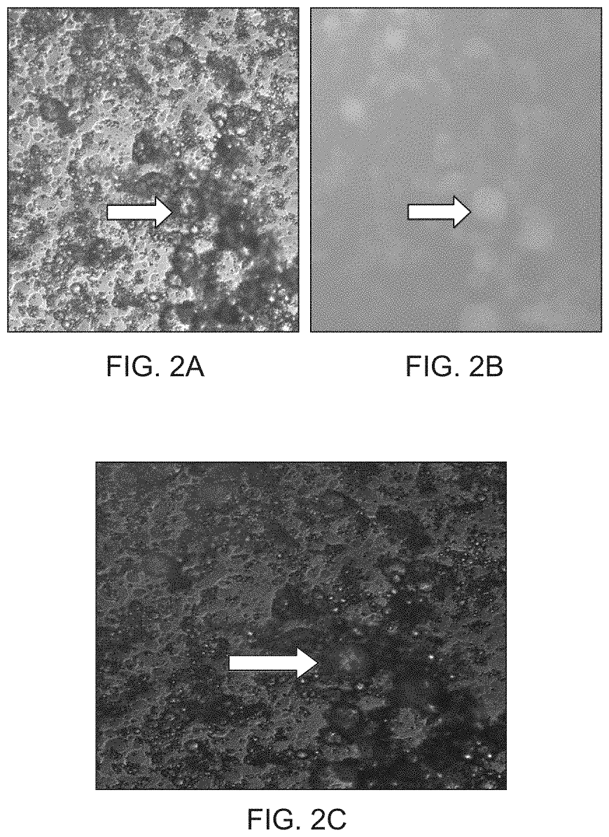

The following morning, particles containing insulin and/or insulin-FITC were imaged using the GE Cytel system in high power (10×) bright field mode. Particles were approximately 50-150 μM in size and formed spontaneously at room temperature after mixing of reagents. FIGS. 2A and 2B show the bright and blue filter images respectively. The blue arrow indicates an approximately 150 μM particle. FIG. 2C shows the merged blue and bright field images of the microparticles. The observed blue fluorescence is indicative of insulin-FITC incorporation into the particles. The nature of the fluorescence emission from these microparticles is consistent with a protein-based composition.

### Example 3

In this example, stable microparticles containing a non-naturally occurring fusion molecule comprising a Cholix carrier molecule having the amino acid sequence of SEQ ID NO: 6 coupled to a red fluorescent protein (“RFP”) molecule was prepared and evaluated using a protamine-zinc coacervate system as described below.

A fusion molecule comprising SEQ ID NO: 6 coupled to red fluorescent protein (“RFP”) was prepared as follows: a plasmid construct encoding SEQ ID NO: 6 was prepared as described herein. Protein expression was achieved using E. coli DH5α cells (Invitrogen, Carlsbad, Calif.) following transformation by heat-shock (1 min at 42° C.) with the appropriate plasmid; transformed cells, selected on antibiotic-containing media, were isolated and grown in Luria-Bertani broth (Difco); protein expression was induced by addition of 1 mM isopropyl-D-thiogalactopyranoside (IPTG); two hours following IPTG induction, cells were harvested by centrifugation at 5,000×g for 10 min at 4° C.; inclusion bodies were isolated following cell lysis and proteins were solubilized in 6 M guanidine HCl and 2 mM EDTA (pH 8.0) plus 65 mM dithiothreitol; following refolding and purification, proteins were stored at ˜5 mg/mL in PBS (pH 7.4) lacking Ca²⁺ and Mg²⁺ at −80° C. All proteins used in these studies were confirmed to be at >90% purity based upon size exclusion chromatography.

The protein as set forth in SEQ ID NO: 6, was then modified at its C-terminus to allow direct chemical coupling through a free sulfhydryl residue located near the C-terminus of the protein. The C-terminal modification includes a cysteine-constrained loop harboring the consensus cleavage sequence for the highly selective protease from the tobacco etch virus (TEV), a second cysteine, and a hexa-histidine (His6) tag (SEQ ID NO: 91). The second Cys was included to form a disulphide bridge with the Cys ultimately used for coupling. Adding the His6 sequence (SEQ ID NO: 91) to the protein simplifies the purification and the TEV cleavage sequence provides a mechanism to selectively remove the terminal Cys residue following mild reduction. TEV cleavage and mild reduction with 0.1 mM dithiothreitol following expression and isolation of the SEQ ID NO: 6 constructs allowed for the direct chemical coupling of a one or more payloads via a maleimide-based reaction as a generic mechanism of payload attachment. Following TEV protease cleavage, reduction, and RFP coupling through a maleimide reaction with the free sulfhydryl, removal of the freed C-terminal sequence was achieved by a second Ni2+ column chromatography step. The fusion molecule will be referred to hereinafter as FM001.

The generation of protamine-based microparticles was performed as follows: (a) the FM001 was added to 0.1N HCl to final concentration of 1 mg/mL FM001 in a 1.5 mL microfuge tube, and then 0.02 mL ZnCl₂ (10 mg/mL in H20) was added to the FM001 solution; in a second preparation, the ZnCl₂ was omitted; (b) 2.0 mL protamine sulfate (0.6 mg/mL) was added into 2.0 mL 0.1M NaPO4; and (c) the two mixtures of step (a) were each combined with the mixture of step (b) and the combined mixtures allowed to stand overnight at room temperature. A precipitate formed immediately upon mixing (a) and (b) as evidenced by a clear to cloudy transition.

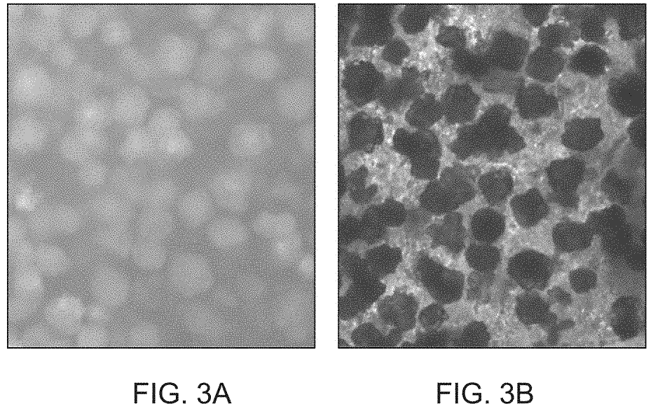

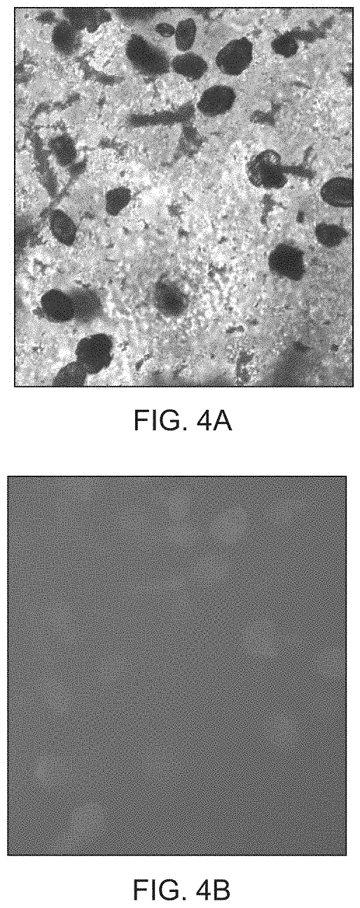

The following morning, digital images of the particles were collected using the GE Cytel system. Red fluorescence was imaged by exciting the sample at 481 nm and recording the fluorescent emission at 535 nm. At 4× magnification, particles containing protamine, zinc, and FM001 are uniform in size at approximately 150 μM (FIG. 3A). When excited with 481 nm light the particles emit a red fluorescence (FIG. 3B). The observed red fluorescence is indicative of FM001 incorporation into the particles. Fluorescence was uniform in distribution indicating that the FM001 was homogeneously distributed and that protein structure was not disrupted during particle formation. At 10× magnification, omission of zinc from the coacervate led to formation of particles with similar size (˜150 μM), however, the particles were more ovoid in shape (FIG. 4A). These particles had similar fluorescent properties to those containing zinc (FIG. 4B), indicating that zinc is not required to maintain protein structure during coacervation, and that the composition of the coacervate can determine the shape of the particles.

### Example 4

In this example, a range of different conditions and formulations were tested for producing spray dried particles with desired properties. In this example a number of different particle formulations were produced. These particles were generally made of carrier, drug, polymer matrix, PEG, surfactants, and zinc.

An example of a detailed protocol is provided below for formulation 37-49, comprising hGH/SEQ ID NO: 3/Eudragit FS30D/Polysorbate 20/ZnCl₂. A volume of 15 μL of polysorbate was added to a Falcon tube, followed by 7.6 mL of SEQ ID NO: 3 solution (9.5 mg, 0.27 μmole). Next, hGH Powder 3.0 mg (0.14 μmole) was dissolved in 4.0 mL of DI water in a separate 20 mL glass scintillation vial to yield clear solution, and then transferred to above solution in Falcon tube. A volume of 15 uL of ZnCl₂ stock solution (5 mg/ml) (0.075 mg, 0.55 μmole) was added to the mixture, followed by 82 mg of Eudragit FS 30D suspension. The suspension was shaken on a rotisserie shaker for 20 minutes, then diluted with 50 mM ammonium bicarbonate solution to 25 mL in total volume. The solution was filtered through 0.4 μm disk filter by Pall laboratory before being spray-dried on Buchi B-90 nano spray dryer.

The spray drying was performed using the following conditions: medium nozzle for spray/atomization, inlet temperature 110° C., outlet temperature 49° C., gas flow 130 liter/min., pump speed 12%, and spray ratio 100%. The spray drying completed in 2 hours. The product was collected as white and free-flowing powder. The yield was around 56% by weight. The particle size was estimated at 300-600 nm by machine spectrometry. Other particles were prepared similarly.

The particles produced by the different formulation conditions were first screened by gel electrophoresis to confirm protein quality following the spray drying process. To do this the particles were dissolved and the released protein was analyzed by western blot to ensure that the protein were undamaged.

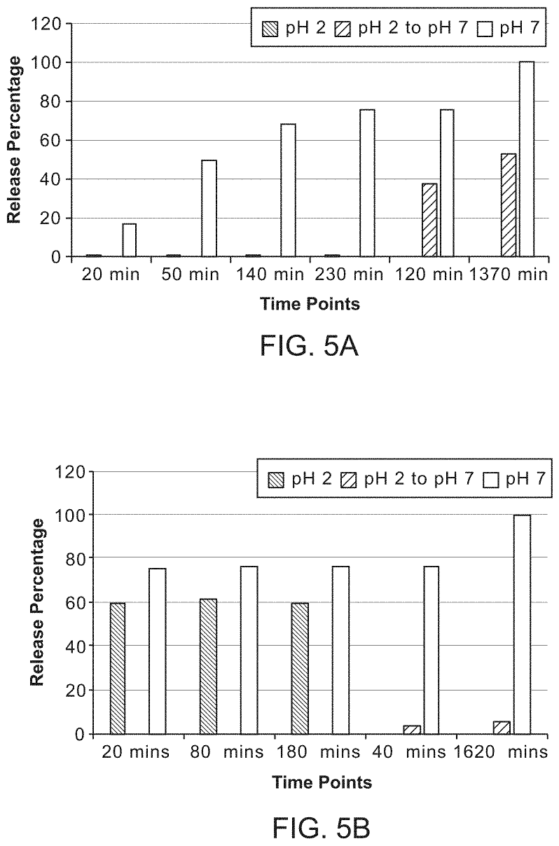

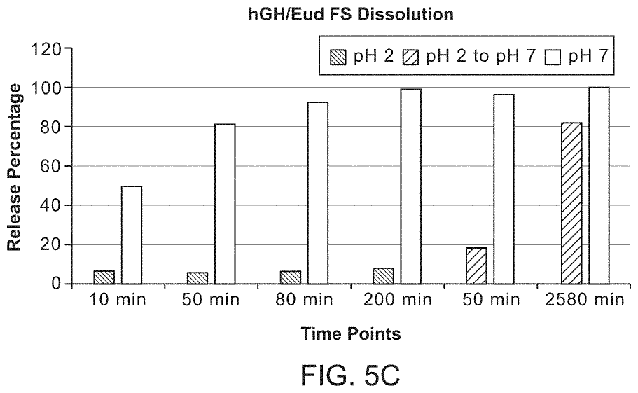

Following this the different particles were screened for encapsulation and release efficiencies at physiological pH. FIG. 5A shows the dissolution assay results for a particle comprising insulin, SEQ ID NO: 3 and Eudragit FS (37-156). This formulation produced a particle with low rates of drug release at pH 2 and gradual drug release at pH 7. Elevating the pH from pH 2 to pH 7 induced controlled release of previously-encapsulated drug. In some cases, this can represent desired encapsulation and release properties. FIG. 5B shows the dissolution results for a particle comprising insulin, SEQ ID NO: 3 and Eudragit L30 (37-167). This formulation produced a particle with fast drug release at both pH 2 and pH 7. In some cases, this may not be a desired encapsulation and release property. FIG. 5C shows the dissolution assay results for a further particle formulation, comprising hGH and Eudragit FS. This formulation resulted in low drug release at pH 2, gradual drug release at pH7 and controlled induction of drug release upon elevation of pH from pH 2 to pH 7. This release profile indicates that the formulation can potentially be suitable for oral administration, for example by oral gavage.

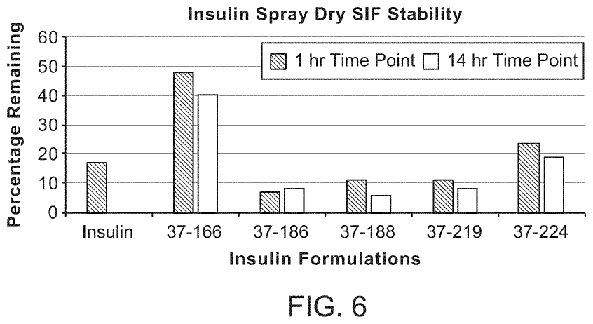

Formulations were next tested for stability in simulated intestinal fluid with pancreatin. The different particles were exposed to the simulated intestinal fluid with pancreatin, and the amount of drug remaining was assessed after 1 hour and 14 hours. FIG. 6 shows percentage of drug remaining after 1 hr and 14 hrs for insulin standard solution and five different formulations. Details of the formulations in FIG. 6 are listed in Table 1. All five of the spray dry particles in FIG. 6 offered long term drug protection from the pancreatin. Two different formulations, 37-166 (Insulin/PEG8K/SEQ ID NO: 3/Eudragit FS/Tween-20/ZnCl₂) and 37-224 (Insulin/SEQ ID NO: 3/2× Eudragit L30/ZnCl₂), resulted in higher insulin concentration at the 1 hr time point than seen with insulin alone.

TABLE 1
Formulations assessed in FIG. 6.

| Formulation | | Drug content |
|---|---|---|
| name | Spary Dry Formulation | (w/w) |
| Insulin | Insulin standard solution | 100% |
| 37-166 | Insulin/PEG8K/SEQ ID NO. 3/Eud | 1.54% |
| | FS/Tw20/ZnCl₂ | |
| 37-186 | Insulin/SEQ ID NO. 3/Eud FS/Tw20 | 1.42% |
| 37-188 | Insulin/PEG8K/SEQ ID NO: 3/Eud | 3.17% |
| | L30/Tw20 | |
| 37-219 | Insulin/SEQ ID NO: 3/Eud L30/ZnCl₂ | 6.17% |
| 37-224 | Insulin/SEQ ID NO: 3/2X Eud L30/ | 4.97% |
| | ZnCl₂ | |

### Example 5

In this example, in vivo absorption of drug from different particle compositions will be assessed. It is hypothesized that intestinal drug retention will correlate with drug absorption, and will be modulated by particle compositions. Fluorescently-labeled drugs will be formulated into particles as described above. The particles will be suspended in a suitable solution for administration by oral gavage. In some cases, the solution can be water. The particle suspension will be administered to rats by oral gavage. Each different fluorescently-labeled drug and formulation combination will be administered to four rats. A further four rats will be administered a solution containing the same total amount of the fluorescently labeled drug without formulation into particles. Whole body imaging scans will be performed on two rats of each four, prior to administration of the fluorescently labeled drug and at 0.5 hrs, 1 hr, 2 hrs, 4 hrs, 6 hrs, 8 hrs, 10 hrs, 12 hrs, and 24 hrs after administration. The remaining two rats of each treatment group will be sacrificed at 4 hrs and 12 hrs and intestines will be collected to confirm drug concentrations.

### Example 6

In this example, particles from formulation 37-49 (hGH/SEQ ID NO: 3/Eudragit FS) were injected into an in vivo model to assess in vivo transcytosis. Formulation 37-49 particles were administered to rats by intraluminal injection at a dose of 34 μg/Kg.

Male Wistar rats were housed 3-5 per cage with a 12/12 h light/dark cycle and were 225-275 g (approximately 6-8 weeks old) when placed on study. All experiments were conducted during the light phase using a non-recovery protocol that used continuous isoflurane anesthesia. A 4-5 cm midline abdominal incision exposed mid-jejunum regions. A stock solution at 3.86×10-5 M of formulation 37-49 particles was prepared in phosphate buffered saline (PBS), with 50 μL (per 250 g rat) being administered by intraluminal injection (ILI) using a 29-gauge needle. The injection site mesentery was marked with a permanent marker. At study termination, a 3-5 mm region that captured the marked intestine segment was isolated and processes for microscopic assessment.

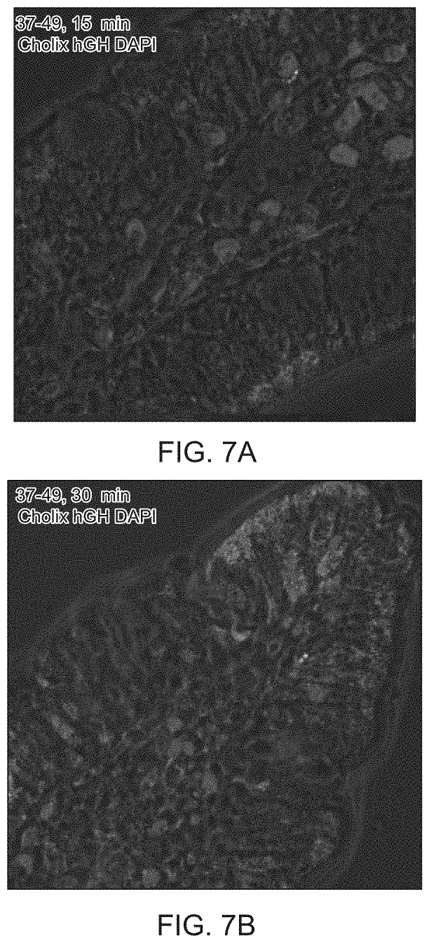

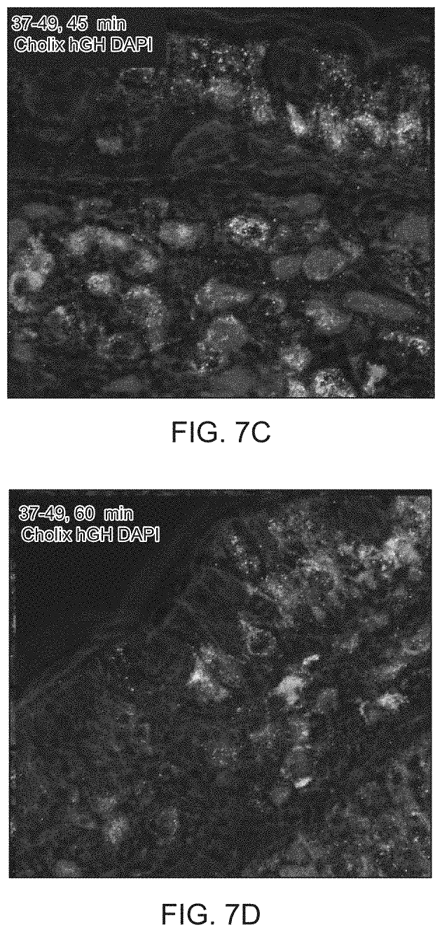

Injected animals were sacrificed at a range of time points after injection and intestines were harvested and sectioned. Sections of the injected region of the intestine were prepared and visualized by immunofluorescence. Uptake of the particles was seen beginning at 15 minutes, as compared to 1-5 minutes seen for cholix constructs. As seen in FIG. 7A hGH (green) and cholix (red) are initially largely co-localized (yellow) at 15 minutes post injection, more free hGH is seen at later time points as seen in FIGS. 7B-D, 30 minutes, 45 minutes, and 60 minutes respectively after injection.

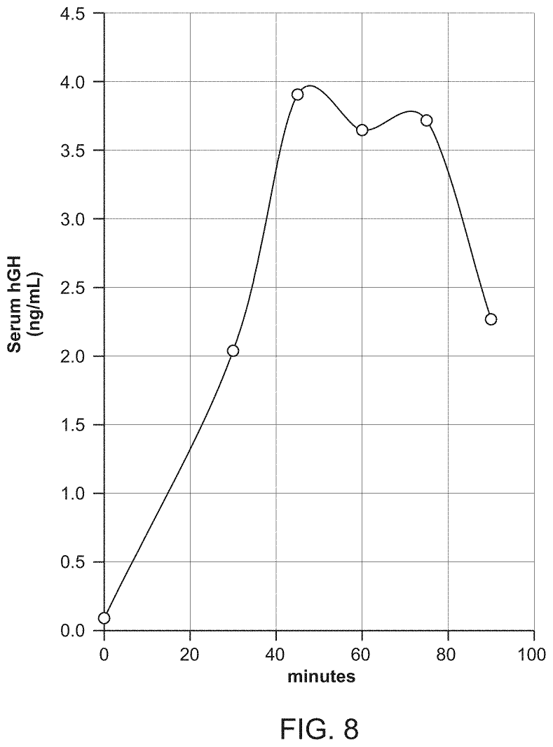

To further assess drug delivery hGH SEQ ID NO: 3 nanoparticles were delivered to three rats (Animals A, B and C) by intrajejunal injection at a dose of 1.93 nmol/Kg, resulting in a dose of about 43 μg/Kg of hGH, and a total amount of 11 μg of hGH per rat. Serum samples were collected at 0, 30, 45, 60, 75, 90, 105 and 120 minutes post injection. After 120 minutes the animals were sacrificed and the intestines and livers were collected. FIG. 8 shows the serum concentration of hGH in Animal A. A rapid rise in serum hGH concentration was seen, peaking around 40 minutes after administration at 4 ng/mL and remaining above 3.5 ng/mL until at least 75 minutes post administration. Animals B and C did not have detectable levels of hGH in the serum. hGH was detected in the serum and intestine of animal A, and in the intestine of animal B though not in the serum. hGH was not detected in either the serum or intestine of animal C, and was not detected in the liver of any of the animals at the 120 minute time point.

### Example 7

In this example, hGH containing particles were applied to Caco-2 cells to assess transport through the cells. Caco-2 cells are a human colon epithelial cancer cell line which can be used as a model for human intestinal absorption of drugs and other compounds. When cultured as a monolayer, Caco-2 cells differentiate and form tight junctions between cells when cultured as a monolayer. This monolayer can be used as a model for paracellular movement of compounds. Caco-2 cells express transporter proteins, efflux proteins, and Phase II conjugation enzymes to model a variety of transcellular pathways. In some cases, the Caco-2 cell monolayer can be used as a mimic of the human intestinal epithelium.

Caco-2 cells were seeded at 1.5×10⁵ cells/mL in transwells. The culture medium was exchanged every 2 days in both the apical (0.5 mL) and the basolateral (1.5 mL) chambers. Experiments were conducted after cells were grown for 21 days and functionally tight monolayers were formed as assessed by trans-epithelial electrical resistance (TEER). On day 21, the transwells were washed once with PBS. 100 uL of suspension containing hGH particles was added to the apical chambers. The basal chamber was added with 0.5 mL of PBS. After 2 h at 37° C., the solution from basolateral chambers were collected and concentrated. The hGH transported across the tissues was evaluated using western blotting. Proteins were separated by 1D gel electrophoresis in a 4-12% NuPAGE gel (BioRad, cat. #5678095). The separated proteins were transferred to PVDF membrane (BioRad, cat. #1704157), incubated with goat anti-hGH polyclonal antibody (1:1000, R&D AF1067), followed by AP-conjugated secondary rabbit anti-goat antibody (1:10000, Abcam ab6742). Proteins bands were visualized using AP Western blotting substrate (Promega S3841).

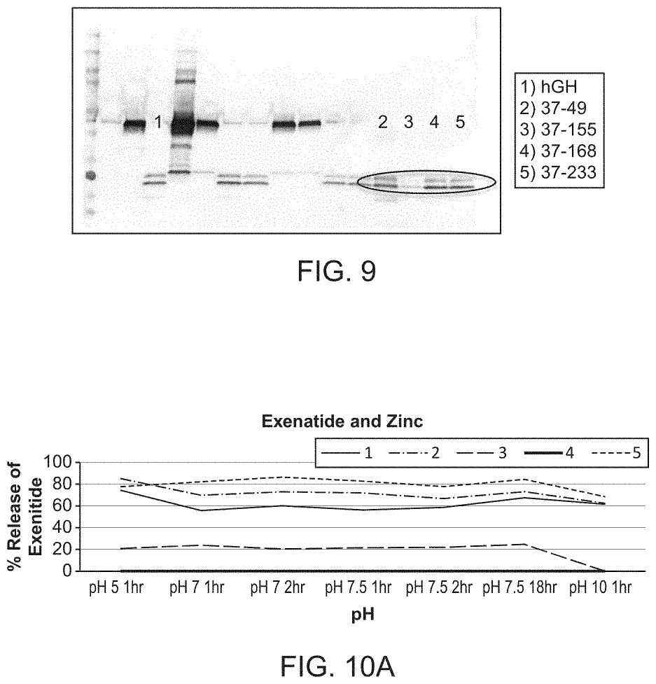

TABLE 2
Particle compositions depicted in FIG. 9

| Formulation | | Drug content |
|---|---|---|
| Number | Spray Dry Formulation | (w/w) |
| 37-49 | hGH/Eudragit FS/SEQ ID NO: | 0.34% |
| | 3/Tween20/ZnCl₂ | |
| 37-155 | *hGH/Eudragit FS/SEQ ID NO: | 0.38% |
| | 3/Tween20/ZnCl₂ | |
| 37-168 | 37-49 Batch 2 | 0.30% |
| 37-233 | hGH/Eudragit FS/SEQ ID NO: | 1.77% |
| | 3/Tween20/ZnCl₂ | |

### Example 8

In this example, several different microparticles were produced by mixing SEQ ID NO: 3, a cation and exenatide as indicated in Table 3 and Table 4. Particles thus produced were assayed by HPLC to determine the actual quantities of each component in the microparticles.

TABLE 3
Components for producing particles, and particle analysis

| | | | | | % | | | | |
|---|---|---|---|---|---|---|---|---|---|
| | | | | | Content | % | SEQ ID | | |
| | | SEQ ID | | | of | Content | NO: 3 | Exenatide | Cation |
| | Formulation | NO: 3 | Exenatide | Cation | SEQ ID | of | (mole | (mole | (mole |
| | Code | (mg) | (mg) | (mg) | NO: 3 | Exenatide | ratio) | ratio) | ratio) |
| 1 | 019-08- | 5 | 5 | Zinc | 65.1 | 4.5 | 1 | 7.16 | 32.68 |
| | E1 | | | 1 | | | | | |
| 2 | 019-08- | 5 | 5 | Zinc | 68.9 | 5.4 | 1 | 7.16 | 65.36 |
| | E2 | | | 2 | | | | | |
| 3 | 019-08- | 5 | 5 | Zinc | 59.0 | 11.3 | 1 | 7.16 | 163.39 |
| | E3 | | | 5 | | | | | |
| 4 | 019-08- | 5 | 5 | Zinc | 72.2 | 0.0 | 1 | 7.16 | 326.79 |
| | E4 | | | 10 | | | | | |
| 5 | 019-08- | 5 | 5 | Zinc | 57.5 | 3.4 | 1 | 7.16 | 653.57 |
| | E5 | | | 20 | | | | | |
| 6 | 019-08- | 2.5 | 2.5 | Protamine | 19.7 | 2.1 | 1 | 7.16 | 0.04 |
| | E6 | | | 0.2 | | | | | |
| 7 | 019-08- | 2.5 | 2.5 | Protamine | 7.3 | 2.8 | 1 | 7.16 | 0.09 |
| | E7 | | | 0.4 | | | | | |
| 8 | 019-08- | 2.5 | 2.5 | Protamine | 23.5 | 5.2 | 1 | 7.16 | 0.18 |
| | E8 | | | 0.8 | | | | | |
| 9 | 019-08- | 2.5 | 2.5 | Protamine | 17.1 | 3.1 | 1 | 7.16 | 0.45 |
| | E9 | | | 2 | | | | | |
| 10 | 019-08- | 2.5 | 2.5 | Protamine | 4.9 | 1.0 | 1 | 7.16 | 0.89 |
| | E10 | | | 4 | | | | | |

TABLE 4
Components for producing particles, and particle analysis

| | | | | | | | SEQ | | |
|---|---|---|---|---|---|---|---|---|---|
| | | | | | % of | | ID | | |
| | | amount | amount | amount | SEQ | | NO: | | Zn/ |
| | | of SEQ | of | of | ID | | 3 | Exenatide | Protamine |
| | Formulation | ID NO: | Exenatide | Zn/Pro | NO: | % | (mole | (mole | (mole |
| | Code | 3 (mg) | (mg) | (mg) | 3 | Exe | ratio) | ratio) | ratio) |
| 11 | 019-08-E11 | 0.058 | 5 | Zinc | 0.42 | 13.58 | 1 | 617.06 | 28171.33 |
| | | | | 10 | | | | | |
| 12 | 019-08-E12 | 0.116 | 5 | Zinc | 0.61 | 12.75 | 1 | 308.53 | 14085.67 |
| | | | | 10 | | | | | |
| 13 | 019-08-E13 | 0.29 | 5 | Zinc | 1.42 | 14.25 | 1 | 123.41 | 5634.27 |
| | | | | 10 | | | | | |
| 14 | 019-08-E14 | 0.58 | 5 | Zinc | 19.08 | 27.83 | 1 | 61.71 | 2817.13 |
| | | | | 10 | | | | | |
| 15 | 019-08-E15 | 1.16 | 5 | Zinc | 11.67 | 12.25 | 1 | 30.85 | 1408.57 |
| | | | | 10 | | | | | |
| 16 | 019-08-E16 | 0.058 | 5 | Protamine | 20.75 | 14.11 | 1 | 617.05 | 1.12 |
| | | | | 10 | | | | | |
| 17 | 019-08-E17 | 0.116 | 5 | Protamine | 0.43 | 12.00 | 1 | 14.32 | 1.12 |
| | | | | 10 | | | | | |
| 18 | 019-08-E18 | 0.29 | 5 | Protamine | 3.25 | 19.75 | 1 | 14.32 | 1.12 |
| | | | | 10 | | | | | |
| 19 | 019-08-E19 | 0.58 | 5 | Protamine | 0.90 | 11.17 | 1 | 14.32 | 1.12 |
| | | | | 10 | | | | | |
| 20 | 019-08-E20 | 1.16 | 5 | Protamine | 17.83 | 9.17 | 1 | 14.32 | 1.12 |
| | | | | 10 | | | | | |

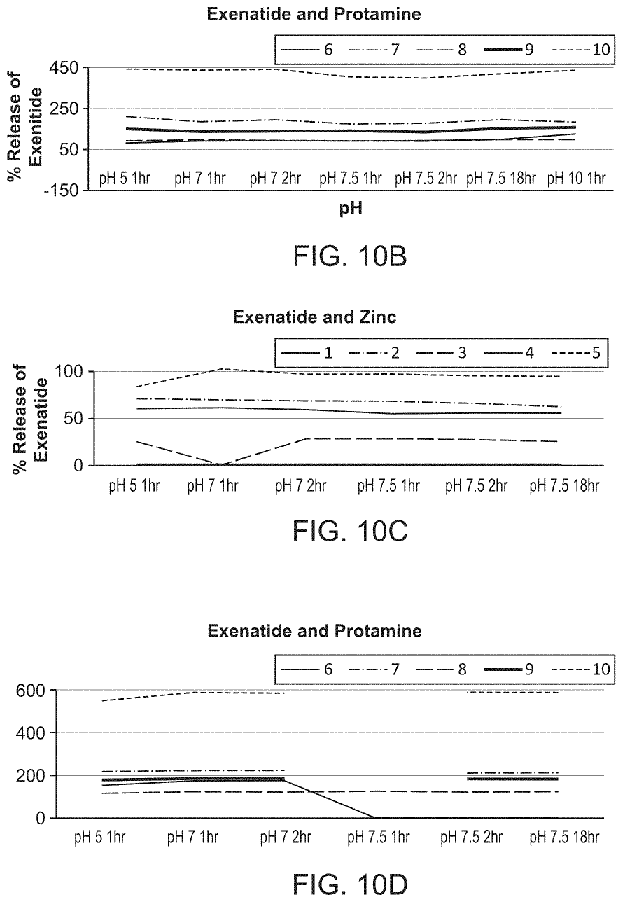

In vitro release of exenatide from the particles produced above was assessed by incubating the particles in different pH solutions for up to 18 hrs. The amount of released exenatide was quantified by reverse phase liquid chromatography (RPLC, FIGS. 10A and 10B) or size exclusion chromatography (SEC, FIGS. 10C and 10D). As seen in FIGS. 10A-D the compositions formed a pH stable mixture.

### Example 9

To determine pancreatin stability of different SEQ ID NO: 3 and exenatide complexes were incubated with pancreatin enzymes for 0, 30, 60 or 120 minutes as described previously. FIGS. 11A-C show the amounts of SEQ ID NO: 3 (˜30,162 da), exenatide (˜7,000 da) and protamine (˜4,186 da) remaining for different compositions at different time points. Table 5, Table 6, and Table 7 below show the formulations and timepoints of each lane in FIGS. 11A-C.

TABLE 5
Description of lanes in FIG. 11A

| | Formulation: ratio of | Timepoint |
|---|---|---|
| Lane | carrier:zinc:exenatide | (minutes) |
| 1 | 1:0:0 | 0 |
| 2 | | 30 |
| 3 | | 60 |
| 4 | | 120 |
| 5 | 1:1:0 | 0 |
| 6 | | 30 |
| 7 | | 60 |
| 8 | | 120 |
| 9 | 1:1:1 | 0 |
| 10 | | 30 |
| 11 | | 60 |
| 12 | | 120 |
| 13 | 1:2:1 | 0 |
| 14 | | 30 |
| 15 | | 60 |
| 16 | | 120 |

TABLE 6
Description of lanes in FIG. 11B

| | Formulation: ratio of | Timepoint |
|---|---|---|
| Lane | carrier:protamine:exenatide | (minutes) |
| 1 | 0:0:1 | 0 |
| 2 | | 30 |
| 3 | | 60 |
| 4 | | 120 |
| 5 | 0:1:1 | 0 |
| 6 | | 30 |
| 7 | | 60 |
| 8 | | 120 |
| 9 | 1:0.16:1 | 0 |
| 10 | | 30 |
| 11 | | 60 |
| 12 | | 120 |
| 13 | l:0.8:l | 0 |
| 14 | | 30 |
| 15 | | 60 |
| 16 | | 120 |

TABLE 7
Description of lanes in FIG. 11C

| | Formulation: ratio of | Timepoint |
|---|---|---|
| Lane | carrier:zinc:exenatide | (minutes) |
| 1 | 0:1:1 | 0 |
| 2 | | 30 |
| 3 | | 60 |
| 4 | | 120 |

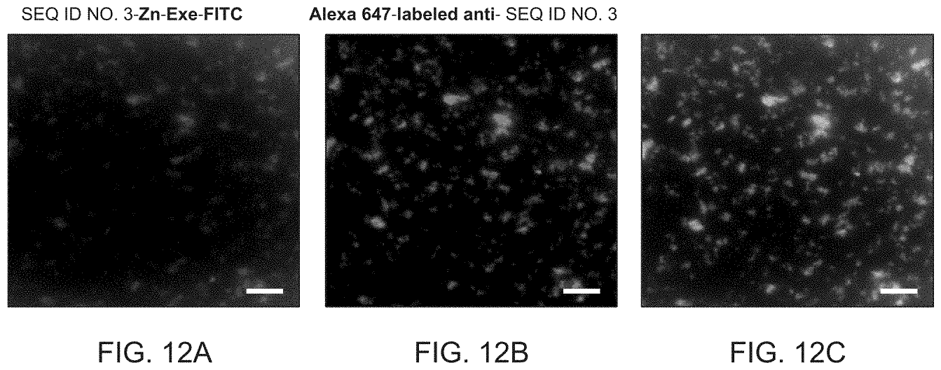

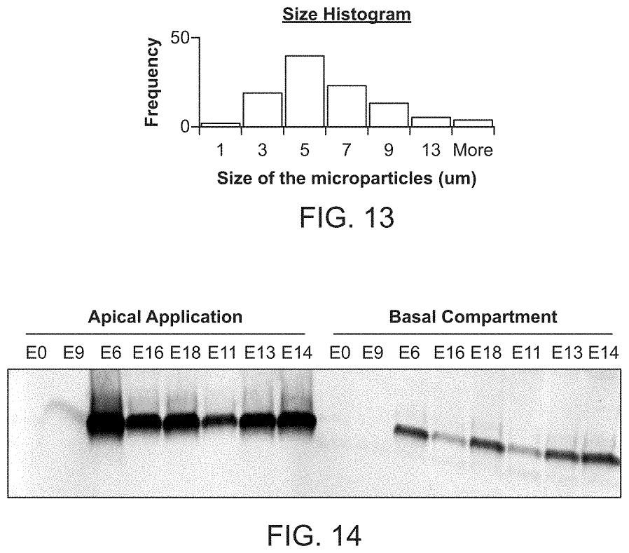

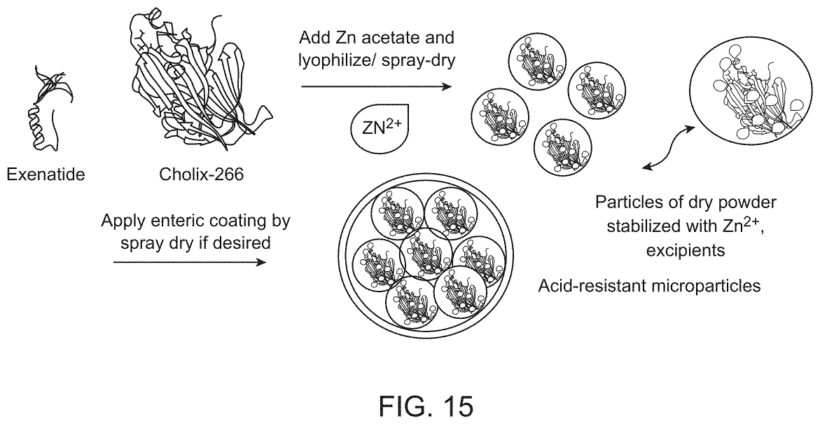

FIGS. 12A-C shows confocal images of microparticles produced from cholix, FITC-labeled exenatide and zinc. A 20 μm scale bar is shown on each image. The size distribution of approximately 100 particles is shown in FIG. 13. The average size of the microparticles was ˜5 μm±2 μm.

To determine the ability of particles to transcytose across human SMI-100 cells, particles were prepared as outlined in Table 8. The resulting particles were dissolved in 10 mL of PBS and 100 uL of the solution was added onto the apical side of the cells and 500 uL of PBS was added in the basal chamber. The proteins in the basal solution after 1 hr at 37° C. was concentrated and analyzed by western blotting. Exenatide in the basal solution was quantified by HPLC. As seen in FIG. 14, and summarized in Table 8, transcytosis was observed in 6 formulations tested. E0 is a control with no proteins, and EP9 was not dissolved in PBS. Higher levels of exenatide transport were seen with the particles that comprised zinc and not protamine (Ell, E13, and E14).

TABLE 8
Formulations used in transcytosis assay

| | SEQ | | | | | Basolateral |
|---|---|---|---|---|---|---|
| | ID | | | | | concentration of |
| | NO: 3 | exenatide | Cation | % of SEQ | % of | exenatide |
| Formulation | (mg) | (mg) | (mg) | ID NO: 3 | exenatide | (ng/mL) |
| E0 | 0 | 35 | Zinc | 0 | 35 | 0 |
| | | | 2 | | | |
| E9 | 2.5 | 2.5 | Protamine | 17.1 | 3.1 | 0 |
| | | | 2 | | | |
| E6 | 2.5 | 2.5 | Protamine | 19.7 | 2.1 | 0 |
| | | | 0.2 | | | |
| E16 | 0.058 | 5 | Protamine | 20.8 | 14.1 | 3 |
| | | | 10 | | | |
| E18 | 0.29 | 5 | Protamine | 3.25 | 19.8 | 3 |
| | | | 10 | | | |
| E11 | 0.058 | 5 | Zinc | 0.42 | 13.6 | 4 |
| | | | 10 | | | |
| E13 | 0.29 | 5 | Zinc | 1.42 | 14.3 | 7 |
| | | | 10 | | | |
| E14 | 0.58 | 5 | Zinc | 19.1 | 27.8 | 8 |
| | | | 10 | | | |

### Example 10: In Vivo Study

Formulations were selected for in vivo testing. These formulations were prepared at larger scale and samples were analyzed for content and purity as before. Formulation details and analytical testing results are shown in Table 9. In a typical preparation, a solution comprising Cholix carrier SEQ ID NO:3 (or if no Cholix, water was substituted) was mixed with an exenatide solution for one minute on a stirrer plate. Zinc or protamine solution was added dropwise to the stirred solution. After 15 minutes of stirring, the whole solution/suspension was lyophilized. Three additional formulations were prepared by the same method using a fluorescein tagged exenatide; these are summarized in Table 10. Fluorescein on the side-chain of lysine was added to the N-terminus of exenatide by solid-phase peptide synthesis.

TABLE 9
Formulations of Exenatide, Cholix (SEQ ID NO: 3), and Cation

| | | amount of | amount of | amount of | % | | |
|---|---|---|---|---|---|---|---|
| | Formulation | SEQ ID | Exenatide | Zn/Pro | SEQ ID | % | % |
| | Code | NO: 3 (mg) | (mg) | (mg) | NO: 3 | Exenatide | Purity |
| 0 | 019-08-E10 | 0 | 10 | Zinc | 0 | 13.45 | >95 |
| | | | | 20 | | | |
| 13 | 019-08-E13 | 0.58 | 10 | Zinc | 3.10 | 37.68 | >95 |
| | | | | 20 | | | |
| 14 | 019-08-E14 | 1.16 | 10 | Zinc | 4.57 | 24.7 | >95 |
| | | | | 20 | | | |
| 18 | 019-08-E18 | 0.58 | 10 | Protamine | 2.75 | 35.65 | >95 |
| | | | | 20 | | | |
| 10 | 019-08-E10 | 0 | 10 | Zinc | | | |
| | | | | 20 | | | |

TABLE 10
Formulations prepared with exenatide-fluorescein.

| | | % SEQ ID | % |
|---|---|---|---|
| Formulation | Components | NO: 3 | Exenatide |
| 019-10-E0 | Exe-fluorescein + Zn (1:2) | 0 | 13.45 |
| FITC | | | |
| 019-10-E14 | SEQ ID NO: 3 + Exe- | 4.57 | 24.7 |
| FITC | fluorescein + Zn (0.116:1:2) | | |
| 019-10-E18 | SEQ ID NO: 3 + Exe- | 2.75 | 35.65 |
| FITC | fluorescein + Pro | | |
| | (0.058:1:2) | | |

The purity of each powder used in preparing the formulations was: Exenatide % content ˜89.0% and SEQ ID NO: 3% content ˜71.8%, determined by calculating the % relative peak area observed from size exclusion chromatography (SEC).

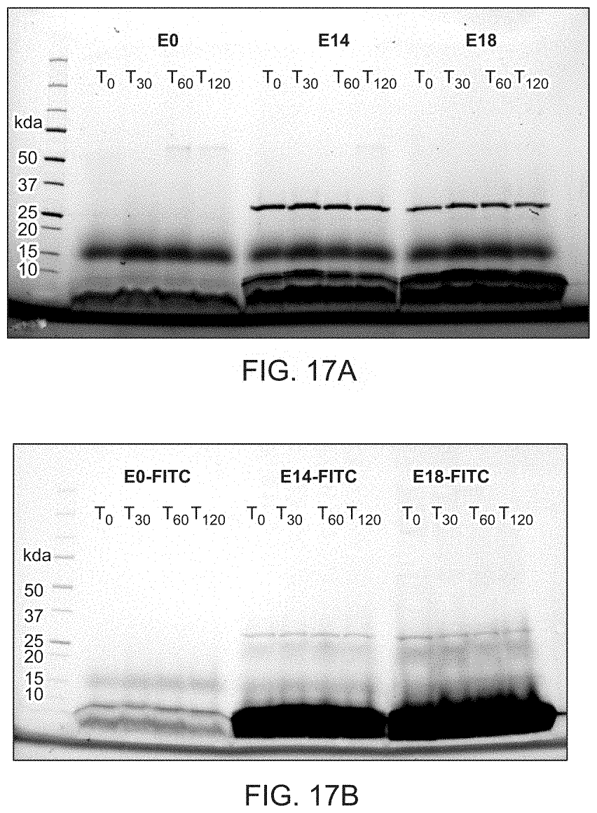

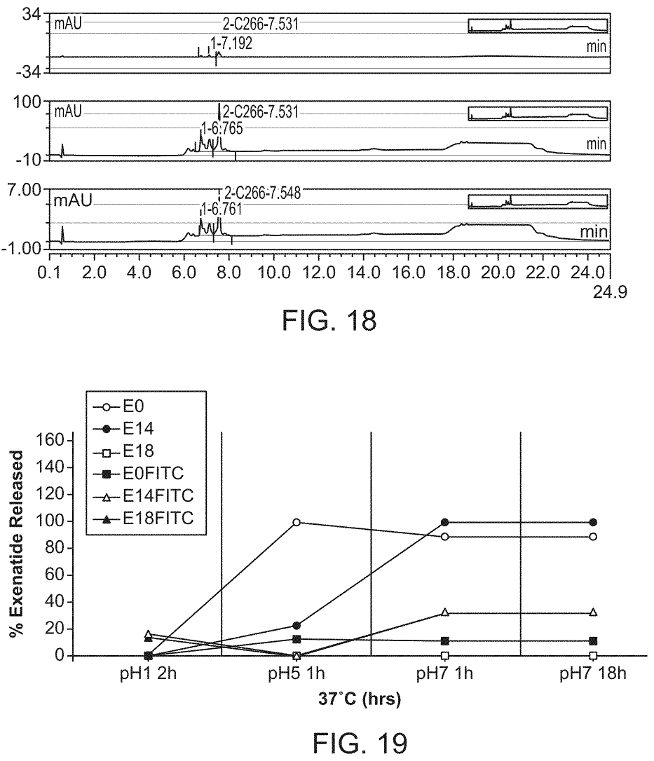

A pancreatin assay was performed to determine the stability of the different formulations; results can be seen in FIGS. 17A and 17B. Samples were analyzed using 4-20% Citerion TGX stain-free precast gels (Bio-Rad, 5678094), Precision Plus unstained standard (Bio-Rad, 161-0375) and analyzed by ThermoFisher Gel Scanner. Formulations E14, E18, E14-FITC, and E18-FITC all showed very little degradation of SEQ ID NO: 3 even after 2 hours of exposure to pancreatin enzymes. FIG. 18 shows a reverse phase chromatogram (RPLC) showing the presence of SEQ ID NO: 3 at the retention time of 6.8 min and exenatide at 7.5 min. These formulations were also assessed for aqueous solubility at a range of different pH values. As seen in FIG. 19 the FITC formulations were less soluble than the non FITC formulations. Formulation E14 showed low solubility at pH 1 and high solubility at pH 7 and greater. Formulation E0 also showed low solubility at pH 1 and high solubility at pH 5 and greater.

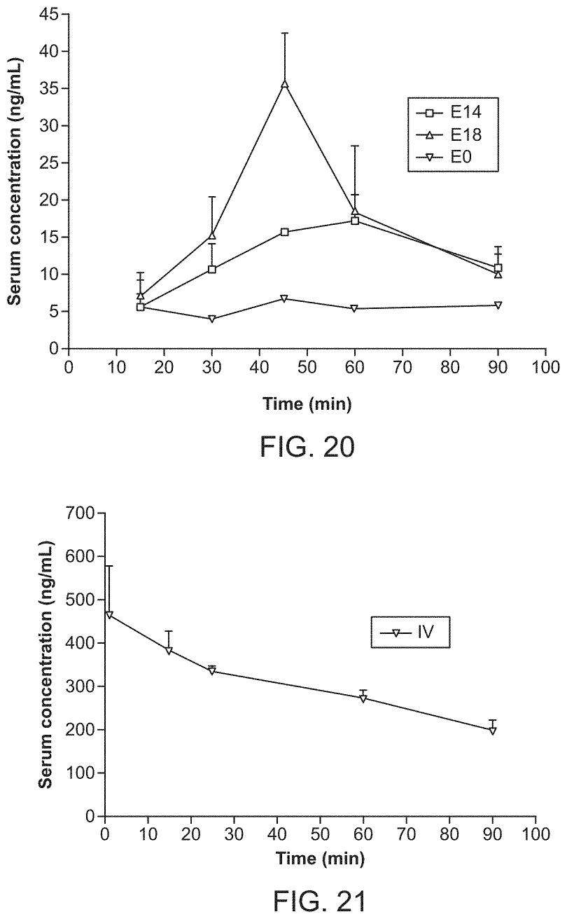

To assess the in vivo pharmacokinetics and pharmacodynamics particles were suspended in PBS and 100 μL of each suspension was injected into a rat intestinal lumen. Four rats were used for each formulation, and the formulations were suspended to produce a dose of 10 μg of exenatide for E14 and E18, and 0 μg of exenatide for E0. 100 μL blood samples were taken at 15, 30, 45, 60 and 90 minutes after the injection. Blood was allowed to clot and then centrifuged to prepare serum, exenatide concentration was measured by ELISA. As seen in FIG. 20 exenatide was detected in the serum of animals injected with both E14 and E18. The E18 formulation resulted in a higher maximum serum concentration and a higher area under the curve. The Cmax for the E14 formulation was 17.3 ng/mL, and for the E18 formulation was 35.7 ng/mL. The Tmax was 60 minutes of E14 and 45 minutes for E18. No changes from baseline were seen in blood glucose levels for any animal. As a comparison an equivalent amount of exenatide was injected into rats intravenously and the serum concentration was assessed, see FIG. 21. The pharmacokinetics of intestinally delivered E14, E18 and intravenously delivered exenatide are compared in Table 11.

TABLE 11
Pharmacokinetics.

| | | AUC | Relative |
|---|---|---|---|
| Formulation | Cmax (ng/mL) | (ng/mL × min) | availability (%) |
| IV | 465.0 | 27250 | 100 |
| E14 | 20.2 | 996.3 | 3.66 |
| E18 | 40.6 | 1386 | 5.09 |

### Example 11: Preparation of Compositions Comprising Exenatide

Exenatide (SEQ ID NO: 11) is a peptide having GLP-1-like biological activity that is stabilized by a C-terminal amine and an N-terminal H. In this Example, two non-naturally occurring isolated constructs comprising: 1) a carrier having SEQ ID NO: 78 processed to a carrier having SEQ ID NO: 70 and crosslinked to SEQ ID NO: 11 and 2) a carrier having SEQ ID NO: 77 processed to carrier having SEQ ID NO: 80 and crosslinked to SEQ ID NO: 11 were prepared and tested for intestinal epithelial transport in vivo. Carriers having SEQ ID NO: 80 and SEQ ID NO: 70 were prepared as described herein and Exenatide (SEQ ID NO: 11) (Cat #HOR-246) was purchased from ProSpec-Tany Technogene Ltd. PO Box 6591, East Brunswick, N.J. 08816. A Pierce™ Controlled Protein-Protein Crosslinking Kit (Cat #23456) comprising the Sulfo-SMCC Crosslinking Agent was purchased from ThermoFisher.

### Payload and Carrier Activation and Crosslinking:

Exenatide (10 mg) was dissolved in 5 mL H₂O to form a 2 mg/mL solution. Sulfo-SMCC (2 mg) was dissolved in 2 mL of PBS. Immediately thereafter, 0.088 mL (˜5-fold molar excess) of the Sulfo-SMCC solution was added to 1.0 mL Exenatide solution and incubated for 30 minutes at room temperature. Nonreacted Sulfo-SMCC was removed by applying 1.0 mL of the maleimide-Exenatide reaction mixture to a desalting column equilibrated with PBS, eluting with PBS, and collecting 0.5 mL fractions. The absorbance at 280 nm of each fraction was measured to locate the protein peak. Peak fractions containing most of the protein were pooled. The concentration of the pooled activated Exenatide was determined by comparing its absorbance at 280 nm with the absorbance of the original protein solution.

Carriers having SEQ ID NO: 77 and SEQ ID NO: 78 have a C-terminal extension comprising a TEV cleavage site flanked by two cysteine residues that form a disulfide bond and a C-terminal His₆ tag (SEQ ID NO: 91). Carriers having SEQ ID NO: 77 and SEQ ID NO: 78 were purified on a HisTrap column using standard methods. 2 mg protein (200 μL of 10 mg/ml) in PBS at pH 7.4 was activated by treatment with 2 μl of 0.1M dithiothreitol and 5 μl of TEV protease for two hours at 30° C. Cleaved and reduced protein was applied to a 1-ml HisTrap column equilibrated with PBS. The C-terminal fragment bound to the column and the activated N-terminal SEQ ID NO: 80 or SEQ ID NO: 70 product with a free cysteine near its C-terminus was collected in the flow through.

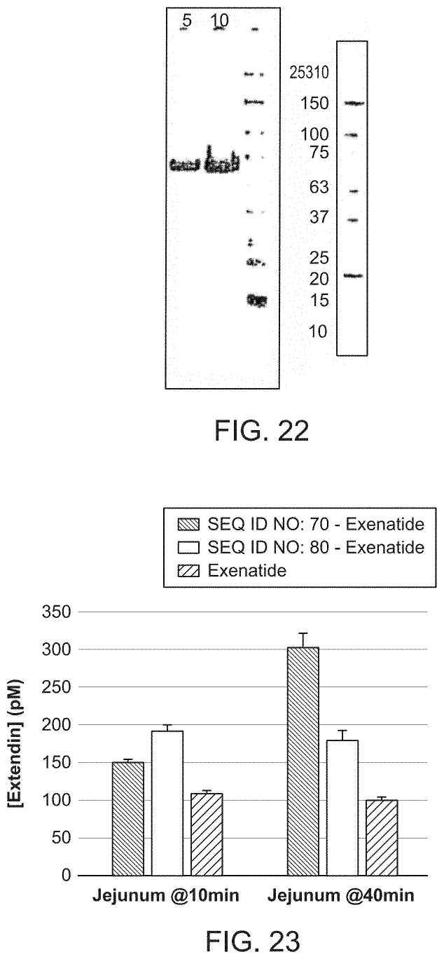

The maleimide-activated Exenatide and carriers (sulfhydryl-SEQ ID NO: 80 protein or sulfhydryl-SEQ ID NO: 70 protein) were mixed in equal molar amounts and then incubated for 60 minutes at room temperature. The purity of the SMCC-crosslinked SEQ ID NO: 70—Exenatide complex was assessed on a Coomassie-stained SDS gel. The complex was approximately the correct molecular weight and had >90% purity (FIG. 22). The crosslinked delivery construct was then stored at 4° C.

### Example 12: In Vivo Transcytosis of Exenatide Delivery Constructs

The delivery constructs from Example 11 were tested for intestinal epithelial transport as follows: wild-type Sprague Dawley® rats (˜200-250 grams, ˜6 weeks old, purchased from Charles River) were fasted overnight to clear their intestines. The following materials were prepared: microfuge tubes containing 4% formaldehyde, tubes for tissue preservation, microfuge tubes for blood collection, microfuge tubes for serum collection, PBS, and test article. Animals were prepped for the experiment by anesthetizing them with Isoflorane and shave their abdomens. Four injections were prepared for each animal (2 per jejunum and 2 per colon). The abdominal cavity was opened. Injection sites were located and marked with distinguishing colors. Test articles were slowly injected into the lumen over 10 minutes for the colon and 40 minutes for the jejunum. Animals received 35 μg of protein per injection at concentration of 1 μg/μL. Animals were euthanized at 50 minutes. Terminal blood was collected via cardiac puncture. The jejunum and colon were removed and placed on a plastic-lined work surface. The contents of the jejunum and colon were flushed using PBS and discarded. A 1 cm length of intestine was excised from the injection site. The excised tissue was cut in half. One section was placed into 4% formaldehyde. The remaining tissue was then sliced lengthwise and immediately placed in a microfuge tube and frozen. This process was repeated for all injection sites. The liver (˜1 cm³) was removed and divided into 2 pieces. For storage, one section of liver was placed in formaldehyde and a second was immediately frozen. Intestinal, liver & blood serum samples were collected at 40 min after injection. Blood samples were centrifuged and the resulting serum was transferred to a container for storage. Samples were transported on dry ice and stored at −80° C. The dosing strategy was as follows:

| | | |
|---|---|---|
| SEQ ID NO: 70-Exenatide | 100 μL, 490 pmol/29.4 μg | (4.9 μM) |
| SEQ ID NO: 80-Exenatide | 100 μL, 490 pmol/30.9 μg | (4.9 μM) |
| SEQ ID NO: 11 | 100 μL, 490 pmol/2 μg | (4.9 μM) |

Bioanalytical analysis of the intestinal epithelial transport of SEQ ID NO: 70-Exenatide, SEQ ID NO: 80-Exenatide, and SEQ ID NO: 11 (Exenatide) was performed using an Exendin-4 ELISA kit (Phoenix Pharma, Cat #EK-070-94) as follows: tissue samples were obtained from Brains On-line; 300 μL assay buffer (1×) was added to each tube containing a tissue sample; tissue was removed from the assay buffer and placed on a sterile, clean cell culture lid plate; intestinal samples were gently scrapped with a cell scrapper, being careful to avoid collection of the mesentery; liver samples were treated in a similar manner with additional maceration and homogenization; the resulting cellular homogenate was transferred back into the original tube; remaining tissue samples and the work area were rinsed with 100 μL buffer (2λ); cellular homogenate solution was centrifuged at maximum force for 5 minutes; supernatant was applied to an ELISA plate, which was processed according to the manufacturer's instructions; remaining supernatant was stored at −20° C. for later use.

As depicted in FIG. 23, transport of both SEQ ID NO: 70-Exenatide and SEQ ID NO: 80-Exenatide across intestinal epithelial cells was observed at 10 minutes and at 40 minutes. Moreover, both SEQ ID NO: 70-Exenatide and SEQ ID NO: 80-Exenatide were transported at a higher rate than SEQ ID NO: 11 (Exenatide) alone, especially at 40 minutes.

### Example 13: Glucose Regulatory Activity of a Delivery Construct with an Exenatide Payload

The glucose challenge model used to test SEQ ID NO: 70-Exenatide was designed to examine the capacity of a GLP-1-like activity to enhance the rate of recovery from a glucose excursion. An IP injection of glucose was used to incite the glucose excursion event and Exenatide delivered by IP injection was used as a positive control for timing and extent of effect relative to that observed for the sham IP injection control.

The male CD1 mice used in the glucose challenge test were 9-16 weeks of age. To minimize stress due to handling, animals were acclimatized to the environment, blood sampling, and dosing procedures for 1 week prior to the start of experiments, as plasma glucose can be responsive to handling stress. Mice were fasted for 18 h prior to study. Animals were weighed prior to study. All animals had a baseline blood sugar reading taken prior to receiving 2 mg/kg dose of D-glucose solution (in 50 μL sterile PBS) by intraperitoneal (IP) injection. Animals then received either an IP injection of 10 mg of SEQ ID NO: 14 (positive control), an oral gavage of a test treatment that contained 10 mg of SEQ ID NO: 14 in 200 μl of 0.2 M NaHCO₃ (pH 8.5), or an oral gavage of 200 μl of 0.2 M NaHCO₃ (negative control). Blood samples were taken at t=0, 15, 30, 45, 60 min; 2, 3, and 4 hr. Blood glucose measurements were made using 5 μL, blood sample taken from the tail with a commercial glucometer that was calibrated with glucose standards prior to the start of the study.

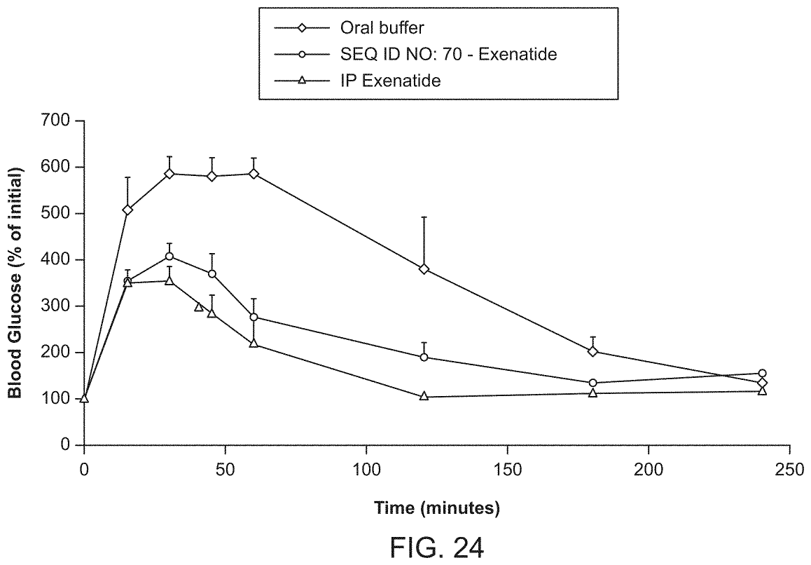

A time-concentration profile of blood glucose levels for animals receiving the three different treatments is shown in FIG. 24. Correction of the blood glucose excursion began as early as 15 min after a 10 μg IP injection of commercial Exenatide (1-40)-Gly, a correction profile that was completed by 120 min. Oral gavage of SEQ ID NO: 70-Exenatide (10 mg) resulted in a similar time-concentration pattern of blood glucose. By comparison, negative control mice achieved 2-fold higher blood sugar levels that required ˜4 h to fully recover to baseline. These results suggest that the SEQ ID NO: 70 carrier sequence was capable of facilitating epithelial cell transcytosis of biologically active Exenatide that was sufficient to obtain a pharmacodynamic outcome in this glucose challenge model.

### Example 14: GLP-1 Receptor Activation by a Delivery Construct with an Exenatide Payload

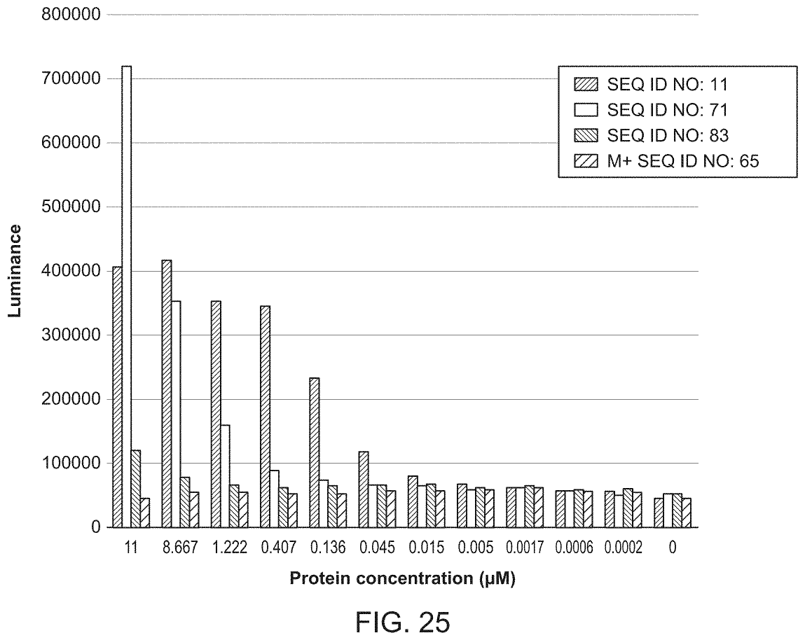

SEQ ID NO: 71 is a fusion protein delivery construct comprising an N-terminal extendin-4 (SEQ ID NO: 14) domain, a spacer (SEQ ID NO: 79) and a C-terminal carrier (SEQ ID NO: 73). SEQ ID NO: 83 is a fusion protein delivery construct comprising an N-terminal carrier (SEQ ID NO: 67), a spacer (SEQ ID NO: 79) and a C-terminal extendin-4 (SEQ ID NO: 14) domain. A PathHunter® β-Arrestin G-Protein Coupled Receptor (GPCR) Assay (DiscoverRx) was used to assay the ability of SEQ ID NO: 71, SEQ ID NO: 11 (Exenatide), and M+SEQ ID NO: 65 to bind the GLP-1 receptor. In the PathHunter assay, ligand binding activates the GLP-1 receptor and leads to β-arrestin recruitment to the receptor. The activation status of the receptor is detected using a gain-of-signal assay based on Enzyme Fragment Complementation. The β-galactosidase enzyme ((3-gal) is split into two fragments, an enzyme donor (ED) and an enzyme acceptor (EA). Independently these fragments have no activity. However, they complement each other to form an active β-gal enzyme when brought together by assembly of a protein complex. The GLP-1 receptor was tagged with the ED fragment and co-expressed in cells stably expressing β-arrestin tagged with with EA. Recruitment of EA-tagged β-arrestin by an activated ED-tagged GLP-1 receptor brings the EA and ED domains together to reconstitute β-gal enzyme activity, which can be detected by release of a luminescent product. FIG. 25 illustrates that SEQ ID NO: 11 and SEQ ID NO: 71 bound the receptor. SEQ ID NO: 83 showed reduced activity relative to SEQ ID NO: 71.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein can be employed in practicing the invention. It is intended that the following claims define the scope of the invention and that methods and structures within the scope of these claims and their equivalents be covered thereby.

TABLE 12
Sequences

| SEQ ID NO | | Sequence |
|---|---|---|
| 1 | | VEEALNIFDECRSPCSLTPEPGKPIQSKLSIPSDVVLDEGVLYYSMTINDEQND |
| | | IKDEDKGESIITIGEFATVRATRHYVNQDAPFGVIHLDITTENGTKTYSYNRKE |
| | | GEFAINWLVPIGEDSPASIKISVDELDQQRNIIEVPKLYSIDLDNQTLEQWKTQ |
| | | GNVSFSVTRPEHNIAISWPSVSYKAAQKEGSRHKRWAHWHTGLALCWLVPMDAI |
| | | YNYITQQNCTLGDNWFGGSYETVAGTPKVITVKQGIEQKPVEQRIHFSKGNAMS |
| | | ALAAHRVCGVPLETLARSRKPRDLTDDLSCAYQAQNIVSLFVATRILFSHLDSV |
| | | FTLNLDEQEPEVAERLSDLRRINENNPGMVTQVLTVARQIYNDYVTHHPGLTPE |
| | | QTSAGAQAADILSLFCPDADKSCVASNNDQANINIESRSGRSYLPENRAVITPQ |
| | | GVTNWTYQELEATHQALTREGYVFVGYHGTNHVAAQTIVNRIAPVPRGNNTENE |
| | | EKWGGLYVATHAEVAHGYARIKEGTGEYGLPTRAERDARGVMLRVYIPRASLER |
| | | FYRTNTPLENAEEHITQVIGHSLPLRNEAFTGPESAGGEDETVIGWDMAIHAVA |
| | | IPSTIPGNAYEELAIDEEAVAKEQSISTKPPYKERKDELK |
| 2 | | VEEALNIFDECRSPCSLTPEPGKPIQSKLSIPSDVVLDEGVLYYSMTINDEQND |
| | | IKDEDKGESIITIGEFATVRATRHYVNQDAPFGVIHLDITTENGTKTYSYNRKE |
| | | GEFAINWLVPIGEDSPASIKISVDELDQQRNIIEVPKLYSIDLDNQTLEQWKTQ |
| | | GNVSFSVTRPEHNIAISWPSVSYKAAQKEGSRHKRWAHWHTGLALCWLVPMDAI |
| | | YNYITQQNCTLGDNWFGGSYETVAGTPKVITVKQGIEQKPVEQRIHFSKGNAMS |
| | | ALAAHRVCGVPLETLARSRKPRDLTDDLSCAYQAQNIVSLFVATRILFSHLDSV |
| | | FTLNLDEQEPEVAERLSDLRRINENNPGMVTQVLTVARQIYNDYVTHHPGLTPE |
| | | QTSAGAQA |
| 3 | | MVEEALNIFDECRSPCSLTPEPGKPIQSKLSIPSDVVLDEGVLYYSMTINDEQN |
| | | DIKDEDKGESIITIGEFATVRATRHYVNQDAPFGVIHLDITTENGTKTYSYNRK |
| | | EGEFAINVVLVPIGEDSPASIKISVDELDQQRNIIEVPKLYSIDLDNQTLEQWK |
| | | TQGNVSFSVTRPEHNIAISWPSVSYKAAQKEGSRHKRWAHWHTGLALCWLVPMD |
| | | AIYNYITQQNCTLGDNVVFGGSYETVAGTPKVITVKQGIEQKPVEQRIHFSKGS |
| | | GGSGGGGSGGGGSPRRRRSSSRPVRRRRRPRVSRRRRRRGGRRRRHHHHHH |
| 4 | | GGGGSGGGGSGGGG |
| 5 | | MFPTIPLSRLFDNAMLRAHRLHQLAFDTYQEFEEAYIPKEQKYSFLQNPQTSLC |
| | | FSESIPTPSNREETQQKSNLELLRISLLLIQSWLEPVQFLRSVFANSLVYGASD |
| | | SNVYDLLKDLEEGIQTLMGRLEDGSPRTGQIFKQTYSKFDTNSHNDDALLKNYG |
| | | LLYCFRKDMDKVETFLRIVQCRSVEGSCGF |
| 6 | | VEEALNIFDECRSPCSLTPEPGKPIQSKLSIPSDVVLDEGVLYYSMTINDEQND |
| | | IKDEDKGESIITIGEFATVRATRHYVNQDAPFGVIHLDITTENGTKTYSYNRKE |
| | | GEFAINWLVPIGEDSPASIKISVDELDQQRNIIEVPKLYSIDLDNQTLEQWKTQ |
| | | GNVSFSVTRPEHNIAISWPSVSYKAAQKEGSRHKRWAHWHTGLALCWLVPMDAI |
| | | YNYITQQNCTLGDNWFGGSYETVAGTPKVITVKQGIEQKPVEQRIHFSKGNAMS |
| | | ALAAHRVCGVPLETLARSRKPRDLTDDLSCAYQAQNIVSLFVATRILFSHLDSV |
| | | FTLNLDEQEPEVAERLSDLRRINENNPGMVTQVLTVARQIYNDYVTHHPGLTPE |
| | | QTSAGAQAADILSLFCPDADKSCVASNNDQANINIES |
| 7 | | X1-E-X3-X4-L-X6-I-F-D-E-C-R-S-P-C-X16-L-T-P-E-X21-G- |
| | | K-X24-I-Q-S-K-L-X30-I-P-X33-D-V-V-L-D-E-G-V-L-Y-Y-S-M- |
| | | T-I-N-D-E-Q-N-D-I-X56-D-E-X59-K-G-E-S-I-I-T-X67-G-E-F- |
| | | A-T-X73-R-A-T-R-H-Y-V-X81-Q-D-A-P-F-G-V-I-X90-L-D-I-T- |
| | | T-E-N-G-T-K-X101-Y-S-X104-N-R-K-X108-X109-E-F-X112-I- |
| | | X114-W-L-V-X118-X119-G-E-D-S-P-A-S-I-K-I-S-X131-D-E- |
| | | X134-D-Q-X137-R-N-I-I-E-V-P-K-L-Y-S-I-D-L-D-N-Q-T-L-E- |
| | | Q-W-X160-X161-Q-G-N-V-X166-F-X168-V-T-R-P-E-X174-X175- |
| | | I-A-I-S-W-P-S-V-S-Y-X186-A-A-X189-K-X191-G-X193-R-H-K- |
| | | R-W-A-X200-W-X202-T-X204-X205-X206-X207-X208-X209-L- |
| | | X211-X212-X213-X214-X215-X216-X217-X218-X219-X220- |
| | | X221-X222-X223-X224-C-T-X227-G-X229-X230-W-X232-G-G- |
| | | X235-Y-X237-T-V-A-G-X242-P-X244-X245-I-X247-V-K-Q-G- |
| | | X252-E-Q-K-X256-V-E-Q-R-I-H-F-S-X265-X266-N-A-X269- |
| | | X270-X271-L-A-A-H-R-V-C-G-V-P-L-E-T-L-A-R-X288-R-K-P- |
| | | R-X293-L-X295-D-D-L-X299-C-X301-Y-X303-A-Q-X306-I-V-S- |
| | | L-F-X312-A-T-R-X316-L-F-X319-H-X321-D-S-X324-F-T-L-N- |
| | | L-X330-X331-Q-X333-P-X335-V-X337-E-R-L-X341-X342-X343- |
| | | R-X345-I-N-E-X349-N-P-G-X353-V-X355-Q-V-L-T-X360-A-R- |
| | | Q-I-Y-N-D-Y-V-T-X371-H-P-X374-L-X376-P-E-Q-T-S-A-X383- |
| | | A-Q-A-A-D-I-L-S-L-X393-X394-P-D-X397-D-X399-X400-C-V- |
| | | A-X404-X405-X406-D-Q-A-N-I-N-X413-E-S-R-S-G-R-S-Y-L- |
| | | X423-E-N-R-A-V-I-T-X431-Q-G-V-T-N-W-T-Y-Q-E-L-X443- |
| | | X444-X445-H-Q-X448-L-T-X451-E-X453-Y-V-F-V-G-Y-H-G-T- |
| | | N-H-X465-A-A-Q-X469-I-V-N-R-I-X475-P-V-P-R-G-X481- |
| | | X482-T-E-X485-E-X487-X488-W-G-G-X492-Y-V-X495-T-X497- |
| | | A-X499-X500-X501-X502-X503-Y-X505-R-X507-X508-X509-G- |
| | | T-X512-X513-X514-X515-X516-X517-T-X519-X520-X521- |
| | | X522-X523-X524-R-G-V-M-L-X530-V-Y-X533-X534-X535-A-S- |
| | | L-E-R-F-Y-R-X544-N-X546-X547-L-E-X550-X551-X552-X553- |
| | | X554-X555-X556-X557-V-I-G-H-X562-L-P-L-R-N-E-A-F-T-G- |
| | | X573-X574-X575-X576-X577-G-X579-X580-E-T-X583-I-G-W- |
| | | D-X588-A-I-X591-X592-V-A-I-P-S-T-I-P-G-N-X603-Y-X605- |
| | | X606-L-X608-X609-X610-E-E-A-X614-A-X616-E-Q-S-I-S- |
| | | X622-K-P-P-Y-K-E-X629-X630-D-E-L-K; wherein X1 is |
| | | selected from the group consisting of V and L; X3 is |
| | | selected from the group consisting of E and D; X4 is |
| | | selected from the group consisting of A and E; X6 is |
| | | selected from the group consisting of N and K; X16 is |
| | | selected from the group consisting of S and L; X21 is |
| | | selected from the group consisting of P and L; X24 is |
| | | selected from the group consisting of P and Q; X30 is |
| | | selected from the group consisting of S and F; X33 is |
| | | selected from the group consisting of S and G; X56 is |
| | | selected from the group consisting of K and M; X59 is |
| | | selected from the group consisting of D and G; X67 is |
| | | selected from the group consisting of I and F; X73 is |
| | | selected from the group consisting of V and I; X81 is |
| | | selected from the group consisting of N and S; X90 is |
| | | selected from the group consisting of H and N; X101 is |
| | | selected from the group consisting of T and M; X104 is |
| | | selected from the group consisting of Y and F; X108 is |
| | | selected from the group consisting of E and D; X109 is |
| | | selected from the group consisting of G and S; X112 is |
| | | selected from the groupconsisting of A and T; X114 is |
| | | selected from the group consisting ofN and H; X118 is |
| | | selected from the group consisting of P and I; X119 is |
| | | selected from the group consisting of I and P; X131 is |
| | | selected from the group consisting of V and I; X134 is |
| | | selected from the group consisting L and I; X137 is |
| | | selected from the group consisting Qand K; X160 is |
| | | selected from the group consisting K and E; X161 is |
| | | selected from the group consisting T and N; X166 is |
| | | selected from the group consisting S and F; X168 is |
| | | selected from the group consisting S and A; X174 is |
| | | selected from the group consisting H and Q; X175 is |
| | | selected from the group consisting N, S, SIAKQS(SEQ |
| | | ID NO: 92), and SIAKQSIAKQS(SEQ ID NO: 93); X186 is |
| | | selected from the group consisting of K and N; X189 is |
| | | selected from the group consisting of Q, E, and H; |
| | | X191 is selected from the group consisting of E, N, |
| | | and D; X193 is selected from the group consisting of S |
| | | and A; X200 is selected from the group consisting of H |
| | | and N; X202 is selected from the group consisting of H, |
| | | L, F, and R; X204 is selected from the group consisting |
| | | of G and T; X205 is selected from the group consisting |
| | | of L and S; X206 is selected from the group consisting |
| | | of A and P; X207 is selected from the group consisting |
| | | of L, E, and K; X208 is selected from the group |
| | | consisting of C and V; X209 is selected from the group |
| | | consisting of W, V, and T; X211 is selected from the |
| | | group consisting of V and no amino acid; X212 is |
| | | selected from the group consisting of P and no amino |
| | | acid; X213 is selected from the group consisting of M, |
| | | I, L, and no amino acid; X214 is selected from the |
| | | group consisting of D and no amino acid; X215 is |
| | | selected from the group consisting of A and no amino |
| | | acid; X216 is selected from the group consisting of I |
| | | and no amino acid; X217 is selected from the group |
| | | consisting of Y and C; X218 is selected from the group |
| | | consisting of N and F; X219 is selected from the group |
| | | consisting of Y and F; X220 is selected from the group |
| | | consisting of I and E; X221 is selected from the group |
| | | consisting of T and D; X222 is selected from the group |
| | | consisting of Qand P; X223 is selected from the group |
| | | consisting of Q, E, and A; X224 is selected from the |
| | | group consisting of N, L, and Q; X227 is selected from |
| | | the group consisting of L and Y; X229 is selected from |
| | | the group consisting of D and E; X230 is selected from |
| | | the group consisting of N and D; X232 is selected from |
| | | the group consisting of F, H, and Y; X235 is selected |
| | | from the group consisting of S and A; X237 is selected |
| | | from the group consisting of E and K; X242 is selected |
| | | from the group consisting of T and I; X244 is selected |
| | | from the group consisting of K, E, and G; X245 is |
| | | selected from the group consisting of V and A; X247 is |
| | | selected from the group consisting of T and M; X252 is |
| | | selected from the group consisting of I and M; X256 is |
| | | selected from the group consisting of P, T, and A; |
| | | X265 is selected from the group consisting of K, Q, |
| | | and N; X266 is selected from the group consisting of G |
| | | and K; X269 is selected from the group consisting of M |
| | | and I; X270 is selected from the group consisting of S |
| | | and E; X271 is selected from the group consisting of A |
| | | and T; X288 is selected from the group consisting of S |
| | | and G; X293 is selected from the group consisting of D |
| | | and Y; X295 is selected from the group consisting of |
| | | T, P, and Q; X299 is selected from the group |
| | | consisting of S and Q; X301 is selected from the group |
| | | consisting of A and V; X303 is selected from the group |
| | | consisting of Qand N; X306 is selected from the group |
| | | consisting of N and Q; X312 is selected from the group |
| | | consisting of V and L; X316 is selected from the group |
| | | consisting of I and M; X319 is selected from the group |
| | | consisting of S and T; X321 is selected from the group |
| | | consisting of L and I; X324 is selected from the group |
| | | consisting of V and I; X330 is selected from the group |
| | | consisting of D, E, and H; X331 is selected from the |
| | | group consisting of E and G; X333 is selected from the |
| | | group consisting of E and A; X335 is selected from the |
| | | group consisting of E and A; X337 is selected from the |
| | | group consisting of A and T; X341 is selected from the |
| | | group consisting of S, D, and T; X342 is selected from |
| | | the group consisting of D and A; X343 is selected from |
| | | the group consisting of L and I; X345 is selected from |
| | | the group consisting of R and Q; X349 is selected from |
| | | the group consisting of N and D; X353 is selected from |
| | | the group consisting of M and V; X355 is selected from |
| | | the group consisting of T and I; X360 is selected from |
| | | the group consisting of V and I; X371 is selected from |
| | | the group consisting of H and E; X374 is selected from |
| | | the group consisting of G and L; X376 is selected from |
| | | the group consisting of T and I; X383 is selected from |
| | | the group consisting of G and S; X393 is selected from |
| | | the group consisting of F and L; X394 is selected from |
| | | the group consisting of C and Y; X397 is selected from |
| | | the group consisting of A and T; X399 is selected from |
| | | the group consisting of K, E, and G; X400 is selected |
| | | from the group consisting of S, P, and H; X404 is |
| | | selected from the group consisting of S and L; X405 is |
| | | selected from the group consisting of N and D; X406 is |
| | | selected from the group consisting of N and S; X413 is |
| | | selected from the group consisting of I and V; X423 is |
| | | selected from the group consisting of P and L; X431 is |
| | | selected from the group consisting of P and Q; X443 is |
| | | selected from the group consisting of E and D; X444 is |
| | | selected from the group consisting of A and T; X445 is |
| | | selected from the group consisting of T and K; X448 is |
| | | selected from the group consisting of A and T; X451 is |
| | | selected from the group consisting of R and Q; X453 is |
| | | selected from the group consisting of G and D; X465 is |
| | | selected from the group consisting of V and A; X469 is |
| | | selected from the group consisting of T, S, and N; |
| | | X475 is selected from the group consisting of A, S, |
| | | and T; X481 is selected from the group consisting of N |
| | | and S; X482 is selected from the group consisting of N |
| | | and D; X485 is selected from the group consisting of |
| | | N, S, and K; X487 is selected from the group |
| | | consisting of E, R, and K; X488 is selected from the |
| | | group consisting of K, A, and E; X492 is selected |
| | | from the group consisting of L and V; X495 is |
| | | selected from the group consisting of A and S; X497 |
| | | is selected from the group consisting of H and D; |
| | | X499 is selected from the group consisting of E and |
| | | S; X500 is selected from the group consisting of V and |
| | | L; X501 is selected from the group consisting of A and |
| | | N; X502 is selected from the group consisting of H and |
| | | Y; X503 is selected from the group consisting of G and |
| | | R; X505 is selected from the group consisting of A and |
| | | T; X507 is selected from the group consisting of I and |
| | | L; X508 is selected from the group consisting of K and |
| | | Q; X509 is selected from the group consisting of E and |
| | | K; X512 is selected from the group consisting of G and |
| | | A; X513 is selected from the group consisting of E, D, |
| | | and N; X514 is selected from the group consisting of |
| | | Y, G, A, and N; X515 is selected from the group |
| | | consisting of G and E; X516 is selected from the group |
| | | consisting of L and G; X517 is selected from the group |
| | | consisting of P and L; X519 is selected from the group |
| | | consisting of R, P, and T; X520 is selected from the |
| | | group consisting of A and E; X521 is selected from the |
| | | group consisting of E and K; X522 is selected from the |
| | | group consisting of R, Q, and K; X523 is selected from |
| | | the group consisting of D, K, and E; X524 is selected |
| | | from the group consisting of A, T, and S; X530 is |
| | | selected from the group consisting of R and K; X533 is |
| | | selected from the group consisting of I and L; X534 is |
| | | selected from the group consisting of P and H; X535 is |
| | | selected from the group consisting of R and Q; X544 is |
| | | selected from the group consisting of T and I; X546 is |
| | | selected from the group consisting of T, A, and I; |
| | | X547 is selected from the group consisting of P and D; |
| | | X550 is selected from the group consisting of N and K; |
| | | X551 is selected from the group consisting of A and E; |
| | | X552 is selected from the group consisting of E, R, |
| | | and D; X553 is selected from the group consisting of |
| | | E, N, and R; X554 is selected from the group |
| | | consisting of H and L; X555 is selected from the group |
| | | consisting of I and V; X556 is selected from the group |
| | | consisting of T and E; X557 is selected from the group |
| | | consisting of Q, R, H, and D; X562 is selected from |
| | | the group consisting of S and P; X573 is selected from |
| | | the group consisting of P and T; X574 is selected from |
| | | the group consisting of E and D; X575 is selected from |
| | | the group consisting of S, A, and R; X576 is selected |
| | | from the group consisting of A, E, and V; X577 is |
| | | selected from the group consisting of G, E, and D; |
| | | X579 is selected from the group consisting of E and S; |
| | | X580 is selected from the group consisting of D and N; |
| | | X583 is selected from the group consisting of V and A; |
| | | X588 is selected from the group consisting of M and I; |
| | | X591 is selected from the group consisting of H and Y; |
| | | X592 is selected from the group consisting of A and G; |
| | | X603 is selected from the group consisting of A and S; |
| | | X605 is selected from the group consisting of E and A; |
| | | X606 is selected from the group consisting of E, A, Q, |
| | | G, V, and R; X608 is selected from the group |
| | | consisting of A, P, and T; X609 is selected from the |
| | | group consisting of I, T, and P; X610 is selected from |
| | | the group consisting of D and A; X614 is selected from |
| | | the group consisting of V and VVKEAI(SEQ ID NO: 94); |
| | | X616 is selected from the group consisting of K and E; |
| | | X622 is selected from the group consisting of T, A, |
| | | and P; and X629 is selected from the group consisting |
| | | of R, Q, and H; and X630 is selected from the group |
| | | consisting of K and no amino acid. |
| 8 | | VLYYSMTINDEQNDIKDEDKGESIITIGEFATVRATRHYVNQDAPFGVIHLDIT |
| | | TENGTKTYSYNRKEGEFAINWLVPIGEDSPASIKISVDELDQQRNIIEVPKLYS |
| | | IDLDNQTLEQWKTQGNVSFSVTRPEHNIAISWPSVSYKA |
| 9 | | GVLYYSMTINDEQNDIKDEDKGESIITIGEFATVRATRHYVNQDAPFGVIHLDI |
| | | TTENGTKTYSYNRKEGEFAINWLVPIGEDSPASIKISVDELDQQRNIIEVPKLY |
| | | SIDLDNQTLEQWKTQGNVSFSVTRPEHNIAISWPSVSYKAAQKEGSRHKRWAHW |
| | | HTGL |
| 10 | | VEEALNIFDECRSPCSLTPEPGKPIQSKLSIPSDVVLDEGVLYYSMTINDEQND |
| | | IKDEDKGESIITIGEFATVRATRHYVNQDAPFGVIHLDITTENGTKTYSYNRKE |
| | | GEFAINWLVPIGEDSPASIKISVDELDQQRNIIEVPKLYSIDL |
| 11 | Exenatide | H-HisGlyGluGlyThrPheThrSerAspLeuSerLysGlnMetGluGluGlu |
| | (Byetta) | AlaValArgLeuPheIleGluTrpLeuLysAsnGlyGlyProSerSerGlyAla |
| | | ProProProSer-NH₂ |
| 12 | | LEEALNIFDECRSPCSLTPEPGKPIQSKLSIPSDVVLDEGVLYYSMTINDEQND |
| | | IKDEDKGESIITIGEFATVRATRHYVNQDAPFGVIHLDITTENGTKTYSYNRKE |
| | | GEFAINWLVPIGEDSPASIKISVDELDQQRNIIEVPKLYSIDLDNQTLEQWKTQ |
| | | GNVSFSVTRPEHNIAISWPSVSYKAAQKEGSRHKRWAHWHTGLALCWLVPMDAI |
| | | YNYITQQNCTLGDNWFGGSYETVAGTPKVITVKQGIEQKPVEQRIHFSKGNAMS |
| | | ALAAHRVCGVPLETLARSRKPRDLTDDLSCAYQAQNIVSLFVATRILFSHLDSV |
| | | FTLNLDEQEPEVAERLSDLRRINENNPGMVTQVLTVARQIYNDYVTHHPGLTPE |
| | | QTSAGAQAADILSLFCPDADKSCVASNNDQANINIESRSGRSYLPENRAVITPQ |
| | | GVTNWTYQELEATHQALTREGYVFVGYHGTNHVAAQTIVNRIAPVPRGNNTENE |
| | | EKWGGLYVATHAEVAHGYARIKEGTGEYGLPTRAERDARGVMLRVYIPRASLER |
| | | FYRTNTPLENAEEHITQVIGHSLPLRNEAFTGPESAGGEDETVIGWDMAIHAVA |
| | | IPSTIPGNAYEELAIDEEAVAKEQSISTKPPYKERKDELK |
| 13 | | LEEALNIFDECRSPCSLTPEPGKPIQSKLSIPSDVVLDEGVLYYSMTINDEQND |
| | | IKDEDKGESIITIGEFATVRATRHYVNQDAPFGVIHLDITTENGTKTYSYNRKE |
| | | GEFAINWLVPIGEDSPASIKISVDELDQQRNIIEVPKLYSIDLDNQTLEQWKTQ |
| | | GNVSFSVTRPEHNIAISWPSVSYKAAQKEGSRHKRWAHWHTGLALCWLVPMDAI |
| | | YNYITQQNCTLGDNWFGGSYETVAGTPKVITVKQGIEQKPVEQRIHFSKGNAMS |
| | | ALAAHRVCGVPLETLARSRKPRDLTDDLSCAYQAQNIVSLFVATRILFSHLDSV |
| | | FTLNLDEQEPEVAERLSDLRRINENNPGMVTQVLTVARQIYNDYVTHHPGLTPE |
| | | QTSAGAQA |
| 14 | Exendin-4 | HGEGTFTSDLSKQMEEEAVRLFIEWLKNGGPSSGAPPPS |
| 15 | Lixisenatide | HGEGTFTSDLSKQMEEEAVRLFIEWLKNGGPSSGAPPSKKKKKK |
| | (Adlyxin) | |
| 16 | Liraglutide | HAEGTFTSDVSSYLEGQAAKEEFIAWLVRGRG (gamma-E-palmitoyl |
| | | at E21) |
| 17 | Dulaglutide | HGEGTFTSDVSSYLEEQAAKEFIAWLVKGGGGGGGSGGGGSGGGGSAESKYGPP |
| | | CPPCPAPEAAGGPSVFLFPPKPKDTLMISRTPEVTCVVVDVSQEDPEVQFNWYV |
| | | DGVEVHNAKTKPREEQFNSTYRVVSVLTVLHQDWLNGKEYKCKVSNKGLPSSIE |
| | | KTISKAKGQPREPQVYTLPPSQEEMTKNQVSLTCLVKGFYPSDIAVEWESNGQP |
| | | ENNYKTTPPVLDSDGSFFLYSRLTVDKSRWQEGNVFSCSVMHEALHNHYTQKSL |
| | | SLSLG |
| 18 | Teduglutide | HGDGSFSDEMNTILDNLAARDFINWLIQTKITD |
| 19 | Teduglutide | H-HisGlyAspGlySerPheSerAspGluMetAsnThrIleLeuAspAsnLeu |
| | | AlaAlaArgAspPheIleAsnTrpLeuIleGlnThrLysIleThrAsp-OH |
| 20 | hGH | FPTIPLSRLFDNAMLRAHRLHQLAFDTYQEFEEAYIPKEQKYSFLQNPQTSLCF |
| | | SESIPTPSNREETQQKSNLELLRISLLLIQSWLEPVQFLRSVFANSLVYGASDS |
| | | NVYDLLKDLEEGIQTLMGRLEDGSPRTGQIFKQTYSKFDTNSHNDDALLKNYGL |
| | | LYCFRKDMDKVETFLRIVQCRSVEGSCGF |
| 21 | IL-22 | APISSHCRLDKSNFQQPYITNRTFMLAKEASLADNNTDVRLIGEKLFHGVSMSE |
| | | RCYLMKQVLNFTLEEVLFPQSDRFQPYMQEVVPFLARLSNRLSTCHIEGDDLHI |
| | | QRNVQKLKDTVKKLGESGEIKAIGELDLLFMSLRNACI |
| 22 | IL-10 | SPGQGTQSENSCTHFPGNLPNMLRDLRDAFSRVKTFFQMKDQLDNLLLKESLLE |
| | | DFKGYLGCQALSEMIQFYLEEVMPQAENQDPDIKAHVNSLGENLKTLRLRLRRC |
| | | HRFLPCENKSKAVEQVKNAFNKLQEKGIYKAMSEFDIFINYIEAYMTMKIRN |
| 23 | Preproglucagon | MKSIYFVAGLFVMLVQGSWQRSLQDTEEKSRSFSASQADPLSDPDQMNEDKRHS |
| | | QGTFTSDYSKYLDSRRAQDFVQWLMNTKRNRNNIAKRHDEFERHAEGTFTSDVS |
| | | SYLEGQAAKEFIAWLVKGRGRRDFPEEVAIVEELGRRHADGSFSDEMNTILDNL |
| | | AARDFINVVLIQTKITDRK |
| 24 | Glucagon | RSLQDTEEKSRSFSASQADPLSDPDQMNEDKRHSQGTFTSDYSKYLDSRRAQDF |
| | proprotein | VQWLMNTKRNRNNIAKRHDEFERHAEGTFTSDVSSYLEGQAAKEFIAWLVKGRG |
| | | RRDFPEEVAIVEELGRRHADGSFSDEMNTILDNLAARDFINWLIQTKITDRK |
| 25 | Glucagon | HSQGTFTSDYSKYLDSRRAQDFVQWLMNT |
| | peptide | |
| 26 | Glucagon-like | HAEGTFTSDVSSYLEGQAAKEFIAWLVKGR |
| | peptide 1 | |
| | (GLP-1) | |
| 27 | Glucagon-like | HADGSFSDEMNTILDNLAARDFINWLIQTKITD |
| | peptide 2 | |
| | (GLP-2) | |
| 28 | Exendin-3 | HSDGTFTSDLSKQMEEEAVRLFIEWLKNGGPSSGAPPPS |
| 29 | Efpeglenatide | 4-imidazoacetyl-GEGTFTSDLSKQMEEEAVRLFIEWL-(K-PEG-Fc)- |
| | | NGGPSSGAPPPS-NH2 |
| 30 | Semaglutide | HXEGTFTSDVSSYLEGQAAKEFIAWLVRGRG (acylated) |
| | (Ozempic) | |
| 31 | GLP-1R | YAEGTFISDYSIAMDKIHQQDFVNWLLAQKGKKNDWK |
| | agonist (aa | |
| | 1-37 of GIP) | |
| 32 | GLP-1R | ISDYSIAMDKIHQQDFVNWLLAQKGKKNDW |
| | agonist (aa | |
| | 7-36 of GIP) | |
| 33 | Tirzepatide | YXEGTFTSDYSIXLDKIAQKAFVQWLIAGGPSSGAPPPS |
| 34 | Oxyntomodulin | HSQGTFTSDYSKYLDSRRAQDFVQWLMNTKRNKNNIA |
| 35 | Gastric | MVATKTFALLLLSLFLAVGLGEKKEGHFSALPSLPVGSHAKVSSPQPRGPRYAE |
| | inhibitory | GTFISDYSIAMDKIHQQDFVNWLLAQKGKKNDWKHNITQREARALELASQANRK |
| | polypeptide | EEEAVEPQSSPAKNPSDEDLLRDLLIQELLACLLDQTNLCRLRSR |
| | preprotein | |
| 36 | Gastric | YAEGTFISDYSIAMDKIHQQDFVNWLLAQKGKKNDWKHNITQ |
| | inhibitory | |
| | polypeptide | |
| | (GIP) | |
| 37 | GIPR | YEGTFISDYSIAMDKIHQQDFVNWLLAQK |
| | agonist | |
| | (Des-Ala2- | |
| | GIP1-30) | |
| 38 | GIPR | YAEGTFISDYSIAMDKIHQQDFVNWLLAQK |
| | agonist- | |
| | Truncated | |
| | GIP1-30 | |
| 39 | Glicentin | RSLQDTEEKSRSFSASQADPLSDPDQMNEDKRHSQGTFTSDYSKYLDSRRAQDF |
| | | VQWLMNTKRNRNNIA |
| 40 | Glicentin- | RSLQDTEEKSRSFSASQADPLSDPDQMNED |
| | related | |
| | polypeptide | |
| 41 | Dual | Ac-CSNLSTCMLGRLSQDLHRLQTYPKTDVGANAP |
| | Amylin | |
| | Calcitonin | |
| | Receptor | |
| | Agonist | |
| 42 | Preproinsulin | MALWMRLLPLLALLALWGPDPAAAFVNQHLCGSHLVEALYLVCGERGFFYTPKT |
| | | RREAEDLQVGQVELGGGPGAGSLQPLALEGSLQKRGIVEQCCTSICSLYQLENY |
| | | CN |
| 43 | Insulin A | GIVEQCCTSICSLYQLENYCN |
| | chain | |
| 44 | Insulin B | FVNQHLCGSHLVEALYLVCGERGFFYTPKT |
| | chain | |
| 45 | Insulin | GIVEQCCTSICSLYQLENYCN |
| | Aspart A | |
| | chain | |
| 46 | Insulin | FVNQHLCGSHLVEALYLVCGERGFFYTDKT |
| | Aspart B | |
| | chain | |
| 47 | Insulin | GIVEQCCTSICSLYQLENYCG |
| | glargine A | |
| | chain | |
| 48 | Insulin | FVNQHLCGSHLVEALYLVCGERGFFYTPKTRR |
| | glargine B | |
| | chain | |
| 49 | Insulin | GIVEQCCTSICSLYQLENYCN |
| | lispro A | |
| | chain | |
| 50 | Insulin | FVNQHLCGSHLVEALYLVCGERGFFYTKPT |
| | lispro B | |
| | chain | |
| 51 | Insulin | MASPPESDGFSDVRKVGYLRKPKSMHKRFFVLRAASEAGGPARLEYYENEKKWR |
| | receptor | HKSSAPKRSIPLESCFNINKRADSKNKHLVALYTRDEHFAIAADSEAEQDSWYQ |
| | substrate 1 | ALLQLHNRAKGHHDGAAALGAGGGGGSCSGSSGLGEAGEDLSYGDVPPGPAFKE |
| | | VWQVILKPKGLGQTKNLIGIYRLCLTSKTISFVKLNSEAAAVVLQLMNIRRCGH |
| | | SENFFFIEVGRSAVTGPGEFWMQVDDSVVAQNMHETILEAMRAMSDEFRPRSKS |
| | | QSSSNCSNPISVPLRRHHLNNPPPSQVGLTRRSRTESITATSPASMVGGKPGSF |
| | | RVRASSDGEGTMSRPASVDGSPVSPSTNRTHAHRHRGSARLHPPLNHSRSIPMP |
| | | ASRCSPSATSPVSLSSSSTSGHGSTSDCLFPRRSSASVSGSPSDGGFISSDEYG |
| | | SSPCDFRSSFRSVTPDSLGHTPPARGEEELSNYICMGGKGPSTLTAPNGHYILS |
| | | RGGNGHRCTPGTGLGTSPALAGDEAASAADLDNRFRKRTHSAGTSPTITHQKTP |
| | | SQSSVASIEEYTEMMPAYPPGGGSGGRLPGHRHSAFVPTRSYPEEGLEMHPLER |
| | | RGGHHRPDSSTLHTDDGYMPMSPGVAPVPSGRKGSGDYMPMSPKSVSAPQQIIN |
| | | PIRRHPQRVDPNGYMMMSPSGGCSPDIGGGPSSSSSSSNAVPSGTSYGKLWTNG |
| | | VGGHHSHVLPHPKPPVESSGGKLLPCTGDYMNMSPVGDSNTSSPSDCYYGPEDP |
| | | QHKPVLSYYSLPRSFKHTQRPGEPEEGARHQHLRLSTSSGRLLYAATADDSSSS |
| | | TSSDSLGGGYCGARLEPSLPHPHHQVLQPHLPRKVDTAAQTNSRLARPTRLSLG |
| | | DPKASTLPRAREQQQQQQPLLHPPEPKSPGEYVNIEFGSDQSGYLSGPVAFHSS |
| | | PSVRCPSQLQPAPREEETGTEEYMKMDLGPGRRAAWQESTGVEMGRLGPAPPGA |
| | | ASICRPTRAVPSSRGDYMTMQMSCPRQSYVDTSPAAPVSYADMRTGIAAEEVSL |
| | | PRATMAAASSSSAASASPTGPQGAAELAAHSSLLGGPQGPGGMSAFTRVNLSPN |
| | | RNQSAKVIRADPQGCRRRHSSETFSSTPSATRVGNTVPFGAGAAVGGGGGSSSS |
| | | SEDVKRHSSASFENVWLRPGELGGAPKEPAKLCGAAGGLENGLNYIDLDLVKDF |
| | | KQCPQECTPEPQPPPPPPPHQPLGSGESSSTRRSSEDLSAYASISFQKQPEDRQ |
| 52 | Apolipoprotein | MKLLAATVLLLTICSLEGALVRRQAKEPCVESLVSQYFQTVTDYGKDLMEKVKS |
| | A-II | PELQAEAKSYFEKSKEQLTPLIKKAGTELVNFLSYFVELGTQPATQ |
| 53 | Glycogen | MPLNRTLSMSSLPGLEDWEDEFDLENAVLFEVAWEVANKVGGIYTVLQTKAKVT |
| | synthase 1 | GDEWGDNYFLVGPYTEQGVRTQVELLEAPTPALKRTLDSMNSKGCKVYFGRWLI |
| | | EGGPLVVLLDVGASAWALERWKGELWDTCNIGVPWYDREANDAVLFGFLTTWFL |
| | | GEFLAQSEEKPHVVAHFHEWLAGVGLCLCRARRLPVATIFTTHATLLGRYLCAG |
| | | AVDFYNNLENFNVDKEAGERQIYHRYCMERAAAHCAHVFTTVSQITAIEAQHLL |
| | | KRKPDIVTPNGLNVKKFSAMHEFQNLHAQSKARIQEFVRGHFYGHLDFNLDKTL |
| | | YFFIAGRYEFSNKGADVFLEALARLNYLLRVNGSEQTVVAFFIMPARTNNFNVE |
| | | TLKGQAVRKQLWDTANTVKEKFGRKLYESLLVGSLPDMNKMLDKEDFTMMKRAI |
| | | FATQRQSFPPVCTHNMLDDSSDPILTTIRRIGLFNSSADRVKVIFHPEFLSSTS |
| | | PLLPVDYEEFVRGCHLGVFPSYYEPWGYTPAECTVMGIPSISTNLSGFGCFMEE |
| | | HIADPSAYGIYILDRRFRSLDDSCSQLTSFLYSFCQQSRRQRIIQRNRTERLSD |
| | | LLDWKYLGRYYMSARHMALSKAFPEHFTYEPNEADAAQGYRYPRPASVPPSPSL |
| | | SRHSSPHQSEDEEDPRNGPLEEDGERYDEDEEAAKDRRNIRAPEWPRRASCTSS |
| | | TSGSKRNSVDTATSSSLSTPSEPLSPTSSLGEERN |
| 54 | Glycogen | MLRGRSLSVTSLGGLPQWEVEELPVEELLLFEVAWEVTNKVGGIYTVIQTKAKT |
| | synthase 2 | TADEWGENYFLIGPYFEHNMKTQVEQCEPVNDAVRRAVDAMNKHGCQVHFGRWL |
| | | IEGSPYVVLFDIGYSAWNLDRWKGDLWEACSVGIPYHDREANDMLIFGSLTAWF |
| | | LKEVTDHADGKYVVAQFHEWQAGIGLILSRARKLPIATIFTTHATLLGRYLCAA |
| | | NIDFYNHLDKFNIDKEAGERQIYHRYCMERASVHCAHVFTTVSEITAIEAEHML |
| | | KRKPDVVTPNGLNVKKFSAVHEFQNLHAMYKARIQDFVRGHFYGHLDFDLEKTL |
| | | FLFIAGRYEFSNKGADIFLESLSRLNFLLRMHKSDITVMVFFIMPAKTNNFNVE |
| | | TLKGQAVRKQLWDVAHSVKEKFGKKLYDALLRGEIPDLNDILDRDDLTIMKRAI |
| | | FSTQRQSLPPVTTHNMIDDSTDPILSTIRRIGLFNNRTDRVKVILHPEFLSSTS |
| | | PLLPMDYEEFVRGCHLGVFPSYYEPWGYTPAECTVMGIPSVTTNLSGFGCFMQE |
| | | HVADPTAYGIYIVDRRFRSPDDSCNQLTKFLYGFCKQSRRQRIIQRNRTERLSD |
| | | LLDWRYLGRYYQHARHLTLSRAFPDKFHVELTSPPTTEGFKYPRPSSVPPSPSG |
| | | SQASSPQSSDVEDEVEDERYDEEEEAERDRLNIKSPFSLSHVPHGKKKLHGEYK |
| | | N |
| 55 | Tyrosin- | MEMEKEFEQIDKSGSWAAIYQDIRHEASDFPCRVAKLPKNKNRNRYRDVSPFDH |
| | protein | SRIKLHQEDNDYINASLIKMEEAQRSYILTQGPLPNTCGHFWEMVWEQKSRGVV |
| | phosphatase | MLNRVMEKGSLKCAQYWPQKEEKEMIFEDTNLKLTLISEDIKSYYTVRQLELEN |
| | non-receptor | LTTQETREILHFHYTTWPDFGVPESPASFLNFLFKVRESGSLSPEHGPVVVHCS |
| | type 1 | AGIGRSGTFCLADTCLLLMDKRKDPSSVDIKKVLLEMRKFRMGLIQTADQLRFS |
| | | YLAVIEGAKFIMGDSSVQDQWKELSHEDLEPPPEHIPPPPRPPKRILEPHNGKC |
| | | REFFPNHQWVKEETQEDKDCPIKEEKGSPLNAAPYGIESMSQDTEVRSRVVGGS |
| | | LRGAQAASPAKGEPSLPEKDEDHALSYWKPFLVNMCVATVLTAGAYLCYRFLFN |
| | | SNT |
| 56 | RAC- | MSDVAIVKEGWLHKRGEYIKTWRPRYFLLKNDGTFIGYKERPQDVDQREAPLNN |
| | alpha | FSVAQCQLMKTERPRPNTFIIRCLQWTTVIERTFHVETPEEREEWTTAIQTVAD |
| | serine | GLKKQEEEEMDFRSGSPSDNSGAEEMEVSLAKPKHRVTMNEFEYLKLLGKGTFG |
| | threonine- | KVILVKEKATGRYYAMKILKKEVIVAKDEVAHTLTENRVLQNSRHPFLTALKYS |
| | protein | FQTHDRLCFVMEYANGGELFFHLSRERVFSEDRARFkinaseYGAEIVSALDYL |
| | kinase | HSEKNVVYRDLKLENLMLDKDGHIKITDFGLCKEGIKDGATMKTFCGTPEYLAP |
| | | EVLEDNDYGRAVDWWGLGVVMYEMMCGRLPFYNQDHEKLFELILMEEIRFPRTL |
| | | GPEAKSLLSGLLKKDPKQRLGGGSEDAKEIMQHRFFAGIVWQHVYEKKLSPPFK |
| | | PQVTSETDTRYFDEEFTAQMITITPPDQDDSMECVDSERRPHFPQFSYSASGTA |
| 57 | Peroxisome | MGETLGDSPIDPESDSFTDTLSANISQEMTMVDTEMPFWPTNFGISSVDLSVME |
| | proliferator- | DHSHSFDIKPFTTVDFSSISTPHYEDIPFTRTDPVVADYKYDLKLQEYQSAIKV |
| | activated | EPASPPYYSEKTQLYNKPHEEPSNSLMAIECRVCGDKASGFHYGVHACEGCKGF |
| | receptor | FRRTIRLKLIYDRCDLNCRIHKKSRNKCQYCRFQKCLAVGMSHNAIRFGRMPQA |
| | gamma | EKEKLLAEISSDIDQLNPESADLRALAKHLYDSYIKSFPLTKAKARAILTGKTT |
| | | DKSPFVIYDMNSLMMGEDKIKFKHITPLQEQSKEVAIRIFQGCQFRSVEAVQEI |
| | | TEYAKSIPGFVNLDLNDQVTLLKYGVHEIIYTMLASLMNKDGVLISEGQGFMTR |
| | | EFLKSLRKPFGDFMEPKFEFAVKFNALELDDSDLAIFIAVIILSGDRPGLLNVK |
| | | PIEDIQDNLLQALELQLKLNHPESSQLFAKLLQKMTDLRQIVTEHVQLLQVIKK |
| | | TETDMSLHPLLQEIYKDLY |
| 58 | Hexokinase 3 | MDSIGSSGLRQGEETLSCSEEGLPGPSDSSELVQECLQQFKVTRAQLQQIQASL |
| | | LGSMEQALRGQASPAPAVRMLPTYVGSTPHGTEQGDFVVLELGATGASLRVLWV |
| | | TLTGIEGHRVEPRSQEFVIPQEVMLGAGQQLFDFAAHCLSEFLDAQPVNKQGLQ |
| | | LGFSFSFPCHQTGLDRSTLISWTKGFRCSGVEGQDVVQLLRDAIRRQGAYNIDV |
| | | VAVVNDTVGTMMGCEPGVRPCEVGLVVDTGTNACYMEEARHVAVLDEDRGRVCV |
| | | SVEWGSFSDDGALGPVLTTFDHTLDHESLNPGAQRFEKMIGGLYLGELVRLVLA |
| | | HLARCGVLFGGCTSPALLSQGSILLEHVAEMEDPSTGAARVHAILQDLGLSPGA |
| | | SDVELVQHVCAAVCTRAAQLCAAALAAVLSCLQHSREQQTLQVAVATGGRVCER |
| | | HPRFCSVLQGTVMLLAPECDVSLIPSVDGGGRGVAMVTAVAARLAAHRRLLEET |
| | | LAPFRLNHDQLAAVQAQMRKAMAKGLRGEASSLRMLPTFVRATPDGSERGDFLA |
| | | LDLGGTNFRVLLVRVTTGVQITSEIYSIPETVAQGSGQQLFDHIVDCIVDFQQK |
| | | QGLSGQSLPLGFTFSFPCRQLGLDQGILLNWTKGFKASDCEGQDVVSLLREAIT |
| | | RRQAVELNVVAIVNDTVGTMMSCGYEDPRCEIGLIVGTGTNACYMEELRNVAGV |
| | | PGDSGRMCINMEWGAFGDDGSLAMLSTRFDASVDQASINPGKQRFEKMISGMYL |
| | | GEIVRHILLHLTSLGVLFRGQQIQRLQTRDIFKTKFLSEIESDSLALRQVRAIL |
| | | EDLGLPLTSDDALMVLEVCQAVSQRAAQLCGAGVAAVVEKIRENRGLEELAVSV |
| | | GVDGTLYKLHPRFSSLVAATVRELAPRCVVTFLQSEDGSGKGAALVTAVACRLA |
| | | QLTRV |
| 59 | Phosphatidyl- | MTAIIKEIVSRNKRRYQEDGFDLDLTYIYPNIIAMGFPAERLEGVYRNNIDDVV |
| | inositol-3,4,5- | RFLDSKHKNHYKIYNLCAERHYDTAKFNCRVAQYPFEDHNPPQLELIKPFCEDL |
| | triphosphate | DQWLSEDDNHVAAIHCKAGKGRTGVMICAYLLHRGKFLKAQEALDFYGEVRTRD |
| | 3-phosphatase | KKGVTIPSQRRYVYYYSYLLKNHLDYRPVALLFHKMMFETIPMFSGGTCNPQFV |
| | and dual- | VCQLKVKIYSSNSGPTRREDKFMYFEFPQPLPVCGDIKVEFFHKQNKMLKKDKM |
| | specificity | FHFWVNTFFIPGPEETSEKVENGSLCDQEIDSICSIERADNDKEYLVLTLTKND |
| | protein | LDKANKDKANRYFSPNFKVKLYFTKTVEEPSNPEASSSTSVTPDVSDNEPDHYR |
| | | YSDTTDSDPENEPFDEDQHTQITKV |
| 60 | Pyruvate | MRLARLLRGAALAGPGPGLRAAGFSRSFSSDSGSSPASERGVPGQVDFYARFSP |
| | dehydrogenase | SPLSMKQFLDFGSVNACEKTSFMFLRQELPVRLANIMKEISLLPDNLLRTPSVQ |
| | kinase 1 | LVQSWYIQSLQELLDFKDKSAEDAKAIYDFTDTVIRIRNRHNDVIPTMAQGVIE |
| | | YKESFGVDPVTSQNVQYFLDRFYMSRISIRMLLNQHSLLFGGKGKGSPSHRKHI |
| | | GSINPNCNVLEVIKDGYENARRLCDLYYINSPELELEELNAKSPGQPIQVVYVP |
| | | SHLYHMVFELFKNAMRATMEHHANRGVYPPIQVHVTLGNEDLTVKMSDRGGGVP |
| | | LRKIDRLFNYMYSTAPRPRVETSRAVPLAGFGYGLPISRLYAQYFQGDLKLYSL |
| | | EGYGTDAVIYIKALSTDSIERLPVYNKAAWKHYNTNHEADDWCVPSREPKDMTT |
| | | FRSA |
| 61 | Calcium- | MEESPLSRAPSRGGVNFLNVARTYIPNTKVECHYTLPPGTMPSASDWIGIFKVE |
| | binding | AACVRDYHTFVWSSVPESTTDGSPIHTSVQFQASYLPKPGAQLYQFRYVNRQGQ |
| | and | VCGQSPPFQFREPRPMDELVTLEEADGGSDILLVVPKATVLQNQLDESQQERND |
| | coiled-coil | LMQLKLQLEGQVTELRSRVQELERALATARQEHTELMEQYKGISRSHGEITEER |
| | domain- | DILSRQQGDHVARILELEDDIQTISEKVLTKEVELDRLRDTVKALTREQEKLLG |
| | containing | QLKEVQADKEQSEAELQVAQQENHHLNLDLKEAKSWQEEQSAQAQRLKDKVAQM |
| | protein 1 | KDTLGQAQQRVAELEPLKEQLRGAQELAASSQQKATLLGEELASAAAARDRTIA |
| | | ELHRSRLEVAEVNGRLAELGLHLKEEKCQWSKERAGLLQSVEAEKDKILKLSAE |
| | | ILRLEKAVQEERTQNQVFKTELAREKDSSLVQLSESKRELTELRSALRVLQKEK |
| | | EQLQEEKQELLEYMRKLEARLEKVADEKWNEDATTEDEEAAVGLSCPAALTDSE |
| | | DESPEDMRLPPYGLCERGDPGSSPAGPREASPLVVISQPAPISPHLSGPAEDSS |
| | | SDSEAEDEKSVLMAAVQSGGEEANLLLPELGSAFYDMASGFTVGTLSETSTGGP |
| | | ATPTWKECPICKERFPAESDKDALEDHMDGHFFFSTQDPFTFE |
| 62 | Max-like | MTEPGASPEDPWVKASPVGAHAGEGRAGRARARRGAGRRGASLLSPKSPTLSVP |
| | protein X | RGCREDSSHPACAKVEYAYSDNSLDPGLFVESTRKGSVVSRANSIGSTSASSVP |
| | | NTDDEDSDYHQEAYKESYKDRRRRAHTQAEQKRRDAIKRGYDDLQTIVPTCQQQ |
| | | DFSIGSQKLSKAIVLQKTIDYIQFLHKEKKKQEEEVSTLRKDVTALKIMKVNYE |
| | | QIVKAHQDNPHEGEDQVSDQVKFNVFQGIMDSLFQSFNASISVASFQELSACVF |
| | | SWIEEHCKPQTLREIVIGVLHQLKNQLY |
| 63 | Fructose- | MPYQYPALTPEQKKELSDIAHRIVAPGKGILAADESTGSIAKRLQSIGTENTEE |
| | biphosphate | NRRFYRQLLLTADDRVNPCIGGVILFHETLYQKADDGRPFPQVIKSKGGVVGIK |
| | alsolase A | VDKGVVPLAGTNGETTTQGLDGLSERCAQYKKDGADFAKWRCVLKIGEHTPSAL |
| | | AIMENANVLARYASICQQNGIVPIVEPEILPDGDHDLKRCQYVTEKVLAAVYKA |
| | | LSDHHIYLEGTLLKPNMVTPGHACTQKFSHEEIAMATVTALRRTVPPAVTGITF |
| | | LSGGQSEEEASINLNAINKCPLLKPWALTFSYGRALQASALKAWGGKKENLKAA |
| | | QEEYVKRALANSLACQGKYTPSGQAGAAASESLFVSNHAY |
| 64 | Dipeptidyl | MKTPWKVLLGLLGAAALVTIITVPVVLLNKGTDDATADSRKTYTLTDYLKNTYR |
| | peptidase 4 | LKLYSLRWISDHEYLYKQENNILVFNAEYGNSSVFLENSTFDEFGHSINDYSIS |
| | | PDGQFILLEYNYVKQWRHSYTASYDIYDLNKRQLITEERIPNNTQWVTWSPVGH |
| | | KLAYVWNNDIYVKIEPNLPSYRITWTGKEDIIYNGITDWVYEEEVFSAYSALWW |
| | | SPNGTFLAYAQFNDTEVPLIEYSFYSDESLQYPKTVRVPYPKAGAVNPTVKFFV |
| | | VNTDSLSSVTNATSIQITAPASMLIGDHYLCDVTWATQERISLQWLRRIQNYSV |
| | | MDICDYDESSGRWNCLVARQHIEMSTTGWVGRFRPSEPHFTLDGNSFYKIISNE |
| | | EGYRHICYFQIDKKDCTFITKGTWEVIGIEALTSDYLYYISNEYKGMPGGRNLY |
| | | KIQLSDYTKVTCLSCELNPERCQYYSVSFSKEAKYYQLRCSGPGLPLYTLHSSV |
| | | NDKGLRVLEDNSALDKMLQNVQMPSKKLDFIILNETKFWYQMILPPHFDKSKKY |
| | | PLLLDVYAGPCSQKADTVFRLNWATYLASTENIIVASFDGRGSGYQGDKIMHAI |
| | | NRRLGTFEVEDQIEAARQFSKMGFVDNKRIAIWGWSYGGYVTSMVLGSGSGVFK |
| | | CGIAVAPVSRWEYYDSVYTERYMGLPTPEDNLDHYRNSTVMSRAENFKQVEYLL |
| | | IHGTADDNVHFQQSAQISKALVDVGVDFQAMWYTDEDHGIASSTAHQHIYTHMS |
| | | HFIKQCFSLP |
| 65 | Cholix¹⁻²⁶⁶ | VEEALNIFDECRSPCSLTPEPGKPIQSKLSIPSDVVLDEGVLYYSMTINDEQND |
| | carrier | IKDEDKGESIITIGEFATVRATRHYVNQDAPFGVIHLDITTENGTKTYSYNRKE |
| | | GEFAINWLVPIGEDSPASIKISVDELDQQRNIIEVPKLYSIDLDNQTLEQWKTQ |
| | | GNVSFSVTRPEHNIAISWPSVSYKAAQKEGSRHKRWAHWHTGLALCWLVPMDAI |
| | | YNYITQQNCTLGDNWFGGSYETVAGTPKVITVKQGIEQKPVEQRIHFSKG |
| 66 | Cholix¹⁻²⁶⁶ | LEEALNIFDECRSPCSLTPEPGKPIQSKLSIPSDVVLDEGVLYYSMTINDEQND |
| | carrier | IKDEDKGESIITIGEFATVRATRHYVNQDAPFGVIHLDITTENGTKTYSYNRKE |
| | | GEFAINWLVPIGEDSPASIKISVDELDQQRNIIEVPKLYSIDLDNQTLEQWKTQ |
| | | GNVSFSVTRPEHNIAISWPSVSYKAAQKEGSRHKRWAHWHTGLALCWLVPMDAI |
| | | YNYITQQNCTLGDNWFGGSYETVAGTPKVITVKQGIEQKPVEQRIHFSKG |
| 67 | Cholix¹⁻²⁶⁶ | VEDELNIFDECRSPCSLTPEPGKPIQSKLSIPSDVVLDEGVLYYSMTINDEQND |
| | carrier | IKDEDKGESIITIGEFATVRATRHYVNQDAPFGVIHLDITTENGTKTYSYNRKE |
| | | GEFAINWLVPIGEDSPASIKISVDELDQQRNIIEVPKLYSIDLDNQTLEQWKTQ |
| | | GNVSFSVTRPEHNIAISWPSVSYKAAQKEGSRHKRWAHWHTGLALCWLVPMDAI |
| | | YNYITQQNCTLGDNWFGGSYETVAGTPKVITVKQGIEQKPVEQRIHFSKG |
| 68 | Pseudomonas | GTCGAAGAAGCTTTAAACATCTTTGATGAATGCCGTTCGCCATGTTCGTTGACC |
| | exotoxin | CCGGAACCGGGTAAGCCGATTCAATCAAAACTGTCTATCCCTAGTGATGTTGTT |
| | A gene | CTGGATGAAGGTGTTCTGTATTACTCGATGACGATTAATGATGAGCAGAATGAT |
| | | ATTAAGGATGAGGACAAAGGCGAGTCCATTATCACTATTGGTGAATTTGCCACA |
| | | GTACGCGCGACTAGACATTATGTTAATCAAGATGCGCCTTTTGGTGTCATCCAT |
| | | TTAGATATTACGACAGAAAATGGTACAAAAACGTACTCTTATAACCGCAAAGAG |
| | | GGTGAATTTGCAATCAATTGGTTAGTGCCTATTGGTGAAGATTCTCCTGCAAGC |
| | | ATCAAAATCTCCGTTGATGAGCTCGATCAGCAACGCAATATCATCGAGGTGCCT |
| | | AAACTGTATAGTATTGATCTCGATAACCAAACGTTAGAGCAGTGGAAAACCCAA |
| | | GGTAATGTTTCTTTTTCGGTAACGCGTCCTGAACATAATATCGCTATCTCTTGG |
| | | CCAAGCGTGAGTTACAAAGCAGCGCAGAAAGAGGGTTCACGCCATAAGCGTTGG |
| | | GCTCATTGGCATACAGGCTTAGCACTGTGTTGGCTTGTGCCAATGGATGCTATC |
| | | TATAACTATATCACCCAGCAAAATTGTACTTTAGGGGATAATTGGTTTGGTGGC |
| | | TCTTATGAGACTGTTGCAGGCACTCCGAAGGTGATTACGGTTAAGCAAGGGATT |
| | | GAACAAAAGCCAGTTGAGCAGCGCATCCATTTCTCCAAGGGGAATGCGATGAGC |
| | | GCACTTGCTGCTCATCGCGTCTGTGGTGTGCCATTAGAAACTTTGGCGCGCAGT |
| | | CGCAAACCTCGTGATCTGACGGATGATTTATCATGTGCCTATCAAGCGCAGAAT |
| | | ATCGTGAGTTTATTTGTCGCGACGCGTATCCTGTTCTCTCATCTGGATAGCGTA |
| | | TTTACTCTGAATCTTGACGAACAAGAACCAGAGGTGGCTGAACGTCTAAGTGAT |
| | | CTTCGCCGTATCAATGAAAATAACCCGGGCATGGTTACACAGGTTTTAACCGTT |
| | | GCTCGTCAGATCTATAACGATTATGTCACTCACCATCCGGGCTTAACTCCTGAG |
| | | CAAACCAGTGCGGGTGCACAAGCTGCCGATATCCTCTCTTTATTTTGCCCAGAT |
| | | GCTGATAAGTCTTGTGTGGCTTCAAACAACGATCAAGCCAATATCAACATCGAG |
| | | TCTCGTTCTGGCCGTTCATATTTGCCTGAAAACCGTGCGGTAATCACCCCTCAA |
| | | GGCGTCACAAATTGGACTTACCAGGAACTCGAAGCAACACATCAAGCTCTGACT |
| | | CGTGAGGGTTATGTGTTCGTGGGTTACCATGGTACGAATCATGTCGCTGCGCAA |
| | | ACCATCGTGAATCGCATTGCCCCTGTTCCGCGCGGCAACAACACTGAAAACGAG |
| | | GAAAAGTGGGGCGGGTTATATGTTGCAACTCACGCTGAAGTTGCCCATGGTTAT |
| | | GCTCGCATCAAAGAAGGGACAGGGGAGTATGGCCTTCCGACCCGTGCTGAGCGC |
| | | GACGCTCGTGGGGTAATGCTGCGCGTGTATATCCCTCGTGCTTCATTAGAACGT |
| | | TTTTATCGCACGAATACACCTTTGGAAAATGCTGAGGAGCATATCACGCAAGTG |
| | | ATTGGTCATTCTTTGCCATTACGCAATGAAGCATTTACTGGTCCAGAAAGTGCG |
| | | GGCGGGGAAGACGAAACTGTCATTGGCTGGGATATGGCGATTCATGCAGTTGCG |
| | | ATCCCTTCGACTATCCCAGGGAACGCTTACGAAGAATTGGCGATTGATGAGGAG |
| | | GCTGTTGCAAAAGAGCAATCGATTAGCACAAAACCACCTTATAAAGAGCGCAAA |
| | | GATGAACTTAAG |
| 69 | Pseudomonas | AEEAFDLWNECAKACVLDLKDGVRSSRMSVDPAIADTNGQGVLHYSMVLEGGND |
| | exotoxin A | ALKLAIDNALSITSDGLTIRLEGGVEPNKPVRYSYTRQARGSWSLNWLVPIGHE |
| | | KPSNIKVFIHELNAGNQLSHMSPIYTIEMGDELLAKLARDATFFVRAHESNEMQ |
| | | PTLAISHAGVSVVMAQAQPRREKRWSEWASGKVLCLLDPLDGVYNYLAQQRCNL |
| | | DDTWEGKIYRVLAGNPAKHDLDIKPTVISHRLHFPEGGSLAALTAHQACHLPLE |
| | | TFTRHRQPRGWEQLEQCGYPVQRLVALYLAARLSWNQVDQVIRNALASPGSGGD |
| | | LGEAIREQPEQARLALTLAAAESERFVRQGTGNDEAGAASADVVSLTCPVAAGE |
| | | CAGPADSGDALLERNYPTGAEFLGDGGDVSFSTRGTQNWTVERLLQAHRQLEER |
| | | GYVFVGYHGTFLEAAQSIVFGGVRARSQDLDAIWRGFYIAGDPALAYGYAQDQE |
| | | PDARGRIRNGALLRVYVPRSSLPGFYRTGLTLAAPEAAGEVERLIGHPLPLRLD |
| | | AITGPEEEGGRLETILGWPLAERTVVIPSAIPTDPRNVGGDLDPSSIPDKEQAI |
| | | SALPDYASQPGKPPREDLK |
| 70 | Cholix | MVEEALNIFDECRSPCSLTPEPGKPIQSKLSIPSDVVLDEGVLYYSMTINDEQN |
| | variant | DIKDEDKGESIITIGEFATVRATRHYVNQDAPFGVIHLDITTENGTKTYSYNRK |
| | TEV | EGEFAINVVLVPIGEDSPASIKISVDELDQQRNIIEVPKLYSIDLDNQTLEQWK |
| | cleavage | TQGNVSFSVTRPEproductHNIAISWPSVSYKAAQKEGSRHKRWAHWHTGLAL |
| | product | CWLVPMDAIYNYITQQNCTLGDNVVFGGSYETVAGTPKVITVKQGIEQKPVEQR |
| | | IHFSKGNAMSALAAHRVCGVPLETLARSRKPRDLTDDLSCAYQAQNIVSLFVAT |
| | | RILFSHLDSVFTLNLDEQEPEVAERLSDLRRINENNPGMVTQVLTVARQIYNDY |
| | | VTHHPGLTPEQTSAGAQACENLFQ |
| 71 | Exenatide- | MHGEGTFTSDLSKQMEEEAVRLFIEWLKNGGPSSGAPPPSGGGGGSGGGGSGGG |
| | Cholix | GSVEEALNIFDECRSPCSLTPEPGKPIQSKLSIPSDVVLDEGVLYYSMTINDEQ |
| | variant | NDIKDEDKGESIITIGEFATVRATRHYVNQDAPFGVIHLDITTENGTKTYSYNR |
| | | KEGEFAINWLVPIGEDSPASIKISVDELDQQRNIIEVPKLYSIDLDNQTLEQWK |
| | | TQGNVSFSVTRPEHNIAISWPSVSYKAAQKEGSRHKRWAHWHTGLA |
| 72 | N-terminus | VEDE |
| | of a natural | |
| | Cholix | |
| | variant | |
| 73 | Cholix | VEEALNIFDECRSPCSLTPEPGKPIQSKLSIPSDVVLDEGVLYYSMTINDEQND |
| | variant | IKDEDKGESIITIGEFATVRATRHYVNQDAPFGVIHLDITTENGTKTYSYNRKE |
| | | GEFAINWLVPIGEDSPASIKISVDELDQQRNIIEVPKLYSIDLDNQTLEQWKTQ |
| | | GNVSFSVTRPEHNIAISWPSVSYKAAQKEGSRHKRWAHWHTGLA |
| 74 | Cholix | MVEEALNIFDECRSPCSLTPEPGKPIQSKLSIPSDVVLDEGVLYYSMTINDEQN |
| | variant | DIKDEDKGESIITIGEFATVRATRHYVNQDAPFGVIHLDITTENGTKTYSYNRK |
| | | EGEFAINVVLVPIGEDSPASIKISVDELDQQRNIIEVPKLYSIDLDNQTLEQWK |
| | | TQGNVSFSVTRPEHNIAISWPSVSYKAAQKEGSRHKRWAHWHTGLALCWLVPMD |
| | | AIYNYITQQNCTLGDNVVFGGSYETVAGTPKVITVKQGIEQKPVEQRIHFSKGN |
| | | AMSALAAHRVCGVPLETLARSRKPRDLTDDLSCAYQAQNIVSLFVATRILFSHL |
| | | DSVFTLNLDEQEPEVAERLSDLRRINENNPGMVTQVLTVARQIYNDYVTHHPGL |
| | | TPEQTSAGAQA |
| 75 | Cholix | VEDELNIFDECRSPCSLTPEPGKPIQSKLSIPSDVVLDEGVLYYSMTINDEQND |
| | variant | IKDEDKGESIITIGEFATVRATRHYVNQDAPFGVIHLDITTENGTKTYSYNRKE |
| | | GEFAINWLVPIGEDSPASIKISVDELDQQRNIIEVPKLYSIDLDNQTLEQWKTQ |
| | | GNVSFSVTRPEHNIAISWPSVSYKAAQKEGSRHKRWAHWHTGLALCWLVPMDAI |
| | | YNYITQQNCTLGDNWFGGSYETVAGTPKVITVKQGIEQKPVEQRIHFSKGNAMS |
| | | ALAAHRVCGVPLETLARSRKPRDLTDDLSCAYQAQNIVSLFVATRILFSHLDSV |
| | | FTLNLDEQEPEVAERLSDLRRINENNPGMVTQVLTVARQIYNDYVTHHPGLTPE |
| | | QTSAGAQAADILSLFCPDADKSCVASNNDQANINIESRSGRSYLPENRAVITPQ |
| | | GVTNWTYQELEATHQALTREGYVFVGYHGTNHVAAQTIVNRIAPVPRGNNTENE |
| | | EKWGGLYVATHAEVAHGYARIKEGTGEYGLPTRAERDARGVMLRVYIPRASLER |
| | | FYRTNTPLENAEEHITQVIGHSLPLRNEAFTGPESAGGEDETVIGWDMAIHAVA |
| | | IPSTIPGNAYEELAIDEEAVAKEQSISTKPPYKERKDELK |
| 76 | Cholix | VEEALNIFDECRSPCSLTPEPGKPIQSKLSIPGDVVLDEGVLYYSMTINDEQND |
| | variant | IKDEDKGESIITIGEFATVRATRHYVSQDAPFGVINLDITTENGTKTYSFNRKE |
| | | SEFAINWLVPIGEDSPASIKISIDELDQQRNIIEVPKLYSIDLDNQTLEQWKTQ |
| | | GNVSFSVTRPEHNIAISWPSVSYKAAQKEGSRHKRWAHWHTGLALCWLVPIDAI |
| | | YNYITQQNCTLGDNWFGGSYETVAGTPKAITVKQGIEQKPVEQRIHFSKKNAME |
| | | ALAAHRVCGVPLETLARSRKPRDLPDDLSCAYNAQQIVSLFLATRILFTHIDSI |
| | | FTLNLDGQEPEVAERLDDLRRINENNPGMVIQVLTVARQIYNDYVTHHPGLTPE |
| | | QTSAGAQAADILSLFCPDADKSCVASNSDQANINIES |
| 77 | Cholix | MVEEALNIFDECRSPCSLTPEPGKPIQSKLSIPSDVVLDEGVLYYSMTINDEQN |
| | variant | DIKDEDKGESIITIGEFATVRATRHYVNQDAPFGVIHLDITTENGTKTYSYNRK |
| | TEV | EGEFAINVVLVPIGEDSPASIKISVDELDQQRNIIEVPKLYSIDLDNQTLEQWK |
| | | TQGNVSFSVTRPEHNIAISWPSVSYKAAQKEGSRHKRWAHWHTGLALCWLVPMD |
| | | AIYNYITQQNCTLGDNVVFGGSYETVAGTPKVITVKQGIEQKPVEQRIHFSKGN |
| | | AMSALAAHRVCGVPLETLARSRKPRDLTDDLSCAYQAQNIVSLFVATRILFSHL |
| | | DSVFTLNLDEQEPEVAERLSDLRRINENNPGMVTQVLTVARQIYNDYVTHHPGL |
| | | TPEQTSAGAQAADILSLFCPDADKSCVASNNDQANINIESCENLFQSGTCHHHH |
| | | HH |
| 78 | Cholix | MVEEALNIFDECRSPCSLTPEPGKPIQSKLSIPSDVVLDEGVLYYSMTINDEQN |
| | variant | DIKDEDKGESIITIGEFATVRATRHYVNQDAPFGVIHLDITTENGTKTYSYNRK |
| | TEV | EGEFAINVVLVPIGEDSPASIKISVDELDQQRNIIEVPKLYSIDLDNQTLEQWK |
| | | TQGNVSFSVTRPEHNIAISWPSVSYKAAQKEGSRHKRWAHWHTGLALCWLVPMD |
| | | AIYNYITQQNCTLGDNVVFGGSYETVAGTPKVITVKQGIEQKPVEQRIHFSKGN |
| | | AMSALAAHRVCGVPLETLARSRKPRDLTDDLSCAYQAQNIVSLFVATRILFSHL |
| | | DSVFTLNLDEQEPEVAERLSDLRRINENNPGMVTQVLTVARQIYNDYVTHHPGL |
| | | TPEQTSAGAQACENLFQSGTCHHHHHH |
| 79 | (G5S)(G4S)2 | GGGGGSGGGGSGGGGS |
| | spacer | |
| 80 | Cholix | MVEEALNIFDECRSPCSLTPEPGKPIQSKLSIPSDVVLDEGVLYYSMTINDEQN |
| | variant | DIKDEDKGESIITIGEFATVRATRHYVNQDAPFGVIHLDITTENGTKTYSYNRK |
| | TEV | EGEFAINVVLVPIGEDSPASIKISVDELDQQRNIIEVPKLYSIDLDNQTLEQWK |
| | cleavage | TQGNVSFSVTRPEHNIAISWPSVSYKAAQKEGSRHKRWAHWHTGLALCWLVPMD |
| | product | AIYNYITQQNCTLGDNVVFGGSYETVAGTPKVITVKQGIEQKPVEQRIHFSKGN |
| | | AMSALAAHRVCGVPLETLARSRKPRDLTDDLSCAYQAQNIVSLFVATRILFSHL |
| | | DSVFTLNLDEQEPEVAERLSDLRRINENNPGMVTQVLTVARQIYNDYVTHHPGL |
| | | TPEQTSAGAQAADILSLFCPDADKSCVASNNDQANINIESCENLFQ |
| 81 | Cholix | V-E-X3-X4-L-X6-I-F-D-E-C-R-S-P-C-X16-L-T-P-E-X21-G-K- |
| | | variantX24-I-Q-S-K-L-X30-I-P-X33-D-V-V-L-D-E-G-V-L-Y- |
| | | Y-S-M-T-I-N-D-E-Q-N-D-I-X56-D-E-X59-K-G-E-S-I-I-T-X67- |
| | | G-E-F-A-T-X73-R-A-T-R-H-Y-V-X81-Q-D-A-P-F-G-V-I-X90-L- |
| | | D-I-T-T-E-N-G-T-K-X101-Y-S-X104-N-R-K-X108-X109-E-F- |
| | | X112-I-X114-W-L-V-X118-X119-G-E-D-S-P-A-S-I-K-I-S- |
| | | X131-D-E-X134-D-Q-X137-R-N-I-I-E-V-P-K-L-Y-S-I-D-L-D- |
| | | N-Q-T-L-E-Q-W-X160-X161-Q-G-N-V-X166-F-X168-V-T-R-P-E- |
| | | X174-X175-I-A-I-S-W-P-S-V-S-Y-X186-A-A-X189-K-X191-G- |
| | | X193-R-H-K-R-W-A-X200-W-X202-T-X204-X205-X206-X207- |
| | | X208-X209-L-X211-X212-X213-X214-X215-X216-X217-X218- |
| | | X219-X220-X221-X222-X223-X224-C-T-X227-G-X229-X230-W- |
| | | X232-G-G-X235-Y-X237-T-V-A-G-X242-P-X244-X245-I-X247- |
| | | V-K-Q-G-X252-E-Q-K-X256-V-E-Q-R-I-H-F-S-X265-X266-N-A- |
| | | X269-X270-X271-L-A-A-H-R-V-C-G-V-P-L-E-T-L-A-R-X288-R- |
| | | K-P-R-X293-L-X295-D-D-L-X299-C-X301-Y-X303-A-Q-X306-I- |
| | | V-S-L-F-X312-A-T-R-X316-L-F-X319-H-X321-D-S-X324-F-T- |
| | | L-N-L-X330-X331-Q-X333-P-X335-V-X337-E-R-L-X341-X342- |
| | | X343-R-X345-I-N-E-X349-N-P-G-X353-V-X355-Q-V-L-T-X360- |
| | | A-R-Q-I-Y-N-D-Y-V-T-X371-H-P-X374-L-X376-P-E-Q-T-S-A- |
| | | X383-A-Q-A-A-D-I-L-S-L-X393-X394-P-D-X397-D-X399-X400- |
| | | C-V-A-X404-X405-X406-D-Q-A-N-I-N-X413-E-S-R-S-G-R-S-Y- |
| | | L-X423-E-N-R-A-V-I-T-X431-Q-G-V-T-N-W-T-Y-Q-E-L-X443- |
| | | X444-X445-H-Q-X448-L-T-X451-E-X453-Y-V-F-V-G-Y-H-G-T- |
| | | N-H-X465-A-A-Q-X469-I-V-N-R-I-X475-P-V-P-R-G-X481- |
| | | X482-T-E-X485-E-X487-X488-W-G-G-X492-Y-V-X495-T-X497- |
| | | A-X499-X500-X501-X502-X503-Y-X505-R-X507-X508-X509-G- |
| | | T-X512-X513-X514-X515-X516-X517-T-X519-X520-X521-X522- |
| | | X523-X524-R-G-V-M-L-X530-V-Y-X533-X534-X535-A-S-L-E-R- |
| | | F-Y-R-X544-N-X546-X547-L-E-X550-X551-X552-X553-X554- |
| | | X555-X556-X557-V-I-G-H-X562-L-P-L-R-N-E-A-F-T-G-X573- |
| | | X574-X575-X576-X577-G-X579-X580-E-T-X583-I-G-W-D-X588- |
| | | A-I-X591-X592-V-A-I-P-S-T-I-P-G-N-X603-Y-X605-X606-L- |
| | | X608-X609-X610-E-E-A-X614-A-X616-E-Q-S-I-S-X622-K-P-P- |
| | | Y-K-E-X629-X630-D-E-L-K;wherein X3 is selected from |
| | | the group consisting of E and D; X4 is selected from |
| | | the group consisting of A and E; X6 is selected from |
| | | the group consisting of N and K; X16 is selected from |
| | | the group consisting of S and L; X21 is selected from |
| | | the group consisting of P and L; X24 is selected from |
| | | the group consisting of P and Q; X30 is selected from |
| | | the group consisting of S and F; X33 is selected from |
| | | the group consisting of S and G; X56 is selected from |
| | | the group consisting of K and M; X59 is selected from |
| | | the group consisting of D and G; X67 is selected from |
| | | the group consisting of I and F; X73 is selected from |
| | | the group consisting of V and I; X81 is selected from |
| | | the group consisting of N and S; X90 is selected from |
| | | the group consisting of H and N; X101 is selected |
| | | from the group consisting of T and M; X104 is |
| | | selected from the group consisting of Y and F; X108 |
| | | is selected from the group consisting of E and D; |
| | | X109 is selected from the group consisting of G and |
| | | S; X112 is selected from the group consisting of A |
| | | and T; X114 is selected from the group consisting of |
| | | N and H; X118 is selected from the group consisting |
| | | of P and I; X119 is selected from the group |
| | | consisting of I and P; X131 is selected from the |
| | | group consisting of V and I; X134 is selected from |
| | | the group consisting L and I; X137 is selected from |
| | | the group consisting Qand K; X160 is selected from |
| | | the group consisting K and E; X161 is selected from |
| | | the group consisting T and N; X166 is selected from |
| | | the group consisting S and F; X168 is selected from |
| | | the group consisting S and A; X174 is selected from |
| | | the group consisting H and Q; X175 is selected from |
| | | the group consisting N, S, SIAKQS (SEQ ID NO: 92), |
| | | and SIAKQSIAKQS (SEQ ID NO: 93); X186 is selected |
| | | from the group consisting of K and N; X189 is |
| | | selected from the group consisting of Q, E, and H; |
| | | X191 is selected from the group consisting of E, N, |
| | | and D; X193 is selected from the group consisting of |
| | | S and A; X200 is selected from the group consisting |
| | | of H and N; X202 is selected from the group |
| | | consisting of H, L, F, and R; X204 is selected from |
| | | the group consisting of G and T; X205 is selected |
| | | from the group consisting of L and S; X206 is |
| | | selected from the group consisting of A and P; X207 |
| | | is selected from the group consisting of L, E, and K; |
| | | X208 is selected from the group consisting of C and |
| | | V; X209 is selected from the group consisting of W, |
| | | V, and T; X211 is selected from the group consisting |
| | | of V and no amino acid; X212 is selected from the |
| | | group consisting of P and no amino acid; X213 is |
| | | selected from the group consisting of M, I, L, and |
| | | no amino acid; X214 is selected from the group |
| | | consisting of D and no amino acid; X215 is selected |
| | | from the group consisting of A and no amino acid; |
| | | X216 is selected from the group consisting of I and |
| | | no amino acid; X217 is selected from the group |
| | | consisting of Y and C; X218 is selected from the |
| | | group consisting of N and F; X219 is selected from |
| | | the group consisting of Y and F; X220 is selected |
| | | from the group consisting of I and E; X221 is |
| | | selected from the group consisting of T and D; X222 |
| | | is selected from the group consisting of Qand P; X223 |
| | | is selected from the group consisting of Q, E, and A; |
| | | X224 is selected from the group consisting of N, L, |
| | | and Q; X227 is selected from the group consisting of |
| | | L and Y; X229 is selected from the group consisting |
| | | of D and E; X230 is selected from the group |
| | | consisting of N and D; X232 is selected from the |
| | | group consisting of F, H, and Y; X235 is selected |
| | | from the group consisting of S and A; X237 is |
| | | selected from the group consisting of E and K; X242 |
| | | is selected from the group consisting of T and I; |
| | | X244 is selected from the group consisting of K, E, |
| | | and G; X245 is selected from the group consisting of |
| | | V and A; X247 is selected from the group consisting |
| | | of T and M; X252 is selected from the group |
| | | consisting of I and M; X256 is selected from the |
| | | group consisting of P, T, and A; X265 is selected |
| | | from the group consisting of K, Q, and N; X266 is |
| | | selected from the group consisting of G and K; X269 |
| | | is selected from the group consisting of M and I; |
| | | X270 is selected from the group consisting of S and |
| | | E; X271 is selected from the group consisting of A |
| | | and T; X288 is selected from the group consisting of |
| | | S and G; X293 is selected from the group consisting |
| | | of D and Y; X295 is selected from the group |
| | | consisting of T, P, and Q; X299 is selected from the |
| | | group consisting of S and Q; X301 is selected from |
| | | the group consisting of A and V; X303 is selected |
| | | from the group consisting of Qand N; X306 is |
| | | selected from the group consisting of N and Q; X312 |
| | | is selected from the group consisting of V and L; |
| | | X316 is selected from the group consisting of I and |
| | | M; X319 is selected from the group consisting of S |
| | | and T; X321 is selected from the group consisting of |
| | | L and I; X324 is selected from the group consisting |
| | | of V and I; X330 is selected from the group |
| | | consisting of D, E, and H; X331 is selected from the |
| | | group consisting of E and G; X333 is selected from |
| | | the group consisting of E and A; X335 is selected |
| | | from the group consisting of E and A; X337 is |
| | | selected from the group consisting of A and T; X341 |
| | | is selected from the group consisting of S, D, and T; |
| | | X342 is selected from the group consisting of D and |
| | | A; X343 is selected from the group consisting of L |
| | | and I; X345 is selected from the group consisting of |
| | | R and Q; X349 is selected from the group consisting |
| | | of N and D; X353 is selected from the group |
| | | consisting of M and V; X355 is selected from the |
| | | group consisting of T and I; X360 is selected from |
| | | the group consisting of V and I; X371 is selected |
| | | from the group consisting of H and E; X374 is |
| | | selected from the group consisting of G and L; X376 |
| | | is selected from the group consisting of T and I; |
| | | X383 is selected from the group consisting of G and |
| | | S; X393 is selected from the group consisting of F |
| | | and L; X394 is selected from the group consisting of |
| | | C and Y; X397 is selected from the group consisting |
| | | of A and T; X399 is selected from the group |
| | | consisting of K, E, and G; X400 is selected from the |
| | | group consisting of S, P, and H; X404 is selected |
| | | from the group consisting of S and L; X405 is |
| | | selected from the group consisting of N and D; X406 |
| | | is selected from the group consisting of N and S; |
| | | X413 is selected from the group consisting of I and |
| | | V; X423 is selected from the group consisting of P |
| | | and L; X431 is selected from the group consisting of |
| | | P and Q; X443 is selected from the group consisting |
| | | of E and D; X444 is selected from the group |
| | | consisting of A and T; X445 is selected from the |
| | | group consisting of T and K; X448 is selected from |
| | | the group consisting of A and T; X451 is selected |
| | | from the group consisting of R and Q; X453 is |
| | | selected from the group consisting of G and D; X465 |
| | | is selected from the group consisting of V and A; |
| | | X469 is selected from the group consisting of T, S, |
| | | and N; X475 is selected from the group consisting of |
| | | A, S, and T; X481 is selected from the group |
| | | consisting of N and S; X482 is selected from the |
| | | group consisting of N and D; X485 is selected from |
| | | the group consisting of N, S, and K; X487 is selected |
| | | from the group consisting of E, R, and K; X488 is |
| | | selected from the group consisting of K, A, and E; |
| | | X492 is selected from the group consisting of L and |
| | | V; X495 is selected from the group consisting of A |
| | | and S; X497 is selected from the group consisting of |
| | | H and D; X499 is selected from the group consisting |
| | | of E and S; X500 is selected from the group |
| | | consisting of V and L; X501 is selected from the |
| | | group consisting of A and N; X502 is selected from |
| | | the group consisting of H and Y; X503 is selected |
| | | from the group consisting of G and R; X505 is |
| | | selected from the group consisting of A and T; X507 |
| | | is selected from the group consisting of I and L; |
| | | X508 is selected from the group consisting of K and |
| | | Q; X509 is selected from the group consisting of E |
| | | and K; X512 is selected from the group consisting of |
| | | G and A; X513 is selected from the group consisting |
| | | of E, D, and N; X514 is selected from the group |
| | | consisting of Y, G, A, and N; X515 is selected from |
| | | the group consisting of G and E; X516 is selected |
| | | from the group consisting of L and G; X517 is |
| | | selected from the group consisting of P and L; X519 |
| | | is selected from the group consisting of R, P, and T; |
| | | X520 is selected from the group consisting of A and |
| | | E; X521 is selected from the group consisting of E |
| | | and K; X522 is selected from the group consisting of |
| | | R, Q, and K; X523 is selected from the group |
| | | consisting of D, K, and E; X524 is selected from the |
| | | group consisting of A, T, and S; X530 is selected |
| | | from the group consisting of R and K; X533 is |
| | | selected from the group consisting of I and L; X534 |
| | | is selected from the group consisting of P and H; |
| | | X535 is selected from the group consisting of R and |
| | | Q; X544 is selected from the group consisting of T |
| | | and I; X546 is selected from the group consisting of |
| | | T, A, and I; X547 is selected from the group |
| | | consisting of P and D; X550 is selected from the |
| | | group consisting of N and K; X551 is selected from |
| | | the group consisting of A and E; X552 is selected |
| | | from the group consisting of E, R, and D; X553 is |
| | | selected from the group consisting of E, N, and R; |
| | | X554 is selected from the group consisting of H and |
| | | L; X555 is selected from the group consisting of I |
| | | and V; X556 is selected from the group consisting of |
| | | T and E; X557 is selected from the group consisting |
| | | of Q, R, H, and D; X562 is selected from the group |
| | | consisting of S and P; X573 is selected from the |
| | | group consisting of P and T; X574 is selected from |
| | | the group consisting of E and D; X575 is selected |
| | | from the group consisting of S, A, and R; X576 is |
| | | selected from the group consisting of A, E, and V; |
| | | X577 is selected from the group consisting of G, E, |
| | | and D; X579 is selected from the group consisting of |
| | | E and S; X580 is selected from the group consisting |
| | | of D and N; X583 is selected from the group |
| | | consisting of V and A; X588 is selected from the |
| | | group consisting of M and I; X591 is selected from |
| | | the group consisting of H and Y; X592 is selected |
| | | from the group consisting of A and G; X603 is |
| | | selected from the group consisting of A and S; X605 |
| | | is selected from the group consisting of E and A; |
| | | X606 is selected from the group consisting of E, A, |
| | | Q, G, V, and R; X608 is selected from the group |
| | | consisting of A, P, and T; X609 is selected from the |
| | | group consisting of I, T, and P; X610 is selected |
| | | from the group consisting of D and A; X614 is |
| | | selected from the group consisting of V and VVKEAI |
| | | (SEQ ID NO: 94); X616 is selected from the group |
| | | consisting of K and E; X622 is selected from the |
| | | group consisting of T, A, and P; andX629 is selected |
| | | from the group consisting of R, Q, and H; and X630 |
| | | is selected from the group consisting of K and no |
| | | amino acid. |
| 82 | Pseudomonas | AEEAFDLWNECAKACVLDLKDGVRSSRMSVDPAIADTNGQGVLHYSMVLEGGN |
| | exotoxin | DALKLAIDNALSITSDGLTIRLEGGVEPNKPVRYSYTRQARGSWSLNWLVPIG |
| | A variant | HEKPSNIKVFIHELNAGNQLSHMSPIYTIEMGDELLAKLARDATFFVRAHESN |
| | | EMQPTLAISHAGVSVVMAQAQPRREKRWSEWASGKVLCLLDPLDGVYNYLAQQ |
| | | RCNLDDTWEGKIYRVLAGNPAKHDLDIKPTVISHRLHFPE |
| 83 | | MVEEALNIFDECRSPCSLTPEPGKPIQSKLSIPSDVVLDEGVLYYSMTINDEQ |
| | | NDIKDEDKGESIITIGEFATVRATRHYVNQDAPFGVIHLDITTENGTKTYSYN |
| | | RKEGEFAINVVLVPIGEDSPASIKISVDELDQQRNIIEVPKLYSIDLDNQTLE |
| | | QWKTQGNVSFSVTRPEHNIAISWPSVSYKAAQKEGSRHKRWAHWHTGLALCWL |
| | | VPMDAIYNYITQQNCTLGDNVVFGGSYETVAGTPKVITVKQGIEQKPVEQRIH |
| | | FSKGGGGGSGGGGSGGGGSHGEGTFTSDLSKQMEEEAVRLFIEWLKNGGPSSG |
| | | APPPS |
| 84 | | MAEEAFDLWNECAKACVLDLKDGVRSSRMSVDPAIADTNGQGVLHYSMVLEGG |
| | | NDALKLAIDNALSITSDGLTIRLEGGVEPNKPVRYSYTRQARGSWSLNWLVPI |
| | | GHEKPSNIKVFIHELNAGNQLSHMSPIYTIEMGDELLAKLARDATFFVRAHES |
| | | NEMQPTLAISHAGVSVVMAQAQPRREKRWSEWASGKVLCLLDPLDGVYNYLAQ |
| | | QRCNLDDTWEGKIYRVLAGNPAKHDLDIKPTVISHRLHFPEG |

TABLE 13
Exemplary Transcytosing
Carriers Identifying Amino Acid
Residues of SEQ ID NO: 69
AA residues
1-252
1-253
1-254
1-255
1-256
1-257
1-258
1-259
1-260
1-261
1-262
1-263
1-264
1-265
1-266
1-267
1-268
1-269
1-270
1-271
1-272
1-273
1-274
1-275
1-276
1-277
1-278
1-279
1-280
1-281
1-282
1-283
1-284
1-285
1-286
1-287
1-288
1-289
1-290
1-291
1-292
1-293
1-294
1-295
1-296
1-297
1-298
1-299
1-300
1-301
1-302
1-303
1-304
1-305
1-306
1-307
1-308
1-309
1-310
1-311
1-312
1-313
1-314
1-315
1-316
1-317
1-318
1-319
1-320
1-321
1-322
1-323
1-324
1-325
1-326
1-327
1-328
1-329
1-330
1-331
1-332
1-333
1-334
1-335
1-336
1-337
1-338
1-339
1-340
1-341
1-342
1-343
1-344
1-345
1-346
1-347
1-348
1-349
1-350
1-350
1-351
1-352
1-353
1-354
1-355
1-356
1-357
1-358
1-359
1-360
1-361
1-362
1-363
1-364
1-365
1-366
1-367

TABLE 14
Exemplary Transcytosing Carriers
Identifying Amino Acid Residues
of SEQ ID NO: 7
AA residues
1-195
1-196
1-197
1-198
1-199
1-200
1-201
1-202
1-203
1-204
1-205
1-206
1-207
1-208
1-209
1-210
1-211
1-212
1-213
1-214
1-215
1-216
1-217
1-218
1-219
1-220
1-221
1-222
1-223
1-224
1-225
1-226
1-227
1-228
1-229
1-230
1-231
1-232
1-233
1-234
1-235
1-236
1-237
1-238
1-239
1-240
1-241
1-242
1-243
1-244
1-245
1-246
1-247
1-248
1-249
1-250
1-251
1-252
1-253
1-254
1-255
1-256
1-257
1-258
1-259
1-260
1-261
1-262
1-263
1-264
1-265
1-266
1-267
1-268
1-269
1-270
1-271
1-272
1-273
1-274
1-275
1-276
1-277
1-278
1-279
1-280
1-281
1-282
1-283
1-284
1-285
1-286
1-287
1-288
1-289
1-290
1-291
1-292
1-293
1-294
1-295
1-296
1-297
1-298
1-299
1-300
1-301
1-302
1-303
1-304
1-305
1-306
1-307
1-308
1-309
1-310
1-311
1-312
1-313
1-314
1-315
1-316
1-317
1-318
1-319
1-320
1-321
1-322
1-323
1-324
1-325
1-326
1-327
1-328
1-329
1-330
1-331
1-332
1-333
1-334
1-335
1-336
1-337
1-338
1-339
1-340
1-341
1-342
1-343
1-344
1-345
1-346
1-347

TABLE 15
Exemplary Amino Acid Residues
of Cholix Carriers of SEQ ID NO: 7
Cholix AA residues
1-150
1-151
1-152
1-153
1-154
1-155
1-156
1-157
1-158
1-159
1-160
1-161
1-162
1-163
1-164
1-165
1-166
1-167
1-168
1-169
1-170
1-171
1-172
1-173
1-174
1-175
1-176
1-177
1-178
1-179
1-180
1-181
1-182
1-183
1-184
1-185
1-186
1-187
22-187
23-187
24-187
25-187
26-187
27-187
28-187
29-187
30-187
31-187
32-187
33-187
34-187
35-187
38-187
39-187
40-187
41-187

1.-145. (canceled)

146. A composition comprising
a carrier derived from a bacterial toxin capable of entering a polarized epithelial cell or transcytosing across a polarized epithelial cell; and
a transition metal cation or a polycation, wherein the polycation is a molecule or chemical complex having more than 2 positive charges.

147. The composition of claim 146, wherein the composition comprises the transition metal cation.

148. The composition of claim 147, wherein the transition metal cation is selected from the group consisting of Fe²⁺, Mn²⁺, Zn²⁺, Co²⁺, Ni²⁺, and Cu²⁺.

149. The composition of claim 148, wherein the transition metal cation comprises Zn²⁺.

150. The composition of claim 146, wherein the composition comprises the polycation.

151. The composition of claim 150, wherein the polycation comprises protamine, poly-lysine, poly-ornithine, poly-ethylene-imine (PEI), prolamin, protamine, polyvinyl pyrrolidone (PVP), polyarginine, polyvinylamine, or a combination thereof.

152. The composition of claim 150, wherein the polycation comprises a protamine salt.

153. The composition of claim 152, wherein the protamine salt comprises protamine sulfate, protamine acetate, protamine bromide, protamine chloride, protamine caproate, protamine trifluoroacetate, protamine HCO₃, protamine propionate, protamine lactate, protamine formate, protamine nitrate, protamine citrate, protamine monohydrogenphosphate, protamine dihydrogenphosphate, protamine tartrate, or protamine perchlorate.

154. The composition of claim 152, wherein the protamine salt comprises protamine sulfate.

155. The composition of claim 146, further comprising a heterologous payload.

156. The composition of claim 155, wherein the heterologous payload comprises insulin or human growth hormone.

157. The composition of claim 146, wherein the carrier comprises Pseudomonas exotoxin A or a portion of the Pseudomonas exotoxin A.

158. The composition of claim 157, wherein the carrier comprises the sequence set forth in SEQ ID NO: 69 or a portion of the sequence set forth in SEQ ID NO: 69.

159. The composition of claim 146, wherein the carrier comprises a Cholix polypeptide.

160. The composition of claim 159, wherein the Cholix polypeptide consists of an amino acid sequence with a C-terminus at any one of amino acid positions 206 to 425, wherein the amino acid positions are based on alignment of the Cholix polypeptide to the sequence set forth in SEQ ID NO: 7, starting with position 1 at the N-terminus.

161. The composition of claim 159, wherein the Cholix polypeptide consists of an amino acid sequence with a C-terminus at any one of amino acid positions 150 to 205, wherein the amino acid positions are based on alignment of the Cholix polypeptide to the sequence set forth in SEQ ID NO: 7, starting with position 1 at the N-terminus.

162. The composition of claim 159, wherein the Cholix polypeptide consists of an amino acid sequence with a C-terminus at any one of amino acids 150 to 195, wherein the amino acid positions are based on alignment of the Cholix polypeptide to the sequence set forth in SEQ ID NO: 7, starting with position 1 at the N-terminus.

163. The composition of claim 159, wherein the Cholix polypeptide consists of an amino acid sequence with an N-terminus at any one of amino acid positions 1 to 41, wherein the amino acid positions are based on alignment of the Cholix polypeptide to the sequence set forth in SEQ ID NO: 7, starting with position 1 at the N-terminus.

164. The composition of claim 159, wherein the Cholix polypeptide consists of an amino acid sequence with an N-terminus at any one of amino acid positions 35 to 40, wherein the amino acid positions are based on alignment of the Cholix polypeptide to the sequence set forth in SEQ ID NO: 7, starting with position 1 at the N-terminus.

165. The composition of claim 159, wherein the Cholix polypeptide consists of an amino acid sequence from amino acid position 40 to any one of amino acid positions 150 to 205, wherein the amino acid positions are based on alignment of the Cholix polypeptide to the sequence set forth in SEQ ID NO: 7, starting with position 1 at the N-terminus.

166. The composition of claim 159, wherein the Cholix polypeptide has a C-terminus at any one of amino acid positions 150 to 187, wherein the amino acid positions are based on alignment of the Cholix polypeptide to the sequence set forth in SEQ ID NO: 7, starting with position 1 at the N-terminus.

167. The composition of claim 159, wherein the Cholix polypeptide consists of the amino acid sequence set forth in SEQ ID NO: 66, and the carrier further comprises a methionine N-terminal to the sequence set forth in SEQ ID NO: 66.

168. The composition of claim 159, wherein the Cholix polypeptide consists of the amino acid sequence set forth in SEQ ID NO: 2, and the carrier further comprises a methionine N-terminal to the sequence set forth in SEQ ID NO: 2.

169. The composition of claim 155, wherein the heterologous payload comprises an incretin.

170. The composition of claim 169, wherein the incretin comprises glucagon-like peptide-1 (GLP-1) or gastric inhibitory peptide (GIP).

171. The composition of claim 155, wherein the heterologous payload comprises an incretin mimetic peptide.

172. The composition of claim 171, wherein the incretin mimetic peptide comprises liraglutide, dulaglutide, semaglutide, exenatide, albiglutide, or lixisenatide.

173. The composition of claim 155, wherein the heterologous payload comprises tirzepatide.

174. The composition of claim 155, wherein the heterologous payload comprises glucagon-like peptide 2 (GLP-2) or a GLP-2 analog.

175. The composition of claim 174, wherein the heterologous payload comprises the GLP-2 analog, wherein the GLP-2 analog is teduglutide.


SEQUENCE LISTING (94 sequences)

SEQ ID NO: 1 - protein, 634 aa, Unknown
  Description of Unknown Cholix polypeptide
VEEALNIFDECRSPCSLTPEPGKPIQSKLSIPSDVVLDEGVLYYSMTINDEQNDIKDEDKGESIITIGEFATVRATRHYVNQDAPFGVIHLDITTENGTKTYSYNRKEGEFAINWLVPIGEDSPASIKISVDELDQQRNIIEVPKLYSIDLDNQTLEQWKTQGNVSFSVTRPEHNIAISWPSVSYKAAQKEGSRHKRWAHWHTGLALCWLVPMDAIYNYITQQNCTLGDNWFGGSYETVAGTPKVITVKQGIEQKPVEQRIHFSKGNAMSALAAHRVCGVPLETLARSRKPRDLTDDLSCAYQAQNIVSLFVATRILFSHLDSVFTLNLDEQEPEVAERLSDLRRINENNPGMVTQVLTVARQIYNDYVTHHPGLTPEQTSAGAQAADILSLFCPDADKSCVASNNDQANINIESRSGRSYLPENRAVITPQGVTNWTYQELEATHQALTREGYVFVGYHGTNHVAAQTIVNRIAPVPRGNNTENEEKWGGLYVATHAEVAHGYARIKEGTGEYGLPTRAERDARGVMLRVYIPRASLERFYRTNTPLENAEEHITQVIGHSLPLRNEAFTGPESAGGEDETVIGWDMAIHAVAIPSTIPGNAYEELAIDEEAVAKEQSISTKPPYKERKDELK

SEQ ID NO: 2 - protein, 386 aa, Unknown
  Description of Unknown Cholix polypeptide
VEEALNIFDECRSPCSLTPEPGKPIQSKLSIPSDVVLDEGVLYYSMTINDEQNDIKDEDKGESIITIGEFATVRATRHYVNQDAPFGVIHLDITTENGTKTYSYNRKEGEFAINWLVPIGEDSPASIKISVDELDQQRNIIEVPKLYSIDLDNQTLEQWKTQGNVSFSVTRPEHNIAISWPSVSYKAAQKEGSRHKRWAHWHTGLALCWLVPMDAIYNYITQQNCTLGDNWFGGSYETVAGTPKVITVKQGIEQKPVEQRIHFSKGNAMSALAAHRVCGVPLETLARSRKPRDLTDDLSCAYQAQNIVSLFVATRILFSHLDSVFTLNLDEQEPEVAERLSDLRRINENNPGMVTQVLTVARQIYNDYVTHHPGLTPEQTSAGAQA

SEQ ID NO: 3 - protein, 319 aa, Artificial Sequence
  Description of Artificial Sequence Synthetic polypeptide
MVEEALNIFDECRSPCSLTPEPGKPIQSKLSIPSDVVLDEGVLYYSMTINDEQNDIKDEDKGESIITIGEFATVRATRHYVNQDAPFGVIHLDITTENGTKTYSYNRKEGEFAINWLVPIGEDSPASIKISVDELDQQRNIIEVPKLYSIDLDNQTLEQWKTQGNVSFSVTRPEHNIAISWPSVSYKAAQKEGSRHKRWAHWHTGLALCWLVPMDAIYNYITQQNCTLGDNWFGGSYETVAGTPKVITVKQGIEQKPVEQRIHFSKGSGGSGGGGSGGGGSPRRRRSSSRPVRRRRRPRVSRRRRRRGGRRRRHHHHHH

SEQ ID NO: 4 - protein, 14 aa, Artificial Sequence
  Description of Artificial Sequence Synthetic peptide
GGGGSGGGGSGGGG

SEQ ID NO: 5 - protein, 192 aa, Homo sapiens
MFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF

SEQ ID NO: 6 - protein, 415 aa, Unknown
  Description of Unknown Cholix polypeptide
VEEALNIFDECRSPCSLTPEPGKPIQSKLSIPSDVVLDEGVLYYSMTINDEQNDIKDEDKGESIITIGEFATVRATRHYVNQDAPFGVIHLDITTENGTKTYSYNRKEGEFAINWLVPIGEDSPASIKISVDELDQQRNIIEVPKLYSIDLDNQTLEQWKTQGNVSFSVTRPEHNIAISWPSVSYKAAQKEGSRHKRWAHWHTGLALCWLVPMDAIYNYITQQNCTLGDNWFGGSYETVAGTPKVITVKQGIEQKPVEQRIHFSKGNAMSALAAHRVCGVPLETLARSRKPRDLTDDLSCAYQAQNIVSLFVATRILFSHLDSVFTLNLDEQEPEVAERLSDLRRINENNPGMVTQVLTVARQIYNDYVTHHPGLTPEQTSAGAQAADILSLFCPDADKSCVASNNDQANINIES

SEQ ID NO: 7 - protein, 649 aa, Artificial Sequence
  Description of Artificial Sequence Synthetic polypeptide
  MOD_RES (1)..(1): V or L
  MOD_RES (3)..(3): E or D
  MOD_RES (4)..(4): A or E
  MOD_RES (6)..(6): N or K
  MOD_RES (16)..(16): S or L
  MOD_RES (21)..(21): P or L
  MOD_RES (24)..(24): P or Q
  MOD_RES (30)..(30): S or F
  MOD_RES (33)..(33): S or G
  MOD_RES (56)..(56): K or M
  MOD_RES (59)..(59): D or G
  MOD_RES (67)..(67): I or F
  MOD_RES (73)..(73): V or I
  MOD_RES (81)..(81): N or S
  MOD_RES (90)..(90): H or N
  MOD_RES (101)..(101): T or M
  MOD_RES (104)..(104): Y or F
  MOD_RES (108)..(108): E or D
  MOD_RES (109)..(109): G or S
  MOD_RES (112)..(112): A or T
  MOD_RES (114)..(114): N or H
  MOD_RES (118)..(118): P or I
  MOD_RES (119)..(119): I or P
  MOD_RES (131)..(131): V or I
  MOD_RES (134)..(134): L or I
  MOD_RES (137)..(137): Q or K
  MOD_RES (160)..(160): K or E
  MOD_RES (161)..(161): T or N
  MOD_RES (166)..(166): S or F
  MOD_RES (168)..(168): S or A
  MOD_RES (174)..(174): H or Q
  MOD_RES (175)..(185): This region may encompass one of the following sequences “N”, “S”, “SIAKQS”, or “SIAKQSIAKQS”
  MOD_RES (196)..(196): K or N
  MOD_RES (199)..(199): Q, E, or H
  MOD_RES (201)..(201): E, N, or D
  MOD_RES (203)..(203): S or A
  MOD_RES (210)..(210): H or N
  MOD_RES (212)..(212): H, L, F, or R
  MOD_RES (214)..(214): G or T
  MOD_RES (215)..(215): L or S
  MOD_RES (216)..(216): A or P
  MOD_RES (217)..(217): L, E, or K
  MOD_RES (218)..(218): C or V
  MOD_RES (219)..(219): W, V, or T
  MOD_RES (221)..(221): V or absent
  MOD_RES (222)..(222): P or absent
  MOD_RES (223)..(223): M, I, L, or absent
  MOD_RES (224)..(224): D or absent
  MOD_RES (225)..(225): A or absent
  MOD_RES (226)..(226): I or absent
  MOD_RES (227)..(227): Y or C
  MOD_RES (228)..(228): N or F
  MOD_RES (229)..(229): Y or F
  MOD_RES (230)..(230): I or E
  MOD_RES (231)..(231): T or D
  MOD_RES (232)..(232): Q or P
  MOD_RES (233)..(233): Q, E, or A
  MOD_RES (234)..(234): N, L, or Q
  MOD_RES (237)..(237): L or Y
  MOD_RES (239)..(239): D or E
  MOD_RES (240)..(240): N or D
  MOD_RES (242)..(242): F, H, or Y
  MOD_RES (245)..(245): S or A
  MOD_RES (247)..(247): E or K
  MOD_RES (252)..(252): T or I
  MOD_RES (254)..(254): K, E, or G
  MOD_RES (255)..(255): V or A
  MOD_RES (257)..(257): T or M
  MOD_RES (262)..(262): I or M
  MOD_RES (266)..(266): P, T, or A
  MOD_RES (275)..(275): K, Q, or N
  MOD_RES (276)..(276): G or K
  MOD_RES (279)..(279): M or I
  MOD_RES (280)..(280): S or E
  MOD_RES (281)..(281): A or T
  MOD_RES (298)..(298): S or G
  MOD_RES (303)..(303): D or Y
  MOD_RES (305)..(305): T, P, or Q
  MOD_RES (309)..(309): S or Q
  MOD_RES (311)..(311): A or V
  MOD_RES (313)..(313): Q or N
  MOD_RES (316)..(316): N or Q
  MOD_RES (322)..(322): V or L
  MOD_RES (326)..(326): I or M
  MOD_RES (329)..(329): S or T
  MOD_RES (331)..(331): L or I
  MOD_RES (334)..(334): V or I
  MOD_RES (340)..(340): D, E, or H
  MOD_RES (341)..(341): E or G
  MOD_RES (343)..(343): E or A
  MOD_RES (345)..(345): E or A
  MOD_RES (347)..(347): A or T
  MOD_RES (351)..(351): S, D, or T
  MOD_RES (352)..(352): D or A
  MOD_RES (353)..(353): L or I
  MOD_RES (355)..(355): R or Q
  MOD_RES (359)..(359): N or D
  MOD_RES (363)..(363): M or V
  MOD_RES (365)..(365): T or I
  MOD_RES (370)..(370): V or I
  MOD_RES (381)..(381): H or E
  MOD_RES (384)..(384): G or L
  MOD_RES (386)..(386): T or I
  MOD_RES (393)..(393): G or S
  MOD_RES (403)..(403): F or L
  MOD_RES (404)..(404): C or Y
  MOD_RES (407)..(407): A or T
  MOD_RES (409)..(409): K, E, or G
  MOD_RES (410)..(410): S, P, or H
  MOD_RES (414)..(414): S or L
  MOD_RES (415)..(415): N or D
  MOD_RES (416)..(416): N or S
  MOD_RES (423)..(423): I or V
  MOD_RES (433)..(433): P or L
  MOD_RES (441)..(441): P or Q
  MOD_RES (453)..(453): E or D
  MOD_RES (454)..(454): A or T
  MOD_RES (455)..(455): T or K
  MOD_RES (458)..(458): A or T
  MOD_RES (461)..(461): R or Q
  MOD_RES (463)..(463): G or D
  MOD_RES (475)..(475): V or A
  MOD_RES (479)..(479): T, S, or N
  MOD_RES (485)..(485): A, S, or T
  MOD_RES (491)..(491): N or S
  MOD_RES (492)..(492): N or D
  MOD_RES (495)..(495): N, S, or K
  MOD_RES (497)..(497): E, R, or K
  MOD_RES (498)..(498): K, A, or E
  MOD_RES (502)..(502): L or V
  MOD_RES (505)..(505): A or S
  MOD_RES (507)..(507): H or D
  MOD_RES (509)..(509): E or S
  MOD_RES (510)..(510): V or L
  MOD_RES (511)..(511): A or N
  MOD_RES (512)..(512): H or Y
  MOD_RES (513)..(513): G or R
  MOD_RES (515)..(515): A or T
  MOD_RES (517)..(517): I or L
  MOD_RES (518)..(518): K or Q
  MOD_RES (519)..(519): E or K
  MOD_RES (522)..(522): G or A
  MOD_RES (523)..(523): E, D, or N
  MOD_RES (524)..(524): Y, G, A, or N
  MOD_RES (525)..(525): G or E
  MOD_RES (526)..(526): L or G
  MOD_RES (527)..(527): P or L
  MOD_RES (529)..(529): R, P, or T
  MOD_RES (530)..(530): A or E
  MOD_RES (531)..(531): E or K
  MOD_RES (532)..(532): R, Q, or K
  MOD_RES (533)..(533): D, K, or E
  MOD_RES (534)..(534): A, T, or S
  MOD_RES (540)..(540): R or K
  MOD_RES (543)..(543): I or L
  MOD_RES (544)..(544): P or H
  MOD_RES (545)..(545): R or Q
  MOD_RES (554)..(554): T or I
  MOD_RES (556)..(556): T, A, or I
  MOD_RES (557)..(557): P or D
  MOD_RES (560)..(560): N or K
  MOD_RES (561)..(561): A or E
  MOD_RES (562)..(562): E, R, or D
  MOD_RES (563)..(563): E, N, or R
  MOD_RES (564)..(564): H or L
  MOD_RES (565)..(565): I or V
  MOD_RES (566)..(566): T or E
  MOD_RES (567)..(567): Q, R, H, or D
  MOD_RES (572)..(572): S or P
  MOD_RES (583)..(583): P or T
  MOD_RES (584)..(584): E or D
  MOD_RES (585)..(585): S, A, or R
  MOD_RES (586)..(586): A, E, or V
  MOD_RES (587)..(587): G, E, or D
  MOD_RES (589)..(589): E or S
  MOD_RES (590)..(590): D or N
  MOD_RES (593)..(593): V or A
  MOD_RES (598)..(598): M or I
  MOD_RES (601)..(601): H or Y
  MOD_RES (602)..(602): A or G
  MOD_RES (613)..(613): A or S
  MOD_RES (615)..(615): E or A
  MOD_RES (616)..(616): E, A, Q, G, V, or R
  MOD_RES (618)..(618): A, P, or T
  MOD_RES (619)..(619): I, T, or P
  MOD_RES (620)..(620): D or A
  MOD_RES (624)..(629): This region may encompass one of the following sequences “V” or “VVKEAI”
  MOD_RES (631)..(631): K or E
  MOD_RES (637)..(637): T, A, or P
  MOD_RES (644)..(644): R, Q, or H
  MOD_RES (645)..(645): K or absent
  See specification as filed for detailed description of substitutions and preferred embodiments
XEXXLXIFDECRSPCXLTPEXGKXIQSKLXIPXDVVLDEGVLYYSMTINDEQNDIXDEXKGESIITXGEFATXRATRHYVXQDAPFGVIXLDITTENGTKXYSXNRKXXEFXIXWLVXXGEDSPASIKISXDEXDQXRNIIEVPKLYSIDLDNQTLEQWXXQGNVXFXVTRPEXXXXXXXXXXXXIAISWPSVSYXAAXKXGXRHKRWAXWXTXXXXXXLXXXXXXXXXXXXXXCTXGXXWXGGXYXTVAGXPXXIXVKQGXEQKXVEQRIHFSXXNAXXXLAAHRVCGVPLETLARXRKPRXLXDDLXCXYXAQXIVSLFXATRXLFXHXDSXFTLNLXXQXPXVXERLXXXRXINEXNPGXVXQVLTXARQIYNDYVTXHPXLXPEQTSAXAQAADILSLXXPDXDXXCVAXXXDQANINXESRSGRSYLXENRAVITXQGVTNWTYQELXXXHQXLTXEXYVFVGYHGTNHXAAQXIVNRIXPVPRGXXTEXEXXWGGXYVXTXAXXXXXYXRXXXGTXXXXXXTXXXXXXRGVMLXVYXXXASLERFYRXNXXLEXXXXXXXXVIGHXLPLRNEAFTGXXXXXGXXETXIGWDXAIXXVAIPSTIPGNXYXXLXXXEEAXXXXXXAXEQSISXKPPYKEXXDELK

SEQ ID NO: 8 - protein, 147 aa, Unknown
  Description of Unknown Cholix polypeptide
VLYYSMTINDEQNDIKDEDKGESIITIGEFATVRATRHYVNQDAPFGVIHLDITTENGTKTYSYNRKEGEFAINWLVPIGEDSPASIKISVDELDQQRNIIEVPKLYSIDLDNQTLEQWKTQGNVSFSVTRPEHNIAISWPSVSYKA

SEQ ID NO: 9 - protein, 166 aa, Unknown
  Description of Unknown Cholix polypeptide
GVLYYSMTINDEQNDIKDEDKGESIITIGEFATVRATRHYVNQDAPFGVIHLDITTENGTKTYSYNRKEGEFAINWLVPIGEDSPASIKISVDELDQQRNIIEVPKLYSIDLDNQTLEQWKTQGNVSFSVTRPEHNIAISWPSVSYKAAQKEGSRHKRWAHWHTGL

SEQ ID NO: 10 - protein, 151 aa, Unknown
  Description of Unknown Cholix polypeptide
VEEALNIFDECRSPCSLTPEPGKPIQSKLSIPSDVVLDEGVLYYSMTINDEQNDIKDEDKGESIITIGEFATVRATRHYVNQDAPFGVIHLDITTENGTKTYSYNRKEGEFAINWLVPIGEDSPASIKISVDELDQQRNIIEVPKLYSIDL

SEQ ID NO: 11 - protein, 39 aa, Artificial Sequence
  Description of Artificial Sequence Synthetic polypeptide
HGEGTFTSDLSKQMEEEAVRLFIEWLKNGGPSSGAPPPS

SEQ ID NO: 12 - protein, 634 aa, Unknown
  Description of Unknown Cholix polypeptide
LEEALNIFDECRSPCSLTPEPGKPIQSKLSIPSDVVLDEGVLYYSMTINDEQNDIKDEDKGESIITIGEFATVRATRHYVNQDAPFGVIHLDITTENGTKTYSYNRKEGEFAINWLVPIGEDSPASIKISVDELDQQRNIIEVPKLYSIDLDNQTLEQWKTQGNVSFSVTRPEHNIAISWPSVSYKAAQKEGSRHKRWAHWHTGLALCWLVPMDAIYNYITQQNCTLGDNWFGGSYETVAGTPKVITVKQGIEQKPVEQRIHFSKGNAMSALAAHRVCGVPLETLARSRKPRDLTDDLSCAYQAQNIVSLFVATRILFSHLDSVFTLNLDEQEPEVAERLSDLRRINENNPGMVTQVLTVARQIYNDYVTHHPGLTPEQTSAGAQAADILSLFCPDADKSCVASNNDQANINIESRSGRSYLPENRAVITPQGVTNWTYQELEATHQALTREGYVFVGYHGTNHVAAQTIVNRIAPVPRGNNTENEEKWGGLYVATHAEVAHGYARIKEGTGEYGLPTRAERDARGVMLRVYIPRASLERFYRTNTPLENAEEHITQVIGHSLPLRNEAFTGPESAGGEDETVIGWDMAIHAVAIPSTIPGNAYEELAIDEEAVAKEQSISTKPPYKERKDELK

SEQ ID NO: 13 - protein, 386 aa, Unknown
  Description of Unknown Cholix polypeptide
LEEALNIFDECRSPCSLTPEPGKPIQSKLSIPSDVVLDEGVLYYSMTINDEQNDIKDEDKGESIITIGEFATVRATRHYVNQDAPFGVIHLDITTENGTKTYSYNRKEGEFAINWLVPIGEDSPASIKISVDELDQQRNIIEVPKLYSIDLDNQTLEQWKTQGNVSFSVTRPEHNIAISWPSVSYKAAQKEGSRHKRWAHWHTGLALCWLVPMDAIYNYITQQNCTLGDNWFGGSYETVAGTPKVITVKQGIEQKPVEQRIHFSKGNAMSALAAHRVCGVPLETLARSRKPRDLTDDLSCAYQAQNIVSLFVATRILFSHLDSVFTLNLDEQEPEVAERLSDLRRINENNPGMVTQVLTVARQIYNDYVTHHPGLTPEQTSAGAQA

SEQ ID NO: 14 - protein, 39 aa, Artificial Sequence; identical to SEQ ID NO: 11
  Description of Artificial Sequence Synthetic polypeptide

SEQ ID NO: 15 - protein, 44 aa, Artificial Sequence
  Description of Artificial Sequence Synthetic polypeptide
HGEGTFTSDLSKQMEEEAVRLFIEWLKNGGPSSGAPPSKKKKKK

SEQ ID NO: 16 - protein, 32 aa, Artificial Sequence
  Description of Artificial Sequence Synthetic polypeptide
  MOD_RES (21)..(21): gamma-E-palmitoyl
HAEGTFTSDVSSYLEGQAAKEEFIAWLVRGRG

SEQ ID NO: 17 - protein, 275 aa, Artificial Sequence
  Description of Artificial Sequence Synthetic polypeptide
HGEGTFTSDVSSYLEEQAAKEFIAWLVKGGGGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 18 - protein, 33 aa, Unknown
  Description of Unknown Teduglutide sequence
HGDGSFSDEMNTILDNLAARDFINWLIQTKITD

SEQ ID NO: 19 - protein, 33 aa, Unknown; identical to SEQ ID NO: 18
  Description of Unknown Teduglutide sequence

SEQ ID NO: 20 - protein, 191 aa, Homo sapiens [sequence omitted]

SEQ ID NO: 21 - protein, 146 aa, Unknown
  Description of Unknown IL-22 sequence
APISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 22 - protein, 160 aa, Unknown
  Description of Unknown IL-10 sequence
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 23 - protein, 180 aa, Unknown
  Description of Unknown Preproglucagon sequence
MKSIYFVAGLFVMLVQGSWQRSLQDTEEKSRSFSASQADPLSDPDQMNEDKRHSQGTFTSDYSKYLDSRRAQDFVQWLMNTKRNRNNIAKRHDEFERHAEGTFTSDVSSYLEGQAAKEFIAWLVKGRGRRDFPEEVAIVEELGRRHADGSFSDEMNTILDNLAARDFINWLIQTKITDRK

SEQ ID NO: 24 - protein, 160 aa, Unknown
  Description of Unknown Glucagon proprotein sequence
RSLQDTEEKSRSFSASQADPLSDPDQMNEDKRHSQGTFTSDYSKYLDSRRAQDFVQWLMNTKRNRNNIAKRHDEFERHAEGTFTSDVSSYLEGQAAKEFIAWLVKGRGRRDFPEEVAIVEELGRRHADGSFSDEMNTILDNLAARDFINWLIQTKITDRK

SEQ ID NO: 25 - protein, 29 aa, Unknown
  Description of Unknown Glucagon peptide sequence
HSQGTFTSDYSKYLDSRRAQDFVQWLMNT

SEQ ID NO: 26 - protein, 30 aa, Unknown
  Description of Unknown Glucagon-like peptide 1 (GLP-1) sequence
HAEGTFTSDVSSYLEGQAAKEFIAWLVKGR

SEQ ID NO: 27 - protein, 33 aa, Unknown
  Description of Unknown Glucagon-like peptide 2 (GLP-2) sequence
HADGSFSDEMNTILDNLAARDFINWLIQTKITD

SEQ ID NO: 28 - protein, 39 aa, Unknown
  Description of Unknown Exendin-3 sequence
HSDGTFTSDLSKQMEEEAVRLFIEWLKNGGPSSGAPPPS

SEQ ID NO: 29 - protein, 38 aa, Artificial Sequence
  Description of Artificial Sequence Synthetic polypeptide
  MOD_RES (26)..(26): K-PEG-Fc
GEGTFTSDLSKQMEEEAVRLFIEWLKNGGPSSGAPPPS

SEQ ID NO: 30 - protein, 31 aa, Artificial Sequence
  Description of Artificial Sequence Synthetic polypeptide
  MOD_RES (2)..(2): Any amino acid
HXEGTFTSDVSSYLEGQAAKEFIAWLVRGRG

SEQ ID NO: 31 - protein, 37 aa, Unknown
  Description of Unknown GLP-1R agonist (aa 1-37 of GIP) sequence
YAEGTFISDYSIAMDKIHQQDFVNWLLAQKGKKNDWK

SEQ ID NO: 32 - protein, 30 aa, Unknown
  Description of Unknown GLP-1R agonist (aa 7-36 of GIP) sequence
ISDYSIAMDKIHQQDFVNWLLAQKGKKNDW

SEQ ID NO: 33 - protein, 39 aa, Artificial Sequence
  Description of Artificial Sequence Synthetic polypeptide
  MOD_RES (2)..(2): Any amino acid
  MOD_RES (13)..(13): Any amino acid
YXEGTFTSDYSIXLDKIAQKAFVQWLIAGGPSSGAPPPS

SEQ ID NO: 34 - protein, 37 aa, Unknown
  Description of Unknown Oxyntomodulin sequence
HSQGTFTSDYSKYLDSRRAQDFVQWLMNTKRNKNNIA

SEQ ID NO: 35 - protein, 153 aa, Unknown
  Description of Unknown Gastric inhibitory polypeptide preprotein sequence
MVATKTFALLLLSLFLAVGLGEKKEGHFSALPSLPVGSHAKVSSPQPRGPRYAEGTFISDYSIAMDKIHQQDFVNWLLAQKGKKNDWKHNITQREARALELASQANRKEEEAVEPQSSPAKNPSDEDLLRDLLIQELLACLLDQTNLCRLRSR

SEQ ID NO: 36 - protein, 42 aa, Unknown
  Description of Unknown Gastric inhibitory polypeptide (GIP) sequence
YAEGTFISDYSIAMDKIHQQDFVNWLLAQKGKKNDWKHNITQ

SEQ ID NO: 37 - protein, 29 aa, Unknown
  Description of Unknown GIPR agonist (Des-Ala2-GIP1-30 ) sequence
YEGTFISDYSIAMDKIHQQDFVNWLLAQK

SEQ ID NO: 38 - protein, 30 aa, Unknown
  Description of Unknown GIPR agonist-Truncated GIP1-30 sequence
YAEGTFISDYSIAMDKIHQQDFVNWLLAQK

SEQ ID NO: 39 - protein, 69 aa, Unknown
  Description of Unknown Glicentin sequence
RSLQDTEEKSRSFSASQADPLSDPDQMNEDKRHSQGTFTSDYSKYLDSRRAQDFVQWLMNTKRNRNNIA

SEQ ID NO: 40 - protein, 30 aa, Unknown
  Description of Unknown Glicentin-related polypeptide sequence
RSLQDTEEKSRSFSASQADPLSDPDQMNED

SEQ ID NO: 41 - protein, 32 aa, Unknown
  Description of Unknown Dual Amylin Calcitonin Receptor Agonist sequence
CSNLSTCMLGRLSQDLHRLQTYPKTDVGANAP

SEQ ID NO: 42 - protein, 110 aa, Unknown
  Description of Unknown Preproinsulin sequence
MALWMRLLPLLALLALWGPDPAAAFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 43 - protein, 21 aa, Homo sapiens
GIVEQCCTSICSLYQLENYCN

SEQ ID NO: 44 - protein, 30 aa, Homo sapiens
FVNQHLCGSHLVEALYLVCGERGFFYTPKT

SEQ ID NO: 45 - protein, 21 aa, Unknown
  Description of Unknown Insulin Aspart A chain sequence
GIVEQCCTSICSLYQLENYCN

SEQ ID NO: 46 - protein, 30 aa, Unknown
  Description of Unknown Insulin Aspart B chain sequence
FVNQHLCGSHLVEALYLVCGERGFFYTDKT

SEQ ID NO: 47 - protein, 21 aa, Unknown
  Description of Unknown Insulin glargine A chain sequence
GIVEQCCTSICSLYQLENYCG

SEQ ID NO: 48 - protein, 32 aa, Unknown
  Description of Unknown Insulin glargine B chain sequence
FVNQHLCGSHLVEALYLVCGERGFFYTPKTRR

SEQ ID NO: 49 - protein, 21 aa, Unknown
  Description of Unknown Insulin lispro A chain sequence
GIVEQCCTSICSLYQLENYCN

SEQ ID NO: 50 - protein, 30 aa, Unknown
  Description of Unknown Insulin lispro B chain sequence
FVNQHLCGSHLVEALYLVCGERGFFYTKPT

SEQ ID NO: 51 - protein, 1242 aa, Unknown
  Description of Unknown Insulin receptor substrate 1 sequence
MASPPESDGFSDVRKVGYLRKPKSMHKRFFVLRAASEAGGPARLEYYENEKKWRHKSSAPKRSIPLESCFNINKRADSKNKHLVALYTRDEHFAIAADSEAEQDSWYQALLQLHNRAKGHHDGAAALGAGGGGGSCSGSSGLGEAGEDLSYGDVPPGPAFKEVWQVILKPKGLGQTKNLIGIYRLCLTSKTISFVKLNSEAAAVVLQLMNIRRCGHSENFFFIEVGRSAVTGPGEFWMQVDDSVVAQNMHETILEAMRAMSDEFRPRSKSQSSSNCSNPISVPLRRHHLNNPPPSQVGLTRRSRTESITATSPASMVGGKPGSFRVRASSDGEGTMSRPASVDGSPVSPSTNRTHAHRHRGSARLHPPLNHSRSIPMPASRCSPSATSPVSLSSSSTSGHGSTSDCLFPRRSSASVSGSPSDGGFISSDEYGSSPCDFRSSFRSVTPDSLGHTPPARGEEELSNYICMGGKGPSTLTAPNGHYILSRGGNGHRCTPGTGLGTSPALAGDEAASAADLDNRFRKRTHSAGTSPTITHQKTPSQSSVASIEEYTEMMPAYPPGGGSGGRLPGHRHSAFVPTRSYPEEGLEMHPLERRGGHHRPDSSTLHTDDGYMPMSPGVAPVPSGRKGSGDYMPMSPKSVSAPQQIINPIRRHPQRVDPNGYMMMSPSGGCSPDIGGGPSSSSSSSNAVPSGTSYGKLWTNGVGGHHSHVLPHPKPPVESSGGKLLPCTGDYMNMSPVGDSNTSSPSDCYYGPEDPQHKPVLSYYSLPRSFKHTQRPGEPEEGARHQHLRLSTSSGRLLYAATADDSSSSTSSDSLGGGYCGARLEPSLPHPHHQVLQPHLPRKVDTAAQTNSRLARPTRLSLGDPKASTLPRAREQQQQQQPLLHPPEPKSPGEYVNIEFGSDQSGYLSGPVAFHSSPSVRCPSQLQPAPREEETGTEEYMKMDLGPGRRAAWQESTGVEMGRLGPAPPGAASICRPTRAVPSSRGDYMTMQMSCPRQSYVDTSPAAPVSYADMRTGIAAEEVSLPRATMAAASSSSAASASPTGPQGAAELAAHSSLLGGPQGPGGMSAFTRVNLSPNRNQSAKVIRADPQGCRRRHSSETFSSTPSATRVGNTVPFGAGAAVGGGGGSSSSSEDVKRHSSASFENVWLRPGELGGAPKEPAKLCGAAGGLENGLNYIDLDLVKDFKQCPQECTPEPQPPPPPPPHQPLGSGESSSTRRSSEDLSAYASISFQKQPEDRQ

SEQ ID NO: 52 - protein, 100 aa, Unknown
  Description of Unknown Apolipoprotein A-II sequence
MKLLAATVLLLTICSLEGALVRRQAKEPCVESLVSQYFQTVTDYGKDLMEKVKSPELQAEAKSYFEKSKEQLTPLIKKAGTELVNFLSYFVELGTQPATQ

SEQ ID NO: 53 - protein, 737 aa, Unknown
  Description of Unknown Glycogen synthase 1 sequence
MPLNRTLSMSSLPGLEDWEDEFDLENAVLFEVAWEVANKVGGIYTVLQTKAKVTGDEWGDNYFLVGPYTEQGVRTQVELLEAPTPALKRTLDSMNSKGCKVYFGRWLIEGGPLVVLLDVGASAWALERWKGELWDTCNIGVPWYDREANDAVLFGFLTTWFLGEFLAQSEEKPHVVAHFHEWLAGVGLCLCRARRLPVATIFTTHATLLGRYLCAGAVDFYNNLENFNVDKEAGERQIYHRYCMERAAAHCAHVFTTVSQITAIEAQHLLKRKPDIVTPNGLNVKKFSAMHEFQNLHAQSKARIQEFVRGHFYGHLDFNLDKTLYFFIAGRYEFSNKGADVFLEALARLNYLLRVNGSEQTVVAFFIMPARTNNFNVETLKGQAVRKQLWDTANTVKEKFGRKLYESLLVGSLPDMNKMLDKEDFTMMKRAIFATQRQSFPPVCTHNMLDDSSDPILTTIRRIGLFNSSADRVKVIFHPEFLSSTSPLLPVDYEEFVRGCHLGVFPSYYEPWGYTPAECTVMGIPSISTNLSGFGCFMEEHIADPSAYGIYILDRRFRSLDDSCSQLTSFLYSFCQQSRRQRIIQRNRTERLSDLLDWKYLGRYYMSARHMALSKAFPEHFTYEPNEADAAQGYRYPRPASVPPSPSLSRHSSPHQSEDEEDPRNGPLEEDGERYDEDEEAAKDRRNIRAPEWPRRASCTSSTSGSKRNSVDTATSSSLSTPSEPLSPTSSLGEERN

SEQ ID NO: 54 - protein, 703 aa, Unknown
  Description of Unknown Glycogen synthase 2 sequence
MLRGRSLSVTSLGGLPQWEVEELPVEELLLFEVAWEVTNKVGGIYTVIQTKAKTTADEWGENYFLIGPYFEHNMKTQVEQCEPVNDAVRRAVDAMNKHGCQVHFGRWLIEGSPYVVLFDIGYSAWNLDRWKGDLWEACSVGIPYHDREANDMLIFGSLTAWFLKEVTDHADGKYVVAQFHEWQAGIGLILSRARKLPIATIFTTHATLLGRYLCAANIDFYNHLDKFNIDKEAGERQIYHRYCMERASVHCAHVFTTVSEITAIEAEHMLKRKPDVVTPNGLNVKKFSAVHEFQNLHAMYKARIQDFVRGHFYGHLDFDLEKTLFLFIAGRYEFSNKGADIFLESLSRLNFLLRMHKSDITVMVFFIMPAKTNNFNVETLKGQAVRKQLWDVAHSVKEKFGKKLYDALLRGEIPDLNDILDRDDLTIMKRAIFSTQRQSLPPVTTHNMIDDSTDPILSTIRRIGLFNNRTDRVKVILHPEFLSSTSPLLPMDYEEFVRGCHLGVFPSYYEPWGYTPAECTVMGIPSVTTNLSGFGCFMQEHVADPTAYGIYIVDRRFRSPDDSCNQLTKFLYGFCKQSRRQRIIQRNRTERLSDLLDWRYLGRYYQHARHLTLSRAFPDKFHVELTSPPTTEGFKYPRPSSVPPSPSGSQASSPQSSDVEDEVEDERYDEEEEAERDRLNIKSPFSLSHVPHGKKKLHGEYKN

SEQ ID NO: 55 - protein, 435 aa, Unknown
  Description of Unknown Tyrosin-protein phosphatase non-receptor type 1 sequence
MEMEKEFEQIDKSGSWAAIYQDIRHEASDFPCRVAKLPKNKNRNRYRDVSPFDHSRIKLHQEDNDYINASLIKMEEAQRSYILTQGPLPNTCGHFWEMVWEQKSRGVVMLNRVMEKGSLKCAQYWPQKEEKEMIFEDTNLKLTLISEDIKSYYTVRQLELENLTTQETREILHFHYTTWPDFGVPESPASFLNFLFKVRESGSLSPEHGPVVVHCSAGIGRSGTFCLADTCLLLMDKRKDPSSVDIKKVLLEMRKFRMGLIQTADQLRFSYLAVIEGAKFIMGDSSVQDQWKELSHEDLEPPPEHIPPPPRPPKRILEPHNGKCREFFPNHQWVKEETQEDKDCPIKEEKGSPLNAAPYGIESMSQDTEVRSRVVGGSLRGAQAASPAKGEPSLPEKDEDHALSYWKPFLVNMCVATVLTAGAYLCYRFLFNSNT

SEQ ID NO: 56 - protein, 480 aa, Unknown [sequence omitted]

SEQ ID NO: 57 - protein, 505 aa, Unknown
  Description of Unknown Peroxisome proliferator-activated receptor gamma sequence
MGETLGDSPIDPESDSFTDTLSANISQEMTMVDTEMPFWPTNFGISSVDLSVMEDHSHSFDIKPFTTVDFSSISTPHYEDIPFTRTDPVVADYKYDLKLQEYQSAIKVEPASPPYYSEKTQLYNKPHEEPSNSLMAIECRVCGDKASGFHYGVHACEGCKGFFRRTIRLKLIYDRCDLNCRIHKKSRNKCQYCRFQKCLAVGMSHNAIRFGRMPQAEKEKLLAEISSDIDQLNPESADLRALAKHLYDSYIKSFPLTKAKARAILTGKTTDKSPFVIYDMNSLMMGEDKIKFKHITPLQEQSKEVAIRIFQGCQFRSVEAVQEITEYAKSIPGFVNLDLNDQVTLLKYGVHEIIYTMLASLMNKDGVLISEGQGFMTREFLKSLRKPFGDFMEPKFEFAVKFNALELDDSDLAIFIAVIILSGDRPGLLNVKPIEDIQDNLLQALELQLKLNHPESSQLFAKLLQKMTDLRQIVTEHVQLLQVIKKTETDMSLHPLLQEIYKDLY

SEQ ID NO: 58 - protein, 923 aa, Unknown
  Description of Unknown Hexokinase 3 sequence
MDSIGSSGLRQGEETLSCSEEGLPGPSDSSELVQECLQQFKVTRAQLQQIQASLLGSMEQALRGQASPAPAVRMLPTYVGSTPHGTEQGDFVVLELGATGASLRVLWVTLTGIEGHRVEPRSQEFVIPQEVMLGAGQQLFDFAAHCLSEFLDAQPVNKQGLQLGFSFSFPCHQTGLDRSTLISWTKGFRCSGVEGQDVVQLLRDAIRRQGAYNIDVVAVVNDTVGTMMGCEPGVRPCEVGLVVDTGTNACYMEEARHVAVLDEDRGRVCVSVEWGSFSDDGALGPVLTTFDHTLDHESLNPGAQRFEKMIGGLYLGELVRLVLAHLARCGVLFGGCTSPALLSQGSILLEHVAEMEDPSTGAARVHAILQDLGLSPGASDVELVQHVCAAVCTRAAQLCAAALAAVLSCLQHSREQQTLQVAVATGGRVCERHPRFCSVLQGTVMLLAPECDVSLIPSVDGGGRGVAMVTAVAARLAAHRRLLEETLAPFRLNHDQLAAVQAQMRKAMAKGLRGEASSLRMLPTFVRATPDGSERGDFLALDLGGTNFRVLLVRVTTGVQITSEIYSIPETVAQGSGQQLFDHIVDCIVDFQQKQGLSGQSLPLGFTFSFPCRQLGLDQGILLNWTKGFKASDCEGQDVVSLLREAITRRQAVELNVVAIVNDTVGTMMSCGYEDPRCEIGLIVGTGTNACYMEELRNVAGVPGDSGRMCINMEWGAFGDDGSLAMLSTRFDASVDQASINPGKQRFEKMISGMYLGEIVRHILLHLTSLGVLFRGQQIQRLQTRDIFKTKFLSEIESDSLALRQVRAILEDLGLPLTSDDALMVLEVCQAVSQRAAQLCGAGVAAVVEKIRENRGLEELAVSVGVDGTLYKLHPRFSSLVAATVRELAPRCVVTFLQSEDGSGKGAALVTAVACRLAQLTRV

SEQ ID NO: 59 - protein, 403 aa, Unknown [sequence omitted]

SEQ ID NO: 60 - protein, 436 aa, Unknown
  Description of Unknown Pyruvate dehydrogenase kinase 1 sequence
MRLARLLRGAALAGPGPGLRAAGFSRSFSSDSGSSPASERGVPGQVDFYARFSPSPLSMKQFLDFGSVNACEKTSFMFLRQELPVRLANIMKEISLLPDNLLRTPSVQLVQSWYIQSLQELLDFKDKSAEDAKAIYDFTDTVIRIRNRHNDVIPTMAQGVIEYKESFGVDPVTSQNVQYFLDRFYMSRISIRMLLNQHSLLFGGKGKGSPSHRKHIGSINPNCNVLEVIKDGYENARRLCDLYYINSPELELEELNAKSPGQPIQVVYVPSHLYHMVFELFKNAMRATMEHHANRGVYPPIQVHVTLGNEDLTVKMSDRGGGVPLRKIDRLFNYMYSTAPRPRVETSRAVPLAGFGYGLPISRLYAQYFQGDLKLYSLEGYGTDAVIYIKALSTDSIERLPVYNKAAWKHYNTNHEADDWCVPSREPKDMTTFRSA

SEQ ID NO: 61 - protein, 691 aa, Unknown
  Description of Unknown Calcium-binding and coiled-coil domain-containing protein 1 sequence
MEESPLSRAPSRGGVNFLNVARTYIPNTKVECHYTLPPGTMPSASDWIGIFKVEAACVRDYHTFVWSSVPESTTDGSPIHTSVQFQASYLPKPGAQLYQFRYVNRQGQVCGQSPPFQFREPRPMDELVTLEEADGGSDILLVVPKATVLQNQLDESQQERNDLMQLKLQLEGQVTELRSRVQELERALATARQEHTELMEQYKGISRSHGEITEERDILSRQQGDHVARILELEDDIQTISEKVLTKEVELDRLRDTVKALTREQEKLLGQLKEVQADKEQSEAELQVAQQENHHLNLDLKEAKSWQEEQSAQAQRLKDKVAQMKDTLGQAQQRVAELEPLKEQLRGAQELAASSQQKATLLGEELASAAAARDRTIAELHRSRLEVAEVNGRLAELGLHLKEEKCQWSKERAGLLQSVEAEKDKILKLSAEILRLEKAVQEERTQNQVFKTELAREKDSSLVQLSESKRELTELRSALRVLQKEKEQLQEEKQELLEYMRKLEARLEKVADEKWNEDATTEDEEAAVGLSCPAALTDSEDESPEDMRLPPYGLCERGDPGSSPAGPREASPLVVISQPAPISPHLSGPAEDSSSDSEAEDEKSVLMAAVQSGGEEANLLLPELGSAFYDMASGFTVGTLSETSTGGPATPTWKECPICKERFPAESDKDALEDHMDGHFFFSTQDPFTFE

SEQ ID NO: 62 - protein, 298 aa, Unknown
  Description of Unknown Max-like protein X sequence
MTEPGASPEDPWVKASPVGAHAGEGRAGRARARRGAGRRGASLLSPKSPTLSVPRGCREDSSHPACAKVEYAYSDNSLDPGLFVESTRKGSVVSRANSIGSTSASSVPNTDDEDSDYHQEAYKESYKDRRRRAHTQAEQKRRDAIKRGYDDLQTIVPTCQQQDFSIGSQKLSKAIVLQKTIDYIQFLHKEKKKQEEEVSTLRKDVTALKIMKVNYEQIVKAHQDNPHEGEDQVSDQVKFNVFQGIMDSLFQSFNASISVASFQELSACVFSWIEEHCKPQTLREIVIGVLHQLKNQLY

SEQ ID NO: 63 - protein, 364 aa, Unknown
  Description of Unknown Fructose-bisphosphate aldolase A sequence
MPYQYPALTPEQKKELSDIAHRIVAPGKGILAADESTGSIAKRLQSIGTENTEENRRFYRQLLLTADDRVNPCIGGVILFHETLYQKADDGRPFPQVIKSKGGVVGIKVDKGVVPLAGTNGETTTQGLDGLSERCAQYKKDGADFAKWRCVLKIGEHTPSALAIMENANVLARYASICQQNGIVPIVEPEILPDGDHDLKRCQYVTEKVLAAVYKALSDHHIYLEGTLLKPNMVTPGHACTQKFSHEEIAMATVTALRRTVPPAVTGITFLSGGQSEEEASINLNAINKCPLLKPWALTFSYGRALQASALKAWGGKKENLKAAQEEYVKRALANSLACQGKYTPSGQAGAAASESLFVSNHAY

SEQ ID NO: 64 - protein, 766 aa, Unknown
  Description of Unknown Dipeptidyl peptidase 4 sequence
MKTPWKVLLGLLGAAALVTIITVPVVLLNKGTDDATADSRKTYTLTDYLKNTYRLKLYSLRWISDHEYLYKQENNILVFNAEYGNSSVFLENSTFDEFGHSINDYSISPDGQFILLEYNYVKQWRHSYTASYDIYDLNKRQLITEERIPNNTQWVTWSPVGHKLAYVWNNDIYVKIEPNLPSYRITWTGKEDIIYNGITDWVYEEEVFSAYSALWWSPNGTFLAYAQFNDTEVPLIEYSFYSDESLQYPKTVRVPYPKAGAVNPTVKFFVVNTDSLSSVTNATSIQITAPASMLIGDHYLCDVTWATQERISLQWLRRIQNYSVMDICDYDESSGRWNCLVARQHIEMSTTGWVGRFRPSEPHFTLDGNSFYKIISNEEGYRHICYFQIDKKDCTFITKGTWEVIGIEALTSDYLYYISNEYKGMPGGRNLYKIQLSDYTKVTCLSCELNPERCQYYSVSFSKEAKYYQLRCSGPGLPLYTLHSSVNDKGLRVLEDNSALDKMLQNVQMPSKKLDFIILNETKFWYQMILPPHFDKSKKYPLLLDVYAGPCSQKADTVFRLNWATYLASTENIIVASFDGRGSGYQGDKIMHAINRRLGTFEVEDQIEAARQFSKMGFVDNKRIAIWGWSYGGYVTSMVLGSGSGVFKCGIAVAPVSRWEYYDSVYTERYMGLPTPEDNLDHYRNSTVMSRAENFKQVEYLLIHGTADDNVHFQQSAQISKALVDVGVDFQAMWYTDEDHGIASSTAHQHIYTHMSHFIKQCFSLP

SEQ ID NO: 65 - protein, 266 aa, Unknown
  Description of Unknown Cholix1-266 carrier sequence
VEEALNIFDECRSPCSLTPEPGKPIQSKLSIPSDVVLDEGVLYYSMTINDEQNDIKDEDKGESIITIGEFATVRATRHYVNQDAPFGVIHLDITTENGTKTYSYNRKEGEFAINWLVPIGEDSPASIKISVDELDQQRNIIEVPKLYSIDLDNQTLEQWKTQGNVSFSVTRPEHNIAISWPSVSYKAAQKEGSRHKRWAHWHTGLALCWLVPMDAIYNYITQQNCTLGDNWFGGSYETVAGTPKVITVKQGIEQKPVEQRIHFSKG

SEQ ID NO: 66 - protein, 266 aa, Unknown
  Description of Unknown Cholix1-266 carrier sequence
LEEALNIFDECRSPCSLTPEPGKPIQSKLSIPSDVVLDEGVLYYSMTINDEQNDIKDEDKGESIITIGEFATVRATRHYVNQDAPFGVIHLDITTENGTKTYSYNRKEGEFAINWLVPIGEDSPASIKISVDELDQQRNIIEVPKLYSIDLDNQTLEQWKTQGNVSFSVTRPEHNIAISWPSVSYKAAQKEGSRHKRWAHWHTGLALCWLVPMDAIYNYITQQNCTLGDNWFGGSYETVAGTPKVITVKQGIEQKPVEQRIHFSKG

SEQ ID NO: 67 - protein, 266 aa, Unknown
  Description of Unknown Cholix1-266 carrier sequence
VEDELNIFDECRSPCSLTPEPGKPIQSKLSIPSDVVLDEGVLYYSMTINDEQNDIKDEDKGESIITIGEFATVRATRHYVNQDAPFGVIHLDITTENGTKTYSYNRKEGEFAINWLVPIGEDSPASIKISVDELDQQRNIIEVPKLYSIDLDNQTLEQWKTQGNVSFSVTRPEHNIAISWPSVSYKAAQKEGSRHKRWAHWHTGLALCWLVPMDAIYNYITQQNCTLGDNWFGGSYETVAGTPKVITVKQGIEQKPVEQRIHFSKG

SEQ ID NO: 68 - DNA, 1902 bp, Pseudomonas aeruginosa
gtcgaagaagctttaaacatctttgatgaatgccgttcgccatgttcgttgaccccggaaccgggtaagccgattcaatcaaaactgtctatccctagtgatgttgttctggatgaaggtgttctgtattactcgatgacgattaatgatgagcagaatgatattaaggatgaggacaaaggcgagtccattatcactattggtgaatttgccacagtacgcgcgactagacattatgttaatcaagatgcgccttttggtgtcatccatttagatattacgacagaaaatggtacaaaaacgtactcttataaccgcaaagagggtgaatttgcaatcaattggttagtgcctattggtgaagattctcctgcaagcatcaaaatctccgttgatgagctcgatcagcaacgcaatatcatcgaggtgcctaaactgtatagtattgatctcgataaccaaacgttagagcagtggaaaacccaaggtaatgtttctttttcggtaacgcgtcctgaacataatatcgctatctcttggccaagcgtgagttacaaagcagcgcagaaagagggttcacgccataagcgttgggctcattggcatacaggcttagcactgtgttggcttgtgccaatggatgctatctataactatatcacccagcaaaattgtactttaggggataattggtttggtggctcttatgagactgttgcaggcactccgaaggtgattacggttaagcaagggattgaacaaaagccagttgagcagcgcatccatttctccaaggggaatgcgatgagcgcacttgctgctcatcgcgtctgtggtgtgccattagaaactttggcgcgcagtcgcaaacctcgtgatctgacggatgatttatcatgtgcctatcaagcgcagaatatcgtgagtttatttgtcgcgacgcgtatcctgttctctcatctggatagcgtatttactctgaatcttgacgaacaagaaccagaggtggctgaacgtctaagtgatcttcgccgtatcaatgaaaataacccgggcatggttacacaggttttaaccgttgctcgtcagatctataacgattatgtcactcaccatccgggcttaactcctgagcaaaccagtgcgggtgcacaagctgccgatatcctctctttattttgcccagatgctgataagtcttgtgtggcttcaaacaacgatcaagccaatatcaacatcgagtctcgttctggccgttcatatttgcctgaaaaccgtgcggtaatcacccctcaaggcgtcacaaattggacttaccaggaactcgaagcaacacatcaagctctgactcgtgagggttatgtgttcgtgggttaccatggtacgaatcatgtcgctgcgcaaaccatcgtgaatcgcattgcccctgttccgcgcggcaacaacactgaaaacgaggaaaagtggggcgggttatatgttgcaactcacgctgaagttgcccatggttatgctcgcatcaaagaagggacaggggagtatggccttccgacccgtgctgagcgcgacgctcgtggggtaatgctgcgcgtgtatatccctcgtgcttcattagaacgtttttatcgcacgaatacacctttggaaaatgctgaggagcatatcacgcaagtgattggtcattctttgccattacgcaatgaagcatttactggtccagaaagtgcgggcggggaagacgaaactgtcattggctgggatatggcgattcatgcagttgcgatcccttcgactatcccagggaacgcttacgaagaattggcgattgatgaggaggctgttgcaaaagagcaatcgattagcacaaaaccaccttataaagagcgcaaagatgaacttaag

SEQ ID NO: 69 - protein, 613 aa, Pseudomonas aeruginosa
AEEAFDLWNECAKACVLDLKDGVRSSRMSVDPAIADTNGQGVLHYSMVLEGGNDALKLAIDNALSITSDGLTIRLEGGVEPNKPVRYSYTRQARGSWSLNWLVPIGHEKPSNIKVFIHELNAGNQLSHMSPIYTIEMGDELLAKLARDATFFVRAHESNEMQPTLAISHAGVSVVMAQAQPRREKRWSEWASGKVLCLLDPLDGVYNYLAQQRCNLDDTWEGKIYRVLAGNPAKHDLDIKPTVISHRLHFPEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAASADVVSLTCPVAAGECAGPADSGDALLERNYPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTGLTLAAPEAAGEVERLIGHPLPLRLDAITGPEEEGGRLETILGWPLAERTVVIPSAIPTDPRNVGGDLDPSSIPDKEQAISALPDYASQPGKPPREDLK

SEQ ID NO: 70 - protein, 393 aa, Artificial Sequence
  Description of Artificial Sequence Synthetic polypeptide
MVEEALNIFDECRSPCSLTPEPGKPIQSKLSIPSDVVLDEGVLYYSMTINDEQNDIKDEDKGESIITIGEFATVRATRHYVNQDAPFGVIHLDITTENGTKTYSYNRKEGEFAINWLVPIGEDSPASIKISVDELDQQRNIIEVPKLYSIDLDNQTLEQWKTQGNVSFSVTRPEHNIAISWPSVSYKAAQKEGSRHKRWAHWHTGLALCWLVPMDAIYNYITQQNCTLGDNWFGGSYETVAGTPKVITVKQGIEQKPVEQRIHFSKGNAMSALAAHRVCGVPLETLARSRKPRDLTDDLSCAYQAQNIVSLFVATRILFSHLDSVFTLNLDEQEPEVAERLSDLRRINENNPGMVTQVLTVARQIYNDYVTHHPGLTPEQTSAGAQACENLFQ

SEQ ID NO: 71 - protein, 262 aa, Artificial Sequence
  Description of Artificial Sequence Synthetic polypeptide
MHGEGTFTSDLSKQMEEEAVRLFIEWLKNGGPSSGAPPPSGGGGGSGGGGSGGGGSVEEALNIFDECRSPCSLTPEPGKPIQSKLSIPSDVVLDEGVLYYSMTINDEQNDIKDEDKGESIITIGEFATVRATRHYVNQDAPFGVIHLDITTENGTKTYSYNRKEGEFAINWLVPIGEDSPASIKISVDELDQQRNIIEVPKLYSIDLDNQTLEQWKTQGNVSFSVTRPEHNIAISWPSVSYKAAQKEGSRHKRWAHWHTGLA

SEQ ID NO: 72 - protein, 4 aa, Unknown
  Description of Unknown N-terminus of a natural Cholix variant sequence
VEDE

SEQ ID NO: 73 - protein, 206 aa, Unknown
  Description of Unknown Cholix variant sequence
VEEALNIFDECRSPCSLTPEPGKPIQSKLSIPSDVVLDEGVLYYSMTINDEQNDIKDEDKGESIITIGEFATVRATRHYVNQDAPFGVIHLDITTENGTKTYSYNRKEGEFAINWLVPIGEDSPASIKISVDELDQQRNIIEVPKLYSIDLDNQTLEQWKTQGNVSFSVTRPEHNIAISWPSVSYKAAQKEGSRHKRWAHWHTGLA

SEQ ID NO: 74 - protein, 387 aa, Unknown
  Description of Unknown Cholix variant sequence
MVEEALNIFDECRSPCSLTPEPGKPIQSKLSIPSDVVLDEGVLYYSMTINDEQNDIKDEDKGESIITIGEFATVRATRHYVNQDAPFGVIHLDITTENGTKTYSYNRKEGEFAINWLVPIGEDSPASIKISVDELDQQRNIIEVPKLYSIDLDNQTLEQWKTQGNVSFSVTRPEHNIAISWPSVSYKAAQKEGSRHKRWAHWHTGLALCWLVPMDAIYNYITQQNCTLGDNWFGGSYETVAGTPKVITVKQGIEQKPVEQRIHFSKGNAMSALAAHRVCGVPLETLARSRKPRDLTDDLSCAYQAQNIVSLFVATRILFSHLDSVFTLNLDEQEPEVAERLSDLRRINENNPGMVTQVLTVARQIYNDYVTHHPGLTPEQTSAGAQA

SEQ ID NO: 75 - protein, 634 aa, Unknown
  Description of Unknown Cholix variant sequence
VEDELNIFDECRSPCSLTPEPGKPIQSKLSIPSDVVLDEGVLYYSMTINDEQNDIKDEDKGESIITIGEFATVRATRHYVNQDAPFGVIHLDITTENGTKTYSYNRKEGEFAINWLVPIGEDSPASIKISVDELDQQRNIIEVPKLYSIDLDNQTLEQWKTQGNVSFSVTRPEHNIAISWPSVSYKAAQKEGSRHKRWAHWHTGLALCWLVPMDAIYNYITQQNCTLGDNWFGGSYETVAGTPKVITVKQGIEQKPVEQRIHFSKGNAMSALAAHRVCGVPLETLARSRKPRDLTDDLSCAYQAQNIVSLFVATRILFSHLDSVFTLNLDEQEPEVAERLSDLRRINENNPGMVTQVLTVARQIYNDYVTHHPGLTPEQTSAGAQAADILSLFCPDADKSCVASNNDQANINIESRSGRSYLPENRAVITPQGVTNWTYQELEATHQALTREGYVFVGYHGTNHVAAQTIVNRIAPVPRGNNTENEEKWGGLYVATHAEVAHGYARIKEGTGEYGLPTRAERDARGVMLRVYIPRASLERFYRTNTPLENAEEHITQVIGHSLPLRNEAFTGPESAGGEDETVIGWDMAIHAVAIPSTIPGNAYEELAIDEEAVAKEQSISTKPPYKERKDELK

SEQ ID NO: 76 - protein, 415 aa, Unknown
  Description of Unknown Cholix variant sequence
VEEALNIFDECRSPCSLTPEPGKPIQSKLSIPGDVVLDEGVLYYSMTINDEQNDIKDEDKGESIITIGEFATVRATRHYVSQDAPFGVINLDITTENGTKTYSFNRKESEFAINWLVPIGEDSPASIKISIDELDQQRNIIEVPKLYSIDLDNQTLEQWKTQGNVSFSVTRPEHNIAISWPSVSYKAAQKEGSRHKRWAHWHTGLALCWLVPIDAIYNYITQQNCTLGDNWFGGSYETVAGTPKAITVKQGIEQKPVEQRIHFSKKNAMEALAAHRVCGVPLETLARSRKPRDLPDDLSCAYNAQQIVSLFLATRILFTHIDSIFTLNLDGQEPEVAERLDDLRRINENNPGMVIQVLTVARQIYNDYVTHHPGLTPEQTSAGAQAADILSLFCPDADKSCVASNSDQANINIES

SEQ ID NO: 77 - protein, 432 aa, Artificial Sequence
  Description of Artificial Sequence Synthetic polypeptide
MVEEALNIFDECRSPCSLTPEPGKPIQSKLSIPSDVVLDEGVLYYSMTINDEQNDIKDEDKGESIITIGEFATVRATRHYVNQDAPFGVIHLDITTENGTKTYSYNRKEGEFAINWLVPIGEDSPASIKISVDELDQQRNIIEVPKLYSIDLDNQTLEQWKTQGNVSFSVTRPEHNIAISWPSVSYKAAQKEGSRHKRWAHWHTGLALCWLVPMDAIYNYITQQNCTLGDNWFGGSYETVAGTPKVITVKQGIEQKPVEQRIHFSKGNAMSALAAHRVCGVPLETLARSRKPRDLTDDLSCAYQAQNIVSLFVATRILFSHLDSVFTLNLDEQEPEVAERLSDLRRINENNPGMVTQVLTVARQIYNDYVTHHPGLTPEQTSAGAQAADILSLFCPDADKSCVASNNDQANINIESCENLFQSGTCHHHHHH

SEQ ID NO: 78 - protein, 403 aa, Artificial Sequence
  Description of Artificial Sequence Synthetic polypeptide
MVEEALNIFDECRSPCSLTPEPGKPIQSKLSIPSDVVLDEGVLYYSMTINDEQNDIKDEDKGESIITIGEFATVRATRHYVNQDAPFGVIHLDITTENGTKTYSYNRKEGEFAINWLVPIGEDSPASIKISVDELDQQRNIIEVPKLYSIDLDNQTLEQWKTQGNVSFSVTRPEHNIAISWPSVSYKAAQKEGSRHKRWAHWHTGLALCWLVPMDAIYNYITQQNCTLGDNWFGGSYETVAGTPKVITVKQGIEQKPVEQRIHFSKGNAMSALAAHRVCGVPLETLARSRKPRDLTDDLSCAYQAQNIVSLFVATRILFSHLDSVFTLNLDEQEPEVAERLSDLRRINENNPGMVTQVLTVARQIYNDYVTHHPGLTPEQTSAGAQACENLFQSGTCHHHHHH

SEQ ID NO: 79 - protein, 16 aa, Artificial Sequence
  Description of Artificial Sequence Synthetic peptide
GGGGGSGGGGSGGGGS

SEQ ID NO: 80 - protein, 422 aa, Artificial Sequence
  Description of Artificial Sequence Synthetic polypeptide
MVEEALNIFDECRSPCSLTPEPGKPIQSKLSIPSDVVLDEGVLYYSMTINDEQNDIKDEDKGESIITIGEFATVRATRHYVNQDAPFGVIHLDITTENGTKTYSYNRKEGEFAINWLVPIGEDSPASIKISVDELDQQRNIIEVPKLYSIDLDNQTLEQWKTQGNVSFSVTRPEHNIAISWPSVSYKAAQKEGSRHKRWAHWHTGLALCWLVPMDAIYNYITQQNCTLGDNWFGGSYETVAGTPKVITVKQGIEQKPVEQRIHFSKGNAMSALAAHRVCGVPLETLARSRKPRDLTDDLSCAYQAQNIVSLFVATRILFSHLDSVFTLNLDEQEPEVAERLSDLRRINENNPGMVTQVLTVARQIYNDYVTHHPGLTPEQTSAGAQAADILSLFCPDADKSCVASNNDQANINIESCENLFQ

SEQ ID NO: 81 - protein, 649 aa, Artificial Sequence
  Description of Artificial Sequence Synthetic polypeptide
  MOD_RES (3)..(3): E or D
  MOD_RES (4)..(4): A or E
  MOD_RES (6)..(6): N or K
  MOD_RES (16)..(16): S or L
  MOD_RES (21)..(21): P or L
  MOD_RES (24)..(24): P or Q
  MOD_RES (30)..(30): S or F
  MOD_RES (33)..(33): S or G
  MOD_RES (56)..(56): K or M
  MOD_RES (59)..(59): D or G
  MOD_RES (67)..(67): I or F
  MOD_RES (73)..(73): V or I
  MOD_RES (81)..(81): N or S
  MOD_RES (90)..(90): H or N
  MOD_RES (101)..(101): T or M
  MOD_RES (104)..(104): Y or F
  MOD_RES (108)..(108): E or D
  MOD_RES (109)..(109): G or S
  MOD_RES (112)..(112): A or T
  MOD_RES (114)..(114): N or H
  MOD_RES (118)..(118): P or I
  MOD_RES (119)..(119): I or P
  MOD_RES (131)..(131): V or I
  MOD_RES (134)..(134): L or I
  MOD_RES (137)..(137): Q or K
  MOD_RES (160)..(160): K or E
  MOD_RES (161)..(161): T or N
  MOD_RES (166)..(166): S or F
  MOD_RES (168)..(168): S or A
  MOD_RES (174)..(174): H or Q
  MOD_RES (175)..(185): This region may encompass one of the following sequences “N”, “S”, “SIAKQS”, or “SIAKQSIAKQS”
  MOD_RES (196)..(196): K or N
  MOD_RES (199)..(199): Q, E, or H
  MOD_RES (201)..(201): E, N, or D
  MOD_RES (203)..(203): S or A
  MOD_RES (210)..(210): H or N
  MOD_RES (212)..(212): H, L, F, or R
  MOD_RES (214)..(214): G or T
  MOD_RES (215)..(215): L or S
  MOD_RES (216)..(216): A or P
  MOD_RES (217)..(217): L, E, or K
  MOD_RES (218)..(218): C or V
  MOD_RES (219)..(219): W, V, or T
  MOD_RES (221)..(221): V or absent
  MOD_RES (222)..(222): P or absent
  MOD_RES (223)..(223): M, I, L, or absent
  MOD_RES (224)..(224): D or absent
  MOD_RES (225)..(225): A or absent
  MOD_RES (226)..(226): I or absent
  MOD_RES (227)..(227): Y or C
  MOD_RES (228)..(228): N or F
  MOD_RES (229)..(229): Y or F
  MOD_RES (230)..(230): I or E
  MOD_RES (231)..(231): T or D
  MOD_RES (232)..(232): Q or P
  MOD_RES (233)..(233): Q, E, or A
  MOD_RES (234)..(234): N, L, or Q
  MOD_RES (237)..(237): L or Y
  MOD_RES (239)..(239): D or E
  MOD_RES (240)..(240): N or D
  MOD_RES (242)..(242): F, H, or Y
  MOD_RES (245)..(245): S or A
  MOD_RES (247)..(247): E or K
  MOD_RES (252)..(252): T or I
  MOD_RES (254)..(254): K, E, or G
  MOD_RES (255)..(255): V or A
  MOD_RES (257)..(257): T or M
  MOD_RES (262)..(262): I or M
  MOD_RES (266)..(266): P, T, or A
  MOD_RES (275)..(275): K, Q, or N
  MOD_RES (276)..(276): G or K
  MOD_RES (279)..(279): M or I
  MOD_RES (280)..(280): S or E
  MOD_RES (281)..(281): A or T
  MOD_RES (298)..(298): S or G
  MOD_RES (303)..(303): D or Y
  MOD_RES (305)..(305): T, P, or Q
  MOD_RES (309)..(309): S or Q
  MOD_RES (311)..(311): A or V
  MOD_RES (313)..(313): Q or N
  MOD_RES (316)..(316): N or Q
  MOD_RES (322)..(322): V or L
  MOD_RES (326)..(326): I or M
  MOD_RES (329)..(329): S or T
  MOD_RES (331)..(331): L or I
  MOD_RES (334)..(334): V or I
  MOD_RES (340)..(340): D, E, or H
  MOD_RES (341)..(341): E or G
  MOD_RES (343)..(343): E or A
  MOD_RES (345)..(345): E or A
  MOD_RES (347)..(347): A or T
  MOD_RES (351)..(351): S, D, or T
  MOD_RES (352)..(352): D or A
  MOD_RES (353)..(353): L or I
  MOD_RES (355)..(355): R or Q
  MOD_RES (359)..(359): N or D
  MOD_RES (363)..(363): M or V
  MOD_RES (365)..(365): T or I
  MOD_RES (370)..(370): V or I
  MOD_RES (381)..(381): H or E
  MOD_RES (384)..(384): G or L
  MOD_RES (386)..(386): T or I
  MOD_RES (393)..(393): G or S
  MOD_RES (403)..(403): F or L
  MOD_RES (404)..(404): C or Y
  MOD_RES (407)..(407): A or T
  MOD_RES (409)..(409): K, E, or G
  MOD_RES (410)..(410): S, P, or H
  MOD_RES (414)..(414): S or L
  MOD_RES (415)..(415): N or D
  MOD_RES (416)..(416): N or S
  MOD_RES (423)..(423): I or V
  MOD_RES (433)..(433): P or L
  MOD_RES (441)..(441): P or Q
  MOD_RES (453)..(453): E or D
  MOD_RES (454)..(454): A or T
  MOD_RES (455)..(455): T or K
  MOD_RES (458)..(458): A or T
  MOD_RES (461)..(461): R or Q
  MOD_RES (463)..(463): G or D
  MOD_RES (475)..(475): V or A
  MOD_RES (479)..(479): T, S, or N
  MOD_RES (485)..(485): A, S, or T
  MOD_RES (491)..(491): N or S
  MOD_RES (492)..(492): N or D
  MOD_RES (495)..(495): N, S, or K
  MOD_RES (497)..(497): E, R, or K
  MOD_RES (498)..(498): K, A, or E
  MOD_RES (502)..(502): L or V
  MOD_RES (505)..(505): A or S
  MOD_RES (507)..(507): H or D
  MOD_RES (509)..(509): E or S
  MOD_RES (510)..(510): V or L
  MOD_RES (511)..(511): A or N
  MOD_RES (512)..(512): H or Y
  MOD_RES (513)..(513): G or R
  MOD_RES (515)..(515): A or T
  MOD_RES (517)..(517): I or L
  MOD_RES (518)..(518): K or Q
  MOD_RES (519)..(519): E or K
  MOD_RES (522)..(522): G or A
  MOD_RES (523)..(523): E, D, or N
  MOD_RES (524)..(524): Y, G, A, or N
  MOD_RES (525)..(525): G or E
  MOD_RES (526)..(526): L or G
  MOD_RES (527)..(527): P or L
  MOD_RES (529)..(529): R, P, or T
  MOD_RES (530)..(530): A or E
  MOD_RES (531)..(531): E or K
  MOD_RES (532)..(532): R, Q, or K
  MOD_RES (533)..(533): D, K, or E
  MOD_RES (534)..(534): A, T, or S
  MOD_RES (540)..(540): R or K
  MOD_RES (543)..(543): I or L
  MOD_RES (544)..(544): P or H
  MOD_RES (545)..(545): R or Q
  MOD_RES (554)..(554): T or I
  MOD_RES (556)..(556): T, A, or I
  MOD_RES (557)..(557): P or D
  MOD_RES (560)..(560): N or K
  MOD_RES (561)..(561): A or E
  MOD_RES (562)..(562): E, R, or D
  MOD_RES (563)..(563): E, N, or R
  MOD_RES (564)..(564): H or L
  MOD_RES (565)..(565): I or V
  MOD_RES (566)..(566): T or E
  MOD_RES (567)..(567): Q, R, H, or D
  MOD_RES (572)..(572): S or P
  MOD_RES (583)..(583): P or T
  MOD_RES (584)..(584): E or D
  MOD_RES (585)..(585): S, A, or R
  MOD_RES (586)..(586): A, E, or V
  MOD_RES (587)..(587): G, E, or D
  MOD_RES (589)..(589): E or S
  MOD_RES (590)..(590): D or N
  MOD_RES (593)..(593): V or A
  MOD_RES (598)..(598): M or I
  MOD_RES (601)..(601): H or Y
  MOD_RES (602)..(602): A or G
  MOD_RES (613)..(613): A or S
  MOD_RES (615)..(615): E or A
  MOD_RES (616)..(616): E, A, Q, G, V, or R
  MOD_RES (618)..(618): A, P, or T
  MOD_RES (619)..(619): I, T, or P
  MOD_RES (620)..(620): D or A
  MOD_RES (624)..(629): This region may encompass one of the following sequences “V” or “VVKEAI”
  MOD_RES (631)..(631): K or E
  MOD_RES (637)..(637): T, A, or P
  MOD_RES (644)..(644): R, Q, or H
  MOD_RES (645)..(645): K or absent
  See specification as filed for detailed description of substitutions and preferred embodiments
VEXXLXIFDECRSPCXLTPEXGKXIQSKLXIPXDVVLDEGVLYYSMTINDEQNDIXDEXKGESIITXGEFATXRATRHYVXQDAPFGVIXLDITTENGTKXYSXNRKXXEFXIXWLVXXGEDSPASIKISXDEXDQXRNIIEVPKLYSIDLDNQTLEQWXXQGNVXFXVTRPEXXXXXXXXXXXXIAISWPSVSYXAAXKXGXRHKRWAXWXTXXXXXXLXXXXXXXXXXXXXXCTXGXXWXGGXYXTVAGXPXXIXVKQGXEQKXVEQRIHFSXXNAXXXLAAHRVCGVPLETLARXRKPRXLXDDLXCXYXAQXIVSLFXATRXLFXHXDSXFTLNLXXQXPXVXERLXXXRXINEXNPGXVXQVLTXARQIYNDYVTXHPXLXPEQTSAXAQAADILSLXXPDXDXXCVAXXXDQANINXESRSGRSYLXENRAVITXQGVTNWTYQELXXXHQXLTXEXYVFVGYHGTNHXAAQXIVNRIXPVPRGXXTEXEXXWGGXYVXTXAXXXXXYXRXXXGTXXXXXXTXXXXXXRGVMLXVYXXXASLERFYRXNXXLEXXXXXXXXVIGHXLPLRNEAFTGXXXXXGXXETXIGWDXAIXXVAIPSTIPGNXYXXLXXXEEAXXXXXXAXEQSISXKPPYKEXXDELK

SEQ ID NO: 82 - protein, 252 aa, Pseudomonas aeruginosa
AEEAFDLWNECAKACVLDLKDGVRSSRMSVDPAIADTNGQGVLHYSMVLEGGNDALKLAIDNALSITSDGLTIRLEGGVEPNKPVRYSYTRQARGSWSLNWLVPIGHEKPSNIKVFIHELNAGNQLSHMSPIYTIEMGDELLAKLARDATFFVRAHESNEMQPTLAISHAGVSVVMAQAQPRREKRWSEWASGKVLCLLDPLDGVYNYLAQQRCNLDDTWEGKIYRVLAGNPAKHDLDIKPTVISHRLHFPE

SEQ ID NO: 83 - protein, 321 aa, Artificial Sequence
  Description of Artificial Sequence Synthetic polypeptide
MVEEALNIFDECRSPCSLTPEPGKPIQSKLSIPSDVVLDEGVLYYSMTINDEQNDIKDEDKGESIITIGEFATVRATRHYVNQDAPFGVIHLDITTENGTKTYSYNRKEGEFAINWLVPIGEDSPASIKISVDELDQQRNIIEVPKLYSIDLDNQTLEQWKTQGNVSFSVTRPEHNIAISWPSVSYKAAQKEGSRHKRWAHWHTGLALCWLVPMDAIYNYITQQNCTLGDNWFGGSYETVAGTPKVITVKQGIEQKPVEQRIHFSKGGGGGSGGGGSGGGGSHGEGTFTSDLSKQMEEEAVRLFIEWLKNGGPSSGAPPPS

SEQ ID NO: 84 - protein, 254 aa, Artificial Sequence
  Description of Artificial Sequence Synthetic polypeptide
MAEEAFDLWNECAKACVLDLKDGVRSSRMSVDPAIADTNGQGVLHYSMVLEGGNDALKLAIDNALSITSDGLTIRLEGGVEPNKPVRYSYTRQARGSWSLNWLVPIGHEKPSNIKVFIHELNAGNQLSHMSPIYTIEMGDELLAKLARDATFFVRAHESNEMQPTLAISHAGVSVVMAQAQPRREKRWSEWASGKVLCLLDPLDGVYNYLAQQRCNLDDTWEGKIYRVLAGNPAKHDLDIKPTVISHRLHFPEG

SEQ ID NO: 85 - protein, 30 aa, Artificial Sequence
  Description of Artificial Sequence Synthetic polypeptide
  MISC_FEATURE (1)..(30): This sequence may encompass 1-15 “Gly Ser” repeating units
GSGSGSGSGSGSGSGSGSGSGSGSGSGSGS

SEQ ID NO: 86 - protein, 45 aa, Artificial Sequence
  Description of Artificial Sequence Synthetic polypeptide
  MISC_FEATURE (1)..(45): This sequence may encompass 1-15 “Gly Gly Ser” repeating units
GGSGGSGGSGGSGGSGGSGGSGGSGGSGGSGGSGGSGGSGGSGGS

SEQ ID NO: 87 - protein, 60 aa, Artificial Sequence
  Description of Artificial Sequence Synthetic polypeptide
  MISC_FEATURE (1)..(60): This sequence may encompass 1-15 “Gly Gly Gly Ser” repeating units
GGGSGGGSGGGSGGGSGGGSGGGSGGGSGGGSGGGSGGGSGGGSGGGSGGGSGGGSGGGS

SEQ ID NO: 88 - protein, 75 aa, Artificial Sequence
  Description of Artificial Sequence Synthetic polypeptide
  MISC_FEATURE (1)..(75): This sequence may encompass 1-15 “Gly Gly Gly Gly Ser” repeating units
GGGGSGGGGSGGGGSGGGGSGGGGSGGGGSGGGGSGGGGSGGGGSGGGGSGGGGSGGGGSGGGGSGGGGSGGGGS

SEQ ID NO: 89 - protein, 90 aa, Artificial Sequence
  Description of Artificial Sequence Synthetic polypeptide
  MISC_FEATURE (1)..(90): This sequence may encompass 1-15 “Gly Gly Gly Gly Gly Ser” repeating units
GGGGGSGGGGGSGGGGGSGGGGGSGGGGGSGGGGGSGGGGGSGGGGGSGGGGGSGGGGGSGGGGGSGGGGGSGGGGGSGGGGGSGGGGGS

SEQ ID NO: 90 - protein, 6 aa, Artificial Sequence
  Description of Artificial Sequence Synthetic peptide
ENLFQS

SEQ ID NO: 91 - protein, 6 aa, Artificial Sequence
  Description of Artificial Sequence Synthetic 6xHis tag
HHHHHH

SEQ ID NO: 92 - protein, 6 aa, Artificial Sequence
  Description of Artificial Sequence Synthetic peptide
SIAKQS

SEQ ID NO: 93 - protein, 11 aa, Artificial Sequence
  Description of Artificial Sequence Synthetic peptide
SIAKQSIAKQS

SEQ ID NO: 94 - protein, 6 aa, Artificial Sequence
  Description of Artificial Sequence Synthetic peptide
VVKEAI